SEQUENCE LISTING (893 sequences)

SEQ ID NO: 1 - RNA, 15 nt, Homo sapiens
  source 1..15: mol_type = transcribed RNA
cagtaaagagattaa

SEQ ID NO: 2 - RNA, 15 nt, Homo sapiens
  source 1..15: mol_type = transcribed RNA
atatcagtaaagaga

SEQ ID NO: 3 - RNA, 15 nt, Homo sapiens
  source 1..15: mol_type = transcribed RNA
ctcaggatttaaaat

SEQ ID NO: 4 - RNA, 20 nt, synthetic construct
  misc_feature 1..20: note = Description of Artificial Sequence: Synthetic oligonucleotide
  source 1..20: mol_type = other RNA
taaatttggagatccgagag

SEQ ID NO: 5 - RNA, 15 nt, synthetic construct
  misc_feature 1..15: note = Description of Artificial Sequence: Synthetic oligonucleotide
  source 1..15: mol_type = other RNA
ggatctccaaattta

SEQ ID NO: 6 - RNA, 20 nt, synthetic construct
  misc_feature 1..20: note = Description of Artificial Sequence: Synthetic oligonucleotide
  source 1..20: mol_type = other RNA
tataaatggtagctatgatg

SEQ ID NO: 7 - RNA, 15 nt, synthetic construct
  misc_feature 1..15: note = Description of Artificial Sequence: Synthetic oligonucleotide
  source 1..15: mol_type = other RNA
tagctaccatttata

SEQ ID NO: 8 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 9 - RNA, 15 nt, synthetic construct
  misc_feature 1..15: note = Description of Artificial Sequence: Synthetic oligonucleotide
  source 1..15: mol_type = other RNA
cagtaaagagattaa

SEQ ID NO: 10 - DNA, 20 bp, Homo sapiens
  source 1..20: mol_type = genomic DNA
ctctcggatctccaaattta

SEQ ID NO: 11 - DNA, 20 bp, Homo sapiens
  source 1..20: mol_type = genomic DNA
catcatagctaccatttatt

SEQ ID NO: 12 - DNA, 20 bp, Homo sapiens
  source 1..20: mol_type = genomic DNA
attgtaccacacaaagtaat

SEQ ID NO: 13 - DNA, 20 bp, Homo sapiens
  source 1..20: mol_type = genomic DNA
gagccaagacaatcataaca

SEQ ID NO: 14 - DNA, 456 bp, synthetic construct
  misc_feature 1..456: note = Description of Artificial Sequence: Synthetic polynucleotide
  source 1..456: mol_type = other DNA
gaagaaagaaattacaatcagaggtgagcactgcaacaaaaaggctgttttctctcggatctccaaatttaaaagcacaaggaatgattgtaccacacaaagtaatgtaaaacattaaaggactcattaaaaagtaacagttgtctcatatcatcttgatttattgtcactgttgctaactttcaggctcggaggagatgctcctcccaaaatgagttcggagatgatagcagtaataatgagacccccgggctccagctctgggccccccattcaggccgagggggctgctccggggggccgacttggtgcacgtttggatttggaggatccctgcactgccttctctgtgtttgttgctcttgctgttttctcctgcctgataaacaacaacttgggatgatcctttccattttgatgccaacctctttttatttttaagcggcgccctatagt

SEQ ID NO: 15 - DNA, 418 bp, synthetic construct
  misc_feature 1..418: note = Description of Artificial Sequence: Synthetic polynucleotide
  source 1..418: mol_type = other DNA
acaacaagagcctgaactgtatacatcaacgtcaccatcgtcatcgtcatcatcaccattgtcatcatcatcatcatcgtcatcatcatcatcatcatagctatcatcattatcatcatcatcatcatcatcatcatagctaccatttattgaaaactattatgtgtcaacttcaaagaacttatcctttagttggagagccaagacaatcataacaataacaaatggccgggcatggtggctcacgcctgtaatcccagcactttgggaggccaaggcaggtggatcatttgaggtcaggagtccaagaccagcctgaccaagatggtgaaatgctgtctctattaaaaatacaaaattagccaggcatggtggctcatgcctgtaatgccagctactcgggaggctgagacaggag

SEQ ID NO: 16 - RNA, 15 nt, synthetic construct
  misc_feature 1..15: note = Description of Artificial Sequence: Synthetic oligonucleotide
  source 1..15: mol_type = other RNA
cagtaaagagattaa

SEQ ID NO: 17 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 18 - RNA, 15 nt, synthetic construct
  misc_feature 1..15: note = Description of Artificial Sequence: Synthetic oligonucleotide
  source 1..15: mol_type = other RNA
cagtaaagagattaa

SEQ ID NO: 19 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 20 - RNA, 15 nt, synthetic construct
  misc_feature 1..15: note = Description of Artificial Sequence: Synthetic oligonucleotide
  source 1..15: mol_type = other RNA
cagtaaagagattaa

SEQ ID NO: 21 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 22 - RNA, 20 nt, Homo sapiens
  source 1..20: mol_type = transcribed RNA
aatcagaggtgagcactgca

SEQ ID NO: 23 - RNA, 20 nt, Homo sapiens
  source 1..20: mol_type = transcribed RNA
gaggtgagcactgcaacaaa

SEQ ID NO: 24 - RNA, 20 nt, Homo sapiens
  source 1..20: mol_type = transcribed RNA
aggtgagcactgcaacaaaa

SEQ ID NO: 25 - RNA, 20 nt, Homo sapiens
  source 1..20: mol_type = transcribed RNA
tgagcactgcaacaaaaagg

SEQ ID NO: 26 - RNA, 20 nt, Homo sapiens
  source 1..20: mol_type = transcribed RNA
ttttctctcggatctccaaa

SEQ ID NO: 27 - RNA, 20 nt, Homo sapiens
  source 1..20: mol_type = transcribed RNA
tttctctcggatctccaaat

SEQ ID NO: 28 - RNA, 20 nt, Homo sapiens
  source 1..20: mol_type = transcribed RNA
ctctcggatctccaaattta

SEQ ID NO: 29 - RNA, 20 nt, Homo sapiens
  source 1..20: mol_type = transcribed RNA
tctcggatctccaaatttaa

SEQ ID NO: 30 - RNA, 20 nt, Homo sapiens
  source 1..20: mol_type = transcribed RNA
tcggatctccaaatttaaaa

SEQ ID NO: 31 - RNA, 20 nt, Homo sapiens
  source 1..20: mol_type = transcribed RNA
tccaaatttaaaagcacaag

SEQ ID NO: 32 - RNA, 20 nt, Homo sapiens
  source 1..20: mol_type = transcribed RNA
ccaaatttaaaagcacaagg

SEQ ID NO: 33 - RNA, 20 nt, Homo sapiens
  source 1..20: mol_type = transcribed RNA
caaatttaaaagcacaagga

SEQ ID NO: 34 - RNA, 20 nt, Homo sapiens
  source 1..20: mol_type = transcribed RNA
aagcacaaggaatgattgta

SEQ ID NO: 35 - RNA, 20 nt, Homo sapiens
  source 1..20: mol_type = transcribed RNA
gaatgattgtaccacacaaa

SEQ ID NO: 36 - RNA, 20 nt, Homo sapiens
  source 1..20: mol_type = transcribed RNA
attgtaccacacaaagtaat

SEQ ID NO: 37 - RNA, 20 nt, Homo sapiens
  source 1..20: mol_type = transcribed RNA
gtaccacacaaagtaatgta

SEQ ID NO: 38 - RNA, 20 nt, Homo sapiens
  source 1..20: mol_type = transcribed RNA
taccacacaaagtaatgtaa

SEQ ID NO: 39 - RNA, 20 nt, Homo sapiens
  source 1..20: mol_type = transcribed RNA
ccacacaaagtaatgtaaaa

SEQ ID NO: 40 - RNA, 20 nt, Homo sapiens
  source 1..20: mol_type = transcribed RNA
acacaaagtaatgtaaaaca

SEQ ID NO: 41 - RNA, 20 nt, Homo sapiens
  source 1..20: mol_type = transcribed RNA
agtaatgtaaaacattaaag

SEQ ID NO: 42 - RNA, 20 nt, Homo sapiens
  source 1..20: mol_type = transcribed RNA
gtaatgtaaaacattaaagg

SEQ ID NO: 43 - RNA, 20 nt, Homo sapiens
  source 1..20: mol_type = transcribed RNA
taaaacattaaaggactcat

SEQ ID NO: 44 - RNA, 20 nt, Homo sapiens
  source 1..20: mol_type = transcribed RNA
acattaaaggactcattaaa

SEQ ID NO: 45 - RNA, 20 nt, Homo sapiens
  source 1..20: mol_type = transcribed RNA
ggactcattaaaaagtaaca

SEQ ID NO: 46 - RNA, 20 nt, Homo sapiens
  source 1..20: mol_type = transcribed RNA
actcattaaaaagtaacagt

SEQ ID NO: 47 - RNA, 20 nt, Homo sapiens
  source 1..20: mol_type = transcribed RNA
aaagtaacagttgtctcata

SEQ ID NO: 48 - RNA, 20 nt, Homo sapiens
  source 1..20: mol_type = transcribed RNA
catcatcatcatcatagcta

SEQ ID NO: 49 - RNA, 20 nt, Homo sapiens
  source 1..20: mol_type = transcribed RNA
catcatcatcatagctatca

SEQ ID NO: 50 - RNA, 20 nt, Homo sapiens
  source 1..20: mol_type = transcribed RNA
catcatcatagctatcatca

SEQ ID NO: 51 - RNA, 20 nt, Homo sapiens
  source 1..20: mol_type = transcribed RNA
atcatcatcatcatcatagc

SEQ ID NO: 52 - RNA, 20 nt, Homo sapiens
  source 1..20: mol_type = transcribed RNA
catcatcatcatcatagcta

SEQ ID NO: 53 - RNA, 20 nt, Homo sapiens
  source 1..20: mol_type = transcribed RNA
catcatcatcatagctacca

SEQ ID NO: 54 - RNA, 20 nt, Homo sapiens
  source 1..20: mol_type = transcribed RNA
tcatcatagctaccatttat

SEQ ID NO: 55 - RNA, 20 nt, Homo sapiens
  source 1..20: mol_type = transcribed RNA
catcatagctaccatttatt

SEQ ID NO: 56 - RNA, 20 nt, Homo sapiens
  source 1..20: mol_type = transcribed RNA
agctaccatttattgaaaac

SEQ ID NO: 57 - RNA, 20 nt, Homo sapiens
  source 1..20: mol_type = transcribed RNA
taccatttattgaaaactat

SEQ ID NO: 58 - RNA, 20 nt, Homo sapiens
  source 1..20: mol_type = transcribed RNA
aacttcaaagaacttatcct

SEQ ID NO: 59 - RNA, 20 nt, Homo sapiens
  source 1..20: mol_type = transcribed RNA
caaagaacttatcctttagt

SEQ ID NO: 60 - RNA, 20 nt, Homo sapiens
  source 1..20: mol_type = transcribed RNA
tcctttagttggagagccaa

SEQ ID NO: 61 - RNA, 20 nt, Homo sapiens
  source 1..20: mol_type = transcribed RNA
ctttagttggagagccaaga

SEQ ID NO: 62 - RNA, 20 nt, Homo sapiens
  source 1..20: mol_type = transcribed RNA
ttagttggagagccaagaca

SEQ ID NO: 63 - RNA, 20 nt, Homo sapiens
  source 1..20: mol_type = transcribed RNA
tagttggagagccaagacaa

SEQ ID NO: 64 - RNA, 20 nt, Homo sapiens
  source 1..20: mol_type = transcribed RNA
ttggagagccaagacaatca

SEQ ID NO: 65 - RNA, 20 nt, Homo sapiens
  source 1..20: mol_type = transcribed RNA
ggagagccaagacaatcata

SEQ ID NO: 66 - RNA, 20 nt, Homo sapiens
  source 1..20: mol_type = transcribed RNA
gagccaagacaatcataaca

SEQ ID NO: 67 - RNA, 20 nt, Homo sapiens
  source 1..20: mol_type = transcribed RNA
ccaagacaatcataacaata

SEQ ID NO: 68 - RNA, 20 nt, Homo sapiens
  source 1..20: mol_type = transcribed RNA
aagacaatcataacaataac

SEQ ID NO: 69 - RNA, 20 nt, Homo sapiens
  source 1..20: mol_type = transcribed RNA
agctgtctgcttctcacagg

SEQ ID NO: 70 - RNA, 20 nt, Homo sapiens
  source 1..20: mol_type = transcribed RNA
gatcctgaactgagtttaaa

SEQ ID NO: 71 - RNA, 20 nt, Homo sapiens
  source 1..20: mol_type = transcribed RNA
atcctgaactgagtttaaaa

SEQ ID NO: 72 - RNA, 20 nt, Homo sapiens
  source 1..20: mol_type = transcribed RNA
tgaactgagtttaaaaggca

SEQ ID NO: 73 - RNA, 20 nt, Homo sapiens
  source 1..20: mol_type = transcribed RNA
gtttaaaaggcacccagcac

SEQ ID NO: 74 - RNA, 20 nt, Homo sapiens
  source 1..20: mol_type = transcribed RNA
atcaaatgcaacgtacaaag

SEQ ID NO: 75 - RNA, 20 nt, Homo sapiens
  source 1..20: mol_type = transcribed RNA
tcaaatgcaacgtacaaaga

SEQ ID NO: 76 - RNA, 20 nt, Homo sapiens
  source 1..20: mol_type = transcribed RNA
gttgtatggttaaaagatgg

SEQ ID NO: 77 - RNA, 20 nt, Homo sapiens
  source 1..20: mol_type = transcribed RNA
tttaaaaacctcactgccac

SEQ ID NO: 78 - RNA, 20 nt, Homo sapiens
  source 1..20: mol_type = transcribed RNA
taaaaacctcactgccactc

SEQ ID NO: 79 - RNA, 20 nt, Homo sapiens
  source 1..20: mol_type = transcribed RNA
aaaacctcactgccactcta

SEQ ID NO: 80 - RNA, 20 nt, Homo sapiens
  source 1..20: mol_type = transcribed RNA
gaaacagaattgagagcatc

SEQ ID NO: 81 - RNA, 30 nt, Homo sapiens
  source 1..30: mol_type = transcribed RNA
attacaatcagaggtgagcactgcaacaaa

SEQ ID NO: 82 - RNA, 30 nt, Homo sapiens
  source 1..30: mol_type = transcribed RNA
aatcagaggtgagcactgcaacaaaaaggc

SEQ ID NO: 83 - RNA, 30 nt, Homo sapiens
  source 1..30: mol_type = transcribed RNA
atcagaggtgagcactgcaacaaaaaggct

SEQ ID NO: 84 - RNA, 30 nt, Homo sapiens
  source 1..30: mol_type = transcribed RNA
agaggtgagcactgcaacaaaaaggctgtt

SEQ ID NO: 85 - RNA, 30 nt, Homo sapiens
  source 1..30: mol_type = transcribed RNA
ggctgttttctctcggatctccaaatttaa

SEQ ID NO: 86 - RNA, 30 nt, Homo sapiens
  source 1..30: mol_type = transcribed RNA
gctgttttctctcggatctccaaatttaaa

SEQ ID NO: 87 - RNA, 30 nt, Homo sapiens
  source 1..30: mol_type = transcribed RNA
gttttctctcggatctccaaatttaaaagc

SEQ ID NO: 88 - RNA, 30 nt, Homo sapiens
  source 1..30: mol_type = transcribed RNA
ttttctctcggatctccaaatttaaaagca

SEQ ID NO: 89 - RNA, 30 nt, Homo sapiens
  source 1..30: mol_type = transcribed RNA
ttctctcggatctccaaatttaaaagcaca

SEQ ID NO: 90 - RNA, 30 nt, Homo sapiens
  source 1..30: mol_type = transcribed RNA
ggatctccaaatttaaaagcacaaggaatg

SEQ ID NO: 91 - RNA, 30 nt, Homo sapiens
  source 1..30: mol_type = transcribed RNA
gatctccaaatttaaaagcacaaggaatga

SEQ ID NO: 92 - RNA, 30 nt, Homo sapiens
  source 1..30: mol_type = transcribed RNA
atctccaaatttaaaagcacaaggaatgat

SEQ ID NO: 93 - RNA, 30 nt, Homo sapiens
  source 1..30: mol_type = transcribed RNA
tttaaaagcacaaggaatgattgtaccaca

SEQ ID NO: 94 - RNA, 30 nt, Homo sapiens
  source 1..30: mol_type = transcribed RNA
acaaggaatgattgtaccacacaaagtaat

SEQ ID NO: 95 - RNA, 30 nt, Homo sapiens
  source 1..30: mol_type = transcribed RNA
gaatgattgtaccacacaaagtaatgtaaa

SEQ ID NO: 96 - RNA, 30 nt, Homo sapiens
  source 1..30: mol_type = transcribed RNA
tgattgtaccacacaaagtaatgtaaaaca

SEQ ID NO: 97 - RNA, 30 nt, Homo sapiens
  source 1..30: mol_type = transcribed RNA
gattgtaccacacaaagtaatgtaaaacat

SEQ ID NO: 98 - RNA, 30 nt, Homo sapiens
  source 1..30: mol_type = transcribed RNA
ttgtaccacacaaagtaatgtaaaacatta

SEQ ID NO: 99 - RNA, 30 nt, Homo sapiens
  source 1..30: mol_type = transcribed RNA
gtaccacacaaagtaatgtaaaacattaaa

SEQ ID NO: 100 - RNA, 30 nt, Homo sapiens
  source 1..30: mol_type = transcribed RNA
cacaaagtaatgtaaaacattaaaggactc

SEQ ID NO: 101 - RNA, 30 nt, Homo sapiens
  source 1..30: mol_type = transcribed RNA
acaaagtaatgtaaaacattaaaggactca

SEQ ID NO: 102 - RNA, 30 nt, Homo sapiens
  source 1..30: mol_type = transcribed RNA
taatgtaaaacattaaaggactcattaaaa

SEQ ID NO: 103 - RNA, 30 nt, Homo sapiens
  source 1..30: mol_type = transcribed RNA
gtaaaacattaaaggactcattaaaaagta

SEQ ID NO: 104 - RNA, 30 nt, Homo sapiens
  source 1..30: mol_type = transcribed RNA
ttaaaggactcattaaaaagtaacagttgt

SEQ ID NO: 105 - RNA, 30 nt, Homo sapiens
  source 1..30: mol_type = transcribed RNA
aaaggactcattaaaaagtaacagttgtct

SEQ ID NO: 106 - RNA, 30 nt, Homo sapiens
  source 1..30: mol_type = transcribed RNA
attaaaaagtaacagttgtctcatatcatc

SEQ ID NO: 107 - RNA, 30 nt, Homo sapiens
  source 1..30: mol_type = transcribed RNA
gtcatcatcatcatcatcatagctatcatc

SEQ ID NO: 108 - RNA, 30 nt, Homo sapiens
  source 1..30: mol_type = transcribed RNA
atcatcatcatcatcatagctatcatcatt

SEQ ID NO: 109 - RNA, 30 nt, Homo sapiens
  source 1..30: mol_type = transcribed RNA
atcatcatcatcatagctatcatcattatc

SEQ ID NO: 110 - RNA, 30 nt, Homo sapiens
  source 1..30: mol_type = transcribed RNA
tcatcatcatcatcatcatcatagctacca

SEQ ID NO: 111 - RNA, 30 nt, Homo sapiens
  source 1..30: mol_type = transcribed RNA
atcatcatcatcatcatcatagctaccatt

SEQ ID NO: 112 - RNA, 30 nt, Homo sapiens
  source 1..30: mol_type = transcribed RNA
atcatcatcatcatcatagctaccatttat

SEQ ID NO: 113 - RNA, 30 nt, Homo sapiens
  source 1..30: mol_type = transcribed RNA
catcatcatcatagctaccatttattgaaa

SEQ ID NO: 114 - RNA, 30 nt, Homo sapiens
  source 1..30: mol_type = transcribed RNA
atcatcatcatagctaccatttattgaaaa

SEQ ID NO: 115 - RNA, 30 nt, Homo sapiens
  source 1..30: mol_type = transcribed RNA
atcatagctaccatttattgaaaactatta

SEQ ID NO: 116 - RNA, 30 nt, Homo sapiens
  source 1..30: mol_type = transcribed RNA
atagctaccatttattgaaaactattatgt

SEQ ID NO: 117 - RNA, 30 nt, Homo sapiens
  source 1..30: mol_type = transcribed RNA
gtgtcaacttcaaagaacttatcctttagt

SEQ ID NO: 118 - RNA, 30 nt, Homo sapiens
  source 1..30: mol_type = transcribed RNA
aacttcaaagaacttatcctttagttggag

SEQ ID NO: 119 - RNA, 30 nt, Homo sapiens
  source 1..30: mol_type = transcribed RNA
acttatcctttagttggagagccaagacaa

SEQ ID NO: 120 - RNA, 30 nt, Homo sapiens
  source 1..30: mol_type = transcribed RNA
ttatcctttagttggagagccaagacaatc

SEQ ID NO: 121 - RNA, 30 nt, Homo sapiens
  source 1..30: mol_type = transcribed RNA
atcctttagttggagagccaagacaatcat

SEQ ID NO: 122 - RNA, 30 nt, Homo sapiens
  source 1..30: mol_type = transcribed RNA
tcctttagttggagagccaagacaatcata

SEQ ID NO: 123 - RNA, 30 nt, Homo sapiens
  source 1..30: mol_type = transcribed RNA
tttagttggagagccaagacaatcataaca

SEQ ID NO: 124 - RNA, 30 nt, Homo sapiens
  source 1..30: mol_type = transcribed RNA
tagttggagagccaagacaatcataacaat

SEQ ID NO: 125 - RNA, 30 nt, Homo sapiens
  source 1..30: mol_type = transcribed RNA
ttggagagccaagacaatcataacaataac

SEQ ID NO: 126 - RNA, 30 nt, Homo sapiens
  source 1..30: mol_type = transcribed RNA
gagagccaagacaatcataacaataacaaa

SEQ ID NO: 127 - RNA, 30 nt, Homo sapiens
  source 1..30: mol_type = transcribed RNA
gagccaagacaatcataacaataacaaatg

SEQ ID NO: 128 - RNA, 30 nt, Homo sapiens
  source 1..30: mol_type = transcribed RNA
tgctcagctgtctgcttctcacaggatcta

SEQ ID NO: 129 - RNA, 30 nt, Homo sapiens
  source 1..30: mol_type = transcribed RNA
taaaagatcctgaactgagtttaaaaggca

SEQ ID NO: 130 - RNA, 30 nt, Homo sapiens
  source 1..30: mol_type = transcribed RNA
aaaagatcctgaactgagtttaaaaggcac

SEQ ID NO: 131 - RNA, 30 nt, Homo sapiens
  source 1..30: mol_type = transcribed RNA
gatcctgaactgagtttaaaaggcacccag

SEQ ID NO: 132 - RNA, 30 nt, Homo sapiens
  source 1..30: mol_type = transcribed RNA
actgagtttaaaaggcacccagcacatcat

SEQ ID NO: 133 - RNA, 30 nt, Homo sapiens
  source 1..30: mol_type = transcribed RNA
atcatatcaaatgcaacgtacaaagaaata

SEQ ID NO: 134 - RNA, 30 nt, Homo sapiens
  source 1..30: mol_type = transcribed RNA
tcatatcaaatgcaacgtacaaagaaatag

SEQ ID NO: 135 - RNA, 30 nt, Homo sapiens
  source 1..30: mol_type = transcribed RNA
cggaagttgtatggttaaaagatgggttac

SEQ ID NO: 136 - RNA, 30 nt, Homo sapiens
  source 1..30: mol_type = transcribed RNA
atgtgtttaaaaacctcactgccactctaa

SEQ ID NO: 137 - RNA, 30 nt, Homo sapiens
  source 1..30: mol_type = transcribed RNA
gtgtttaaaaacctcactgccactctaatt

SEQ ID NO: 138 - RNA, 30 nt, Homo sapiens
  source 1..30: mol_type = transcribed RNA
gtttaaaaacctcactgccactctaattgt

SEQ ID NO: 139 - RNA, 30 nt, Homo sapiens
  source 1..30: mol_type = transcribed RNA
catgggaaacagaattgagagcatcactca

SEQ ID NO: 140 - RNA, 20 nt, synthetic construct
  misc_feature 1..20: note = Description of Artificial Sequence: Synthetic oligonucleotide
  source 1..20: mol_type = other RNA
aatcagaggtgagcactgca

SEQ ID NO: 141 - RNA, 20 nt, synthetic construct
  misc_feature 1..20: note = Description of Artificial Sequence: Synthetic oligonucleotide
  source 1..20: mol_type = other RNA
gaggtgagcactgcaacaaa

SEQ ID NO: 142 - RNA, 20 nt, synthetic construct
  misc_feature 1..20: note = Description of Artificial Sequence: Synthetic oligonucleotide
  source 1..20: mol_type = other RNA
aggtgagcactgcaacaaaa

SEQ ID NO: 143 - RNA, 20 nt, synthetic construct
  misc_feature 1..20: note = Description of Artificial Sequence: Synthetic oligonucleotide
  source 1..20: mol_type = other RNA
tgagcactgcaacaaaaagg

SEQ ID NO: 144 - RNA, 20 nt, synthetic construct
  misc_feature 1..20: note = Description of Artificial Sequence: Synthetic oligonucleotide
  source 1..20: mol_type = other RNA
ttttctctcggatctccaaa

SEQ ID NO: 145 - RNA, 20 nt, synthetic construct
  misc_feature 1..20: note = Description of Artificial Sequence: Synthetic oligonucleotide
  source 1..20: mol_type = other RNA
tttctctcggatctccaaat

SEQ ID NO: 146 - RNA, 20 nt, synthetic construct
  misc_feature 1..20: note = Description of Artificial Sequence: Synthetic oligonucleotide
  source 1..20: mol_type = other RNA
ctctcggatctccaaattta

SEQ ID NO: 147 - RNA, 20 nt, synthetic construct
  misc_feature 1..20: note = Description of Artificial Sequence: Synthetic oligonucleotide
  source 1..20: mol_type = other RNA
tctcggatctccaaatttaa

SEQ ID NO: 148 - RNA, 20 nt, synthetic construct
  misc_feature 1..20: note = Description of Artificial Sequence: Synthetic oligonucleotide
  source 1..20: mol_type = other RNA
tcggatctccaaatttaaaa

SEQ ID NO: 149 - RNA, 20 nt, synthetic construct
  misc_feature 1..20: note = Description of Artificial Sequence: Synthetic oligonucleotide
  source 1..20: mol_type = other RNA
tccaaatttaaaagcacaag

SEQ ID NO: 150 - RNA, 20 nt, synthetic construct
  misc_feature 1..20: note = Description of Artificial Sequence: Synthetic oligonucleotide
  source 1..20: mol_type = other RNA
ccaaatttaaaagcacaagg

SEQ ID NO: 151 - RNA, 20 nt, synthetic construct
  misc_feature 1..20: note = Description of Artificial Sequence: Synthetic oligonucleotide
  source 1..20: mol_type = other RNA
caaatttaaaagcacaagga

SEQ ID NO: 152 - RNA, 20 nt, synthetic construct
  misc_feature 1..20: note = Description of Artificial Sequence: Synthetic oligonucleotide
  source 1..20: mol_type = other RNA
aagcacaaggaatgattgta

SEQ ID NO: 153 - RNA, 20 nt, synthetic construct
  misc_feature 1..20: note = Description of Artificial Sequence: Synthetic oligonucleotide
  source 1..20: mol_type = other RNA
gaatgattgtaccacacaaa

SEQ ID NO: 154 - RNA, 20 nt, synthetic construct
  misc_feature 1..20: note = Description of Artificial Sequence: Synthetic oligonucleotide
  source 1..20: mol_type = other RNA
attgtaccacacaaagtaat

SEQ ID NO: 155 - RNA, 20 nt, synthetic construct
  misc_feature 1..20: note = Description of Artificial Sequence: Synthetic oligonucleotide
  source 1..20: mol_type = other RNA
gtaccacacaaagtaatgta

SEQ ID NO: 156 - RNA, 20 nt, synthetic construct
  misc_feature 1..20: note = Description of Artificial Sequence: Synthetic oligonucleotide
  source 1..20: mol_type = other RNA
taccacacaaagtaatgtaa

SEQ ID NO: 157 - RNA, 20 nt, synthetic construct
  misc_feature 1..20: note = Description of Artificial Sequence: Synthetic oligonucleotide
  source 1..20: mol_type = other RNA
ccacacaaagtaatgtaaaa

SEQ ID NO: 158 - RNA, 20 nt, synthetic construct
  misc_feature 1..20: note = Description of Artificial Sequence: Synthetic oligonucleotide
  source 1..20: mol_type = other RNA
acacaaagtaatgtaaaaca

SEQ ID NO: 159 - RNA, 20 nt, synthetic construct
  misc_feature 1..20: note = Description of Artificial Sequence: Synthetic oligonucleotide
  source 1..20: mol_type = other RNA
agtaatgtaaaacattaaag

SEQ ID NO: 160 - RNA, 20 nt, synthetic construct
  misc_feature 1..20: note = Description of Artificial Sequence: Synthetic oligonucleotide
  source 1..20: mol_type = other RNA
gtaatgtaaaacattaaagg

SEQ ID NO: 161 - RNA, 20 nt, synthetic construct
  misc_feature 1..20: note = Description of Artificial Sequence: Synthetic oligonucleotide
  source 1..20: mol_type = other RNA
taaaacattaaaggactcat

SEQ ID NO: 162 - RNA, 20 nt, synthetic construct
  misc_feature 1..20: note = Description of Artificial Sequence: Synthetic oligonucleotide
  source 1..20: mol_type = other RNA
acattaaaggactcattaaa

SEQ ID NO: 163 - RNA, 20 nt, synthetic construct
  misc_feature 1..20: note = Description of Artificial Sequence: Synthetic oligonucleotide
  source 1..20: mol_type = other RNA
ggactcattaaaaagtaaca

SEQ ID NO: 164 - RNA, 20 nt, synthetic construct
  misc_feature 1..20: note = Description of Artificial Sequence: Synthetic oligonucleotide
  source 1..20: mol_type = other RNA
actcattaaaaagtaacagt

SEQ ID NO: 165 - RNA, 20 nt, synthetic construct
  misc_feature 1..20: note = Description of Artificial Sequence: Synthetic oligonucleotide
  source 1..20: mol_type = other RNA
aaagtaacagttgtctcata

SEQ ID NO: 166 - RNA, 20 nt, synthetic construct
  misc_feature 1..20: note = Description of Artificial Sequence: Synthetic oligonucleotide
  source 1..20: mol_type = other RNA
catcatcatcatcatagcta

SEQ ID NO: 167 - RNA, 20 nt, synthetic construct
  misc_feature 1..20: note = Description of Artificial Sequence: Synthetic oligonucleotide
  source 1..20: mol_type = other RNA
catcatcatcatagctatca

SEQ ID NO: 168 - RNA, 20 nt, synthetic construct
  misc_feature 1..20: note = Description of Artificial Sequence: Synthetic oligonucleotide
  source 1..20: mol_type = other RNA
catcatcatagctatcatca

SEQ ID NO: 169 - RNA, 20 nt, synthetic construct
  misc_feature 1..20: note = Description of Artificial Sequence: Synthetic oligonucleotide
  source 1..20: mol_type = other RNA
atcatcatcatcatcatagc

SEQ ID NO: 170 - RNA, 20 nt, synthetic construct
  misc_feature 1..20: note = Description of Artificial Sequence: Synthetic oligonucleotide
  source 1..20: mol_type = other RNA
catcatcatcatcatagcta

SEQ ID NO: 171 - RNA, 20 nt, synthetic construct
  misc_feature 1..20: note = Description of Artificial Sequence: Synthetic oligonucleotide
  source 1..20: mol_type = other RNA
catcatcatcatagctacca

SEQ ID NO: 172 - RNA, 20 nt, synthetic construct
  misc_feature 1..20: note = Description of Artificial Sequence: Synthetic oligonucleotide
  source 1..20: mol_type = other RNA
tcatcatagctaccatttat

SEQ ID NO: 173 - RNA, 20 nt, synthetic construct
  misc_feature 1..20: note = Description of Artificial Sequence: Synthetic oligonucleotide
  source 1..20: mol_type = other RNA
catcatagctaccatttatt

SEQ ID NO: 174 - RNA, 20 nt, synthetic construct
  misc_feature 1..20: note = Description of Artificial Sequence: Synthetic oligonucleotide
  source 1..20: mol_type = other RNA
agctaccatttattgaaaac

SEQ ID NO: 175 - RNA, 20 nt, synthetic construct
  misc_feature 1..20: note = Description of Artificial Sequence: Synthetic oligonucleotide
  source 1..20: mol_type = other RNA
taccatttattgaaaactat

SEQ ID NO: 176 - RNA, 20 nt, synthetic construct
  misc_feature 1..20: note = Description of Artificial Sequence: Synthetic oligonucleotide
  source 1..20: mol_type = other RNA
aacttcaaagaacttatcct

SEQ ID NO: 177 - RNA, 20 nt, synthetic construct
  misc_feature 1..20: note = Description of Artificial Sequence: Synthetic oligonucleotide
  source 1..20: mol_type = other RNA
caaagaacttatcctttagt

SEQ ID NO: 178 - RNA, 20 nt, synthetic construct
  misc_feature 1..20: note = Description of Artificial Sequence: Synthetic oligonucleotide
  source 1..20: mol_type = other RNA
tcctttagttggagagccaa

SEQ ID NO: 179 - RNA, 20 nt, synthetic construct
  misc_feature 1..20: note = Description of Artificial Sequence: Synthetic oligonucleotide
  source 1..20: mol_type = other RNA
ctttagttggagagccaaga

SEQ ID NO: 180 - RNA, 20 nt, synthetic construct
  misc_feature 1..20: note = Description of Artificial Sequence: Synthetic oligonucleotide
  source 1..20: mol_type = other RNA
ttagttggagagccaagaca

SEQ ID NO: 181 - RNA, 20 nt, synthetic construct
  misc_feature 1..20: note = Description of Artificial Sequence: Synthetic oligonucleotide
  source 1..20: mol_type = other RNA
tagttggagagccaagacaa

SEQ ID NO: 182 - RNA, 20 nt, synthetic construct
  misc_feature 1..20: note = Description of Artificial Sequence: Synthetic oligonucleotide
  source 1..20: mol_type = other RNA
ttggagagccaagacaatca

SEQ ID NO: 183 - RNA, 20 nt, synthetic construct
  misc_feature 1..20: note = Description of Artificial Sequence: Synthetic oligonucleotide
  source 1..20: mol_type = other RNA
ggagagccaagacaatcata

SEQ ID NO: 184 - RNA, 20 nt, synthetic construct
  misc_feature 1..20: note = Description of Artificial Sequence: Synthetic oligonucleotide
  source 1..20: mol_type = other RNA
gagccaagacaatcataaca

SEQ ID NO: 185 - RNA, 20 nt, synthetic construct
  misc_feature 1..20: note = Description of Artificial Sequence: Synthetic oligonucleotide
  source 1..20: mol_type = other RNA
ccaagacaatcataacaata

SEQ ID NO: 186 - RNA, 20 nt, synthetic construct
  misc_feature 1..20: note = Description of Artificial Sequence: Synthetic oligonucleotide
  source 1..20: mol_type = other RNA
aagacaatcataacaataac

SEQ ID NO: 187 - RNA, 20 nt, synthetic construct
  misc_feature 1..20: note = Description of Artificial Sequence: Synthetic oligonucleotide
  source 1..20: mol_type = other RNA
agctgtctgcttctcacagg

SEQ ID NO: 188 - RNA, 20 nt, synthetic construct
  misc_feature 1..20: note = Description of Artificial Sequence: Synthetic oligonucleotide
  source 1..20: mol_type = other RNA
gatcctgaactgagtttaaa

SEQ ID NO: 189 - RNA, 20 nt, synthetic construct
  misc_feature 1..20: note = Description of Artificial Sequence: Synthetic oligonucleotide
  source 1..20: mol_type = other RNA
atcctgaactgagtttaaaa

SEQ ID NO: 190 - RNA, 20 nt, synthetic construct
  misc_feature 1..20: note = Description of Artificial Sequence: Synthetic oligonucleotide
  source 1..20: mol_type = other RNA
tgaactgagtttaaaaggca

SEQ ID NO: 191 - RNA, 20 nt, synthetic construct
  misc_feature 1..20: note = Description of Artificial Sequence: Synthetic oligonucleotide
  source 1..20: mol_type = other RNA
gtttaaaaggcacccagcac

SEQ ID NO: 192 - RNA, 20 nt, synthetic construct
  misc_feature 1..20: note = Description of Artificial Sequence: Synthetic oligonucleotide
  source 1..20: mol_type = other RNA
atcaaatgcaacgtacaaag

SEQ ID NO: 193 - RNA, 20 nt, synthetic construct
  misc_feature 1..20: note = Description of Artificial Sequence: Synthetic oligonucleotide
  source 1..20: mol_type = other RNA
tcaaatgcaacgtacaaaga

SEQ ID NO: 194 - RNA, 20 nt, synthetic construct
  misc_feature 1..20: note = Description of Artificial Sequence: Synthetic oligonucleotide
  source 1..20: mol_type = other RNA
gttgtatggttaaaagatgg

SEQ ID NO: 195 - RNA, 20 nt, synthetic construct
  misc_feature 1..20: note = Description of Artificial Sequence: Synthetic oligonucleotide
  source 1..20: mol_type = other RNA
tttaaaaacctcactgccac

SEQ ID NO: 196 - RNA, 20 nt, synthetic construct
  misc_feature 1..20: note = Description of Artificial Sequence: Synthetic oligonucleotide
  source 1..20: mol_type = other RNA
taaaaacctcactgccactc

SEQ ID NO: 197 - RNA, 20 nt, synthetic construct
  misc_feature 1..20: note = Description of Artificial Sequence: Synthetic oligonucleotide
  source 1..20: mol_type = other RNA
aaaacctcactgccactcta

SEQ ID NO: 198 - RNA, 20 nt, synthetic construct
  misc_feature 1..20: note = Description of Artificial Sequence: Synthetic oligonucleotide
  source 1..20: mol_type = other RNA
gaaacagaattgagagcatc

SEQ ID NO: 199 - RNA, 20 nt, synthetic construct
  misc_feature 1..20: note = Description of Artificial Sequence: Synthetic oligonucleotide
  source 1..20: mol_type = other RNA
tgcagtgctcacctctgatt

SEQ ID NO: 200 - RNA, 20 nt, synthetic construct
  misc_feature 1..20: note = Description of Artificial Sequence: Synthetic oligonucleotide
  source 1..20: mol_type = other RNA
tttgttgcagtgctcacctc

SEQ ID NO: 201 - RNA, 20 nt, synthetic construct
  misc_feature 1..20: note = Description of Artificial Sequence: Synthetic oligonucleotide
  source 1..20: mol_type = other RNA
ttttgttgcagtgctcacct

SEQ ID NO: 202 - RNA, 20 nt, synthetic construct
  misc_feature 1..20: note = Description of Artificial Sequence: Synthetic oligonucleotide
  source 1..20: mol_type = other RNA
cctttttgttgcagtgctca

SEQ ID NO: 203 - RNA, 20 nt, synthetic construct
  misc_feature 1..20: note = Description of Artificial Sequence: Synthetic oligonucleotide
  source 1..20: mol_type = other RNA
tttggagatccgagagaaaa

SEQ ID NO: 204 - RNA, 20 nt, synthetic construct
  misc_feature 1..20: note = Description of Artificial Sequence: Synthetic oligonucleotide
  source 1..20: mol_type = other RNA
atttggagatccgagagaaa

SEQ ID NO: 205 - RNA, 20 nt, synthetic construct
  misc_feature 1..20: note = Description of Artificial Sequence: Synthetic oligonucleotide
  source 1..20: mol_type = other RNA
taaatttggagatccgagag

SEQ ID NO: 206 - RNA, 20 nt, synthetic construct
  misc_feature 1..20: note = Description of Artificial Sequence: Synthetic oligonucleotide
  source 1..20: mol_type = other RNA
ttaaatttggagatccgaga

SEQ ID NO: 207 - RNA, 20 nt, synthetic construct
  misc_feature 1..20: note = Description of Artificial Sequence: Synthetic oligonucleotide
  source 1..20: mol_type = other RNA
ttttaaatttggagatccga

SEQ ID NO: 208 - RNA, 20 nt, synthetic construct
  misc_feature 1..20: note = Description of Artificial Sequence: Synthetic oligonucleotide
  source 1..20: mol_type = other RNA
cttgtgcttttaaatttgga

SEQ ID NO: 209 - RNA, 20 nt, synthetic construct
  misc_feature 1..20: note = Description of Artificial Sequence: Synthetic oligonucleotide
  source 1..20: mol_type = other RNA
ccttgtgcttttaaatttgg

SEQ ID NO: 210 - RNA, 20 nt, synthetic construct
  misc_feature 1..20: note = Description of Artificial Sequence: Synthetic oligonucleotide
  source 1..20: mol_type = other RNA
tccttgtgcttttaaatttg

SEQ ID NO: 211 - RNA, 20 nt, synthetic construct
  misc_feature 1..20: note = Description of Artificial Sequence: Synthetic oligonucleotide
  source 1..20: mol_type = other RNA
tacaatcattccttgtgctt

SEQ ID NO: 212 - RNA, 20 nt, synthetic construct
  misc_feature 1..20: note = Description of Artificial Sequence: Synthetic oligonucleotide
  source 1..20: mol_type = other RNA
tttgtgtggtacaatcattc

SEQ ID NO: 213 - RNA, 20 nt, synthetic construct
  misc_feature 1..20: note = Description of Artificial Sequence: Synthetic oligonucleotide
  source 1..20: mol_type = other RNA
attactttgtgtggtacaat

SEQ ID NO: 214 - RNA, 20 nt, synthetic construct
  misc_feature 1..20: note = Description of Artificial Sequence: Synthetic oligonucleotide
  source 1..20: mol_type = other RNA
tacattactttgtgtggtac

SEQ ID NO: 215 - RNA, 20 nt, synthetic construct
  misc_feature 1..20: note = Description of Artificial Sequence: Synthetic oligonucleotide
  source 1..20: mol_type = other RNA
ttacattactttgtgtggta

SEQ ID NO: 216 - RNA, 20 nt, synthetic construct
  misc_feature 1..20: note = Description of Artificial Sequence: Synthetic oligonucleotide
  source 1..20: mol_type = other RNA
ttttacattactttgtgtgg

SEQ ID NO: 217 - RNA, 20 nt, synthetic construct
  misc_feature 1..20: note = Description of Artificial Sequence: Synthetic oligonucleotide
  source 1..20: mol_type = other RNA
tgttttacattactttgtgt

SEQ ID NO: 218 - RNA, 20 nt, synthetic construct
  misc_feature 1..20: note = Description of Artificial Sequence: Synthetic oligonucleotide
  source 1..20: mol_type = other RNA
ctttaatgttttacattact

SEQ ID NO: 219 - RNA, 20 nt, synthetic construct
  misc_feature 1..20: note = Description of Artificial Sequence: Synthetic oligonucleotide
  source 1..20: mol_type = other RNA
cctttaatgttttacattac

SEQ ID NO: 220 - RNA, 20 nt, synthetic construct
  misc_feature 1..20: note = Description of Artificial Sequence: Synthetic oligonucleotide
  source 1..20: mol_type = other RNA
atgagtcctttaatgtttta

SEQ ID NO: 221 - RNA, 20 nt, synthetic construct
  misc_feature 1..20: note = Description of Artificial Sequence: Synthetic oligonucleotide
  source 1..20: mol_type = other RNA
tttaatgagtcctttaatgt

SEQ ID NO: 222 - RNA, 20 nt, synthetic construct
  misc_feature 1..20: note = Description of Artificial Sequence: Synthetic oligonucleotide
  source 1..20: mol_type = other RNA
tgttactttttaatgagtcc

SEQ ID NO: 223 - RNA, 20 nt, synthetic construct
  misc_feature 1..20: note = Description of Artificial Sequence: Synthetic oligonucleotide
  source 1..20: mol_type = other RNA
actgttactttttaatgagt

SEQ ID NO: 224 - RNA, 20 nt, synthetic construct
  misc_feature 1..20: note = Description of Artificial Sequence: Synthetic oligonucleotide
  source 1..20: mol_type = other RNA
tatgagacaactgttacttt

SEQ ID NO: 225 - RNA, 20 nt, synthetic construct
  misc_feature 1..20: note = Description of Artificial Sequence: Synthetic oligonucleotide
  source 1..20: mol_type = other RNA
tagctatgatgatgatgatg

SEQ ID NO: 226 - RNA, 20 nt, synthetic construct
  misc_feature 1..20: note = Description of Artificial Sequence: Synthetic oligonucleotide
  source 1..20: mol_type = other RNA
tgatagctatgatgatgatg

SEQ ID NO: 227 - RNA, 20 nt, synthetic construct
  misc_feature 1..20: note = Description of Artificial Sequence: Synthetic oligonucleotide
  source 1..20: mol_type = other RNA
tgatgatagctatgatgatg

SEQ ID NO: 228 - RNA, 20 nt, synthetic construct
  misc_feature 1..20: note = Description of Artificial Sequence: Synthetic oligonucleotide
  source 1..20: mol_type = other RNA
gctatgatgatgatgatgat

SEQ ID NO: 229 - RNA, 20 nt, synthetic construct
  misc_feature 1..20: note = Description of Artificial Sequence: Synthetic oligonucleotide
  source 1..20: mol_type = other RNA
tagctatgatgatgatgatg

SEQ ID NO: 230 - RNA, 20 nt, synthetic construct
  misc_feature 1..20: note = Description of Artificial Sequence: Synthetic oligonucleotide
  source 1..20: mol_type = other RNA
tggtagctatgatgatgatg

SEQ ID NO: 231 - RNA, 20 nt, synthetic construct
  misc_feature 1..20: note = Description of Artificial Sequence: Synthetic oligonucleotide
  source 1..20: mol_type = other RNA
ataaatggtagctatgatga

SEQ ID NO: 232 - RNA, 20 nt, synthetic construct
  misc_feature 1..20: note = Description of Artificial Sequence: Synthetic oligonucleotide
  source 1..20: mol_type = other RNA
aataaatggtagctatgatg

SEQ ID NO: 233 - RNA, 20 nt, synthetic construct
  misc_feature 1..20: note = Description of Artificial Sequence: Synthetic oligonucleotide
  source 1..20: mol_type = other RNA
gttttcaataaatggtagct

SEQ ID NO: 234 - RNA, 20 nt, synthetic construct
  misc_feature 1..20: note = Description of Artificial Sequence: Synthetic oligonucleotide
  source 1..20: mol_type = other RNA
atagttttcaataaatggta

SEQ ID NO: 235 - RNA, 20 nt, synthetic construct
  misc_feature 1..20: note = Description of Artificial Sequence: Synthetic oligonucleotide
  source 1..20: mol_type = other RNA
aggataagttctttgaagtt

SEQ ID NO: 236 - RNA, 20 nt, synthetic construct
  misc_feature 1..20: note = Description of Artificial Sequence: Synthetic oligonucleotide
  source 1..20: mol_type = other RNA
actaaaggataagttctttg

SEQ ID NO: 237 - RNA, 20 nt, synthetic construct
  misc_feature 1..20: note = Description of Artificial Sequence: Synthetic oligonucleotide
  source 1..20: mol_type = other RNA
ttggctctccaactaaagga

SEQ ID NO: 238 - RNA, 20 nt, synthetic construct
  misc_feature 1..20: note = Description of Artificial Sequence: Synthetic oligonucleotide
  source 1..20: mol_type = other RNA
tcttggctctccaactaaag

SEQ ID NO: 239 - RNA, 20 nt, synthetic construct
  misc_feature 1..20: note = Description of Artificial Sequence: Synthetic oligonucleotide
  source 1..20: mol_type = other RNA
tgtcttggctctccaactaa

SEQ ID NO: 240 - RNA, 20 nt, synthetic construct
  misc_feature 1..20: note = Description of Artificial Sequence: Synthetic oligonucleotide
  source 1..20: mol_type = other RNA
ttgtcttggctctccaacta

SEQ ID NO: 241 - RNA, 20 nt, synthetic construct
  misc_feature 1..20: note = Description of Artificial Sequence: Synthetic oligonucleotide
  source 1..20: mol_type = other RNA
tgattgtcttggctctccaa

SEQ ID NO: 242 - RNA, 20 nt, synthetic construct
  misc_feature 1..20: note = Description of Artificial Sequence: Synthetic oligonucleotide
  source 1..20: mol_type = other RNA
tatgattgtcttggctctcc

SEQ ID NO: 243 - RNA, 20 nt, synthetic construct
  misc_feature 1..20: note = Description of Artificial Sequence: Synthetic oligonucleotide
  source 1..20: mol_type = other RNA
tgttatgattgtcttggctc

SEQ ID NO: 244 - RNA, 20 nt, synthetic construct
  misc_feature 1..20: note = Description of Artificial Sequence: Synthetic oligonucleotide
  source 1..20: mol_type = other RNA
tattgttatgattgtcttgg

SEQ ID NO: 245 - RNA, 20 nt, synthetic construct
  misc_feature 1..20: note = Description of Artificial Sequence: Synthetic oligonucleotide
  source 1..20: mol_type = other RNA
gttattgttatgattgtctt

SEQ ID NO: 246 - RNA, 20 nt, synthetic construct
  misc_feature 1..20: note = Description of Artificial Sequence: Synthetic oligonucleotide
  source 1..20: mol_type = other RNA
cctgtgagaagcagacagct

SEQ ID NO: 247 - RNA, 20 nt, synthetic construct
  misc_feature 1..20: note = Description of Artificial Sequence: Synthetic oligonucleotide
  source 1..20: mol_type = other RNA
tttaaactcagttcaggatc

SEQ ID NO: 248 - RNA, 20 nt, synthetic construct
  misc_feature 1..20: note = Description of Artificial Sequence: Synthetic oligonucleotide
  source 1..20: mol_type = other RNA
ttttaaactcagttcaggat

SEQ ID NO: 249 - RNA, 20 nt, synthetic construct
  misc_feature 1..20: note = Description of Artificial Sequence: Synthetic oligonucleotide
  source 1..20: mol_type = other RNA
tgccttttaaactcagttca

SEQ ID NO: 250 - RNA, 20 nt, synthetic construct
  misc_feature 1..20: note = Description of Artificial Sequence: Synthetic oligonucleotide
  source 1..20: mol_type = other RNA
gtgctgggtgccttttaaac

SEQ ID NO: 251 - RNA, 20 nt, synthetic construct
  misc_feature 1..20: note = Description of Artificial Sequence: Synthetic oligonucleotide
  source 1..20: mol_type = other RNA
ctttgtacgttgcatttgat

SEQ ID NO: 252 - RNA, 20 nt, synthetic construct
  misc_feature 1..20: note = Description of Artificial Sequence: Synthetic oligonucleotide
  source 1..20: mol_type = other RNA
tctttgtacgttgcatttga

SEQ ID NO: 253 - RNA, 20 nt, synthetic construct
  misc_feature 1..20: note = Description of Artificial Sequence: Synthetic oligonucleotide
  source 1..20: mol_type = other RNA
ccatcttttaaccatacaac

SEQ ID NO: 254 - RNA, 20 nt, synthetic construct
  misc_feature 1..20: note = Description of Artificial Sequence: Synthetic oligonucleotide
  source 1..20: mol_type = other RNA
gtggcagtgaggtttttaaa

SEQ ID NO: 255 - RNA, 20 nt, synthetic construct
  misc_feature 1..20: note = Description of Artificial Sequence: Synthetic oligonucleotide
  source 1..20: mol_type = other RNA
gagtggcagtgaggttttta

SEQ ID NO: 256 - RNA, 20 nt, synthetic construct
  misc_feature 1..20: note = Description of Artificial Sequence: Synthetic oligonucleotide
  source 1..20: mol_type = other RNA
tagagtggcagtgaggtttt

SEQ ID NO: 257 - RNA, 20 nt, synthetic construct
  misc_feature 1..20: note = Description of Artificial Sequence: Synthetic oligonucleotide
  source 1..20: mol_type = other RNA
gatgctctcaattctgtttc

SEQ ID NO: 258 - RNA, 15 nt, synthetic construct
  misc_feature 1..15: note = Description of Artificial Sequence: Synthetic oligonucleotide
  source 1..15: mol_type = other RNA
aatcagaggtgagca

SEQ ID NO: 259 - RNA, 15 nt, synthetic construct
  misc_feature 1..15: note = Description of Artificial Sequence: Synthetic oligonucleotide
  source 1..15: mol_type = other RNA
gaggtgagcactgca

SEQ ID NO: 260 - RNA, 15 nt, synthetic construct
  misc_feature 1..15: note = Description of Artificial Sequence: Synthetic oligonucleotide
  source 1..15: mol_type = other RNA
aggtgagcactgcaa

SEQ ID NO: 261 - RNA, 15 nt, synthetic construct
  misc_feature 1..15: note = Description of Artificial Sequence: Synthetic oligonucleotide
  source 1..15: mol_type = other RNA
tgagcactgcaacaa

SEQ ID NO: 262 - RNA, 15 nt, synthetic construct
  misc_feature 1..15: note = Description of Artificial Sequence: Synthetic oligonucleotide
  source 1..15: mol_type = other RNA
ttttctctcggatct

SEQ ID NO: 263 - RNA, 15 nt, synthetic construct
  misc_feature 1..15: note = Description of Artificial Sequence: Synthetic oligonucleotide
  source 1..15: mol_type = other RNA
tttctctcggatctc

SEQ ID NO: 264 - RNA, 15 nt, synthetic construct
  misc_feature 1..15: note = Description of Artificial Sequence: Synthetic oligonucleotide
  source 1..15: mol_type = other RNA
ctctcggatctccaa

SEQ ID NO: 265 - RNA, 15 nt, synthetic construct
  misc_feature 1..15: note = Description of Artificial Sequence: Synthetic oligonucleotide
  source 1..15: mol_type = other RNA
tctcggatctccaaa

SEQ ID NO: 266 - RNA, 15 nt, synthetic construct
  misc_feature 1..15: note = Description of Artificial Sequence: Synthetic oligonucleotide
  source 1..15: mol_type = other RNA
tcggatctccaaatt

SEQ ID NO: 267 - RNA, 15 nt, synthetic construct
  misc_feature 1..15: note = Description of Artificial Sequence: Synthetic oligonucleotide
  source 1..15: mol_type = other RNA
tccaaatttaaaagc

SEQ ID NO: 268 - RNA, 15 nt, synthetic construct
  misc_feature 1..15: note = Description of Artificial Sequence: Synthetic oligonucleotide
  source 1..15: mol_type = other RNA
ccaaatttaaaagca

SEQ ID NO: 269 - RNA, 15 nt, synthetic construct
  misc_feature 1..15: note = Description of Artificial Sequence: Synthetic oligonucleotide
  source 1..15: mol_type = other RNA
caaatttaaaagcac

SEQ ID NO: 270 - RNA, 15 nt, synthetic construct
  misc_feature 1..15: note = Description of Artificial Sequence: Synthetic oligonucleotide
  source 1..15: mol_type = other RNA
aagcacaaggaatga

SEQ ID NO: 271 - RNA, 15 nt, synthetic construct
  misc_feature 1..15: note = Description of Artificial Sequence: Synthetic oligonucleotide
  source 1..15: mol_type = other RNA
gaatgattgtaccac

SEQ ID NO: 272 - RNA, 15 nt, synthetic construct
  misc_feature 1..15: note = Description of Artificial Sequence: Synthetic oligonucleotide
  source 1..15: mol_type = other RNA
attgtaccacacaaa

SEQ ID NO: 273 - RNA, 15 nt, synthetic construct
  misc_feature 1..15: note = Description of Artificial Sequence: Synthetic oligonucleotide
  source 1..15: mol_type = other RNA
gtaccacacaaagta

SEQ ID NO: 274 - RNA, 15 nt, synthetic construct
  misc_feature 1..15: note = Description of Artificial Sequence: Synthetic oligonucleotide
  source 1..15: mol_type = other RNA
taccacacaaagtaa

SEQ ID NO: 275 - RNA, 15 nt, synthetic construct
  misc_feature 1..15: note = Description of Artificial Sequence: Synthetic oligonucleotide
  source 1..15: mol_type = other RNA
ccacacaaagtaatg

SEQ ID NO: 276 - RNA, 15 nt, synthetic construct
  misc_feature 1..15: note = Description of Artificial Sequence: Synthetic oligonucleotide
  source 1..15: mol_type = other RNA
acacaaagtaatgta

SEQ ID NO: 277 - RNA, 15 nt, synthetic construct
  misc_feature 1..15: note = Description of Artificial Sequence: Synthetic oligonucleotide
  source 1..15: mol_type = other RNA
agtaatgtaaaacat

SEQ ID NO: 278 - RNA, 15 nt, synthetic construct
  misc_feature 1..15: note = Description of Artificial Sequence: Synthetic oligonucleotide
  source 1..15: mol_type = other RNA
gtaatgtaaaacatt

SEQ ID NO: 279 - RNA, 15 nt, synthetic construct
  misc_feature 1..15: note = Description of Artificial Sequence: Synthetic oligonucleotide
  source 1..15: mol_type = other RNA
taaaacattaaagga

SEQ ID NO: 280 - RNA, 15 nt, synthetic construct
  misc_feature 1..15: note = Description of Artificial Sequence: Synthetic oligonucleotide
  source 1..15: mol_type = other RNA
acattaaaggactca

SEQ ID NO: 281 - RNA, 15 nt, synthetic construct
  misc_feature 1..15: note = Description of Artificial Sequence: Synthetic oligonucleotide
  source 1..15: mol_type = other RNA
ggactcattaaaaag

SEQ ID NO: 282 - RNA, 15 nt, synthetic construct
  misc_feature 1..15: note = Description of Artificial Sequence: Synthetic oligonucleotide
  source 1..15: mol_type = other RNA
actcattaaaaagta

SEQ ID NO: 283 - RNA, 15 nt, synthetic construct
  misc_feature 1..15: note = Description of Artificial Sequence: Synthetic oligonucleotide
  source 1..15: mol_type = other RNA
aaagtaacagttgtc

SEQ ID NO: 284 - RNA, 15 nt, synthetic construct
  misc_feature 1..15: note = Description of Artificial Sequence: Synthetic oligonucleotide
  source 1..15: mol_type = other RNA
catcatcatcatcat

SEQ ID NO: 285 - RNA, 15 nt, synthetic construct
  misc_feature 1..15: note = Description of Artificial Sequence: Synthetic oligonucleotide
  source 1..15: mol_type = other RNA
catcatcatcatagc

SEQ ID NO: 286 - RNA, 15 nt, synthetic construct
  misc_feature 1..15: note = Description of Artificial Sequence: Synthetic oligonucleotide
  source 1..15: mol_type = other RNA
catcatcatagctat

SEQ ID NO: 287 - RNA, 15 nt, synthetic construct
  misc_feature 1..15: note = Description of Artificial Sequence: Synthetic oligonucleotide
  source 1..15: mol_type = other RNA
atcatcatcatcatc

SEQ ID NO: 288 - RNA, 15 nt, synthetic construct
  misc_feature 1..15: note = Description of Artificial Sequence: Synthetic oligonucleotide
  source 1..15: mol_type = other RNA
catcatcatcatcat

SEQ ID NO: 289 - RNA, 15 nt, synthetic construct
  misc_feature 1..15: note = Description of Artificial Sequence: Synthetic oligonucleotide
  source 1..15: mol_type = other RNA
catcatcatcatagc

SEQ ID NO: 290 - RNA, 15 nt, synthetic construct
  misc_feature 1..15: note = Description of Artificial Sequence: Synthetic oligonucleotide
  source 1..15: mol_type = other RNA
tcatcatagctacca

SEQ ID NO: 291 - RNA, 15 nt, synthetic construct
  misc_feature 1..15: note = Description of Artificial Sequence: Synthetic oligonucleotide
  source 1..15: mol_type = other RNA
catcatagctaccat

SEQ ID NO: 292 - RNA, 15 nt, synthetic construct
  misc_feature 1..15: note = Description of Artificial Sequence: Synthetic oligonucleotide
  source 1..15: mol_type = other RNA
agctaccatttattg

SEQ ID NO: 293 - RNA, 15 nt, synthetic construct
  misc_feature 1..15: note = Description of Artificial Sequence: Synthetic oligonucleotide
  source 1..15: mol_type = other RNA
taccatttattgaaa

SEQ ID NO: 294 - RNA, 15 nt, synthetic construct
  misc_feature 1..15: note = Description of Artificial Sequence: Synthetic oligonucleotide
  source 1..15: mol_type = other RNA
aacttcaaagaactt

SEQ ID NO: 295 - RNA, 15 nt, synthetic construct
  misc_feature 1..15: note = Description of Artificial Sequence: Synthetic oligonucleotide
  source 1..15: mol_type = other RNA
caaagaacttatcct

SEQ ID NO: 296 - RNA, 15 nt, synthetic construct
  misc_feature 1..15: note = Description of Artificial Sequence: Synthetic oligonucleotide
  source 1..15: mol_type = other RNA
tcctttagttggaga

SEQ ID NO: 297 - RNA, 15 nt, synthetic construct
  misc_feature 1..15: note = Description of Artificial Sequence: Synthetic oligonucleotide
  source 1..15: mol_type = other RNA
ctttagttggagagc

SEQ ID NO: 298 - RNA, 15 nt, synthetic construct
  misc_feature 1..15: note = Description of Artificial Sequence: Synthetic oligonucleotide
  source 1..15: mol_type = other RNA
ttagttggagagcca

SEQ ID NO: 299 - RNA, 15 nt, synthetic construct
  misc_feature 1..15: note = Description of Artificial Sequence: Synthetic oligonucleotide
  source 1..15: mol_type = other RNA
tagttggagagccaa

SEQ ID NO: 300 - RNA, 15 nt, synthetic construct
  misc_feature 1..15: note = Description of Artificial Sequence: Synthetic oligonucleotide
  source 1..15: mol_type = other RNA
ttggagagccaagac

SEQ ID NO: 301 - RNA, 15 nt, synthetic construct
  misc_feature 1..15: note = Description of Artificial Sequence: Synthetic oligonucleotide
  source 1..15: mol_type = other RNA
ggagagccaagacaa

SEQ ID NO: 302 - RNA, 15 nt, synthetic construct
  misc_feature 1..15: note = Description of Artificial Sequence: Synthetic oligonucleotide
  source 1..15: mol_type = other RNA
gagccaagacaatca

SEQ ID NO: 303 - RNA, 15 nt, synthetic construct
  misc_feature 1..15: note = Description of Artificial Sequence: Synthetic oligonucleotide
  source 1..15: mol_type = other RNA
ccaagacaatcataa

SEQ ID NO: 304 - RNA, 15 nt, synthetic construct
  misc_feature 1..15: note = Description of Artificial Sequence: Synthetic oligonucleotide
  source 1..15: mol_type = other RNA
aagacaatcataaca

SEQ ID NO: 305 - RNA, 15 nt, synthetic construct
  misc_feature 1..15: note = Description of Artificial Sequence: Synthetic oligonucleotide
  source 1..15: mol_type = other RNA
agctgtctgcttctc

SEQ ID NO: 306 - RNA, 15 nt, synthetic construct
  misc_feature 1..15: note = Description of Artificial Sequence: Synthetic oligonucleotide
  source 1..15: mol_type = other RNA
gatcctgaactgagt

SEQ ID NO: 307 - RNA, 15 nt, synthetic construct
  misc_feature 1..15: note = Description of Artificial Sequence: Synthetic oligonucleotide
  source 1..15: mol_type = other RNA
atcctgaactgagtt

SEQ ID NO: 308 - RNA, 15 nt, synthetic construct
  misc_feature 1..15: note = Description of Artificial Sequence: Synthetic oligonucleotide
  source 1..15: mol_type = other RNA
tgaactgagtttaaa

SEQ ID NO: 309 - RNA, 15 nt, synthetic construct
  misc_feature 1..15: note = Description of Artificial Sequence: Synthetic oligonucleotide
  source 1..15: mol_type = other RNA
gtttaaaaggcaccc

SEQ ID NO: 310 - RNA, 15 nt, synthetic construct
  misc_feature 1..15: note = Description of Artificial Sequence: Synthetic oligonucleotide
  source 1..15: mol_type = other RNA
atcaaatgcaacgta

SEQ ID NO: 311 - RNA, 15 nt, synthetic construct
  misc_feature 1..15: note = Description of Artificial Sequence: Synthetic oligonucleotide
  source 1..15: mol_type = other RNA
tcaaatgcaacgtac

SEQ ID NO: 312 - RNA, 15 nt, synthetic construct
  misc_feature 1..15: note = Description of Artificial Sequence: Synthetic oligonucleotide
  source 1..15: mol_type = other RNA
gttgtatggttaaaa

SEQ ID NO: 313 - RNA, 15 nt, synthetic construct
  misc_feature 1..15: note = Description of Artificial Sequence: Synthetic oligonucleotide
  source 1..15: mol_type = other RNA
tttaaaaacctcact

SEQ ID NO: 314 - RNA, 15 nt, synthetic construct
  misc_feature 1..15: note = Description of Artificial Sequence: Synthetic oligonucleotide
  source 1..15: mol_type = other RNA
taaaaacctcactgc

SEQ ID NO: 315 - RNA, 15 nt, synthetic construct
  misc_feature 1..15: note = Description of Artificial Sequence: Synthetic oligonucleotide
  source 1..15: mol_type = other RNA
aaaacctcactgcca

SEQ ID NO: 316 - RNA, 15 nt, synthetic construct
  misc_feature 1..15: note = Description of Artificial Sequence: Synthetic oligonucleotide
  source 1..15: mol_type = other RNA
gaaacagaattgaga

SEQ ID NO: 317 - RNA, 19 nt, synthetic construct
  misc_feature 1..19: note = Description of Artificial Sequence: Synthetic oligonucleotide
  source 1..19: mol_type = other RNA
tcagtgctcacctctgatt

SEQ ID NO: 318 - RNA, 19 nt, synthetic construct
  misc_feature 1..19: note = Description of Artificial Sequence: Synthetic oligonucleotide
  source 1..19: mol_type = other RNA
ttgttgcagtgctcacctc

SEQ ID NO: 319 - RNA, 19 nt, synthetic construct
  misc_feature 1..19: note = Description of Artificial Sequence: Synthetic oligonucleotide
  source 1..19: mol_type = other RNA
tttgttgcagtgctcacct

SEQ ID NO: 320 - RNA, 19 nt, synthetic construct
  misc_feature 1..19: note = Description of Artificial Sequence: Synthetic oligonucleotide
  source 1..19: mol_type = other RNA
ttttttgttgcagtgctca

SEQ ID NO: 321 - RNA, 19 nt, synthetic construct
  misc_feature 1..19: note = Description of Artificial Sequence: Synthetic oligonucleotide
  source 1..19: mol_type = other RNA
ttggagatccgagagaaaa

SEQ ID NO: 322 - RNA, 19 nt, synthetic construct
  misc_feature 1..19: note = Description of Artificial Sequence: Synthetic oligonucleotide
  source 1..19: mol_type = other RNA
tttggagatccgagagaaa

SEQ ID NO: 323 - RNA, 19 nt, synthetic construct
  misc_feature 1..19: note = Description of Artificial Sequence: Synthetic oligonucleotide
  source 1..19: mol_type = other RNA
taatttggagatccgagag

SEQ ID NO: 324 - RNA, 19 nt, synthetic construct
  misc_feature 1..19: note = Description of Artificial Sequence: Synthetic oligonucleotide
  source 1..19: mol_type = other RNA
taaatttggagatccgaga

SEQ ID NO: 325 - RNA, 19 nt, synthetic construct
  misc_feature 1..19: note = Description of Artificial Sequence: Synthetic oligonucleotide
  source 1..19: mol_type = other RNA
tttaaatttggagatccga

SEQ ID NO: 326 - RNA, 19 nt, synthetic construct
  misc_feature 1..19: note = Description of Artificial Sequence: Synthetic oligonucleotide
  source 1..19: mol_type = other RNA
ttgtgcttttaaatttgga

SEQ ID NO: 327 - RNA, 19 nt, synthetic construct
  misc_feature 1..19: note = Description of Artificial Sequence: Synthetic oligonucleotide
  source 1..19: mol_type = other RNA
tttgtgcttttaaatttgg

SEQ ID NO: 328 - RNA, 19 nt, synthetic construct
  misc_feature 1..19: note = Description of Artificial Sequence: Synthetic oligonucleotide
  source 1..19: mol_type = other RNA
tcttgtgcttttaaatttg

SEQ ID NO: 329 - RNA, 19 nt, synthetic construct
  misc_feature 1..19: note = Description of Artificial Sequence: Synthetic oligonucleotide
  source 1..19: mol_type = other RNA
tcaatcattccttgtgctt

SEQ ID NO: 330 - RNA, 19 nt, synthetic construct
  misc_feature 1..19: note = Description of Artificial Sequence: Synthetic oligonucleotide
  source 1..19: mol_type = other RNA
ttgtgtggtacaatcattc

SEQ ID NO: 331 - RNA, 19 nt, synthetic construct
  misc_feature 1..19: note = Description of Artificial Sequence: Synthetic oligonucleotide
  source 1..19: mol_type = other RNA
ttactttgtgtggtacaat

SEQ ID NO: 332 - RNA, 19 nt, synthetic construct
  misc_feature 1..19: note = Description of Artificial Sequence: Synthetic oligonucleotide
  source 1..19: mol_type = other RNA
tcattactttgtgtggtac

SEQ ID NO: 333 - RNA, 19 nt, synthetic construct
  misc_feature 1..19: note = Description of Artificial Sequence: Synthetic oligonucleotide
  source 1..19: mol_type = other RNA
tacattactttgtgtggta

SEQ ID NO: 334 - RNA, 19 nt, synthetic construct
  misc_feature 1..19: note = Description of Artificial Sequence: Synthetic oligonucleotide
  source 1..19: mol_type = other RNA
tttacattactttgtgtgg

SEQ ID NO: 335 - RNA, 19 nt, synthetic construct
  misc_feature 1..19: note = Description of Artificial Sequence: Synthetic oligonucleotide
  source 1..19: mol_type = other RNA
tttttacattactttgtgt

SEQ ID NO: 336 - RNA, 19 nt, synthetic construct
  misc_feature 1..19: note = Description of Artificial Sequence: Synthetic oligonucleotide
  source 1..19: mol_type = other RNA
tttaatgttttacattact

SEQ ID NO: 337 - RNA, 19 nt, synthetic construct
  misc_feature 1..19: note = Description of Artificial Sequence: Synthetic oligonucleotide
  source 1..19: mol_type = other RNA
ttttaatgttttacattac

SEQ ID NO: 338 - RNA, 19 nt, synthetic construct
  misc_feature 1..19: note = Description of Artificial Sequence: Synthetic oligonucleotide
  source 1..19: mol_type = other RNA
tgagtcctttaatgtttta

SEQ ID NO: 339 - RNA, 19 nt, synthetic construct
  misc_feature 1..19: note = Description of Artificial Sequence: Synthetic oligonucleotide
  source 1..19: mol_type = other RNA
ttaatgagtcctttaatgt

SEQ ID NO: 340 - RNA, 19 nt, synthetic construct
  misc_feature 1..19: note = Description of Artificial Sequence: Synthetic oligonucleotide
  source 1..19: mol_type = other RNA
tttactttttaatgagtcc

SEQ ID NO: 341 - RNA, 19 nt, synthetic construct
  misc_feature 1..19: note = Description of Artificial Sequence: Synthetic oligonucleotide
  source 1..19: mol_type = other RNA
ttgttactttttaatgagt

SEQ ID NO: 342 - RNA, 19 nt, synthetic construct
  misc_feature 1..19: note = Description of Artificial Sequence: Synthetic oligonucleotide
  source 1..19: mol_type = other RNA
ttgagacaactgttacttt

SEQ ID NO: 343 - RNA, 19 nt, synthetic construct
  misc_feature 1..19: note = Description of Artificial Sequence: Synthetic oligonucleotide
  source 1..19: mol_type = other RNA
tgctatgatgatgatgatg

SEQ ID NO: 344 - RNA, 19 nt, synthetic construct
  misc_feature 1..19: note = Description of Artificial Sequence: Synthetic oligonucleotide
  source 1..19: mol_type = other RNA
tatagctatgatgatgatg

SEQ ID NO: 345 - RNA, 19 nt, synthetic construct
  misc_feature 1..19: note = Description of Artificial Sequence: Synthetic oligonucleotide
  source 1..19: mol_type = other RNA
tatgatagctatgatgatg

SEQ ID NO: 346 - RNA, 19 nt, synthetic construct
  misc_feature 1..19: note = Description of Artificial Sequence: Synthetic oligonucleotide
  source 1..19: mol_type = other RNA
ttatgatgatgatgatgat

SEQ ID NO: 347 - RNA, 19 nt, synthetic construct
  misc_feature 1..19: note = Description of Artificial Sequence: Synthetic oligonucleotide
  source 1..19: mol_type = other RNA
tgctatgatgatgatgatg

SEQ ID NO: 348 - RNA, 19 nt, synthetic construct
  misc_feature 1..19: note = Description of Artificial Sequence: Synthetic oligonucleotide
  source 1..19: mol_type = other RNA
tgtagctatgatgatgatg

SEQ ID NO: 349 - RNA, 19 nt, synthetic construct
  misc_feature 1..19: note = Description of Artificial Sequence: Synthetic oligonucleotide
  source 1..19: mol_type = other RNA
taaatggtagctatgatga

SEQ ID NO: 350 - RNA, 19 nt, synthetic construct
  misc_feature 1..19: note = Description of Artificial Sequence: Synthetic oligonucleotide
  source 1..19: mol_type = other RNA
ttaaatggtagctatgatg

SEQ ID NO: 351 - RNA, 19 nt, synthetic construct
  misc_feature 1..19: note = Description of Artificial Sequence: Synthetic oligonucleotide
  source 1..19: mol_type = other RNA
ttttcaataaatggtagct

SEQ ID NO: 352 - RNA, 19 nt, synthetic construct
  misc_feature 1..19: note = Description of Artificial Sequence: Synthetic oligonucleotide
  source 1..19: mol_type = other RNA
tagttttcaataaatggta

SEQ ID NO: 353 - RNA, 19 nt, synthetic construct
  misc_feature 1..19: note = Description of Artificial Sequence: Synthetic oligonucleotide
  source 1..19: mol_type = other RNA
tgataagttctttgaagtt

SEQ ID NO: 354 - RNA, 19 nt, synthetic construct
  misc_feature 1..19: note = Description of Artificial Sequence: Synthetic oligonucleotide
  source 1..19: mol_type = other RNA
ttaaaggataagttctttg

SEQ ID NO: 355 - RNA, 19 nt, synthetic construct
  misc_feature 1..19: note = Description of Artificial Sequence: Synthetic oligonucleotide
  source 1..19: mol_type = other RNA
tggctctccaactaaagga

SEQ ID NO: 356 - RNA, 19 nt, synthetic construct
  misc_feature 1..19: note = Description of Artificial Sequence: Synthetic oligonucleotide
  source 1..19: mol_type = other RNA
tttggctctccaactaaag

SEQ ID NO: 357 - RNA, 19 nt, synthetic construct
  misc_feature 1..19: note = Description of Artificial Sequence: Synthetic oligonucleotide
  source 1..19: mol_type = other RNA
ttcttggctctccaactaa

SEQ ID NO: 358 - RNA, 19 nt, synthetic construct
  misc_feature 1..19: note = Description of Artificial Sequence: Synthetic oligonucleotide
  source 1..19: mol_type = other RNA
tgtcttggctctccaacta

SEQ ID NO: 359 - RNA, 19 nt, synthetic construct
  misc_feature 1..19: note = Description of Artificial Sequence: Synthetic oligonucleotide
  source 1..19: mol_type = other RNA
tattgtcttggctctccaa

SEQ ID NO: 360 - RNA, 19 nt, synthetic construct
  misc_feature 1..19: note = Description of Artificial Sequence: Synthetic oligonucleotide
  source 1..19: mol_type = other RNA
ttgattgtcttggctctcc

SEQ ID NO: 361 - RNA, 19 nt, synthetic construct
  misc_feature 1..19: note = Description of Artificial Sequence: Synthetic oligonucleotide
  source 1..19: mol_type = other RNA
tttatgattgtcttggctc

SEQ ID NO: 362 - RNA, 19 nt, synthetic construct
  misc_feature 1..19: note = Description of Artificial Sequence: Synthetic oligonucleotide
  source 1..19: mol_type = other RNA
tttgttatgattgtcttgg

SEQ ID NO: 363 - RNA, 19 nt, synthetic construct
  misc_feature 1..19: note = Description of Artificial Sequence: Synthetic oligonucleotide
  source 1..19: mol_type = other RNA
ttattgttatgattgtctt

SEQ ID NO: 364 - RNA, 19 nt, synthetic construct
  misc_feature 1..19: note = Description of Artificial Sequence: Synthetic oligonucleotide
  source 1..19: mol_type = other RNA
ttgtgagaagcagacagct

SEQ ID NO: 365 - RNA, 19 nt, synthetic construct
  misc_feature 1..19: note = Description of Artificial Sequence: Synthetic oligonucleotide
  source 1..19: mol_type = other RNA
ttaaactcagttcaggatc

SEQ ID NO: 366 - RNA, 19 nt, synthetic construct
  misc_feature 1..19: note = Description of Artificial Sequence: Synthetic oligonucleotide
  source 1..19: mol_type = other RNA
tttaaactcagttcaggat

SEQ ID NO: 367 - RNA, 19 nt, synthetic construct
  misc_feature 1..19: note = Description of Artificial Sequence: Synthetic oligonucleotide
  source 1..19: mol_type = other RNA
tccttttaaactcagttca

SEQ ID NO: 368 - RNA, 19 nt, synthetic construct
  misc_feature 1..19: note = Description of Artificial Sequence: Synthetic oligonucleotide
  source 1..19: mol_type = other RNA
tgctgggtgccttttaaac

SEQ ID NO: 369 - RNA, 19 nt, synthetic construct
  misc_feature 1..19: note = Description of Artificial Sequence: Synthetic oligonucleotide
  source 1..19: mol_type = other RNA
tttgtacgttgcatttgat

SEQ ID NO: 370 - RNA, 19 nt, synthetic construct
  misc_feature 1..19: note = Description of Artificial Sequence: Synthetic oligonucleotide
  source 1..19: mol_type = other RNA
ttttgtacgttgcatttga

SEQ ID NO: 371 - RNA, 19 nt, synthetic construct
  misc_feature 1..19: note = Description of Artificial Sequence: Synthetic oligonucleotide
  source 1..19: mol_type = other RNA
tatcttttaaccatacaac

SEQ ID NO: 372 - RNA, 19 nt, synthetic construct
  misc_feature 1..19: note = Description of Artificial Sequence: Synthetic oligonucleotide
  source 1..19: mol_type = other RNA
tggcagtgaggtttttaaa

SEQ ID NO: 373 - RNA, 19 nt, synthetic construct
  misc_feature 1..19: note = Description of Artificial Sequence: Synthetic oligonucleotide
  source 1..19: mol_type = other RNA
tgtggcagtgaggttttta

SEQ ID NO: 374 - RNA, 19 nt, synthetic construct
  misc_feature 1..19: note = Description of Artificial Sequence: Synthetic oligonucleotide
  source 1..19: mol_type = other RNA
tgagtggcagtgaggtttt

SEQ ID NO: 375 - RNA, 19 nt, synthetic construct
  misc_feature 1..19: note = Description of Artificial Sequence: Synthetic oligonucleotide
  source 1..19: mol_type = other RNA
ttgctctcaattctgtttc

SEQ ID NO: 376 - RNA, 15 nt, synthetic construct
  misc_feature 1..15: note = Description of Artificial Sequence: Synthetic oligonucleotide
  source 1..15: mol_type = other RNA
aatcagaggtgagca

SEQ ID NO: 377 - RNA, 15 nt, synthetic construct
  misc_feature 1..15: note = Description of Artificial Sequence: Synthetic oligonucleotide
  source 1..15: mol_type = other RNA
gaggtgagcactgca

SEQ ID NO: 378 - RNA, 15 nt, synthetic construct
  misc_feature 1..15: note = Description of Artificial Sequence: Synthetic oligonucleotide
  source 1..15: mol_type = other RNA
aggtgagcactgcaa

SEQ ID NO: 379 - RNA, 15 nt, synthetic construct
  misc_feature 1..15: note = Description of Artificial Sequence: Synthetic oligonucleotide
  source 1..15: mol_type = other RNA
tgagcactgcaacaa

SEQ ID NO: 380 - RNA, 15 nt, synthetic construct
  misc_feature 1..15: note = Description of Artificial Sequence: Synthetic oligonucleotide
  source 1..15: mol_type = other RNA
ttttctctcggatca

SEQ ID NO: 381 - RNA, 15 nt, synthetic construct
  misc_feature 1..15: note = Description of Artificial Sequence: Synthetic oligonucleotide
  source 1..15: mol_type = other RNA
tttctctcggatcta

SEQ ID NO: 382 - RNA, 15 nt, synthetic construct
  misc_feature 1..15: note = Description of Artificial Sequence: Synthetic oligonucleotide
  source 1..15: mol_type = other RNA
ctctcggatctccaa

SEQ ID NO: 383 - RNA, 15 nt, synthetic construct
  misc_feature 1..15: note = Description of Artificial Sequence: Synthetic oligonucleotide
  source 1..15: mol_type = other RNA
tctcggatctccaaa

SEQ ID NO: 384 - RNA, 15 nt, synthetic construct
  misc_feature 1..15: note = Description of Artificial Sequence: Synthetic oligonucleotide
  source 1..15: mol_type = other RNA
tcggatctccaaata

SEQ ID NO: 385 - RNA, 15 nt, synthetic construct
  misc_feature 1..15: note = Description of Artificial Sequence: Synthetic oligonucleotide
  source 1..15: mol_type = other RNA
tccaaatttaaaaga

SEQ ID NO: 386 - RNA, 15 nt, synthetic construct
  misc_feature 1..15: note = Description of Artificial Sequence: Synthetic oligonucleotide
  source 1..15: mol_type = other RNA
ccaaatttaaaagca

SEQ ID NO: 387 - RNA, 15 nt, synthetic construct
  misc_feature 1..15: note = Description of Artificial Sequence: Synthetic oligonucleotide
  source 1..15: mol_type = other RNA
caaatttaaaagcaa

SEQ ID NO: 388 - RNA, 15 nt, synthetic construct
  misc_feature 1..15: note = Description of Artificial Sequence: Synthetic oligonucleotide
  source 1..15: mol_type = other RNA
aagcacaaggaatga

SEQ ID NO: 389 - RNA, 15 nt, synthetic construct
  misc_feature 1..15: note = Description of Artificial Sequence: Synthetic oligonucleotide
  source 1..15: mol_type = other RNA
gaatgattgtaccaa

SEQ ID NO: 390 - RNA, 15 nt, synthetic construct
  misc_feature 1..15: note = Description of Artificial Sequence: Synthetic oligonucleotide
  source 1..15: mol_type = other RNA
attgtaccacacaaa

SEQ ID NO: 391 - RNA, 15 nt, synthetic construct
  misc_feature 1..15: note = Description of Artificial Sequence: Synthetic oligonucleotide
  source 1..15: mol_type = other RNA
gtaccacacaaagta

SEQ ID NO: 392 - RNA, 15 nt, synthetic construct
  misc_feature 1..15: note = Description of Artificial Sequence: Synthetic oligonucleotide
  source 1..15: mol_type = other RNA
taccacacaaagtaa

SEQ ID NO: 393 - RNA, 15 nt, synthetic construct
  misc_feature 1..15: note = Description of Artificial Sequence: Synthetic oligonucleotide
  source 1..15: mol_type = other RNA
ccacacaaagtaata

SEQ ID NO: 394 - RNA, 15 nt, synthetic construct
  misc_feature 1..15: note = Description of Artificial Sequence: Synthetic oligonucleotide
  source 1..15: mol_type = other RNA
acacaaagtaatgta

SEQ ID NO: 395 - RNA, 15 nt, synthetic construct
  misc_feature 1..15: note = Description of Artificial Sequence: Synthetic oligonucleotide
  source 1..15: mol_type = other RNA
agtaatgtaaaacaa

SEQ ID NO: 396 - RNA, 15 nt, synthetic construct
  misc_feature 1..15: note = Description of Artificial Sequence: Synthetic oligonucleotide
  source 1..15: mol_type = other RNA
gtaatgtaaaacata

SEQ ID NO: 397 - RNA, 15 nt, synthetic construct
  misc_feature 1..15: note = Description of Artificial Sequence: Synthetic oligonucleotide
  source 1..15: mol_type = other RNA
taaaacattaaagga

SEQ ID NO: 398 - RNA, 15 nt, synthetic construct
  misc_feature 1..15: note = Description of Artificial Sequence: Synthetic oligonucleotide
  source 1..15: mol_type = other RNA
acattaaaggactca

SEQ ID NO: 399 - RNA, 15 nt, synthetic construct
  misc_feature 1..15: note = Description of Artificial Sequence: Synthetic oligonucleotide
  source 1..15: mol_type = other RNA
ggactcattaaaaaa

SEQ ID NO: 400 - RNA, 15 nt, synthetic construct
  misc_feature 1..15: note = Description of Artificial Sequence: Synthetic oligonucleotide
  source 1..15: mol_type = other RNA
actcattaaaaagta

SEQ ID NO: 401 - RNA, 15 nt, synthetic construct
  misc_feature 1..15: note = Description of Artificial Sequence: Synthetic oligonucleotide
  source 1..15: mol_type = other RNA
aaagtaacagttgta

SEQ ID NO: 402 - RNA, 15 nt, synthetic construct
  misc_feature 1..15: note = Description of Artificial Sequence: Synthetic oligonucleotide
  source 1..15: mol_type = other RNA
catcatcatcatcaa

SEQ ID NO: 403 - RNA, 15 nt, synthetic construct
  misc_feature 1..15: note = Description of Artificial Sequence: Synthetic oligonucleotide
  source 1..15: mol_type = other RNA
catcatcatcataga

SEQ ID NO: 404 - RNA, 15 nt, synthetic construct
  misc_feature 1..15: note = Description of Artificial Sequence: Synthetic oligonucleotide
  source 1..15: mol_type = other RNA
catcatcatagctaa

SEQ ID NO: 405 - RNA, 15 nt, synthetic construct
  misc_feature 1..15: note = Description of Artificial Sequence: Synthetic oligonucleotide
  source 1..15: mol_type = other RNA
atcatcatcatcata

SEQ ID NO: 406 - RNA, 15 nt, synthetic construct
  misc_feature 1..15: note = Description of Artificial Sequence: Synthetic oligonucleotide
  source 1..15: mol_type = other RNA
catcatcatcatcaa

SEQ ID NO: 407 - RNA, 15 nt, synthetic construct
  misc_feature 1..15: note = Description of Artificial Sequence: Synthetic oligonucleotide
  source 1..15: mol_type = other RNA
catcatcatcataga

SEQ ID NO: 408 - RNA, 15 nt, synthetic construct
  misc_feature 1..15: note = Description of Artificial Sequence: Synthetic oligonucleotide
  source 1..15: mol_type = other RNA
tcatcatagctacca

SEQ ID NO: 409 - RNA, 15 nt, synthetic construct
  misc_feature 1..15: note = Description of Artificial Sequence: Synthetic oligonucleotide
  source 1..15: mol_type = other RNA
catcatagctaccaa

SEQ ID NO: 410 - RNA, 15 nt, synthetic construct
  misc_feature 1..15: note = Description of Artificial Sequence: Synthetic oligonucleotide
  source 1..15: mol_type = other RNA
agctaccatttatta

SEQ ID NO: 411 - RNA, 15 nt, synthetic construct
  misc_feature 1..15: note = Description of Artificial Sequence: Synthetic oligonucleotide
  source 1..15: mol_type = other RNA
taccatttattgaaa

SEQ ID NO: 412 - RNA, 15 nt, synthetic construct
  misc_feature 1..15: note = Description of Artificial Sequence: Synthetic oligonucleotide
  source 1..15: mol_type = other RNA
aacttcaaagaacta

SEQ ID NO: 413 - RNA, 15 nt, synthetic construct
  misc_feature 1..15: note = Description of Artificial Sequence: Synthetic oligonucleotide
  source 1..15: mol_type = other RNA
caaagaacttatcca

SEQ ID NO: 414 - RNA, 15 nt, synthetic construct
  misc_feature 1..15: note = Description of Artificial Sequence: Synthetic oligonucleotide
  source 1..15: mol_type = other RNA
tcctttagttggaga

SEQ ID NO: 415 - RNA, 15 nt, synthetic construct
  misc_feature 1..15: note = Description of Artificial Sequence: Synthetic oligonucleotide
  source 1..15: mol_type = other RNA
ctttagttggagaga

SEQ ID NO: 416 - RNA, 15 nt, synthetic construct
  misc_feature 1..15: note = Description of Artificial Sequence: Synthetic oligonucleotide
  source 1..15: mol_type = other RNA
ttagttggagagcca

SEQ ID NO: 417 - RNA, 15 nt, synthetic construct
  misc_feature 1..15: note = Description of Artificial Sequence: Synthetic oligonucleotide
  source 1..15: mol_type = other RNA
tagttggagagccaa

SEQ ID NO: 418 - RNA, 15 nt, synthetic construct
  misc_feature 1..15: note = Description of Artificial Sequence: Synthetic oligonucleotide
  source 1..15: mol_type = other RNA
ttggagagccaagaa

SEQ ID NO: 419 - RNA, 15 nt, synthetic construct
  misc_feature 1..15: note = Description of Artificial Sequence: Synthetic oligonucleotide
  source 1..15: mol_type = other RNA
ggagagccaagacaa

SEQ ID NO: 420 - RNA, 15 nt, synthetic construct
  misc_feature 1..15: note = Description of Artificial Sequence: Synthetic oligonucleotide
  source 1..15: mol_type = other RNA
gagccaagacaatca

SEQ ID NO: 421 - RNA, 15 nt, synthetic construct
  misc_feature 1..15: note = Description of Artificial Sequence: Synthetic oligonucleotide
  source 1..15: mol_type = other RNA
ccaagacaatcataa

SEQ ID NO: 422 - RNA, 15 nt, synthetic construct
  misc_feature 1..15: note = Description of Artificial Sequence: Synthetic oligonucleotide
  source 1..15: mol_type = other RNA
aagacaatcataaca

SEQ ID NO: 423 - RNA, 15 nt, synthetic construct
  misc_feature 1..15: note = Description of Artificial Sequence: Synthetic oligonucleotide
  source 1..15: mol_type = other RNA
agctgtctgcttcta

SEQ ID NO: 424 - RNA, 15 nt, synthetic construct
  misc_feature 1..15: note = Description of Artificial Sequence: Synthetic oligonucleotide
  source 1..15: mol_type = other RNA
gatcctgaactgaga

SEQ ID NO: 425 - RNA, 15 nt, synthetic construct
  misc_feature 1..15: note = Description of Artificial Sequence: Synthetic oligonucleotide
  source 1..15: mol_type = other RNA
atcctgaactgagta

SEQ ID NO: 426 - RNA, 15 nt, synthetic construct
  misc_feature 1..15: note = Description of Artificial Sequence: Synthetic oligonucleotide
  source 1..15: mol_type = other RNA
tgaactgagtttaaa

SEQ ID NO: 427 - RNA, 15 nt, synthetic construct
  misc_feature 1..15: note = Description of Artificial Sequence: Synthetic oligonucleotide
  source 1..15: mol_type = other RNA
gtttaaaaggcacca

SEQ ID NO: 428 - RNA, 15 nt, synthetic construct
  misc_feature 1..15: note = Description of Artificial Sequence: Synthetic oligonucleotide
  source 1..15: mol_type = other RNA
atcaaatgcaacgta

SEQ ID NO: 429 - RNA, 15 nt, synthetic construct
  misc_feature 1..15: note = Description of Artificial Sequence: Synthetic oligonucleotide
  source 1..15: mol_type = other RNA
tcaaatgcaacgtaa

SEQ ID NO: 430 - RNA, 15 nt, synthetic construct
  misc_feature 1..15: note = Description of Artificial Sequence: Synthetic oligonucleotide
  source 1..15: mol_type = other RNA
gttgtatggttaaaa

SEQ ID NO: 431 - RNA, 15 nt, synthetic construct
  misc_feature 1..15: note = Description of Artificial Sequence: Synthetic oligonucleotide
  source 1..15: mol_type = other RNA
tttaaaaacctcaca

SEQ ID NO: 432 - RNA, 15 nt, synthetic construct
  misc_feature 1..15: note = Description of Artificial Sequence: Synthetic oligonucleotide
  source 1..15: mol_type = other RNA
taaaaacctcactga

SEQ ID NO: 433 - RNA, 15 nt, synthetic construct
  misc_feature 1..15: note = Description of Artificial Sequence: Synthetic oligonucleotide
  source 1..15: mol_type = other RNA
aaaacctcactgcca

SEQ ID NO: 434 - RNA, 15 nt, synthetic construct
  misc_feature 1..15: note = Description of Artificial Sequence: Synthetic oligonucleotide
  source 1..15: mol_type = other RNA
gaaacagaattgaga

SEQ ID NO: 435 - RNA, 20 nt, synthetic construct
  misc_feature 1..20: note = Description of Artificial Sequence: Synthetic oligonucleotide
  source 1..20: mol_type = other RNA
tgcagtgctcacctctgatt

SEQ ID NO: 436 - RNA, 20 nt, synthetic construct
  misc_feature 1..20: note = Description of Artificial Sequence: Synthetic oligonucleotide
  source 1..20: mol_type = other RNA
tttgttgcagtgctcacctc

SEQ ID NO: 437 - RNA, 20 nt, synthetic construct
  misc_feature 1..20: note = Description of Artificial Sequence: Synthetic oligonucleotide
  source 1..20: mol_type = other RNA
ttttgttgcagtgctcacct

SEQ ID NO: 438 - RNA, 20 nt, synthetic construct
  misc_feature 1..20: note = Description of Artificial Sequence: Synthetic oligonucleotide
  source 1..20: mol_type = other RNA
tctttttgttgcagtgctca

SEQ ID NO: 439 - RNA, 20 nt, synthetic construct
  misc_feature 1..20: note = Description of Artificial Sequence: Synthetic oligonucleotide
  source 1..20: mol_type = other RNA
tttggagatccgagagaaaa

SEQ ID NO: 440 - RNA, 20 nt, synthetic construct
  misc_feature 1..20: note = Description of Artificial Sequence: Synthetic oligonucleotide
  source 1..20: mol_type = other RNA
ttttggagatccgagagaaa

SEQ ID NO: 441 - RNA, 20 nt, synthetic construct
  misc_feature 1..20: note = Description of Artificial Sequence: Synthetic oligonucleotide
  source 1..20: mol_type = other RNA
taaatttggagatccgagag

SEQ ID NO: 442 - RNA, 20 nt, synthetic construct
  misc_feature 1..20: note = Description of Artificial Sequence: Synthetic oligonucleotide
  source 1..20: mol_type = other RNA
ttaaatttggagatccgaga

SEQ ID NO: 443 - RNA, 20 nt, synthetic construct
  misc_feature 1..20: note = Description of Artificial Sequence: Synthetic oligonucleotide
  source 1..20: mol_type = other RNA
ttttaaatttggagatccga

SEQ ID NO: 444 - RNA, 20 nt, synthetic construct
  misc_feature 1..20: note = Description of Artificial Sequence: Synthetic oligonucleotide
  source 1..20: mol_type = other RNA
tttgtgcttttaaatttgga

SEQ ID NO: 445 - RNA, 20 nt, synthetic construct
  misc_feature 1..20: note = Description of Artificial Sequence: Synthetic oligonucleotide
  source 1..20: mol_type = other RNA
tcttgtgcttttaaatttgg

SEQ ID NO: 446 - RNA, 20 nt, synthetic construct
  misc_feature 1..20: note = Description of Artificial Sequence: Synthetic oligonucleotide
  source 1..20: mol_type = other RNA
tccttgtgcttttaaatttg

SEQ ID NO: 447 - RNA, 20 nt, synthetic construct
  misc_feature 1..20: note = Description of Artificial Sequence: Synthetic oligonucleotide
  source 1..20: mol_type = other RNA
tacaatcattccttgtgctt

SEQ ID NO: 448 - RNA, 20 nt, synthetic construct
  misc_feature 1..20: note = Description of Artificial Sequence: Synthetic oligonucleotide
  source 1..20: mol_type = other RNA
tttgtgtggtacaatcattc

SEQ ID NO: 449 - RNA, 20 nt, synthetic construct
  misc_feature 1..20: note = Description of Artificial Sequence: Synthetic oligonucleotide
  source 1..20: mol_type = other RNA
tttactttgtgtggtacaat

SEQ ID NO: 450 - RNA, 20 nt, synthetic construct
  misc_feature 1..20: note = Description of Artificial Sequence: Synthetic oligonucleotide
  source 1..20: mol_type = other RNA
tacattactttgtgtggtac

SEQ ID NO: 451 - RNA, 20 nt, synthetic construct
  misc_feature 1..20: note = Description of Artificial Sequence: Synthetic oligonucleotide
  source 1..20: mol_type = other RNA
ttacattactttgtgtggta

SEQ ID NO: 452 - RNA, 20 nt, synthetic construct
  misc_feature 1..20: note = Description of Artificial Sequence: Synthetic oligonucleotide
  source 1..20: mol_type = other RNA
ttttacattactttgtgtgg

SEQ ID NO: 453 - RNA, 20 nt, synthetic construct
  misc_feature 1..20: note = Description of Artificial Sequence: Synthetic oligonucleotide
  source 1..20: mol_type = other RNA
tgttttacattactttgtgt

SEQ ID NO: 454 - RNA, 20 nt, synthetic construct
  misc_feature 1..20: note = Description of Artificial Sequence: Synthetic oligonucleotide
  source 1..20: mol_type = other RNA
ttttaatgttttacattact

SEQ ID NO: 455 - RNA, 20 nt, synthetic construct
  misc_feature 1..20: note = Description of Artificial Sequence: Synthetic oligonucleotide
  source 1..20: mol_type = other RNA
tctttaatgttttacattac

SEQ ID NO: 456 - RNA, 20 nt, synthetic construct
  misc_feature 1..20: note = Description of Artificial Sequence: Synthetic oligonucleotide
  source 1..20: mol_type = other RNA
ttgagtcctttaatgtttta

SEQ ID NO: 457 - RNA, 20 nt, synthetic construct
  misc_feature 1..20: note = Description of Artificial Sequence: Synthetic oligonucleotide
  source 1..20: mol_type = other RNA
tttaatgagtcctttaatgt

SEQ ID NO: 458 - RNA, 20 nt, synthetic construct
  misc_feature 1..20: note = Description of Artificial Sequence: Synthetic oligonucleotide
  source 1..20: mol_type = other RNA
tgttactttttaatgagtcc

SEQ ID NO: 459 - RNA, 20 nt, synthetic construct
  misc_feature 1..20: note = Description of Artificial Sequence: Synthetic oligonucleotide
  source 1..20: mol_type = other RNA
tctgttactttttaatgagt

SEQ ID NO: 460 - RNA, 20 nt, synthetic construct
  misc_feature 1..20: note = Description of Artificial Sequence: Synthetic oligonucleotide
  source 1..20: mol_type = other RNA
tatgagacaactgttacttt

SEQ ID NO: 461 - RNA, 20 nt, synthetic construct
  misc_feature 1..20: note = Description of Artificial Sequence: Synthetic oligonucleotide
  source 1..20: mol_type = other RNA
tagctatgatgatgatgatg

SEQ ID NO: 462 - RNA, 20 nt, synthetic construct
  misc_feature 1..20: note = Description of Artificial Sequence: Synthetic oligonucleotide
  source 1..20: mol_type = other RNA
tgatagctatgatgatgatg

SEQ ID NO: 463 - RNA, 20 nt, synthetic construct
  misc_feature 1..20: note = Description of Artificial Sequence: Synthetic oligonucleotide
  source 1..20: mol_type = other RNA
tgatgatagctatgatgatg

SEQ ID NO: 464 - RNA, 20 nt, synthetic construct
  misc_feature 1..20: note = Description of Artificial Sequence: Synthetic oligonucleotide
  source 1..20: mol_type = other RNA
tctatgatgatgatgatgat

SEQ ID NO: 465 - RNA, 20 nt, synthetic construct
  misc_feature 1..20: note = Description of Artificial Sequence: Synthetic oligonucleotide
  source 1..20: mol_type = other RNA
tagctatgatgatgatgatg

SEQ ID NO: 466 - RNA, 20 nt, synthetic construct
  misc_feature 1..20: note = Description of Artificial Sequence: Synthetic oligonucleotide
  source 1..20: mol_type = other RNA
tggtagctatgatgatgatg

SEQ ID NO: 467 - RNA, 20 nt, synthetic construct
  misc_feature 1..20: note = Description of Artificial Sequence: Synthetic oligonucleotide
  source 1..20: mol_type = other RNA
ttaaatggtagctatgatga

SEQ ID NO: 468 - RNA, 20 nt, synthetic construct
  misc_feature 1..20: note = Description of Artificial Sequence: Synthetic oligonucleotide
  source 1..20: mol_type = other RNA
tataaatggtagctatgatg

SEQ ID NO: 469 - RNA, 20 nt, synthetic construct
  misc_feature 1..20: note = Description of Artificial Sequence: Synthetic oligonucleotide
  source 1..20: mol_type = other RNA
tttttcaataaatggtagct

SEQ ID NO: 470 - RNA, 20 nt, synthetic construct
  misc_feature 1..20: note = Description of Artificial Sequence: Synthetic oligonucleotide
  source 1..20: mol_type = other RNA
ttagttttcaataaatggta

SEQ ID NO: 471 - RNA, 20 nt, synthetic construct
  misc_feature 1..20: note = Description of Artificial Sequence: Synthetic oligonucleotide
  source 1..20: mol_type = other RNA
tggataagttctttgaagtt

SEQ ID NO: 472 - RNA, 20 nt, synthetic construct
  misc_feature 1..20: note = Description of Artificial Sequence: Synthetic oligonucleotide
  source 1..20: mol_type = other RNA
tctaaaggataagttctttg

SEQ ID NO: 473 - RNA, 20 nt, synthetic construct
  misc_feature 1..20: note = Description of Artificial Sequence: Synthetic oligonucleotide
  source 1..20: mol_type = other RNA
ttggctctccaactaaagga

SEQ ID NO: 474 - RNA, 20 nt, synthetic construct
  misc_feature 1..20: note = Description of Artificial Sequence: Synthetic oligonucleotide
  source 1..20: mol_type = other RNA
tcttggctctccaactaaag

SEQ ID NO: 475 - RNA, 20 nt, synthetic construct
  misc_feature 1..20: note = Description of Artificial Sequence: Synthetic oligonucleotide
  source 1..20: mol_type = other RNA
tgtcttggctctccaactaa

SEQ ID NO: 476 - RNA, 20 nt, synthetic construct
  misc_feature 1..20: note = Description of Artificial Sequence: Synthetic oligonucleotide
  source 1..20: mol_type = other RNA
ttgtcttggctctccaacta

SEQ ID NO: 477 - RNA, 20 nt, synthetic construct
  misc_feature 1..20: note = Description of Artificial Sequence: Synthetic oligonucleotide
  source 1..20: mol_type = other RNA
tgattgtcttggctctccaa

SEQ ID NO: 478 - RNA, 20 nt, synthetic construct
  misc_feature 1..20: note = Description of Artificial Sequence: Synthetic oligonucleotide
  source 1..20: mol_type = other RNA
tatgattgtcttggctctcc

SEQ ID NO: 479 - RNA, 20 nt, synthetic construct
  misc_feature 1..20: note = Description of Artificial Sequence: Synthetic oligonucleotide
  source 1..20: mol_type = other RNA
tgttatgattgtcttggctc

SEQ ID NO: 480 - RNA, 20 nt, synthetic construct
  misc_feature 1..20: note = Description of Artificial Sequence: Synthetic oligonucleotide
  source 1..20: mol_type = other RNA
tattgttatgattgtcttgg

SEQ ID NO: 481 - RNA, 20 nt, synthetic construct
  misc_feature 1..20: note = Description of Artificial Sequence: Synthetic oligonucleotide
  source 1..20: mol_type = other RNA
tttattgttatgattgtctt

SEQ ID NO: 482 - RNA, 20 nt, synthetic construct
  misc_feature 1..20: note = Description of Artificial Sequence: Synthetic oligonucleotide
  source 1..20: mol_type = other RNA
tctgtgagaagcagacagct

SEQ ID NO: 483 - RNA, 20 nt, synthetic construct
  misc_feature 1..20: note = Description of Artificial Sequence: Synthetic oligonucleotide
  source 1..20: mol_type = other RNA
tttaaactcagttcaggatc

SEQ ID NO: 484 - RNA, 20 nt, synthetic construct
  misc_feature 1..20: note = Description of Artificial Sequence: Synthetic oligonucleotide
  source 1..20: mol_type = other RNA
ttttaaactcagttcaggat

SEQ ID NO: 485 - RNA, 20 nt, synthetic construct
  misc_feature 1..20: note = Description of Artificial Sequence: Synthetic oligonucleotide
  source 1..20: mol_type = other RNA
tgccttttaaactcagttca

SEQ ID NO: 486 - RNA, 20 nt, synthetic construct
  misc_feature 1..20: note = Description of Artificial Sequence: Synthetic oligonucleotide
  source 1..20: mol_type = other RNA
ttgctgggtgccttttaaac

SEQ ID NO: 487 - RNA, 20 nt, synthetic construct
  misc_feature 1..20: note = Description of Artificial Sequence: Synthetic oligonucleotide
  source 1..20: mol_type = other RNA
ttttgtacgttgcatttgat

SEQ ID NO: 488 - RNA, 20 nt, synthetic construct
  misc_feature 1..20: note = Description of Artificial Sequence: Synthetic oligonucleotide
  source 1..20: mol_type = other RNA
tctttgtacgttgcatttga

SEQ ID NO: 489 - RNA, 20 nt, synthetic construct
  misc_feature 1..20: note = Description of Artificial Sequence: Synthetic oligonucleotide
  source 1..20: mol_type = other RNA
tcatcttttaaccatacaac

SEQ ID NO: 490 - RNA, 20 nt, synthetic construct
  misc_feature 1..20: note = Description of Artificial Sequence: Synthetic oligonucleotide
  source 1..20: mol_type = other RNA
ttggcagtgaggtttttaaa

SEQ ID NO: 491 - RNA, 20 nt, synthetic construct
  misc_feature 1..20: note = Description of Artificial Sequence: Synthetic oligonucleotide
  source 1..20: mol_type = other RNA
tagtggcagtgaggttttta

SEQ ID NO: 492 - RNA, 20 nt, synthetic construct
  misc_feature 1..20: note = Description of Artificial Sequence: Synthetic oligonucleotide
  source 1..20: mol_type = other RNA
tagagtggcagtgaggtttt

SEQ ID NO: 493 - RNA, 20 nt, synthetic construct
  misc_feature 1..20: note = Description of Artificial Sequence: Synthetic oligonucleotide
  source 1..20: mol_type = other RNA
tatgctctcaattctgtttc

SEQ ID NO: 494 - RNA, 15 nt, synthetic construct
  misc_feature 1..15: note = Description of Artificial Sequence: Synthetic oligonucleotide
  source 1..15: mol_type = other RNA
aatcagaggtgagca

SEQ ID NO: 495 - RNA, 15 nt, synthetic construct
  misc_feature 1..15: note = Description of Artificial Sequence: Synthetic oligonucleotide
  source 1..15: mol_type = other RNA
gaggtgagcactgca

SEQ ID NO: 496 - RNA, 15 nt, synthetic construct
  misc_feature 1..15: note = Description of Artificial Sequence: Synthetic oligonucleotide
  source 1..15: mol_type = other RNA
aggtgagcactgcaa

SEQ ID NO: 497 - RNA, 15 nt, synthetic construct
  misc_feature 1..15: note = Description of Artificial Sequence: Synthetic oligonucleotide
  source 1..15: mol_type = other RNA
tgagcactgcaacaa

SEQ ID NO: 498 - RNA, 15 nt, synthetic construct
  misc_feature 1..15: note = Description of Artificial Sequence: Synthetic oligonucleotide
  source 1..15: mol_type = other RNA
ttttctctcggatca

SEQ ID NO: 499 - RNA, 15 nt, synthetic construct
  misc_feature 1..15: note = Description of Artificial Sequence: Synthetic oligonucleotide
  source 1..15: mol_type = other RNA
tttctctcggatcta

SEQ ID NO: 500 - RNA, 15 nt, synthetic construct
  misc_feature 1..15: note = Description of Artificial Sequence: Synthetic oligonucleotide
  source 1..15: mol_type = other RNA
ctctcggatctccaa

SEQ ID NO: 501 - RNA, 15 nt, synthetic construct
  misc_feature 1..15: note = Description of Artificial Sequence: Synthetic oligonucleotide
  source 1..15: mol_type = other RNA
tctcggatctccaaa

SEQ ID NO: 502 - RNA, 15 nt, synthetic construct
  misc_feature 1..15: note = Description of Artificial Sequence: Synthetic oligonucleotide
  source 1..15: mol_type = other RNA
tcggatctccaaata

SEQ ID NO: 503 - RNA, 15 nt, synthetic construct
  misc_feature 1..15: note = Description of Artificial Sequence: Synthetic oligonucleotide
  source 1..15: mol_type = other RNA
tccaaatttaaaaga

SEQ ID NO: 504 - RNA, 15 nt, synthetic construct
  misc_feature 1..15: note = Description of Artificial Sequence: Synthetic oligonucleotide
  source 1..15: mol_type = other RNA
ccaaatttaaaagca

SEQ ID NO: 505 - RNA, 15 nt, synthetic construct
  misc_feature 1..15: note = Description of Artificial Sequence: Synthetic oligonucleotide
  source 1..15: mol_type = other RNA
caaatttaaaagcaa

SEQ ID NO: 506 - RNA, 15 nt, synthetic construct
  misc_feature 1..15: note = Description of Artificial Sequence: Synthetic oligonucleotide
  source 1..15: mol_type = other RNA
aagcacaaggaatga

SEQ ID NO: 507 - RNA, 15 nt, synthetic construct
  misc_feature 1..15: note = Description of Artificial Sequence: Synthetic oligonucleotide
  source 1..15: mol_type = other RNA
gaatgattgtaccaa

SEQ ID NO: 508 - RNA, 15 nt, synthetic construct
  misc_feature 1..15: note = Description of Artificial Sequence: Synthetic oligonucleotide
  source 1..15: mol_type = other RNA
attgtaccacacaaa

SEQ ID NO: 509 - RNA, 15 nt, synthetic construct
  misc_feature 1..15: note = Description of Artificial Sequence: Synthetic oligonucleotide
  source 1..15: mol_type = other RNA
gtaccacacaaagta

SEQ ID NO: 510 - RNA, 15 nt, synthetic construct
  misc_feature 1..15: note = Description of Artificial Sequence: Synthetic oligonucleotide
  source 1..15: mol_type = other RNA
taccacacaaagtaa

SEQ ID NO: 511 - RNA, 15 nt, synthetic construct
  misc_feature 1..15: note = Description of Artificial Sequence: Synthetic oligonucleotide
  source 1..15: mol_type = other RNA
ccacacaaagtaata

SEQ ID NO: 512 - RNA, 15 nt, synthetic construct
  misc_feature 1..15: note = Description of Artificial Sequence: Synthetic oligonucleotide
  source 1..15: mol_type = other RNA
acacaaagtaatgta

SEQ ID NO: 513 - RNA, 15 nt, synthetic construct
  misc_feature 1..15: note = Description of Artificial Sequence: Synthetic oligonucleotide
  source 1..15: mol_type = other RNA
agtaatgtaaaacaa

SEQ ID NO: 514 - RNA, 15 nt, synthetic construct
  misc_feature 1..15: note = Description of Artificial Sequence: Synthetic oligonucleotide
  source 1..15: mol_type = other RNA
gtaatgtaaaacata

SEQ ID NO: 515 - RNA, 15 nt, synthetic construct
  misc_feature 1..15: note = Description of Artificial Sequence: Synthetic oligonucleotide
  source 1..15: mol_type = other RNA
taaaacattaaagga

SEQ ID NO: 516 - RNA, 15 nt, synthetic construct
  misc_feature 1..15: note = Description of Artificial Sequence: Synthetic oligonucleotide
  source 1..15: mol_type = other RNA
acattaaaggactca

SEQ ID NO: 517 - RNA, 15 nt, synthetic construct
  misc_feature 1..15: note = Description of Artificial Sequence: Synthetic oligonucleotide
  source 1..15: mol_type = other RNA
ggactcattaaaaaa

SEQ ID NO: 518 - RNA, 15 nt, synthetic construct
  misc_feature 1..15: note = Description of Artificial Sequence: Synthetic oligonucleotide
  source 1..15: mol_type = other RNA
actcattaaaaagta

SEQ ID NO: 519 - RNA, 15 nt, synthetic construct
  misc_feature 1..15: note = Description of Artificial Sequence: Synthetic oligonucleotide
  source 1..15: mol_type = other RNA
aaagtaacagttgta

SEQ ID NO: 520 - RNA, 15 nt, synthetic construct
  misc_feature 1..15: note = Description of Artificial Sequence: Synthetic oligonucleotide
  source 1..15: mol_type = other RNA
catcatcatcatcaa

SEQ ID NO: 521 - RNA, 15 nt, synthetic construct
  misc_feature 1..15: note = Description of Artificial Sequence: Synthetic oligonucleotide
  source 1..15: mol_type = other RNA
catcatcatcataga

SEQ ID NO: 522 - RNA, 15 nt, synthetic construct
  misc_feature 1..15: note = Description of Artificial Sequence: Synthetic oligonucleotide
  source 1..15: mol_type = other RNA
catcatcatagctaa

SEQ ID NO: 523 - RNA, 15 nt, synthetic construct
  misc_feature 1..15: note = Description of Artificial Sequence: Synthetic oligonucleotide
  source 1..15: mol_type = other RNA
atcatcatcatcata

SEQ ID NO: 524 - RNA, 15 nt, synthetic construct
  misc_feature 1..15: note = Description of Artificial Sequence: Synthetic oligonucleotide
  source 1..15: mol_type = other RNA
catcatcatcatcaa

SEQ ID NO: 525 - RNA, 15 nt, synthetic construct
  misc_feature 1..15: note = Description of Artificial Sequence: Synthetic oligonucleotide
  source 1..15: mol_type = other RNA
catcatcatcataga

SEQ ID NO: 526 - RNA, 15 nt, synthetic construct
  misc_feature 1..15: note = Description of Artificial Sequence: Synthetic oligonucleotide
  source 1..15: mol_type = other RNA
tcatcatagctacca

SEQ ID NO: 527 - RNA, 15 nt, synthetic construct
  misc_feature 1..15: note = Description of Artificial Sequence: Synthetic oligonucleotide
  source 1..15: mol_type = other RNA
catcatagctaccaa

SEQ ID NO: 528 - RNA, 15 nt, synthetic construct
  misc_feature 1..15: note = Description of Artificial Sequence: Synthetic oligonucleotide
  source 1..15: mol_type = other RNA
agctaccatttatta

SEQ ID NO: 529 - RNA, 15 nt, synthetic construct
  misc_feature 1..15: note = Description of Artificial Sequence: Synthetic oligonucleotide
  source 1..15: mol_type = other RNA
taccatttattgaaa

SEQ ID NO: 530 - RNA, 15 nt, synthetic construct
  misc_feature 1..15: note = Description of Artificial Sequence: Synthetic oligonucleotide
  source 1..15: mol_type = other RNA
aacttcaaagaacta

SEQ ID NO: 531 - RNA, 15 nt, synthetic construct
  misc_feature 1..15: note = Description of Artificial Sequence: Synthetic oligonucleotide
  source 1..15: mol_type = other RNA
caaagaacttatcca

SEQ ID NO: 532 - RNA, 15 nt, synthetic construct
  misc_feature 1..15: note = Description of Artificial Sequence: Synthetic oligonucleotide
  source 1..15: mol_type = other RNA
tcctttagttggaga

SEQ ID NO: 533 - RNA, 15 nt, synthetic construct
  misc_feature 1..15: note = Description of Artificial Sequence: Synthetic oligonucleotide
  source 1..15: mol_type = other RNA
ctttagttggagaga

SEQ ID NO: 534 - RNA, 15 nt, synthetic construct
  misc_feature 1..15: note = Description of Artificial Sequence: Synthetic oligonucleotide
  source 1..15: mol_type = other RNA
ttagttggagagcca

SEQ ID NO: 535 - RNA, 15 nt, synthetic construct
  misc_feature 1..15: note = Description of Artificial Sequence: Synthetic oligonucleotide
  source 1..15: mol_type = other RNA
tagttggagagccaa

SEQ ID NO: 536 - RNA, 15 nt, synthetic construct
  misc_feature 1..15: note = Description of Artificial Sequence: Synthetic oligonucleotide
  source 1..15: mol_type = other RNA
ttggagagccaagaa

SEQ ID NO: 537 - RNA, 15 nt, synthetic construct
  misc_feature 1..15: note = Description of Artificial Sequence: Synthetic oligonucleotide
  source 1..15: mol_type = other RNA
ggagagccaagacaa

SEQ ID NO: 538 - RNA, 15 nt, synthetic construct
  misc_feature 1..15: note = Description of Artificial Sequence: Synthetic oligonucleotide
  source 1..15: mol_type = other RNA
gagccaagacaatca

SEQ ID NO: 539 - RNA, 15 nt, synthetic construct
  misc_feature 1..15: note = Description of Artificial Sequence: Synthetic oligonucleotide
  source 1..15: mol_type = other RNA
ccaagacaatcataa

SEQ ID NO: 540 - RNA, 15 nt, synthetic construct
  misc_feature 1..15: note = Description of Artificial Sequence: Synthetic oligonucleotide
  source 1..15: mol_type = other RNA
aagacaatcataaca

SEQ ID NO: 541 - RNA, 15 nt, synthetic construct
  misc_feature 1..15: note = Description of Artificial Sequence: Synthetic oligonucleotide
  source 1..15: mol_type = other RNA
agctgtctgcttcta

SEQ ID NO: 542 - RNA, 15 nt, synthetic construct
  misc_feature 1..15: note = Description of Artificial Sequence: Synthetic oligonucleotide
  source 1..15: mol_type = other RNA
gatcctgaactgaga

SEQ ID NO: 543 - RNA, 15 nt, synthetic construct
  misc_feature 1..15: note = Description of Artificial Sequence: Synthetic oligonucleotide
  source 1..15: mol_type = other RNA
atcctgaactgagta

SEQ ID NO: 544 - RNA, 15 nt, synthetic construct
  misc_feature 1..15: note = Description of Artificial Sequence: Synthetic oligonucleotide
  source 1..15: mol_type = other RNA
tgaactgagtttaaa

SEQ ID NO: 545 - RNA, 15 nt, synthetic construct
  misc_feature 1..15: note = Description of Artificial Sequence: Synthetic oligonucleotide
  source 1..15: mol_type = other RNA
gtttaaaaggcacca

SEQ ID NO: 546 - RNA, 15 nt, synthetic construct
  misc_feature 1..15: note = Description of Artificial Sequence: Synthetic oligonucleotide
  source 1..15: mol_type = other RNA
atcaaatgcaacgta

SEQ ID NO: 547 - RNA, 15 nt, synthetic construct
  misc_feature 1..15: note = Description of Artificial Sequence: Synthetic oligonucleotide
  source 1..15: mol_type = other RNA
tcaaatgcaacgtaa

SEQ ID NO: 548 - RNA, 15 nt, synthetic construct
  misc_feature 1..15: note = Description of Artificial Sequence: Synthetic oligonucleotide
  source 1..15: mol_type = other RNA
gttgtatggttaaaa

SEQ ID NO: 549 - RNA, 15 nt, synthetic construct
  misc_feature 1..15: note = Description of Artificial Sequence: Synthetic oligonucleotide
  source 1..15: mol_type = other RNA
tttaaaaacctcaca

SEQ ID NO: 550 - RNA, 15 nt, synthetic construct
  misc_feature 1..15: note = Description of Artificial Sequence: Synthetic oligonucleotide
  source 1..15: mol_type = other RNA
taaaaacctcactga

SEQ ID NO: 551 - RNA, 15 nt, synthetic construct
  misc_feature 1..15: note = Description of Artificial Sequence: Synthetic oligonucleotide
  source 1..15: mol_type = other RNA
aaaacctcactgcca

SEQ ID NO: 552 - RNA, 15 nt, synthetic construct
  misc_feature 1..15: note = Description of Artificial Sequence: Synthetic oligonucleotide
  source 1..15: mol_type = other RNA
gaaacagaattgaga

SEQ ID NO: 553 - RNA, 20 nt, synthetic construct
  misc_feature 1..20: note = Description of Artificial Sequence: Synthetic oligonucleotide
  source 1..20: mol_type = other RNA
tgcagtgctcacctctgatt

SEQ ID NO: 554 - RNA, 20 nt, synthetic construct
  misc_feature 1..20: note = Description of Artificial Sequence: Synthetic oligonucleotide
  source 1..20: mol_type = other RNA
tttgttgcagtgctcacctc

SEQ ID NO: 555 - RNA, 20 nt, synthetic construct
  misc_feature 1..20: note = Description of Artificial Sequence: Synthetic oligonucleotide
  source 1..20: mol_type = other RNA
ttttgttgcagtgctcacct

SEQ ID NO: 556 - RNA, 20 nt, synthetic construct
  misc_feature 1..20: note = Description of Artificial Sequence: Synthetic oligonucleotide
  source 1..20: mol_type = other RNA
tctttttgttgcagtgctca

SEQ ID NO: 557 - RNA, 20 nt, synthetic construct
  misc_feature 1..20: note = Description of Artificial Sequence: Synthetic oligonucleotide
  source 1..20: mol_type = other RNA
tttggagatccgagagaaaa

SEQ ID NO: 558 - RNA, 20 nt, synthetic construct
  misc_feature 1..20: note = Description of Artificial Sequence: Synthetic oligonucleotide
  source 1..20: mol_type = other RNA
ttttggagatccgagagaaa

SEQ ID NO: 559 - RNA, 20 nt, synthetic construct
  misc_feature 1..20: note = Description of Artificial Sequence: Synthetic oligonucleotide
  source 1..20: mol_type = other RNA
taaatttggagatccgagag

SEQ ID NO: 560 - RNA, 20 nt, synthetic construct
  misc_feature 1..20: note = Description of Artificial Sequence: Synthetic oligonucleotide
  source 1..20: mol_type = other RNA
ttaaatttggagatccgaga

SEQ ID NO: 561 - RNA, 20 nt, synthetic construct
  misc_feature 1..20: note = Description of Artificial Sequence: Synthetic oligonucleotide
  source 1..20: mol_type = other RNA
ttttaaatttggagatccga

SEQ ID NO: 562 - RNA, 20 nt, synthetic construct
  misc_feature 1..20: note = Description of Artificial Sequence: Synthetic oligonucleotide
  source 1..20: mol_type = other RNA
tttgtgcttttaaatttgga

SEQ ID NO: 563 - RNA, 20 nt, synthetic construct
  misc_feature 1..20: note = Description of Artificial Sequence: Synthetic oligonucleotide
  source 1..20: mol_type = other RNA
tcttgtgcttttaaatttgg

SEQ ID NO: 564 - RNA, 20 nt, synthetic construct
  misc_feature 1..20: note = Description of Artificial Sequence: Synthetic oligonucleotide
  source 1..20: mol_type = other RNA
tccttgtgcttttaaatttg

SEQ ID NO: 565 - RNA, 20 nt, synthetic construct
  misc_feature 1..20: note = Description of Artificial Sequence: Synthetic oligonucleotide
  source 1..20: mol_type = other RNA
tacaatcattccttgtgctt

SEQ ID NO: 566 - RNA, 20 nt, synthetic construct
  misc_feature 1..20: note = Description of Artificial Sequence: Synthetic oligonucleotide
  source 1..20: mol_type = other RNA
tttgtgtggtacaatcattc

SEQ ID NO: 567 - RNA, 20 nt, synthetic construct
  misc_feature 1..20: note = Description of Artificial Sequence: Synthetic oligonucleotide
  source 1..20: mol_type = other RNA
tttactttgtgtggtacaat

SEQ ID NO: 568 - RNA, 20 nt, synthetic construct
  misc_feature 1..20: note = Description of Artificial Sequence: Synthetic oligonucleotide
  source 1..20: mol_type = other RNA
tacattactttgtgtggtac

SEQ ID NO: 569 - RNA, 20 nt, synthetic construct
  misc_feature 1..20: note = Description of Artificial Sequence: Synthetic oligonucleotide
  source 1..20: mol_type = other RNA
ttacattactttgtgtggta

SEQ ID NO: 570 - RNA, 20 nt, synthetic construct
  misc_feature 1..20: note = Description of Artificial Sequence: Synthetic oligonucleotide
  source 1..20: mol_type = other RNA
ttttacattactttgtgtgg

SEQ ID NO: 571 - RNA, 20 nt, synthetic construct
  misc_feature 1..20: note = Description of Artificial Sequence: Synthetic oligonucleotide
  source 1..20: mol_type = other RNA
tgttttacattactttgtgt

SEQ ID NO: 572 - RNA, 20 nt, synthetic construct
  misc_feature 1..20: note = Description of Artificial Sequence: Synthetic oligonucleotide
  source 1..20: mol_type = other RNA
ttttaatgttttacattact

SEQ ID NO: 573 - RNA, 20 nt, synthetic construct
  misc_feature 1..20: note = Description of Artificial Sequence: Synthetic oligonucleotide
  source 1..20: mol_type = other RNA
tctttaatgttttacattac

SEQ ID NO: 574 - RNA, 20 nt, synthetic construct
  misc_feature 1..20: note = Description of Artificial Sequence: Synthetic oligonucleotide
  source 1..20: mol_type = other RNA
ttgagtcctttaatgtttta

SEQ ID NO: 575 - RNA, 20 nt, synthetic construct
  misc_feature 1..20: note = Description of Artificial Sequence: Synthetic oligonucleotide
  source 1..20: mol_type = other RNA
tttaatgagtcctttaatgt

SEQ ID NO: 576 - RNA, 20 nt, synthetic construct
  misc_feature 1..20: note = Description of Artificial Sequence: Synthetic oligonucleotide
  source 1..20: mol_type = other RNA
tgttactttttaatgagtcc

SEQ ID NO: 577 - RNA, 20 nt, synthetic construct
  misc_feature 1..20: note = Description of Artificial Sequence: Synthetic oligonucleotide
  source 1..20: mol_type = other RNA
tctgttactttttaatgagt

SEQ ID NO: 578 - RNA, 20 nt, synthetic construct
  misc_feature 1..20: note = Description of Artificial Sequence: Synthetic oligonucleotide
  source 1..20: mol_type = other RNA
tatgagacaactgttacttt

SEQ ID NO: 579 - RNA, 20 nt, synthetic construct
  misc_feature 1..20: note = Description of Artificial Sequence: Synthetic oligonucleotide
  source 1..20: mol_type = other RNA
tagctatgatgatgatgatg

SEQ ID NO: 580 - RNA, 20 nt, synthetic construct
  misc_feature 1..20: note = Description of Artificial Sequence: Synthetic oligonucleotide
  source 1..20: mol_type = other RNA
tgatagctatgatgatgatg

SEQ ID NO: 581 - RNA, 20 nt, synthetic construct
  misc_feature 1..20: note = Description of Artificial Sequence: Synthetic oligonucleotide
  source 1..20: mol_type = other RNA
tgatgatagctatgatgatg

SEQ ID NO: 582 - RNA, 20 nt, synthetic construct
  misc_feature 1..20: note = Description of Artificial Sequence: Synthetic oligonucleotide
  source 1..20: mol_type = other RNA
tctatgatgatgatgatgat

SEQ ID NO: 583 - RNA, 20 nt, synthetic construct
  misc_feature 1..20: note = Description of Artificial Sequence: Synthetic oligonucleotide
  source 1..20: mol_type = other RNA
tagctatgatgatgatgatg

SEQ ID NO: 584 - RNA, 20 nt, synthetic construct
  misc_feature 1..20: note = Description of Artificial Sequence: Synthetic oligonucleotide
  source 1..20: mol_type = other RNA
tggtagctatgatgatgatg

SEQ ID NO: 585 - RNA, 20 nt, synthetic construct
  misc_feature 1..20: note = Description of Artificial Sequence: Synthetic oligonucleotide
  source 1..20: mol_type = other RNA
ttaaatggtagctatgatga

SEQ ID NO: 586 - RNA, 20 nt, synthetic construct
  misc_feature 1..20: note = Description of Artificial Sequence: Synthetic oligonucleotide
  source 1..20: mol_type = other RNA
tataaatggtagctatgatg

SEQ ID NO: 587 - RNA, 20 nt, synthetic construct
  misc_feature 1..20: note = Description of Artificial Sequence: Synthetic oligonucleotide
  source 1..20: mol_type = other RNA
tttttcaataaatggtagct

SEQ ID NO: 588 - RNA, 20 nt, synthetic construct
  misc_feature 1..20: note = Description of Artificial Sequence: Synthetic oligonucleotide
  source 1..20: mol_type = other RNA
ttagttttcaataaatggta

SEQ ID NO: 589 - RNA, 20 nt, synthetic construct
  misc_feature 1..20: note = Description of Artificial Sequence: Synthetic oligonucleotide
  source 1..20: mol_type = other RNA
tggataagttctttgaagtt

SEQ ID NO: 590 - RNA, 20 nt, synthetic construct
  misc_feature 1..20: note = Description of Artificial Sequence: Synthetic oligonucleotide
  source 1..20: mol_type = other RNA
tctaaaggataagttctttg

SEQ ID NO: 591 - RNA, 20 nt, synthetic construct
  misc_feature 1..20: note = Description of Artificial Sequence: Synthetic oligonucleotide
  source 1..20: mol_type = other RNA
ttggctctccaactaaagga

SEQ ID NO: 592 - RNA, 20 nt, synthetic construct
  misc_feature 1..20: note = Description of Artificial Sequence: Synthetic oligonucleotide
  source 1..20: mol_type = other RNA
tcttggctctccaactaaag

SEQ ID NO: 593 - RNA, 20 nt, synthetic construct
  misc_feature 1..20: note = Description of Artificial Sequence: Synthetic oligonucleotide
  source 1..20: mol_type = other RNA
tgtcttggctctccaactaa

SEQ ID NO: 594 - RNA, 20 nt, synthetic construct
  misc_feature 1..20: note = Description of Artificial Sequence: Synthetic oligonucleotide
  source 1..20: mol_type = other RNA
ttgtcttggctctccaacta

SEQ ID NO: 595 - RNA, 20 nt, synthetic construct
  misc_feature 1..20: note = Description of Artificial Sequence: Synthetic oligonucleotide
  source 1..20: mol_type = other RNA
tgattgtcttggctctccaa

SEQ ID NO: 596 - RNA, 20 nt, synthetic construct
  misc_feature 1..20: note = Description of Artificial Sequence: Synthetic oligonucleotide
  source 1..20: mol_type = other RNA
tatgattgtcttggctctcc

SEQ ID NO: 597 - RNA, 20 nt, synthetic construct
  misc_feature 1..20: note = Description of Artificial Sequence: Synthetic oligonucleotide
  source 1..20: mol_type = other RNA
tgttatgattgtcttggctc

SEQ ID NO: 598 - RNA, 20 nt, synthetic construct
  misc_feature 1..20: note = Description of Artificial Sequence: Synthetic oligonucleotide
  source 1..20: mol_type = other RNA
tattgttatgattgtcttgg

SEQ ID NO: 599 - RNA, 20 nt, synthetic construct
  misc_feature 1..20: note = Description of Artificial Sequence: Synthetic oligonucleotide
  source 1..20: mol_type = other RNA
tttattgttatgattgtctt

SEQ ID NO: 600 - RNA, 20 nt, synthetic construct
  misc_feature 1..20: note = Description of Artificial Sequence: Synthetic oligonucleotide
  source 1..20: mol_type = other RNA
tctgtgagaagcagacagct

SEQ ID NO: 601 - RNA, 20 nt, synthetic construct
  misc_feature 1..20: note = Description of Artificial Sequence: Synthetic oligonucleotide
  source 1..20: mol_type = other RNA
tttaaactcagttcaggatc

SEQ ID NO: 602 - RNA, 20 nt, synthetic construct
  misc_feature 1..20: note = Description of Artificial Sequence: Synthetic oligonucleotide
  source 1..20: mol_type = other RNA
ttttaaactcagttcaggat

SEQ ID NO: 603 - RNA, 20 nt, synthetic construct
  misc_feature 1..20: note = Description of Artificial Sequence: Synthetic oligonucleotide
  source 1..20: mol_type = other RNA
tgccttttaaactcagttca

SEQ ID NO: 604 - RNA, 20 nt, synthetic construct
  misc_feature 1..20: note = Description of Artificial Sequence: Synthetic oligonucleotide
  source 1..20: mol_type = other RNA
ttgctgggtgccttttaaac

SEQ ID NO: 605 - RNA, 20 nt, synthetic construct
  misc_feature 1..20: note = Description of Artificial Sequence: Synthetic oligonucleotide
  source 1..20: mol_type = other RNA
ttttgtacgttgcatttgat

SEQ ID NO: 606 - RNA, 20 nt, synthetic construct
  misc_feature 1..20: note = Description of Artificial Sequence: Synthetic oligonucleotide
  source 1..20: mol_type = other RNA
tctttgtacgttgcatttga

SEQ ID NO: 607 - RNA, 20 nt, synthetic construct
  misc_feature 1..20: note = Description of Artificial Sequence: Synthetic oligonucleotide
  source 1..20: mol_type = other RNA
tcatcttttaaccatacaac

SEQ ID NO: 608 - RNA, 20 nt, synthetic construct
  misc_feature 1..20: note = Description of Artificial Sequence: Synthetic oligonucleotide
  source 1..20: mol_type = other RNA
ttggcagtgaggtttttaaa

SEQ ID NO: 609 - RNA, 20 nt, synthetic construct
  misc_feature 1..20: note = Description of Artificial Sequence: Synthetic oligonucleotide
  source 1..20: mol_type = other RNA
tagtggcagtgaggttttta

SEQ ID NO: 610 - RNA, 20 nt, synthetic construct
  misc_feature 1..20: note = Description of Artificial Sequence: Synthetic oligonucleotide
  source 1..20: mol_type = other RNA
tagagtggcagtgaggtttt

SEQ ID NO: 611 - RNA, 20 nt, synthetic construct
  misc_feature 1..20: note = Description of Artificial Sequence: Synthetic oligonucleotide
  source 1..20: mol_type = other RNA
tatgctctcaattctgtttc

SEQ ID NO: 612 - RNA, 15 nt, Homo sapiens
  source 1..15: mol_type = transcribed RNA
ggtttatgaactgac

SEQ ID NO: 613 - RNA, 15 nt, Homo sapiens
  source 1..15: mol_type = transcribed RNA
tatgaactgacgtta

SEQ ID NO: 614 - RNA, 15 nt, Homo sapiens
  source 1..15: mol_type = transcribed RNA
atgaactgacgttac

SEQ ID NO: 615 - RNA, 15 nt, Homo sapiens
  source 1..15: mol_type = transcribed RNA
aatgttgtgaccgga

SEQ ID NO: 616 - RNA, 15 nt, Homo sapiens
  source 1..15: mol_type = transcribed RNA
tagacggtaccgaca

SEQ ID NO: 617 - RNA, 15 nt, Homo sapiens
  source 1..15: mol_type = transcribed RNA
caaccagtatttggg

SEQ ID NO: 618 - RNA, 15 nt, Homo sapiens
  source 1..15: mol_type = transcribed RNA
tgctcaataatgttg

SEQ ID NO: 619 - RNA, 15 nt, Homo sapiens
  source 1..15: mol_type = transcribed RNA
tcctgctttagtcga

SEQ ID NO: 620 - RNA, 15 nt, Homo sapiens
  source 1..15: mol_type = transcribed RNA
tagtcgagaaccaat

SEQ ID NO: 621 - RNA, 15 nt, Homo sapiens
  source 1..15: mol_type = transcribed RNA
agtacttcaacgcta

SEQ ID NO: 622 - RNA, 15 nt, Homo sapiens
  source 1..15: mol_type = transcribed RNA
ttcagtctcgttgtg

SEQ ID NO: 623 - RNA, 15 nt, Homo sapiens
  source 1..15: mol_type = transcribed RNA
ctagctccatgctta

SEQ ID NO: 624 - RNA, 15 nt, Homo sapiens
  source 1..15: mol_type = transcribed RNA
ctgcgtgaacattca

SEQ ID NO: 625 - RNA, 15 nt, Homo sapiens
  source 1..15: mol_type = transcribed RNA
cagctaccaagaaag

SEQ ID NO: 626 - RNA, 15 nt, Homo sapiens
  source 1..15: mol_type = transcribed RNA
ctgacaatatgtgaa

SEQ ID NO: 627 - RNA, 15 nt, Homo sapiens
  source 1..15: mol_type = transcribed RNA
ggcatcgctatggaa

SEQ ID NO: 628 - RNA, 15 nt, Homo sapiens
  source 1..15: mol_type = transcribed RNA
cgctatggaactttt

SEQ ID NO: 629 - RNA, 15 nt, Homo sapiens
  source 1..15: mol_type = transcribed RNA
tgacaatgaaattaa

SEQ ID NO: 630 - RNA, 15 nt, Homo sapiens
  source 1..15: mol_type = transcribed RNA
gacaatgaaattaag

SEQ ID NO: 631 - RNA, 15 nt, Homo sapiens
  source 1..15: mol_type = transcribed RNA
cttcatagcgaacct

SEQ ID NO: 632 - RNA, 15 nt, Homo sapiens
  source 1..15: mol_type = transcribed RNA
atgtgctcttaggct

SEQ ID NO: 633 - RNA, 15 nt, Homo sapiens
  source 1..15: mol_type = transcribed RNA
tgacaaggaaagaaa

SEQ ID NO: 634 - RNA, 15 nt, Homo sapiens
  source 1..15: mol_type = transcribed RNA
ttgtccaggtttatg

SEQ ID NO: 635 - RNA, 15 nt, Homo sapiens
  source 1..15: mol_type = transcribed RNA
caggtttatgaactg

SEQ ID NO: 636 - RNA, 15 nt, Homo sapiens
  source 1..15: mol_type = transcribed RNA
ttatgaactgacgtt

SEQ ID NO: 637 - RNA, 15 nt, Homo sapiens
  source 1..15: mol_type = transcribed RNA
tgaactgacgttaca

SEQ ID NO: 638 - RNA, 15 nt, Homo sapiens
  source 1..15: mol_type = transcribed RNA
actgacgttacatca

SEQ ID NO: 639 - RNA, 15 nt, Homo sapiens
  source 1..15: mol_type = transcribed RNA
acgttacatcataca

SEQ ID NO: 640 - RNA, 15 nt, Homo sapiens
  source 1..15: mol_type = transcribed RNA
gttacatcatacaca

SEQ ID NO: 641 - RNA, 15 nt, Homo sapiens
  source 1..15: mol_type = transcribed RNA
gttgtgaccggagcc

SEQ ID NO: 642 - RNA, 15 nt, Homo sapiens
  source 1..15: mol_type = transcribed RNA
tattgtggaacttat

SEQ ID NO: 643 - RNA, 15 nt, Homo sapiens
  source 1..15: mol_type = transcribed RNA
aaagtgctcttagga

SEQ ID NO: 644 - RNA, 15 nt, Homo sapiens
  source 1..15: mol_type = transcribed RNA
taccgacaaccagta

SEQ ID NO: 645 - RNA, 15 nt, Homo sapiens
  source 1..15: mol_type = transcribed RNA
ccgacaaccagtatt

SEQ ID NO: 646 - RNA, 15 nt, Homo sapiens
  source 1..15: mol_type = transcribed RNA
ctacatcgatcatgg

SEQ ID NO: 647 - RNA, 15 nt, Homo sapiens
  source 1..15: mol_type = transcribed RNA
tacatcgatcatgga

SEQ ID NO: 648 - RNA, 15 nt, Homo sapiens
  source 1..15: mol_type = transcribed RNA
gtgctcaataatgtt

SEQ ID NO: 649 - RNA, 15 nt, Homo sapiens
  source 1..15: mol_type = transcribed RNA
gttacaacaagtaaa

SEQ ID NO: 650 - RNA, 15 nt, Homo sapiens
  source 1..15: mol_type = transcribed RNA
atcctgctttagtcg

SEQ ID NO: 651 - RNA, 15 nt, Homo sapiens
  source 1..15: mol_type = transcribed RNA
ttagtcgagaaccaa

SEQ ID NO: 652 - RNA, 15 nt, Homo sapiens
  source 1..15: mol_type = transcribed RNA
tcgagaaccaatgat

SEQ ID NO: 653 - RNA, 15 nt, Homo sapiens
  source 1..15: mol_type = transcribed RNA
agaaccaatgatggc

SEQ ID NO: 654 - RNA, 15 nt, Homo sapiens
  source 1..15: mol_type = transcribed RNA
caacaattgttgaag

SEQ ID NO: 655 - RNA, 15 nt, Homo sapiens
  source 1..15: mol_type = transcribed RNA
aacatggtgcaggcg

SEQ ID NO: 656 - RNA, 15 nt, Homo sapiens
  source 1..15: mol_type = transcribed RNA
caaagaaccgtgcag

SEQ ID NO: 657 - RNA, 15 nt, Homo sapiens
  source 1..15: mol_type = transcribed RNA
aagaaccgtgcagat

SEQ ID NO: 658 - RNA, 15 nt, Homo sapiens
  source 1..15: mol_type = transcribed RNA
gaaccgtgcagataa

SEQ ID NO: 659 - RNA, 15 nt, Homo sapiens
  source 1..15: mol_type = transcribed RNA
cctcttgttataaaa

SEQ ID NO: 660 - RNA, 15 nt, Homo sapiens
  source 1..15: mol_type = transcribed RNA
tggctttgtattgaa

SEQ ID NO: 661 - RNA, 15 nt, Homo sapiens
  source 1..15: mol_type = transcribed RNA
ggaattcctaaaatc

SEQ ID NO: 662 - RNA, 15 nt, Homo sapiens
  source 1..15: mol_type = transcribed RNA
tggcatcatggccag

SEQ ID NO: 663 - RNA, 15 nt, Homo sapiens
  source 1..15: mol_type = transcribed RNA
cccagtcaactgaag

SEQ ID NO: 664 - RNA, 15 nt, Homo sapiens
  source 1..15: mol_type = transcribed RNA
agcagcaacatactt

SEQ ID NO: 665 - RNA, 15 nt, Homo sapiens
  source 1..15: mol_type = transcribed RNA
gaatgttccggagaa

SEQ ID NO: 666 - RNA, 15 nt, Homo sapiens
  source 1..15: mol_type = transcribed RNA
gaacattcacagcca

SEQ ID NO: 667 - RNA, 15 nt, Homo sapiens
  source 1..15: mol_type = transcribed RNA
gatttaaaatttaat

SEQ ID NO: 668 - RNA, 15 nt, Homo sapiens
  source 1..15: mol_type = transcribed RNA
taaaatttaattata

SEQ ID NO: 669 - RNA, 15 nt, Homo sapiens
  source 1..15: mol_type = transcribed RNA
aattatatcagtaaa

SEQ ID NO: 670 - RNA, 15 nt, Homo sapiens
  source 1..15: mol_type = transcribed RNA
aacgtaactctttct

SEQ ID NO: 671 - RNA, 15 nt, Homo sapiens
  source 1..15: mol_type = transcribed RNA
ctatgcccgtgtaaa

SEQ ID NO: 672 - RNA, 15 nt, Homo sapiens
  source 1..15: mol_type = transcribed RNA
gcccgtgtaaagtat

SEQ ID NO: 673 - RNA, 15 nt, Homo sapiens
  source 1..15: mol_type = transcribed RNA
agtcaggagagtgca

SEQ ID NO: 674 - RNA, 15 nt, Homo sapiens
  source 1..15: mol_type = transcribed RNA
tgggtattgaatgtg

SEQ ID NO: 675 - RNA, 15 nt, Homo sapiens
  source 1..15: mol_type = transcribed RNA
tggtaagtggaggaa

SEQ ID NO: 676 - RNA, 15 nt, Homo sapiens
  source 1..15: mol_type = transcribed RNA
agtggaggaaatgtt

SEQ ID NO: 677 - RNA, 15 nt, Homo sapiens
  source 1..15: mol_type = transcribed RNA
ttggaactctgtgca

SEQ ID NO: 678 - RNA, 15 nt, Homo sapiens
  source 1..15: mol_type = transcribed RNA
tgaggaggcccttaa

SEQ ID NO: 679 - RNA, 15 nt, Homo sapiens
  source 1..15: mol_type = transcribed RNA
agggaagctactgaa

SEQ ID NO: 680 - RNA, 15 nt, Homo sapiens
  source 1..15: mol_type = transcribed RNA
ataacacgtaagaaa

SEQ ID NO: 681 - RNA, 15 nt, Homo sapiens
  source 1..15: mol_type = transcribed RNA
tacatttgtaagaaa

SEQ ID NO: 682 - RNA, 15 nt, Homo sapiens
  source 1..15: mol_type = transcribed RNA
gtaagaaataacact

SEQ ID NO: 683 - RNA, 15 nt, Homo sapiens
  source 1..15: mol_type = transcribed RNA
cactgtgaatgtaaa

SEQ ID NO: 684 - RNA, 15 nt, Homo sapiens
  source 1..15: mol_type = transcribed RNA
gagctcattagtaaa

SEQ ID NO: 685 - RNA, 15 nt, Homo sapiens
  source 1..15: mol_type = transcribed RNA
cacgcatatacataa

SEQ ID NO: 686 - RNA, 15 nt, Homo sapiens
  source 1..15: mol_type = transcribed RNA
gacacatctataatt

SEQ ID NO: 687 - RNA, 15 nt, Homo sapiens
  source 1..15: mol_type = transcribed RNA
cacacacctctcaag

SEQ ID NO: 688 - RNA, 15 nt, Homo sapiens
  source 1..15: mol_type = transcribed RNA
tatcatgttcctaaa

SEQ ID NO: 689 - RNA, 15 nt, Homo sapiens
  source 1..15: mol_type = transcribed RNA
gcaaatgtgattaat

SEQ ID NO: 690 - RNA, 15 nt, Homo sapiens
  source 1..15: mol_type = transcribed RNA
tgtgattaatttggt

SEQ ID NO: 691 - RNA, 15 nt, Homo sapiens
  source 1..15: mol_type = transcribed RNA
ggttgtcaagttttg

SEQ ID NO: 692 - RNA, 15 nt, Homo sapiens
  source 1..15: mol_type = transcribed RNA
tttcctgctggtaat

SEQ ID NO: 693 - RNA, 15 nt, Homo sapiens
  source 1..15: mol_type = transcribed RNA
cctgctggtaatatc

SEQ ID NO: 694 - RNA, 15 nt, Homo sapiens
  source 1..15: mol_type = transcribed RNA
gggaaagattttaat

SEQ ID NO: 695 - RNA, 15 nt, Homo sapiens
  source 1..15: mol_type = transcribed RNA
aatgaaaccagggta

SEQ ID NO: 696 - RNA, 15 nt, Homo sapiens
  source 1..15: mol_type = transcribed RNA
aaaccagggtagaat

SEQ ID NO: 697 - RNA, 15 nt, Homo sapiens
  source 1..15: mol_type = transcribed RNA
ttggcaatgcactga

SEQ ID NO: 698 - RNA, 15 nt, Homo sapiens
  source 1..15: mol_type = transcribed RNA
cagttgtttctaaga

SEQ ID NO: 699 - RNA, 15 nt, Homo sapiens
  source 1..15: mol_type = transcribed RNA
gacgagagatgtata

SEQ ID NO: 700 - RNA, 15 nt, Homo sapiens
  source 1..15: mol_type = transcribed RNA
gagatgtatatttaa

SEQ ID NO: 701 - RNA, 15 nt, Homo sapiens
  source 1..15: mol_type = transcribed RNA
taactgctgcaaaca

SEQ ID NO: 702 - RNA, 15 nt, Homo sapiens
  source 1..15: mol_type = transcribed RNA
gctgcaaacattgta

SEQ ID NO: 703 - RNA, 13 nt, Homo sapiens
  source 1..13: mol_type = transcribed RNA
acaaatacgatta

SEQ ID NO: 704 - RNA, 15 nt, synthetic construct
  misc_feature 1..15: note = Description of Artificial Sequence: Synthetic oligonucleotide
  source 1..15: mol_type = other RNA
ggtttatgaactgaa

SEQ ID NO: 705 - RNA, 15 nt, synthetic construct
  misc_feature 1..15: note = Description of Artificial Sequence: Synthetic oligonucleotide
  source 1..15: mol_type = other RNA
tatgaactgacgtta

SEQ ID NO: 706 - RNA, 15 nt, synthetic construct
  misc_feature 1..15: note = Description of Artificial Sequence: Synthetic oligonucleotide
  source 1..15: mol_type = other RNA
atgaactgacgttaa

SEQ ID NO: 707 - RNA, 15 nt, synthetic construct
  misc_feature 1..15: note = Description of Artificial Sequence: Synthetic oligonucleotide
  source 1..15: mol_type = other RNA
aatgttgtgaccgga

SEQ ID NO: 708 - RNA, 15 nt, synthetic construct
  misc_feature 1..15: note = Description of Artificial Sequence: Synthetic oligonucleotide
  source 1..15: mol_type = other RNA
tagacggtaccgaca

SEQ ID NO: 709 - RNA, 15 nt, synthetic construct
  misc_feature 1..15: note = Description of Artificial Sequence: Synthetic oligonucleotide
  source 1..15: mol_type = other RNA
caaccagtatttgga

SEQ ID NO: 710 - RNA, 15 nt, synthetic construct
  misc_feature 1..15: note = Description of Artificial Sequence: Synthetic oligonucleotide
  source 1..15: mol_type = other RNA
tgctcaataatgtta

SEQ ID NO: 711 - RNA, 15 nt, synthetic construct
  misc_feature 1..15: note = Description of Artificial Sequence: Synthetic oligonucleotide
  source 1..15: mol_type = other RNA
tcctgctttagtcga

SEQ ID NO: 712 - RNA, 15 nt, synthetic construct
  misc_feature 1..15: note = Description of Artificial Sequence: Synthetic oligonucleotide
  source 1..15: mol_type = other RNA
tagtcgagaaccaaa

SEQ ID NO: 713 - RNA, 15 nt, synthetic construct
  misc_feature 1..15: note = Description of Artificial Sequence: Synthetic oligonucleotide
  source 1..15: mol_type = other RNA
agtacttcaacgcta

SEQ ID NO: 714 - RNA, 15 nt, synthetic construct
  misc_feature 1..15: note = Description of Artificial Sequence: Synthetic oligonucleotide
  source 1..15: mol_type = other RNA
ttcagtctcgttgta

SEQ ID NO: 715 - RNA, 15 nt, synthetic construct
  misc_feature 1..15: note = Description of Artificial Sequence: Synthetic oligonucleotide
  source 1..15: mol_type = other RNA
ctagctccatgctta

SEQ ID NO: 716 - RNA, 15 nt, synthetic construct
  misc_feature 1..15: note = Description of Artificial Sequence: Synthetic oligonucleotide
  source 1..15: mol_type = other RNA
ctgcgtgaacattca

SEQ ID NO: 717 - RNA, 15 nt, synthetic construct
  misc_feature 1..15: note = Description of Artificial Sequence: Synthetic oligonucleotide
  source 1..15: mol_type = other RNA
ctcaggatttaaaaa

SEQ ID NO: 718 - RNA, 15 nt, synthetic construct
  misc_feature 1..15: note = Description of Artificial Sequence: Synthetic oligonucleotide
  source 1..15: mol_type = other RNA
atatcagtaaagaga

SEQ ID NO: 719 - RNA, 15 nt, synthetic construct
  misc_feature 1..15: note = Description of Artificial Sequence: Synthetic oligonucleotide
  source 1..15: mol_type = other RNA
cagtaaagagattaa

SEQ ID NO: 720 - RNA, 15 nt, synthetic construct
  misc_feature 1..15: note = Description of Artificial Sequence: Synthetic oligonucleotide
  source 1..15: mol_type = other RNA
cagctaccaagaaaa

SEQ ID NO: 721 - RNA, 15 nt, synthetic construct
  misc_feature 1..15: note = Description of Artificial Sequence: Synthetic oligonucleotide
  source 1..15: mol_type = other RNA
ctgacaatatgtgaa

SEQ ID NO: 722 - RNA, 15 nt, synthetic construct
  misc_feature 1..15: note = Description of Artificial Sequence: Synthetic oligonucleotide
  source 1..15: mol_type = other RNA
ggcatcgctatggaa

SEQ ID NO: 723 - RNA, 15 nt, synthetic construct
  misc_feature 1..15: note = Description of Artificial Sequence: Synthetic oligonucleotide
  source 1..15: mol_type = other RNA
cgctatggaacttta

SEQ ID NO: 724 - RNA, 15 nt, synthetic construct
  misc_feature 1..15: note = Description of Artificial Sequence: Synthetic oligonucleotide
  source 1..15: mol_type = other RNA
tgacaatgaaattaa

SEQ ID NO: 725 - RNA, 15 nt, synthetic construct
  misc_feature 1..15: note = Description of Artificial Sequence: Synthetic oligonucleotide
  source 1..15: mol_type = other RNA
gacaatgaaattaaa

SEQ ID NO: 726 - RNA, 15 nt, synthetic construct
  misc_feature 1..15: note = Description of Artificial Sequence: Synthetic oligonucleotide
  source 1..15: mol_type = other RNA
cttcatagcgaacca

SEQ ID NO: 727 - RNA, 15 nt, synthetic construct
  misc_feature 1..15: note = Description of Artificial Sequence: Synthetic oligonucleotide
  source 1..15: mol_type = other RNA
atgtgctcttaggca

SEQ ID NO: 728 - RNA, 15 nt, synthetic construct
  misc_feature 1..15: note = Description of Artificial Sequence: Synthetic oligonucleotide
  source 1..15: mol_type = other RNA
tgacaaggaaagaaa

SEQ ID NO: 729 - RNA, 15 nt, synthetic construct
  misc_feature 1..15: note = Description of Artificial Sequence: Synthetic oligonucleotide
  source 1..15: mol_type = other RNA
ttgtccaggtttata

SEQ ID NO: 730 - RNA, 15 nt, synthetic construct
  misc_feature 1..15: note = Description of Artificial Sequence: Synthetic oligonucleotide
  source 1..15: mol_type = other RNA
caggtttatgaacta

SEQ ID NO: 731 - RNA, 15 nt, synthetic construct
  misc_feature 1..15: note = Description of Artificial Sequence: Synthetic oligonucleotide
  source 1..15: mol_type = other RNA
ttatgaactgacgta

SEQ ID NO: 732 - RNA, 15 nt, synthetic construct
  misc_feature 1..15: note = Description of Artificial Sequence: Synthetic oligonucleotide
  source 1..15: mol_type = other RNA
tgaactgacgttaca

SEQ ID NO: 733 - RNA, 15 nt, synthetic construct
  misc_feature 1..15: note = Description of Artificial Sequence: Synthetic oligonucleotide
  source 1..15: mol_type = other RNA
actgacgttacatca

SEQ ID NO: 734 - RNA, 15 nt, synthetic construct
  misc_feature 1..15: note = Description of Artificial Sequence: Synthetic oligonucleotide
  source 1..15: mol_type = other RNA
acgttacatcataca

SEQ ID NO: 735 - RNA, 15 nt, synthetic construct
  misc_feature 1..15: note = Description of Artificial Sequence: Synthetic oligonucleotide
  source 1..15: mol_type = other RNA
gttacatcatacaca

SEQ ID NO: 736 - RNA, 15 nt, synthetic construct
  misc_feature 1..15: note = Description of Artificial Sequence: Synthetic oligonucleotide
  source 1..15: mol_type = other RNA
gttgtgaccggagca

SEQ ID NO: 737 - RNA, 15 nt, synthetic construct
  misc_feature 1..15: note = Description of Artificial Sequence: Synthetic oligonucleotide
  source 1..15: mol_type = other RNA
tattgtggaacttaa

SEQ ID NO: 738 - RNA, 15 nt, synthetic construct
  misc_feature 1..15: note = Description of Artificial Sequence: Synthetic oligonucleotide
  source 1..15: mol_type = other RNA
aaagtgctcttagga

SEQ ID NO: 739 - RNA, 15 nt, synthetic construct
  misc_feature 1..15: note = Description of Artificial Sequence: Synthetic oligonucleotide
  source 1..15: mol_type = other RNA
taccgacaaccagta

SEQ ID NO: 740 - RNA, 15 nt, synthetic construct
  misc_feature 1..15: note = Description of Artificial Sequence: Synthetic oligonucleotide
  source 1..15: mol_type = other RNA
ccgacaaccagtata

SEQ ID NO: 741 - RNA, 15 nt, synthetic construct
  misc_feature 1..15: note = Description of Artificial Sequence: Synthetic oligonucleotide
  source 1..15: mol_type = other RNA
ctacatcgatcatga

SEQ ID NO: 742 - RNA, 15 nt, synthetic construct
  misc_feature 1..15: note = Description of Artificial Sequence: Synthetic oligonucleotide
  source 1..15: mol_type = other RNA
tacatcgatcatgga

SEQ ID NO: 743 - RNA, 15 nt, synthetic construct
  misc_feature 1..15: note = Description of Artificial Sequence: Synthetic oligonucleotide
  source 1..15: mol_type = other RNA
gtgctcaataatgta

SEQ ID NO: 744 - RNA, 15 nt, synthetic construct
  misc_feature 1..15: note = Description of Artificial Sequence: Synthetic oligonucleotide
  source 1..15: mol_type = other RNA
gttacaacaagtaaa

SEQ ID NO: 745 - RNA, 15 nt, synthetic construct
  misc_feature 1..15: note = Description of Artificial Sequence: Synthetic oligonucleotide
  source 1..15: mol_type = other RNA
atcctgctttagtca

SEQ ID NO: 746 - RNA, 15 nt, synthetic construct
  misc_feature 1..15: note = Description of Artificial Sequence: Synthetic oligonucleotide
  source 1..15: mol_type = other RNA
ttagtcgagaaccaa

SEQ ID NO: 747 - RNA, 15 nt, synthetic construct
  misc_feature 1..15: note = Description of Artificial Sequence: Synthetic oligonucleotide
  source 1..15: mol_type = other RNA
tcgagaaccaatgaa

SEQ ID NO: 748 - RNA, 15 nt, synthetic construct
  misc_feature 1..15: note = Description of Artificial Sequence: Synthetic oligonucleotide
  source 1..15: mol_type = other RNA
agaaccaatgatgga

SEQ ID NO: 749 - RNA, 15 nt, synthetic construct
  misc_feature 1..15: note = Description of Artificial Sequence: Synthetic oligonucleotide
  source 1..15: mol_type = other RNA
caacaattgttgaaa

SEQ ID NO: 750 - RNA, 15 nt, synthetic construct
  misc_feature 1..15: note = Description of Artificial Sequence: Synthetic oligonucleotide
  source 1..15: mol_type = other RNA
aacatggtgcaggca

SEQ ID NO: 751 - RNA, 15 nt, synthetic construct
  misc_feature 1..15: note = Description of Artificial Sequence: Synthetic oligonucleotide
  source 1..15: mol_type = other RNA
caaagaaccgtgcaa

SEQ ID NO: 752 - RNA, 15 nt, synthetic construct
  misc_feature 1..15: note = Description of Artificial Sequence: Synthetic oligonucleotide
  source 1..15: mol_type = other RNA
aagaaccgtgcagaa

SEQ ID NO: 753 - RNA, 15 nt, synthetic construct
  misc_feature 1..15: note = Description of Artificial Sequence: Synthetic oligonucleotide
  source 1..15: mol_type = other RNA
gaaccgtgcagataa

SEQ ID NO: 754 - RNA, 15 nt, synthetic construct
  misc_feature 1..15: note = Description of Artificial Sequence: Synthetic oligonucleotide
  source 1..15: mol_type = other RNA
cctcttgttataaaa

SEQ ID NO: 755 - RNA, 15 nt, synthetic construct
  misc_feature 1..15: note = Description of Artificial Sequence: Synthetic oligonucleotide
  source 1..15: mol_type = other RNA
tggctttgtattgaa

SEQ ID NO: 756 - RNA, 15 nt, synthetic construct
  misc_feature 1..15: note = Description of Artificial Sequence: Synthetic oligonucleotide
  source 1..15: mol_type = other RNA
ggaattcctaaaata

SEQ ID NO: 757 - RNA, 15 nt, synthetic construct
  misc_feature 1..15: note = Description of Artificial Sequence: Synthetic oligonucleotide
  source 1..15: mol_type = other RNA
tggcatcatggccaa

SEQ ID NO: 758 - RNA, 15 nt, synthetic construct
  misc_feature 1..15: note = Description of Artificial Sequence: Synthetic oligonucleotide
  source 1..15: mol_type = other RNA
cccagtcaactgaaa

SEQ ID NO: 759 - RNA, 15 nt, synthetic construct
  misc_feature 1..15: note = Description of Artificial Sequence: Synthetic oligonucleotide
  source 1..15: mol_type = other RNA
agcagcaacatacta

SEQ ID NO: 760 - RNA, 15 nt, synthetic construct
  misc_feature 1..15: note = Description of Artificial Sequence: Synthetic oligonucleotide
  source 1..15: mol_type = other RNA
gaatgttccggagaa

SEQ ID NO: 761 - RNA, 15 nt, synthetic construct
  misc_feature 1..15: note = Description of Artificial Sequence: Synthetic oligonucleotide
  source 1..15: mol_type = other RNA
gaacattcacagcca

SEQ ID NO: 762 - RNA, 15 nt, synthetic construct
  misc_feature 1..15: note = Description of Artificial Sequence: Synthetic oligonucleotide
  source 1..15: mol_type = other RNA
gatttaaaatttaaa

SEQ ID NO: 763 - RNA, 15 nt, synthetic construct
  misc_feature 1..15: note = Description of Artificial Sequence: Synthetic oligonucleotide
  source 1..15: mol_type = other RNA
taaaatttaattata

SEQ ID NO: 764 - RNA, 15 nt, synthetic construct
  misc_feature 1..15: note = Description of Artificial Sequence: Synthetic oligonucleotide
  source 1..15: mol_type = other RNA
aattatatcagtaaa

SEQ ID NO: 765 - RNA, 15 nt, synthetic construct
  misc_feature 1..15: note = Description of Artificial Sequence: Synthetic oligonucleotide
  source 1..15: mol_type = other RNA
aacgtaactctttca

SEQ ID NO: 766 - RNA, 15 nt, synthetic construct
  misc_feature 1..15: note = Description of Artificial Sequence: Synthetic oligonucleotide
  source 1..15: mol_type = other RNA
ctatgcccgtgtaaa

SEQ ID NO: 767 - RNA, 15 nt, synthetic construct
  misc_feature 1..15: note = Description of Artificial Sequence: Synthetic oligonucleotide
  source 1..15: mol_type = other RNA
gcccgtgtaaagtaa

SEQ ID NO: 768 - RNA, 15 nt, synthetic construct
  misc_feature 1..15: note = Description of Artificial Sequence: Synthetic oligonucleotide
  source 1..15: mol_type = other RNA
agtcaggagagtgca

SEQ ID NO: 769 - RNA, 15 nt, synthetic construct
  misc_feature 1..15: note = Description of Artificial Sequence: Synthetic oligonucleotide
  source 1..15: mol_type = other RNA
tgggtattgaatgta

SEQ ID NO: 770 - RNA, 15 nt, synthetic construct
  misc_feature 1..15: note = Description of Artificial Sequence: Synthetic oligonucleotide
  source 1..15: mol_type = other RNA
tggtaagtggaggaa

SEQ ID NO: 771 - RNA, 15 nt, synthetic construct
  misc_feature 1..15: note = Description of Artificial Sequence: Synthetic oligonucleotide
  source 1..15: mol_type = other RNA
agtggaggaaatgta

SEQ ID NO: 772 - RNA, 15 nt, synthetic construct
  misc_feature 1..15: note = Description of Artificial Sequence: Synthetic oligonucleotide
  source 1..15: mol_type = other RNA
ttggaactctgtgca

SEQ ID NO: 773 - RNA, 15 nt, synthetic construct
  misc_feature 1..15: note = Description of Artificial Sequence: Synthetic oligonucleotide
  source 1..15: mol_type = other RNA
tgaggaggcccttaa

SEQ ID NO: 774 - RNA, 15 nt, synthetic construct
  misc_feature 1..15: note = Description of Artificial Sequence: Synthetic oligonucleotide
  source 1..15: mol_type = other RNA
agggaagctactgaa

SEQ ID NO: 775 - RNA, 15 nt, synthetic construct
  misc_feature 1..15: note = Description of Artificial Sequence: Synthetic oligonucleotide
  source 1..15: mol_type = other RNA
ataacacgtaagaaa

SEQ ID NO: 776 - RNA, 15 nt, synthetic construct
  misc_feature 1..15: note = Description of Artificial Sequence: Synthetic oligonucleotide
  source 1..15: mol_type = other RNA
tacatttgtaagaaa

SEQ ID NO: 777 - RNA, 15 nt, synthetic construct
  misc_feature 1..15: note = Description of Artificial Sequence: Synthetic oligonucleotide
  source 1..15: mol_type = other RNA
gtaagaaataacaca

SEQ ID NO: 778 - RNA, 15 nt, synthetic construct
  misc_feature 1..15: note = Description of Artificial Sequence: Synthetic oligonucleotide
  source 1..15: mol_type = other RNA
cactgtgaatgtaaa

SEQ ID NO: 779 - RNA, 15 nt, synthetic construct
  misc_feature 1..15: note = Description of Artificial Sequence: Synthetic oligonucleotide
  source 1..15: mol_type = other RNA
gagctcattagtaaa

SEQ ID NO: 780 - RNA, 15 nt, synthetic construct
  misc_feature 1..15: note = Description of Artificial Sequence: Synthetic oligonucleotide
  source 1..15: mol_type = other RNA
cacgcatatacataa

SEQ ID NO: 781 - RNA, 15 nt, synthetic construct
  misc_feature 1..15: note = Description of Artificial Sequence: Synthetic oligonucleotide
  source 1..15: mol_type = other RNA
gacacatctataata

SEQ ID NO: 782 - RNA, 15 nt, synthetic construct
  misc_feature 1..15: note = Description of Artificial Sequence: Synthetic oligonucleotide
  source 1..15: mol_type = other RNA
cacacacctctcaaa

SEQ ID NO: 783 - RNA, 15 nt, synthetic construct
  misc_feature 1..15: note = Description of Artificial Sequence: Synthetic oligonucleotide
  source 1..15: mol_type = other RNA
tatcatgttcctaaa

SEQ ID NO: 784 - RNA, 15 nt, synthetic construct
  misc_feature 1..15: note = Description of Artificial Sequence: Synthetic oligonucleotide
  source 1..15: mol_type = other RNA
gcaaatgtgattaaa

SEQ ID NO: 785 - RNA, 15 nt, synthetic construct
  misc_feature 1..15: note = Description of Artificial Sequence: Synthetic oligonucleotide
  source 1..15: mol_type = other RNA
tgtgattaatttgga

SEQ ID NO: 786 - RNA, 15 nt, synthetic construct
  misc_feature 1..15: note = Description of Artificial Sequence: Synthetic oligonucleotide
  source 1..15: mol_type = other RNA
ggttgtcaagtttta

SEQ ID NO: 787 - RNA, 15 nt, synthetic construct
  misc_feature 1..15: note = Description of Artificial Sequence: Synthetic oligonucleotide
  source 1..15: mol_type = other RNA
tttcctgctggtaaa

SEQ ID NO: 788 - RNA, 15 nt, synthetic construct
  misc_feature 1..15: note = Description of Artificial Sequence: Synthetic oligonucleotide
  source 1..15: mol_type = other RNA
cctgctggtaatata

SEQ ID NO: 789 - RNA, 15 nt, synthetic construct
  misc_feature 1..15: note = Description of Artificial Sequence: Synthetic oligonucleotide
  source 1..15: mol_type = other RNA
gggaaagattttaaa

SEQ ID NO: 790 - RNA, 15 nt, synthetic construct
  misc_feature 1..15: note = Description of Artificial Sequence: Synthetic oligonucleotide
  source 1..15: mol_type = other RNA
aatgaaaccagggta

SEQ ID NO: 791 - RNA, 15 nt, synthetic construct
  misc_feature 1..15: note = Description of Artificial Sequence: Synthetic oligonucleotide
  source 1..15: mol_type = other RNA
aaaccagggtagaaa

SEQ ID NO: 792 - RNA, 15 nt, synthetic construct
  misc_feature 1..15: note = Description of Artificial Sequence: Synthetic oligonucleotide
  source 1..15: mol_type = other RNA
ttggcaatgcactga

SEQ ID NO: 793 - RNA, 15 nt, synthetic construct
  misc_feature 1..15: note = Description of Artificial Sequence: Synthetic oligonucleotide
  source 1..15: mol_type = other RNA
cagttgtttctaaga

SEQ ID NO: 794 - RNA, 15 nt, synthetic construct
  misc_feature 1..15: note = Description of Artificial Sequence: Synthetic oligonucleotide
  source 1..15: mol_type = other RNA
gacgagagatgtata

SEQ ID NO: 795 - RNA, 15 nt, synthetic construct
  misc_feature 1..15: note = Description of Artificial Sequence: Synthetic oligonucleotide
  source 1..15: mol_type = other RNA
gagatgtatatttaa

SEQ ID NO: 796 - RNA, 15 nt, synthetic construct
  misc_feature 1..15: note = Description of Artificial Sequence: Synthetic oligonucleotide
  source 1..15: mol_type = other RNA
taactgctgcaaaca

SEQ ID NO: 797 - RNA, 15 nt, synthetic construct
  misc_feature 1..15: note = Description of Artificial Sequence: Synthetic oligonucleotide
  source 1..15: mol_type = other RNA
gctgcaaacattgta

SEQ ID NO: 798 - RNA, 13 nt, synthetic construct
  misc_feature 1..13: note = Description of Artificial Sequence: Synthetic oligonucleotide
  source 1..13: mol_type = other RNA
acaaatacgatta

SEQ ID NO: 799 - RNA, 20 nt, synthetic construct
  misc_feature 1..20: note = Description of Artificial Sequence: Synthetic oligonucleotide
  source 1..20: mol_type = other RNA
ttcagttcataaacctggac

SEQ ID NO: 800 - RNA, 20 nt, synthetic construct
  misc_feature 1..20: note = Description of Artificial Sequence: Synthetic oligonucleotide
  source 1..20: mol_type = other RNA
taacgtcagttcataaacct

SEQ ID NO: 801 - RNA, 20 nt, synthetic construct
  misc_feature 1..20: note = Description of Artificial Sequence: Synthetic oligonucleotide
  source 1..20: mol_type = other RNA
ttaacgtcagttcataaacc

SEQ ID NO: 802 - RNA, 20 nt, synthetic construct
  misc_feature 1..20: note = Description of Artificial Sequence: Synthetic oligonucleotide
  source 1..20: mol_type = other RNA
tccggtcacaacattgtggt

SEQ ID NO: 803 - RNA, 20 nt, synthetic construct
  misc_feature 1..20: note = Description of Artificial Sequence: Synthetic oligonucleotide
  source 1..20: mol_type = other RNA
tgtcggtaccgtctaacaca

SEQ ID NO: 804 - RNA, 20 nt, synthetic construct
  misc_feature 1..20: note = Description of Artificial Sequence: Synthetic oligonucleotide
  source 1..20: mol_type = other RNA
tccaaatactggttgtcggt

SEQ ID NO: 805 - RNA, 20 nt, synthetic construct
  misc_feature 1..20: note = Description of Artificial Sequence: Synthetic oligonucleotide
  source 1..20: mol_type = other RNA
taacattattgagcactcgt

SEQ ID NO: 806 - RNA, 20 nt, synthetic construct
  misc_feature 1..20: note = Description of Artificial Sequence: Synthetic oligonucleotide
  source 1..20: mol_type = other RNA
tcgactaaagcaggatttca

SEQ ID NO: 807 - RNA, 20 nt, synthetic construct
  misc_feature 1..20: note = Description of Artificial Sequence: Synthetic oligonucleotide
  source 1..20: mol_type = other RNA
tttggttctcgactaaagca

SEQ ID NO: 808 - RNA, 20 nt, synthetic construct
  misc_feature 1..20: note = Description of Artificial Sequence: Synthetic oligonucleotide
  source 1..20: mol_type = other RNA
tagcgttgaagtactgtccc

SEQ ID NO: 809 - RNA, 20 nt, synthetic construct
  misc_feature 1..20: note = Description of Artificial Sequence: Synthetic oligonucleotide
  source 1..20: mol_type = other RNA
tacaacgagactgaattgcc

SEQ ID NO: 810 - RNA, 20 nt, synthetic construct
  misc_feature 1..20: note = Description of Artificial Sequence: Synthetic oligonucleotide
  source 1..20: mol_type = other RNA
taagcatggagctagcaggc

SEQ ID NO: 811 - RNA, 20 nt, synthetic construct
  misc_feature 1..20: note = Description of Artificial Sequence: Synthetic oligonucleotide
  source 1..20: mol_type = other RNA
tgaatgttcacgcagtgggc

SEQ ID NO: 812 - RNA, 20 nt, synthetic construct
  misc_feature 1..20: note = Description of Artificial Sequence: Synthetic oligonucleotide
  source 1..20: mol_type = other RNA
tttttaaatcctgagaagaa

SEQ ID NO: 813 - RNA, 20 nt, synthetic construct
  misc_feature 1..20: note = Description of Artificial Sequence: Synthetic oligonucleotide
  source 1..20: mol_type = other RNA
tctctttactgatataatta

SEQ ID NO: 814 - RNA, 20 nt, synthetic construct
  misc_feature 1..20: note = Description of Artificial Sequence: Synthetic oligonucleotide
  source 1..20: mol_type = other RNA
ttaatctctttactgatata

SEQ ID NO: 815 - RNA, 20 nt, synthetic construct
  misc_feature 1..20: note = Description of Artificial Sequence: Synthetic oligonucleotide
  source 1..20: mol_type = other RNA
ttttcttggtagctgaaagt

SEQ ID NO: 816 - RNA, 20 nt, synthetic construct
  misc_feature 1..20: note = Description of Artificial Sequence: Synthetic oligonucleotide
  source 1..20: mol_type = other RNA
ttcacatattgtcagacaat

SEQ ID NO: 817 - RNA, 20 nt, synthetic construct
  misc_feature 1..20: note = Description of Artificial Sequence: Synthetic oligonucleotide
  source 1..20: mol_type = other RNA
ttccatagcgatgcccagaa

SEQ ID NO: 818 - RNA, 20 nt, synthetic construct
  misc_feature 1..20: note = Description of Artificial Sequence: Synthetic oligonucleotide
  source 1..20: mol_type = other RNA
taaagttccatagcgatgcc

SEQ ID NO: 819 - RNA, 20 nt, synthetic construct
  misc_feature 1..20: note = Description of Artificial Sequence: Synthetic oligonucleotide
  source 1..20: mol_type = other RNA
ttaatttcattgtcatttgc

SEQ ID NO: 820 - RNA, 20 nt, synthetic construct
  misc_feature 1..20: note = Description of Artificial Sequence: Synthetic oligonucleotide
  source 1..20: mol_type = other RNA
tttaatttcattgtcatttg

SEQ ID NO: 821 - RNA, 20 nt, synthetic construct
  misc_feature 1..20: note = Description of Artificial Sequence: Synthetic oligonucleotide
  source 1..20: mol_type = other RNA
tggttcgctatgaaggcctt

SEQ ID NO: 822 - RNA, 20 nt, synthetic construct
  misc_feature 1..20: note = Description of Artificial Sequence: Synthetic oligonucleotide
  source 1..20: mol_type = other RNA
tgcctaagagcacatttagt

SEQ ID NO: 823 - RNA, 20 nt, synthetic construct
  misc_feature 1..20: note = Description of Artificial Sequence: Synthetic oligonucleotide
  source 1..20: mol_type = other RNA
tttctttccttgtcactccg

SEQ ID NO: 824 - RNA, 20 nt, synthetic construct
  misc_feature 1..20: note = Description of Artificial Sequence: Synthetic oligonucleotide
  source 1..20: mol_type = other RNA
tataaacctggacaagctgc

SEQ ID NO: 825 - RNA, 20 nt, synthetic construct
  misc_feature 1..20: note = Description of Artificial Sequence: Synthetic oligonucleotide
  source 1..20: mol_type = other RNA
tagttcataaacctggacaa

SEQ ID NO: 826 - RNA, 20 nt, synthetic construct
  misc_feature 1..20: note = Description of Artificial Sequence: Synthetic oligonucleotide
  source 1..20: mol_type = other RNA
tacgtcagttcataaacctg

SEQ ID NO: 827 - RNA, 20 nt, synthetic construct
  misc_feature 1..20: note = Description of Artificial Sequence: Synthetic oligonucleotide
  source 1..20: mol_type = other RNA
tgtaacgtcagttcataaac

SEQ ID NO: 828 - RNA, 20 nt, synthetic construct
  misc_feature 1..20: note = Description of Artificial Sequence: Synthetic oligonucleotide
  source 1..20: mol_type = other RNA
tgatgtaacgtcagttcata

SEQ ID NO: 829 - RNA, 20 nt, synthetic construct
  misc_feature 1..20: note = Description of Artificial Sequence: Synthetic oligonucleotide
  source 1..20: mol_type = other RNA
tgtatgatgtaacgtcagtt

SEQ ID NO: 830 - RNA, 20 nt, synthetic construct
  misc_feature 1..20: note = Description of Artificial Sequence: Synthetic oligonucleotide
  source 1..20: mol_type = other RNA
tgtgtatgatgtaacgtcag

SEQ ID NO: 831 - RNA, 20 nt, synthetic construct
  misc_feature 1..20: note = Description of Artificial Sequence: Synthetic oligonucleotide
  source 1..20: mol_type = other RNA
tgctccggtcacaacattgt

SEQ ID NO: 832 - RNA, 20 nt, synthetic construct
  misc_feature 1..20: note = Description of Artificial Sequence: Synthetic oligonucleotide
  source 1..20: mol_type = other RNA
ttaagttccacaatactccc

SEQ ID NO: 833 - RNA, 20 nt, synthetic construct
  misc_feature 1..20: note = Description of Artificial Sequence: Synthetic oligonucleotide
  source 1..20: mol_type = other RNA
tcctaagagcactttgcctt

SEQ ID NO: 834 - RNA, 20 nt, synthetic construct
  misc_feature 1..20: note = Description of Artificial Sequence: Synthetic oligonucleotide
  source 1..20: mol_type = other RNA
tactggttgtcggtaccgtc

SEQ ID NO: 835 - RNA, 20 nt, synthetic construct
  misc_feature 1..20: note = Description of Artificial Sequence: Synthetic oligonucleotide
  source 1..20: mol_type = other RNA
tatactggttgtcggtaccg

SEQ ID NO: 836 - RNA, 20 nt, synthetic construct
  misc_feature 1..20: note = Description of Artificial Sequence: Synthetic oligonucleotide
  source 1..20: mol_type = other RNA
tcatgatcgatgtagttcaa

SEQ ID NO: 837 - RNA, 20 nt, synthetic construct
  misc_feature 1..20: note = Description of Artificial Sequence: Synthetic oligonucleotide
  source 1..20: mol_type = other RNA
tccatgatcgatgtagttca

SEQ ID NO: 838 - RNA, 20 nt, synthetic construct
  misc_feature 1..20: note = Description of Artificial Sequence: Synthetic oligonucleotide
  source 1..20: mol_type = other RNA
tacattattgagcactcgtt

SEQ ID NO: 839 - RNA, 20 nt, synthetic construct
  misc_feature 1..20: note = Description of Artificial Sequence: Synthetic oligonucleotide
  source 1..20: mol_type = other RNA
tttacttgttgtaacaggac

SEQ ID NO: 840 - RNA, 20 nt, synthetic construct
  misc_feature 1..20: note = Description of Artificial Sequence: Synthetic oligonucleotide
  source 1..20: mol_type = other RNA
tgactaaagcaggatttcag

SEQ ID NO: 841 - RNA, 20 nt, synthetic construct
  misc_feature 1..20: note = Description of Artificial Sequence: Synthetic oligonucleotide
  source 1..20: mol_type = other RNA
ttggttctcgactaaagcag

SEQ ID NO: 842 - RNA, 20 nt, synthetic construct
  misc_feature 1..20: note = Description of Artificial Sequence: Synthetic oligonucleotide
  source 1..20: mol_type = other RNA
ttcattggttctcgactaaa

SEQ ID NO: 843 - RNA, 20 nt, synthetic construct
  misc_feature 1..20: note = Description of Artificial Sequence: Synthetic oligonucleotide
  source 1..20: mol_type = other RNA
tccatcattggttctcgact

SEQ ID NO: 844 - RNA, 20 nt, synthetic construct
  misc_feature 1..20: note = Description of Artificial Sequence: Synthetic oligonucleotide
  source 1..20: mol_type = other RNA
tttcaacaattgttgaacac

SEQ ID NO: 845 - RNA, 20 nt, synthetic construct
  misc_feature 1..20: note = Description of Artificial Sequence: Synthetic oligonucleotide
  source 1..20: mol_type = other RNA
tgcctgcaccatgttcctca

SEQ ID NO: 846 - RNA, 20 nt, synthetic construct
  misc_feature 1..20: note = Description of Artificial Sequence: Synthetic oligonucleotide
  source 1..20: mol_type = other RNA
ttgcacggttctttgtgaca

SEQ ID NO: 847 - RNA, 20 nt, synthetic construct
  misc_feature 1..20: note = Description of Artificial Sequence: Synthetic oligonucleotide
  source 1..20: mol_type = other RNA
ttctgcacggttctttgtga

SEQ ID NO: 848 - RNA, 20 nt, synthetic construct
  misc_feature 1..20: note = Description of Artificial Sequence: Synthetic oligonucleotide
  source 1..20: mol_type = other RNA
ttatctgcacggttctttgt

SEQ ID NO: 849 - RNA, 20 nt, synthetic construct
  misc_feature 1..20: note = Description of Artificial Sequence: Synthetic oligonucleotide
  source 1..20: mol_type = other RNA
ttttataacaagaggttcaa

SEQ ID NO: 850 - RNA, 20 nt, synthetic construct
  misc_feature 1..20: note = Description of Artificial Sequence: Synthetic oligonucleotide
  source 1..20: mol_type = other RNA
ttcaatacaaagccaataaa

SEQ ID NO: 851 - RNA, 20 nt, synthetic construct
  misc_feature 1..20: note = Description of Artificial Sequence: Synthetic oligonucleotide
  source 1..20: mol_type = other RNA
tattttaggaattccaatga

SEQ ID NO: 852 - RNA, 20 nt, synthetic construct
  misc_feature 1..20: note = Description of Artificial Sequence: Synthetic oligonucleotide
  source 1..20: mol_type = other RNA
ttggccatgatgccatcaca

SEQ ID NO: 853 - RNA, 20 nt, synthetic construct
  misc_feature 1..20: note = Description of Artificial Sequence: Synthetic oligonucleotide
  source 1..20: mol_type = other RNA
tttcagttgactgggaaatc

SEQ ID NO: 854 - RNA, 20 nt, synthetic construct
  misc_feature 1..20: note = Description of Artificial Sequence: Synthetic oligonucleotide
  source 1..20: mol_type = other RNA
tagtatgttgctgctcactc

SEQ ID NO: 855 - RNA, 20 nt, synthetic construct
  misc_feature 1..20: note = Description of Artificial Sequence: Synthetic oligonucleotide
  source 1..20: mol_type = other RNA
ttctccggaacattccagac

SEQ ID NO: 856 - RNA, 20 nt, synthetic construct
  misc_feature 1..20: note = Description of Artificial Sequence: Synthetic oligonucleotide
  source 1..20: mol_type = other RNA
tggctgtgaatgttcacgca

SEQ ID NO: 857 - RNA, 20 nt, synthetic construct
  misc_feature 1..20: note = Description of Artificial Sequence: Synthetic oligonucleotide
  source 1..20: mol_type = other RNA
tttaaattttaaatcctgag

SEQ ID NO: 858 - RNA, 20 nt, synthetic construct
  misc_feature 1..20: note = Description of Artificial Sequence: Synthetic oligonucleotide
  source 1..20: mol_type = other RNA
tataattaaattttaaatcc

SEQ ID NO: 859 - RNA, 20 nt, synthetic construct
  misc_feature 1..20: note = Description of Artificial Sequence: Synthetic oligonucleotide
  source 1..20: mol_type = other RNA
tttactgatataattaaatt

SEQ ID NO: 860 - RNA, 20 nt, synthetic construct
  misc_feature 1..20: note = Description of Artificial Sequence: Synthetic oligonucleotide
  source 1..20: mol_type = other RNA
tgaaagagttacgttaaaat

SEQ ID NO: 861 - RNA, 20 nt, synthetic construct
  misc_feature 1..20: note = Description of Artificial Sequence: Synthetic oligonucleotide
  source 1..20: mol_type = other RNA
tttacacgggcatagaaaga

SEQ ID NO: 862 - RNA, 20 nt, synthetic construct
  misc_feature 1..20: note = Description of Artificial Sequence: Synthetic oligonucleotide
  source 1..20: mol_type = other RNA
ttactttacacgggcataga

SEQ ID NO: 863 - RNA, 20 nt, synthetic construct
  misc_feature 1..20: note = Description of Artificial Sequence: Synthetic oligonucleotide
  source 1..20: mol_type = other RNA
tgcactctcctgactaaaag

SEQ ID NO: 864 - RNA, 20 nt, synthetic construct
  misc_feature 1..20: note = Description of Artificial Sequence: Synthetic oligonucleotide
  source 1..20: mol_type = other RNA
tacattcaatacccaaaaca

SEQ ID NO: 865 - RNA, 20 nt, synthetic construct
  misc_feature 1..20: note = Description of Artificial Sequence: Synthetic oligonucleotide
  source 1..20: mol_type = other RNA
ttcctccacttaccacattc

SEQ ID NO: 866 - RNA, 20 nt, synthetic construct
  misc_feature 1..20: note = Description of Artificial Sequence: Synthetic oligonucleotide
  source 1..20: mol_type = other RNA
tacatttcctccacttacca

SEQ ID NO: 867 - RNA, 20 nt, synthetic construct
  misc_feature 1..20: note = Description of Artificial Sequence: Synthetic oligonucleotide
  source 1..20: mol_type = other RNA
tgcacagagttccaacattt

SEQ ID NO: 868 - RNA, 20 nt, synthetic construct
  misc_feature 1..20: note = Description of Artificial Sequence: Synthetic oligonucleotide
  source 1..20: mol_type = other RNA
ttaagggcctcctcaaacat

SEQ ID NO: 869 - RNA, 20 nt, synthetic construct
  misc_feature 1..20: note = Description of Artificial Sequence: Synthetic oligonucleotide
  source 1..20: mol_type = other RNA
ttcagtagcttcccttaagg

SEQ ID NO: 870 - RNA, 20 nt, synthetic construct
  misc_feature 1..20: note = Description of Artificial Sequence: Synthetic oligonucleotide
  source 1..20: mol_type = other RNA
tttcttacgtgttataattc

SEQ ID NO: 871 - RNA, 20 nt, synthetic construct
  misc_feature 1..20: note = Description of Artificial Sequence: Synthetic oligonucleotide
  source 1..20: mol_type = other RNA
tttcttacaaatgtaaacat

SEQ ID NO: 872 - RNA, 20 nt, synthetic construct
  misc_feature 1..20: note = Description of Artificial Sequence: Synthetic oligonucleotide
  source 1..20: mol_type = other RNA
tgtgttatttcttacaaatg

SEQ ID NO: 873 - RNA, 20 nt, synthetic construct
  misc_feature 1..20: note = Description of Artificial Sequence: Synthetic oligonucleotide
  source 1..20: mol_type = other RNA
tttacattcacagtgttatt

SEQ ID NO: 874 - RNA, 20 nt, synthetic construct
  misc_feature 1..20: note = Description of Artificial Sequence: Synthetic oligonucleotide
  source 1..20: mol_type = other RNA
tttactaatgagctcatatt

SEQ ID NO: 875 - RNA, 20 nt, synthetic construct
  misc_feature 1..20: note = Description of Artificial Sequence: Synthetic oligonucleotide
  source 1..20: mol_type = other RNA
ttatgtatatgcgtgggtga

SEQ ID NO: 876 - RNA, 20 nt, synthetic construct
  misc_feature 1..20: note = Description of Artificial Sequence: Synthetic oligonucleotide
  source 1..20: mol_type = other RNA
tattatagatgtgtctatat

SEQ ID NO: 877 - RNA, 20 nt, synthetic construct
  misc_feature 1..20: note = Description of Artificial Sequence: Synthetic oligonucleotide
  source 1..20: mol_type = other RNA
tttgagaggtgtgtgtgtaa

SEQ ID NO: 878 - RNA, 20 nt, synthetic construct
  misc_feature 1..20: note = Description of Artificial Sequence: Synthetic oligonucleotide
  source 1..20: mol_type = other RNA
tttaggaacatgataaagtc

SEQ ID NO: 879 - RNA, 20 nt, synthetic construct
  misc_feature 1..20: note = Description of Artificial Sequence: Synthetic oligonucleotide
  source 1..20: mol_type = other RNA
tttaatcacatttgcaacaa

SEQ ID NO: 880 - RNA, 20 nt, synthetic construct
  misc_feature 1..20: note = Description of Artificial Sequence: Synthetic oligonucleotide
  source 1..20: mol_type = other RNA
tccaaattaatcacatttgc

SEQ ID NO: 881 - RNA, 20 nt, synthetic construct
  misc_feature 1..20: note = Description of Artificial Sequence: Synthetic oligonucleotide
  source 1..20: mol_type = other RNA
taaaacttgacaaccaaatt

SEQ ID NO: 882 - RNA, 20 nt, synthetic construct
  misc_feature 1..20: note = Description of Artificial Sequence: Synthetic oligonucleotide
  source 1..20: mol_type = other RNA
tttaccagcaggaaaacaaa

SEQ ID NO: 883 - RNA, 20 nt, synthetic construct
  misc_feature 1..20: note = Description of Artificial Sequence: Synthetic oligonucleotide
  source 1..20: mol_type = other RNA
tatattaccagcaggaaaac

SEQ ID NO: 884 - RNA, 20 nt, synthetic construct
  misc_feature 1..20: note = Description of Artificial Sequence: Synthetic oligonucleotide
  source 1..20: mol_type = other RNA
tttaaaatctttcccgatat

SEQ ID NO: 885 - RNA, 20 nt, synthetic construct
  misc_feature 1..20: note = Description of Artificial Sequence: Synthetic oligonucleotide
  source 1..20: mol_type = other RNA
taccctggtttcattaaaat

SEQ ID NO: 886 - RNA, 20 nt, synthetic construct
  misc_feature 1..20: note = Description of Artificial Sequence: Synthetic oligonucleotide
  source 1..20: mol_type = other RNA
tttctaccctggtttcatta

SEQ ID NO: 887 - RNA, 20 nt, synthetic construct
  misc_feature 1..20: note = Description of Artificial Sequence: Synthetic oligonucleotide
  source 1..20: mol_type = other RNA
tcagtgcattgccaaacaat

SEQ ID NO: 888 - RNA, 20 nt, synthetic construct
  misc_feature 1..20: note = Description of Artificial Sequence: Synthetic oligonucleotide
  source 1..20: mol_type = other RNA
tcttagaaacaactgagggg

SEQ ID NO: 889 - RNA, 20 nt, synthetic construct
  misc_feature 1..20: note = Description of Artificial Sequence: Synthetic oligonucleotide
  source 1..20: mol_type = other RNA
tatacatctctcgtcagtcc

SEQ ID NO: 890 - RNA, 20 nt, synthetic construct
  misc_feature 1..20: note = Description of Artificial Sequence: Synthetic oligonucleotide
  source 1..20: mol_type = other RNA
ttaaatatacatctctcgtc

SEQ ID NO: 891 - RNA, 20 nt, synthetic construct
  misc_feature 1..20: note = Description of Artificial Sequence: Synthetic oligonucleotide
  source 1..20: mol_type = other RNA
tgtttgcagcagttaaaaaa

SEQ ID NO: 892 - RNA, 20 nt, synthetic construct
  misc_feature 1..20: note = Description of Artificial Sequence: Synthetic oligonucleotide
  source 1..20: mol_type = other RNA
tacaatgtttgcagcagtta

SEQ ID NO: 893 - RNA, 19 nt, synthetic construct
  misc_feature 1..19: note = Description of Artificial Sequence: Synthetic oligonucleotide
  source 1..19: mol_type = other RNA
taatcgtatttgtcaatca

(19) United States
(12) Patent Application Publication
(10) Pub. No.: US 2025/0188458 A1
(43) Pub. Date: Jun. 12, 2025

(54) FULLY STABILIZED ASYMMETRIC SIRNA

(71) Applicant: UNIVERSITY OF MASSACHUSETTS, Westborough, MA (US)

(72) Inventors: Anastasia Khvorova, Westborough, MA (US); Neil Aronin, Newtonville, MA (US); Matthew Hassler, Boston, MA (US); Julia Alterman, Westborough, MA (US)

(21) Appl. No.: 18/939,685

(22) Filed: Nov. 7, 2024

Related U.S. Application Data
(63) Continuation of application No. 16/927,543, filed on Jul. 13, 2020, now Pat. No. 12,173,286.
Application No. 16/927,543 is a continuation of application No. 15/089,423, filed on Apr. 1, 2016, now abandoned.
(60) Provisional application No. 62/287,255, filed on Jan. 26, 2016.
(60) Provisional application No. 62/205,218, filed on Aug. 14, 2015.
(60) Provisional application No. 62/142,786, filed on Apr. 3, 2015.

(51) Int. Cl. C12N 15/113; C12N 15/11

(52) U.S. Cl. CPC C12N 15/113; C12N 15/111; C12N 15/1138; C12Y 207/10001; C12N 2310/14; C12N 2310/315; C12N 2310/321; C12N 2310/343; C12N 2310/344; C12N 2310/346; C12N 2310/3515; C12N 2320/51; C12N 2320/53

(57) ABSTRACT

Provided herein are self-delivering oligonucleotides that are characterized by efficient RISC entry, minimum immune response and off-target effects, efficient cellular uptake without formulation, and efficient and specific tissue distribution.

### RELATED APPLICATIONS

This application is a continuation of U.S. patent application Ser. No. 16/927,543, filed Jul. 13, 2020, which is a continuation of U.S. patent application Ser. No. 15/089,423, filed Apr. 1, 2016, which claims priority to U.S. Provisional Patent Application No. 62/142,786, filed Apr. 3, 2015, U.S. Provisional Patent Application No. 62/205,218, filed Aug. 14, 2015, and U.S. Provisional Patent Application No. 62/287,255, filed Jan. 26, 2016. The entire contents of these applications are incorporated herein by reference.

### STATEMENT REGARDING FEDERALLY FUNDED RESEARCH

This invention was made with government support under grant numbers NS038194, GM108803 and TR000888 awarded by the National Institutes of Health, and grant number OPP1086170 awarded by the National Science Foundation. The Government has certain rights in the invention.

### SEQUENCE LISTING

The instant application contains a Sequence Listing which has been submitted electronically in XML format and is hereby incorporated by reference in its entirety. Said XML file, created on Nov. 1, 2024, is named 759148_UM9-209CON4_ST26.xml and is 1,076,235 bytes in size.

### TECHNICAL FIELD

This disclosure relates to novel oligonucleotides useful for RNA interference (RNAi), consisting of fully chemically-modified ribonucleotides. The chemically-modified nucleotides and linkers are patterned to achieve unexpectedly high efficacy, uptake and tissue distribution.

### BACKGROUND

Oligonucleotides comprising chemically-modified ribonucleotides (e.g., 2′-fluoro and 2′-methoxy modifications) and/or chemically-modified linkers (e.g., a phosphorothioate modification) are known to exhibit increased nuclease resistance relative to the corresponding unmodified oligonucleotides, while maintaining the ability to promote RNAi. See, e.g., Fosnaugh, et al. (U.S. Publication No. 2003/0143732). Oligonucleotides comprising alternating chemically-modified nucleotides are known. See, e.g., Bhat et al. (U.S. Publication No. 2008/0119427). Hydrophobic modification of therapeutic RNA (e.g., siRNA) is known. See, e.g., Khvorova, et al. (PCT/US2009/005247).

There remains a need for self-delivering siRNA that is characterized by efficient RISC entry, minimum immune response and off-target effects, efficient cellular uptake without formulation and efficient and specific tissue distribution.

### SUMMARY

Accordingly, provided herein in certain embodiments are siRNA compounds having the following properties: (1) fully chemically-stabilized (i.e., no unmodified 2′-OH residues); (2) asymmetry; (3) 11-16 base pair duplexes; (4) alternating pattern of chemically-modified nucleotides (e.g., 2′-fluoro and 2′-methoxy modifications); (5) single-stranded, fully phosphorothioated tails of 5-8 bases. The number of phosphorothioate modifications is varied from 6 to 17 total in different embodiments.

In certain embodiments, the siRNA compounds described herein can be conjugated to a variety of targeting agents, including, but not limited to, cholesterol, DHA, phenyltropanes, cortisol, Vitamin A, Vitamin D, GalNac, and Gangliosides. The cholesterol-modified version showed 5-10 fold improvement in efficacy in vitro versus previously used chemical stabilization patterns (e.g., wherein all purine but not pyrimidines are modified) in wide range of cell types (e.g., HeLa, Neurons, Hepatocytes, Trophoblasts).

Certain compounds of the invention having the structural properties described above and herein may be referred to as “hsiRNA-ASP” (hydrophobically-modified, small interfering RNA, featuring an advanced stabilization pattern), and may also be referred to as “FM-hsiRNA” (Fully Modified hydrophobically-modified, small interfering RNA). In addition, this hsiRNA-ASP pattern showed a dramatically improved distribution through the brain, spinal cord, delivery to liver, placenta, kidney, spleen and several other tissues, making them accessible for therapeutic intervention.

In liver, hsiRNA-ASP is delivered specifically to endothelial and kupffer cells, but not hepatocytes, making this chemical modification pattern a complimentary, rather than competitive, technology to GalNac conjugates.

The compounds of the invention can be described in the following aspects and embodiments.

In a first aspect, provided herein is compound (I): an oligonucleotide of at least 16 contiguous nucleotides, said oligonucleotide having a 5′ end, a 3′ end and complementarity to a target, wherein:
(1) the oligonucleotide comprises alternating 2′-methoxy-ribonucleotides and 2′-fluoro-ribonucleotides;
(2) the nucleotides at positions 2 and 14 from the 5′ end are not 2′-methoxy-ribonucleotides;
(3) the nucleotides are connected via phosphodiester or phosphorothioate linkages; and
(4) the nucleotides at positions 1-6 from the 3′ end, or positions 1-7 from the 3′ end, are connected to adjacent nucleotides via phosphorothioate linkages.

In a second aspect, provided herein is a double-stranded, chemically-modified nucleic acid, comprising a first oligonucleotide compound (I) and a second oligonucleotide compound (II), wherein:
(1) a portion of the first oligonucleotide is complementary to a portion of the second oligonucleotide;
(2) the second oligonucleotide comprises alternating 2′-methoxy-ribonucleotides and 2′-fluoro-ribonucleotides;
(3) the nucleotides at positions 2 and 14 from the 3′ end of the second oligonucleotide are 2′-methoxy-ribonucleotides; and
(4) the nucleotides of the second oligonucleotide are connected via phosphodiester or phosphorothioate linkages.

In a third aspect, provided herein is an oligonucleotide having the structure of compound (Ia):

X(-K-B-K-A)ⱼ(-S-B-S-A)ᵣ(-S-B)ₜ-OR (Ia)
wherein:
X is selected from the group consisting of:

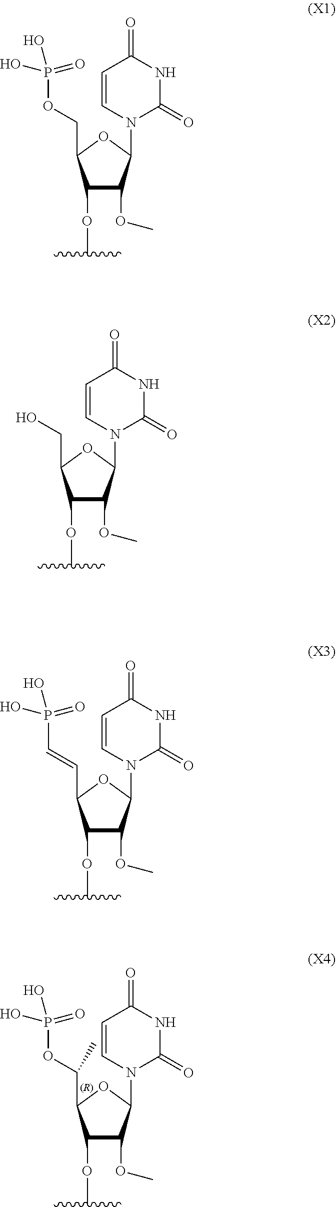

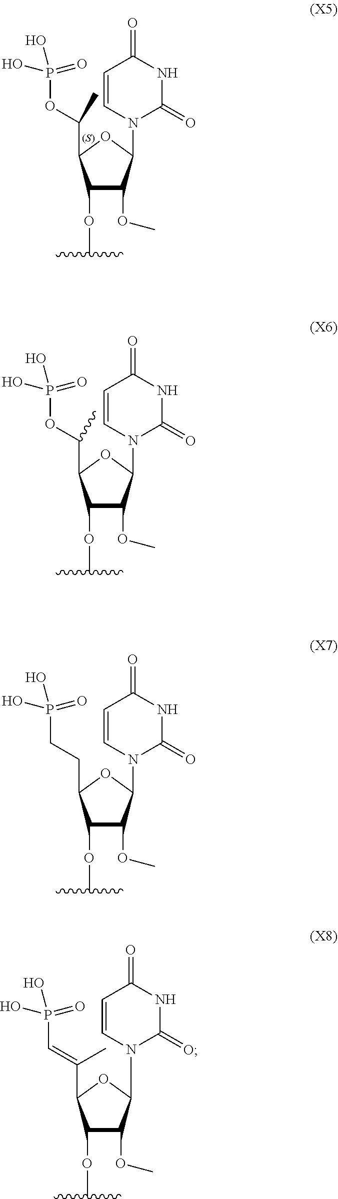

A, for each occurrence, independently is a 2′-methoxy-ribonucleotide; B, for each occurrence, independently is a 2′-fluoro-ribonucleotide; K, for each occurrence independently is a phosphodiester or phosphorothioate linker; S is a phosphorothioate linker; R, for each occurrence, independently is selected from hydrogen and a capping group (e.g., an acyl group such as acetyl); j is 4, 5, 6 or 7; r is 2 or 3; and t is 0 or 1.

In a fourth aspect, provided herein is a double-stranded, chemically-modified nucleic acid comprising a first oligonucleotide and a second oligonucleotide, wherein:
(1) the first oligonucleotide is an oligonucleotide as described herein (e.g., compound (I), (Ia) or (Ib));
(2) a portion of the first oligonucleotide is complementary to a portion of the second oligonucleotide; and
(3) the second oligonucleotide has the structure of compound (IIa):

C-L-B(-S-A-S-B)_{m′}(-P-A-P-B)_{n′}(-P-A-S-B)_{q′}(-S-A)_{r′}(-S-B)_{t′}-OR (IIa)
wherein: C is a hydrophobic molecule; A, for each occurrence, independently is a 2′-methoxy-ribonucleotide; B, for each occurrence, independently is a 2′-fluoro-ribonucleotide; L is a linker comprising one or more moiety selected from the group consisting of: 0-20 repeat units of ethyleneglycol, a phosphodiester, and a phosphorothioate; S is a phosphorothioate linker; P is a phosphodiester linker; R is selected from hydrogen and a capping group (e.g., an acyl group such as acetyl); m′ is 0 or 1; n′ is 4, 5 or 6; q′ is 0 or 1; r′ is 0 or 1; and t′ is 0 or 1.

### BRIEF DESCRIPTION OF THE DRAWINGS

### Color Drawings

The patent or application file contains at least one drawing executed in color. Copies of this patent or patent application publication with color drawing(s) will be provided by the Office upon request and payment of the necessary fec.

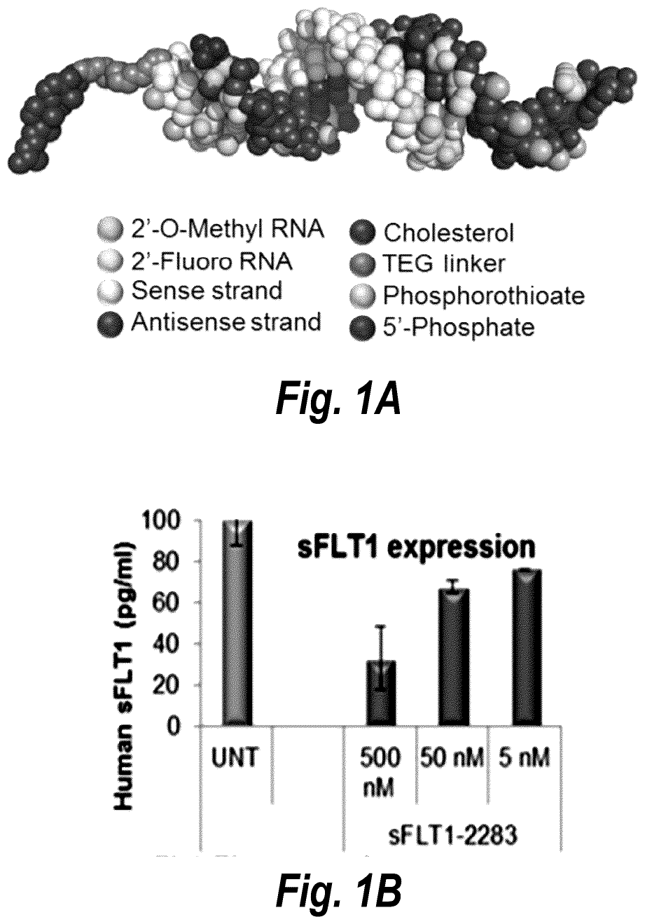

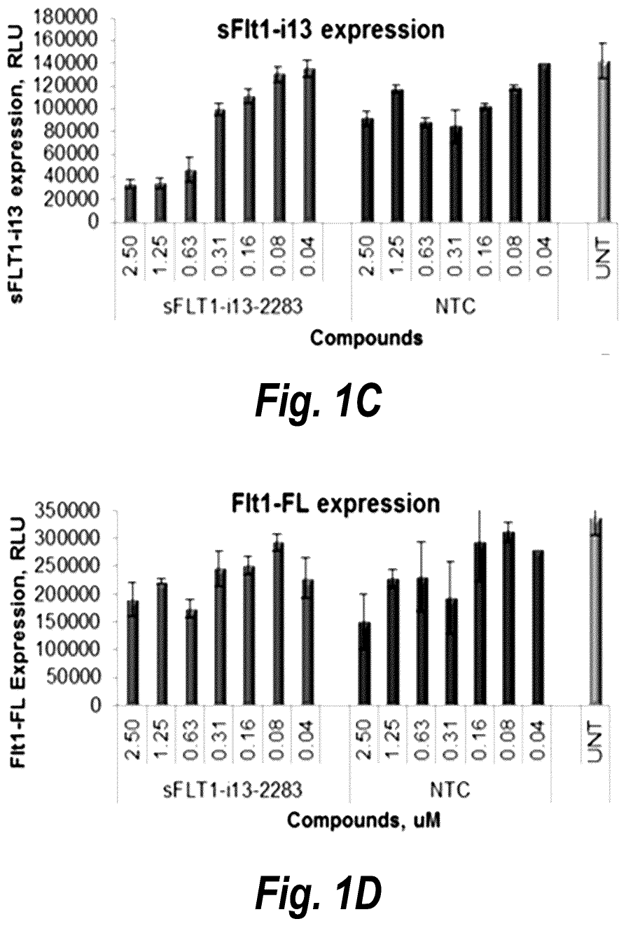

FIGS. 1A-1D depict a hydrophobic siRNA structural/chemical composition, uptake and efficacy in primary human cytotrophoblasts (CTBs). (A) Schematically depicts hydrophobically modified and stabilized siRNAs (hsiRNAs) according to certain embodiments. sFlt1-i13-2283 hsiRNA and matching NTC was added to CTBs at concentration shown. (B) Level of sFLT1 protein was measured by ELISA (#MVR100, R&D systems) in conditioned culture medium after 72 h treatment. (C) depicts sFlt1-i13 mRNA levels, and (D) depicts Flt1-FL mRNA levels that were measured using QUANTIGENE (Affymetrix) at 72 hours, (n=3, mean+/−SD). UNT—untreated cells, NTC—non-targeting control with matching chemistry.

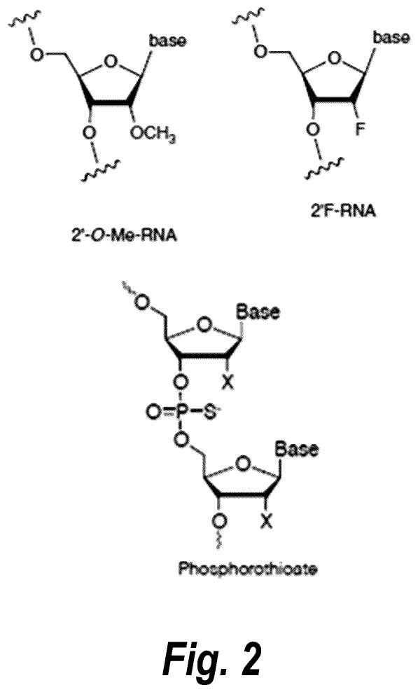

FIG. 2 depicts particular nucleotide and linker chemical modifications.

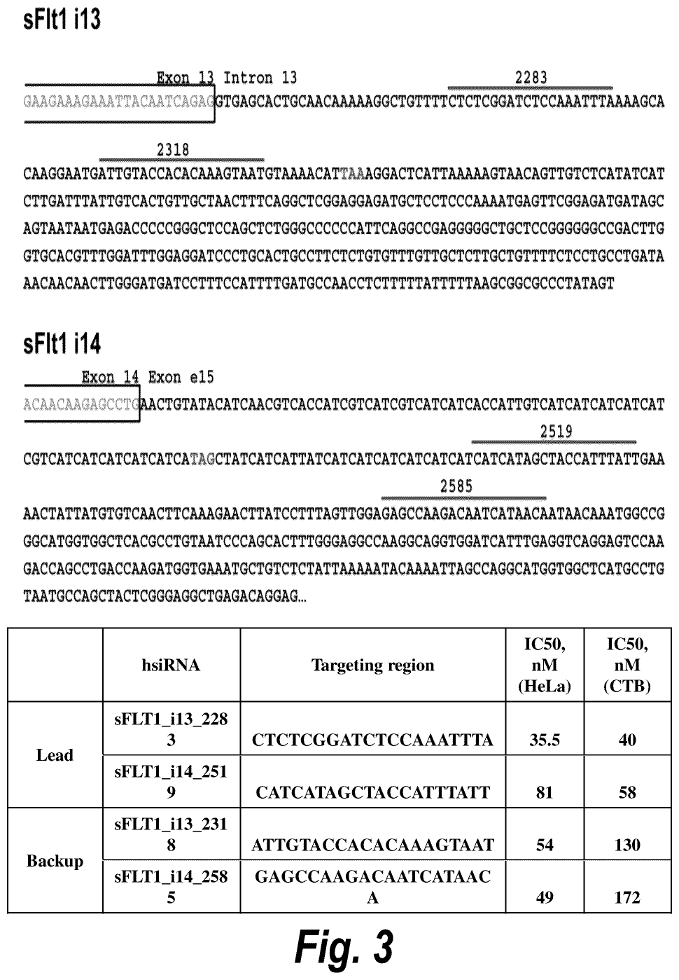

FIG. 3 depicts the identification and validation of compounds of the invention targeting i13 and i14 isoforms of sFLT1. FIG. 3 discloses SEQ ID NOS 14-15 and 10-13, respectively, in order of appearance.

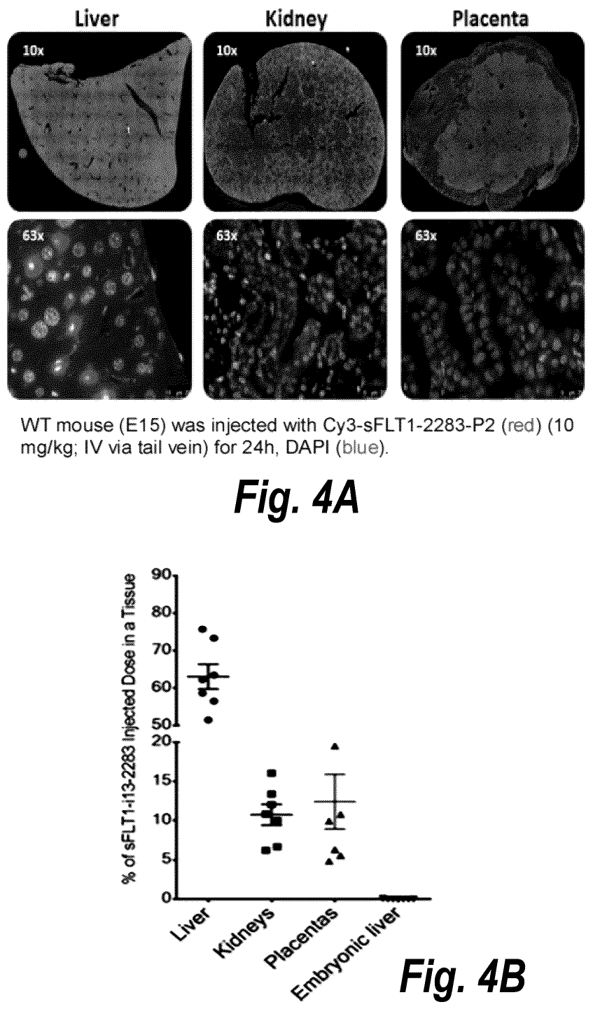

FIGS. 4A-4B depict hsiRNA efficiency of delivery to liver, kidney and placenta. (A) A wild-type pregnant mouse (E15) was injected with Cy3-sFLT1-2283-P2 (red) (10 mg/kg; IV via tail vein). Tissues were fixed after 24 hours, processed and imaged at 10× and 63× on a Leica tiling fluorescent microscope; nuclei stained with DAPI (blue). (B) Shows tissue distribution of sFLT1-2283 (40 mg/kg) 5 days post injection analyzed by PNA assay (n=7, mean+SEM).

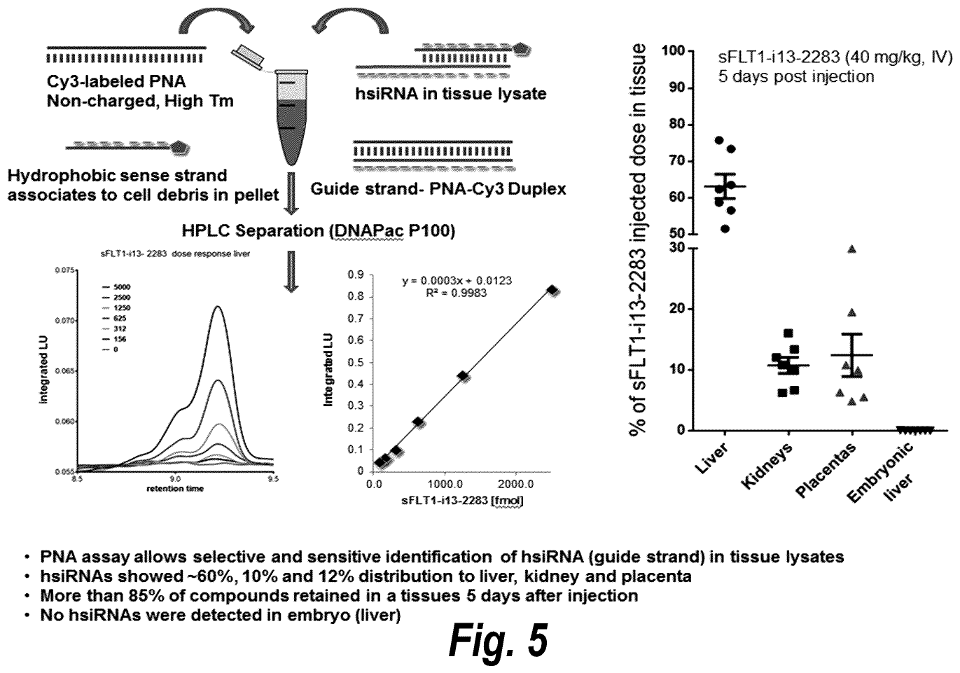

FIG. 5 depicts in vivo quantification of a compound of the invention by PNA assay.

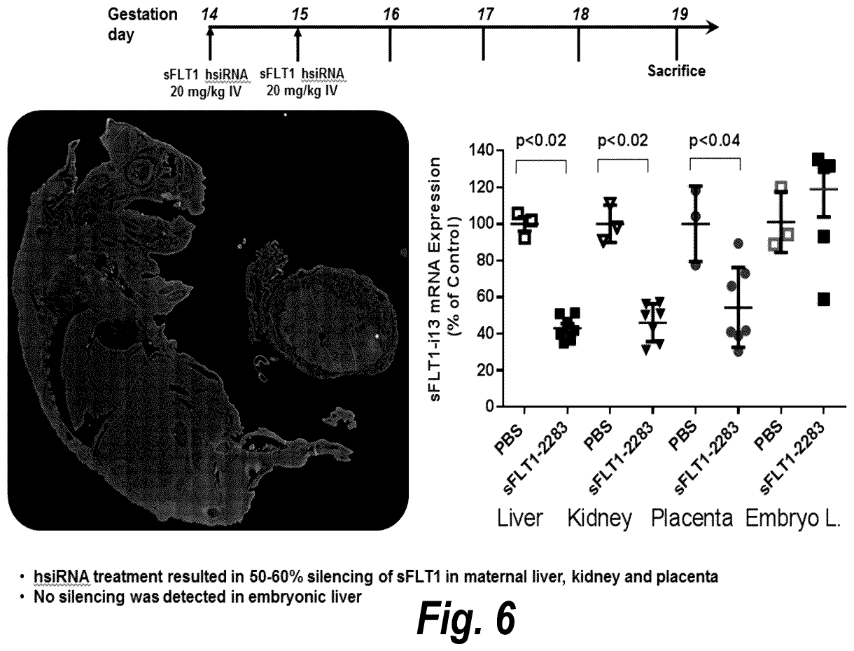

FIG. 6 depicts data showing silencing of Flt1 by a compound of the invention in WT pregnant mice.

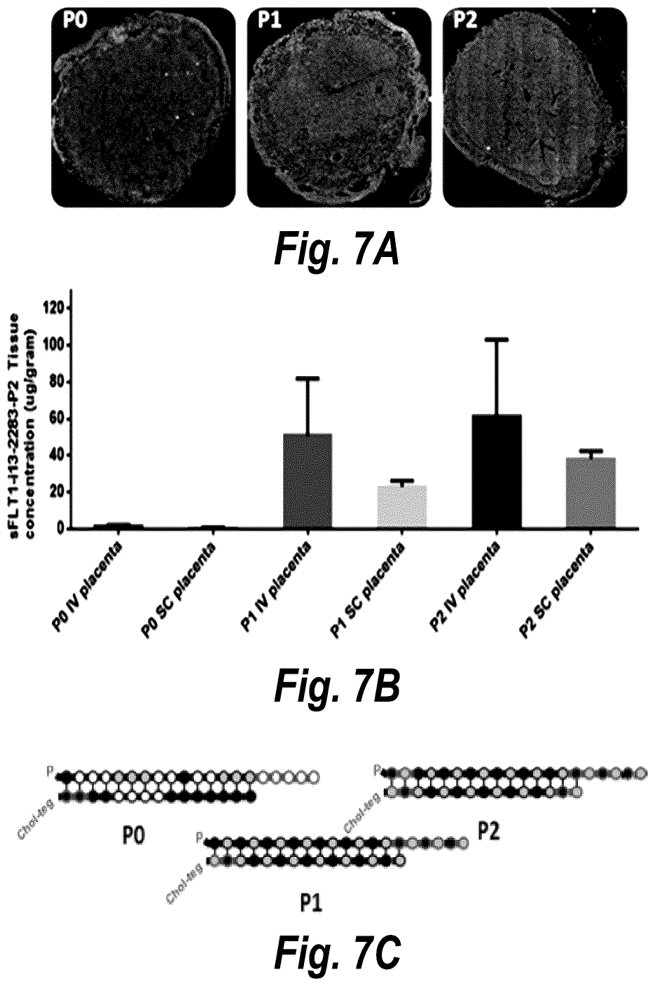

FIGS. 7A-7C depict the impact of hsiRNA chemistry and route of administration on placental accumulation and distribution. (A) A wild-type pregnant mouse (E15) was injected with Cy3-sFLT1-2283 (red) (10 mg/kg; IV via tail vein). Placentas were fixed after 24 hours, processed, and imaged on a Leica tiling fluorescent microscope; nuclei stained with DAPI (blue). (B) Depicts accumulation of sFLT1-i13-2283 (10 mg/kg) after 24 hours, and analyzed by PNA assay (n=3, mean+SEM). (C) Schematically represents different modification patterns of sFLT1-i13-2283 hsiRNA. P—5′-phosphate; Chol-teg—Cholesterol-teg linker; white spheres—RNA; black spheres—2′-O-methyl; grey spheres—2′-Fluoro; red spheres—phosphorothioate.

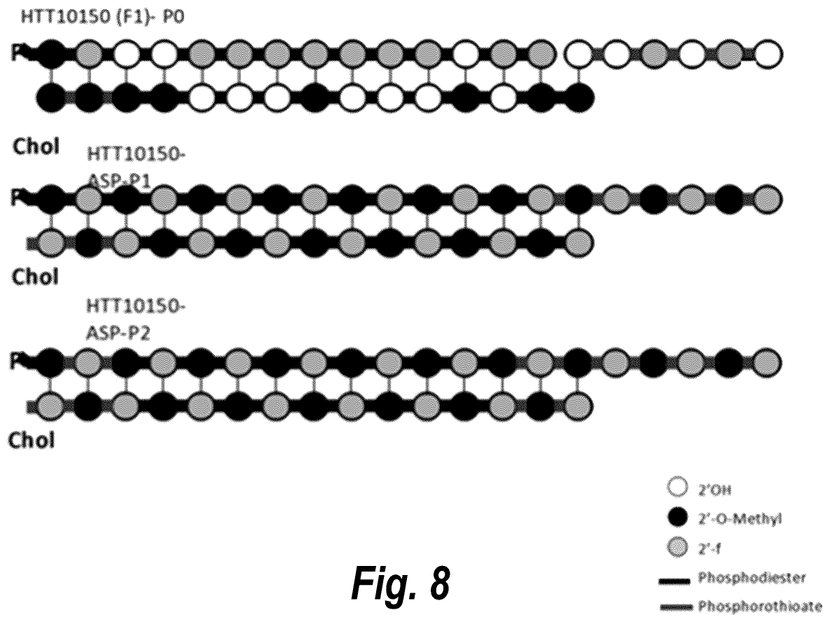

FIG. 8 depicts the structure and stabilization pattern of siRNA compounds of the invention.

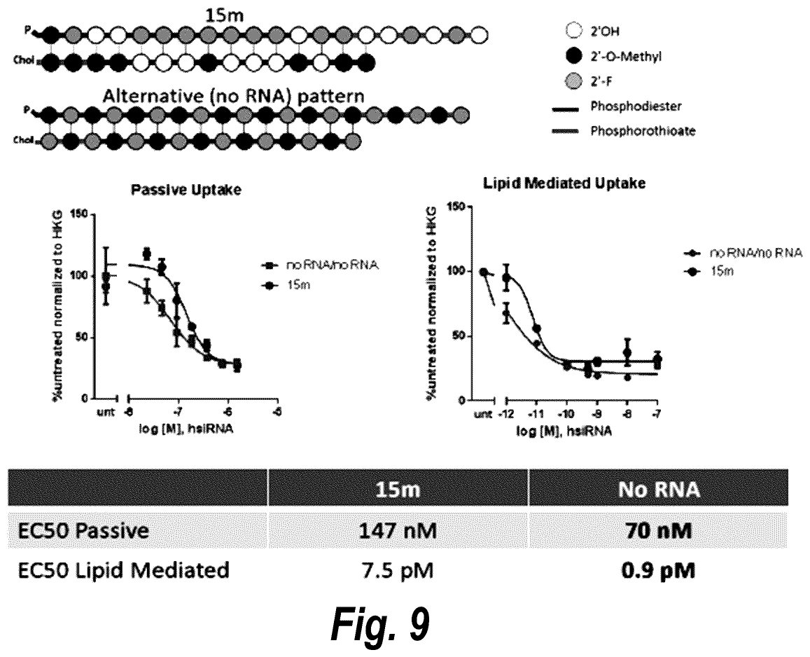

FIG. 9 depicts the modification pattern of compounds of the invention and the resulting increase of in vitro efficacy.

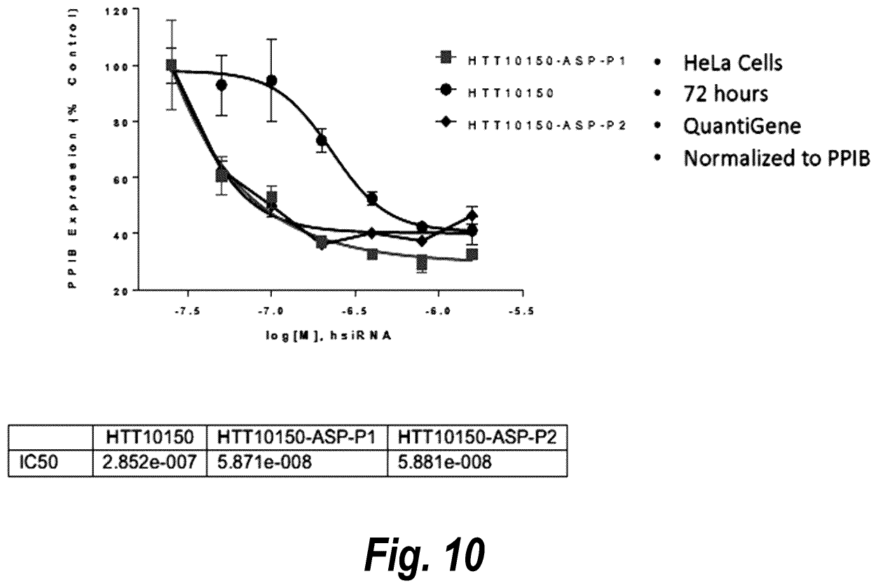

FIG. 10 depicts data showing the increased potency of compounds of the invention versus a comparator compounds that do not feature ASP (advanced stabilization pattern) chemical modifications.

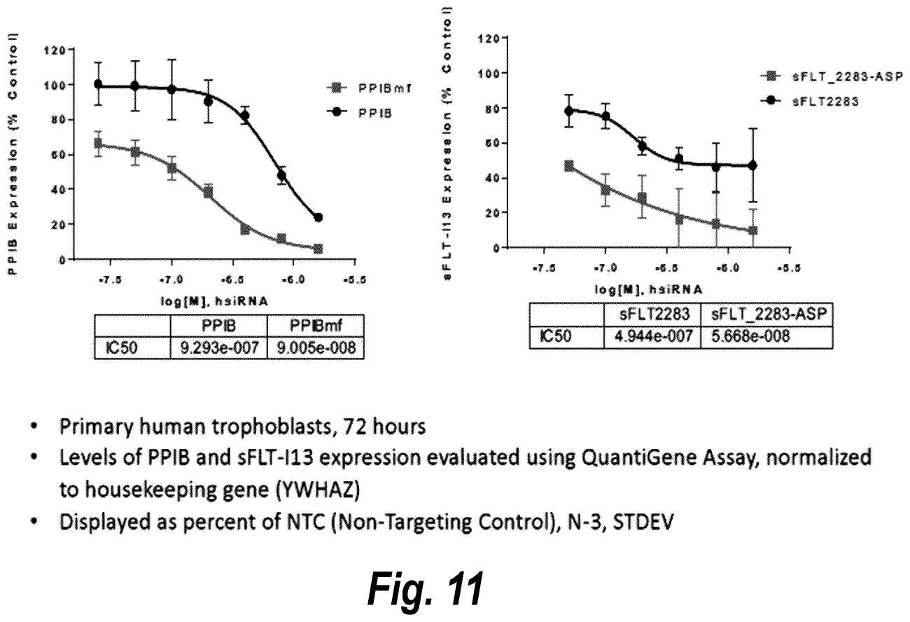

FIG. 11 depicts data showing the increased potency of compounds of the invention versus a comparator compounds that do not feature ASP (advanced stabilization pattern) chemical modifications.

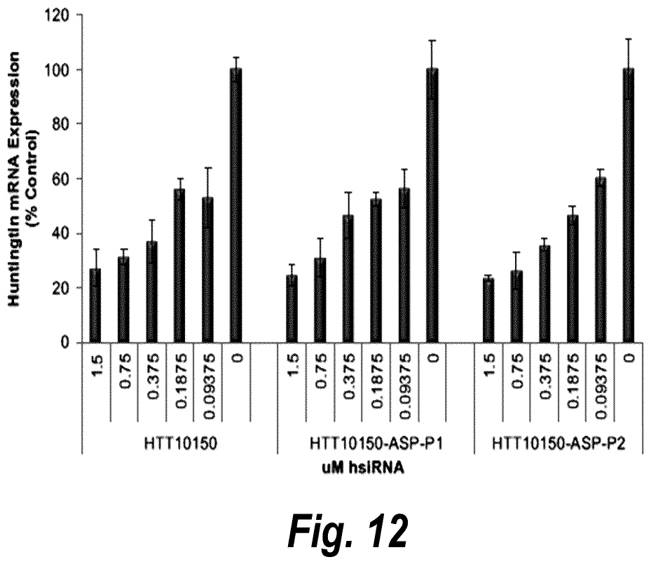

FIG. 12 depicts the efficacy of HTT10150-ASP-P1 and HTT10150-ASP-P2 versus HTT10150 in neurons.

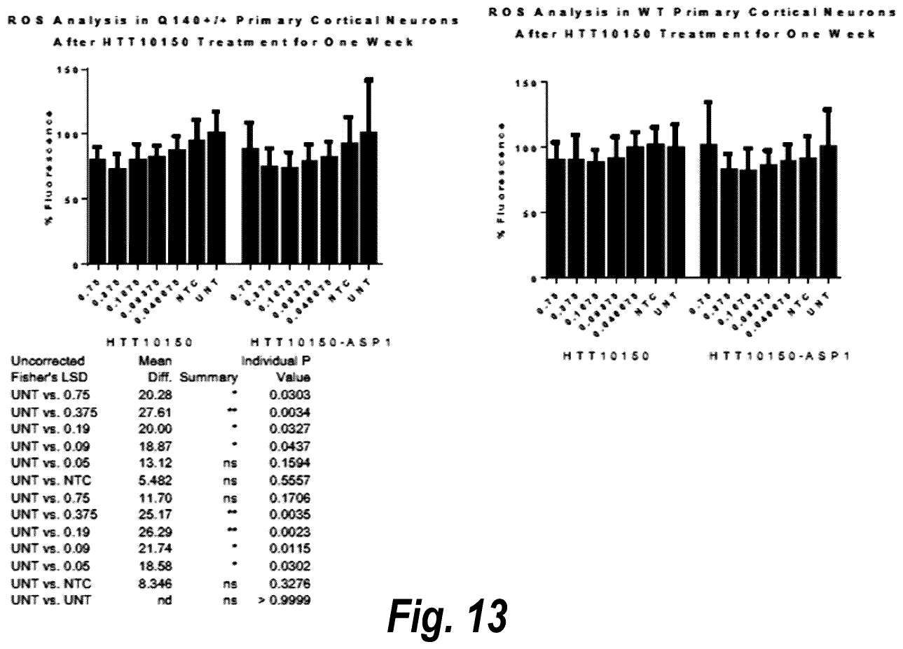

FIG. 13 depicts data showing that HTT10150-ASP is more potent in ROS modulation in Q140.

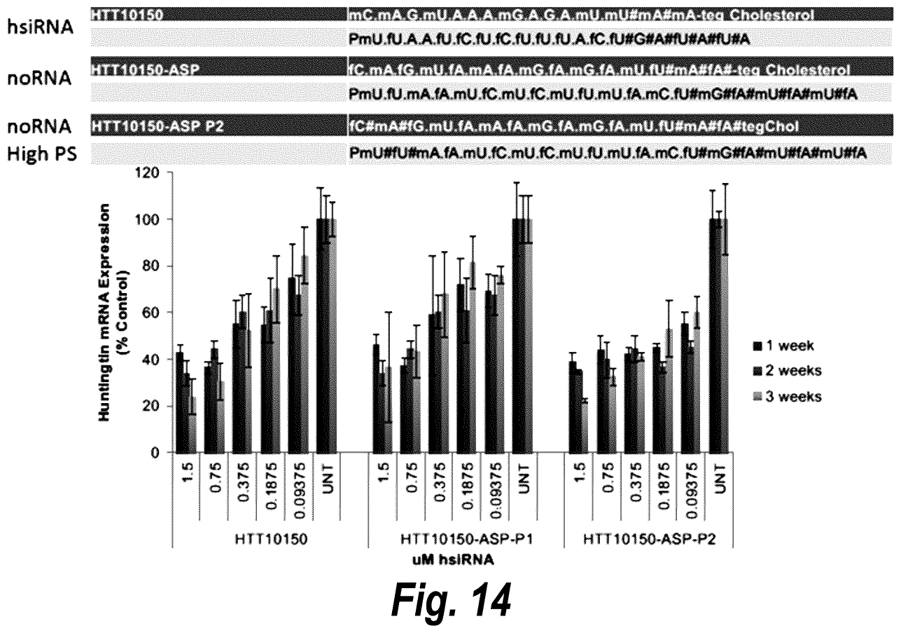

FIG. 14 depicts data showing that HTT10150-ASP-P2 shows better long term silencing and potency in neurons. FIG. 14 discloses SEQ ID NOS 16-21, respectively, in order of appearance.

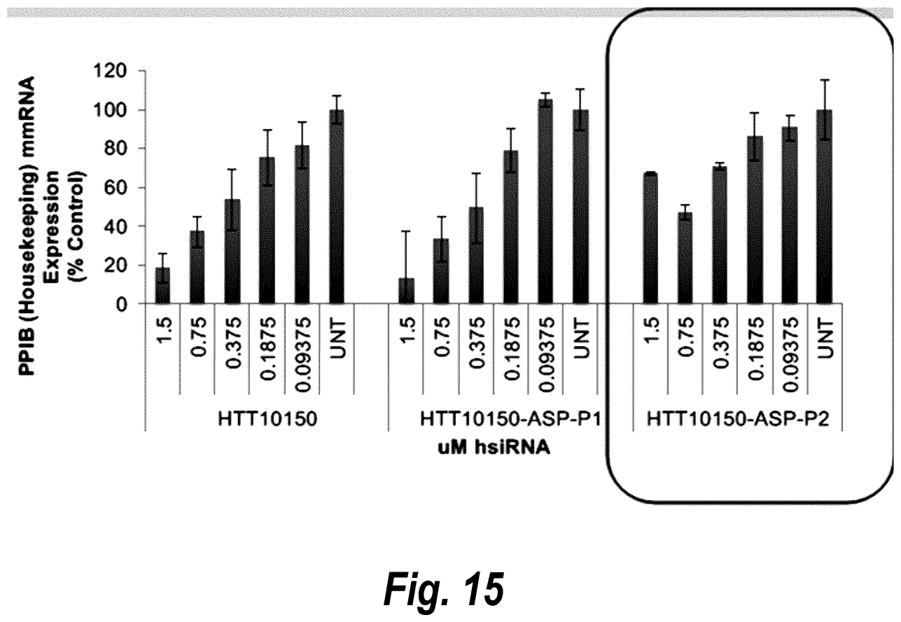

FIG. 15 depicts data showing that HTT10150-ASP-P2 shows reduced toxicity in primary neurons.

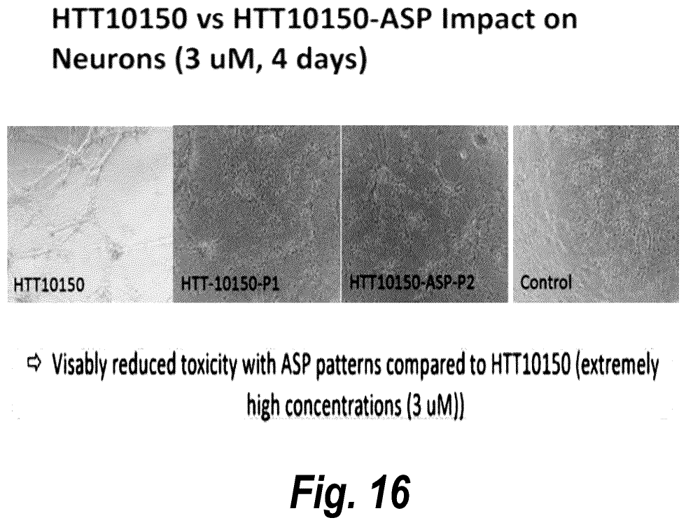

FIG. 16 depicts data showing visibly reduced toxicity of compounds having ASP patterns versus HTT10150.

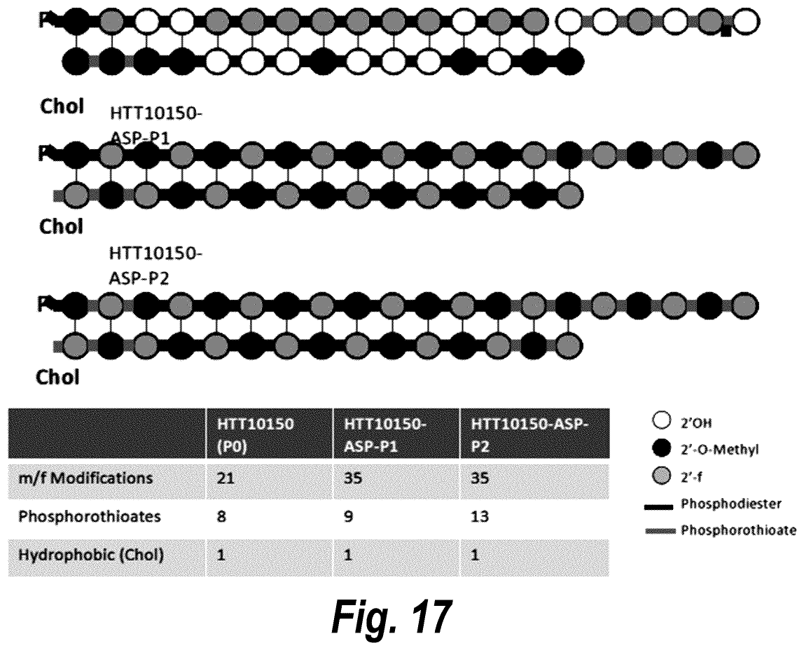

FIG. 17 depicts the number of phosphorothioates and number of modified nucleotide monomers compared to the P0, P1, and P2 modification patterns.

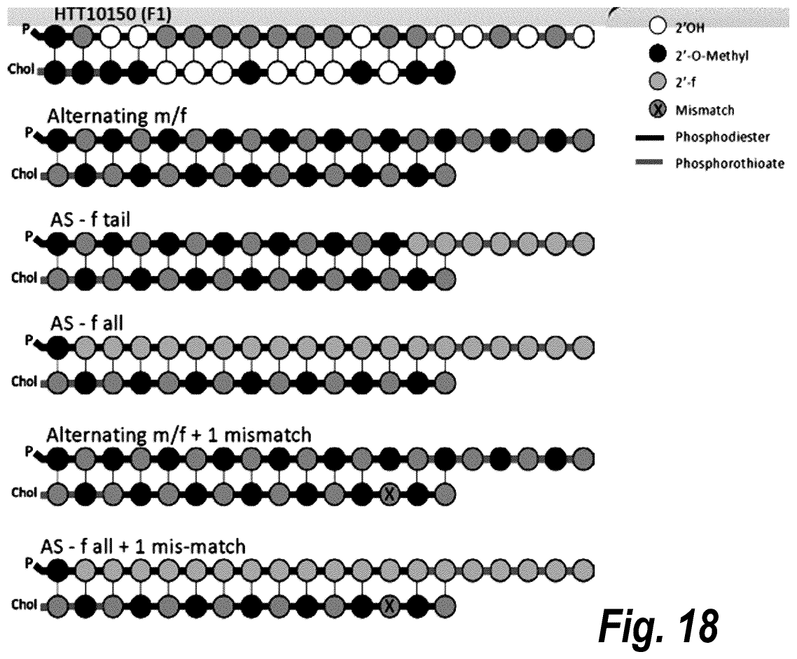

FIG. 18 depicts alternative modification patterns.

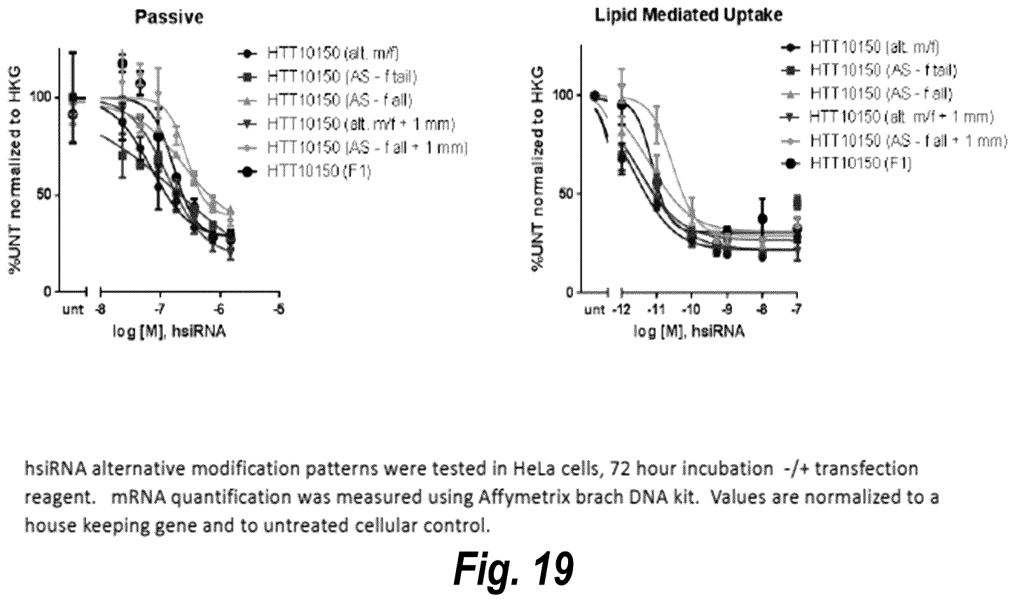

FIG. 19 depicts data related to uptake of siRNA having alternative modification patterns.

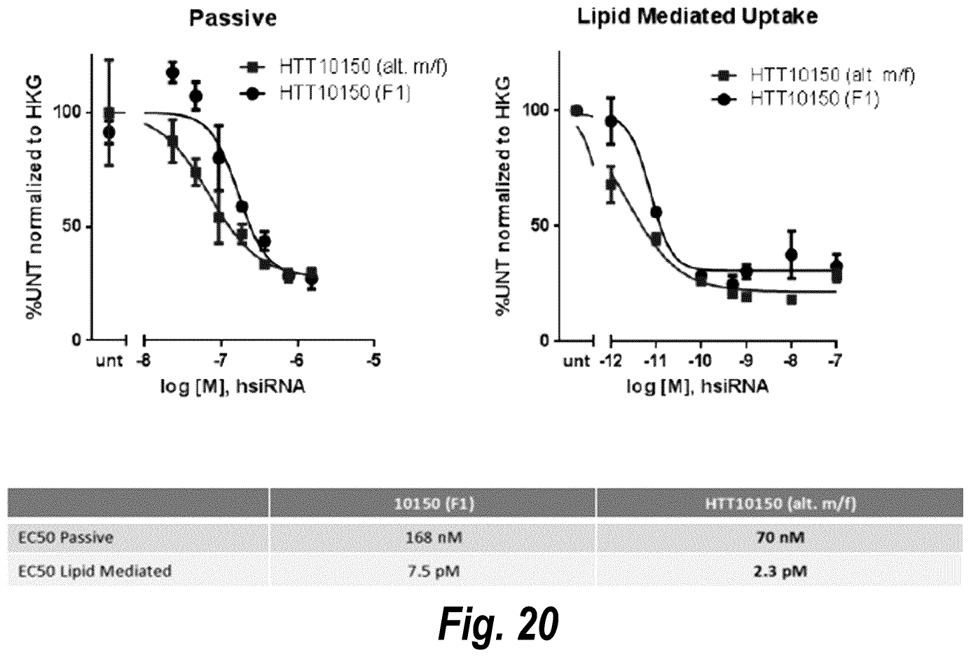

FIG. 20 depicts data showing that siRNA having an alternating 2′-O-Methyl (2′-methoxy)/2′-fluoro pattern show increased efficacy in vitro.

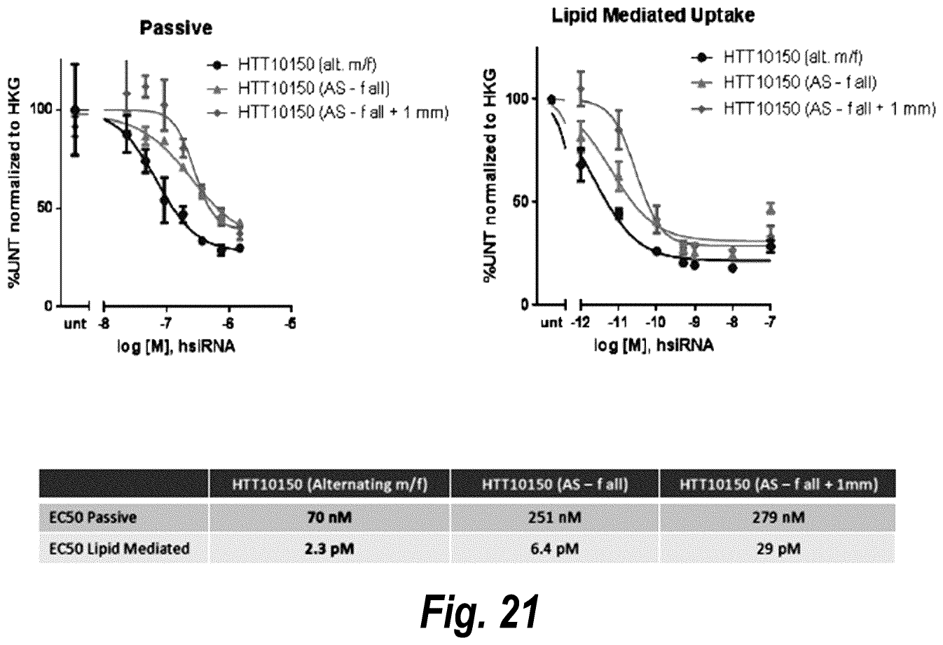

FIG. 21 depicts data showing that siRNA having a fully fluorinated antisense strand exhibit reduced efficacy as compared to the corresponding siRNA having an alternating 2′-methoxy/2′-fluoro pattern.

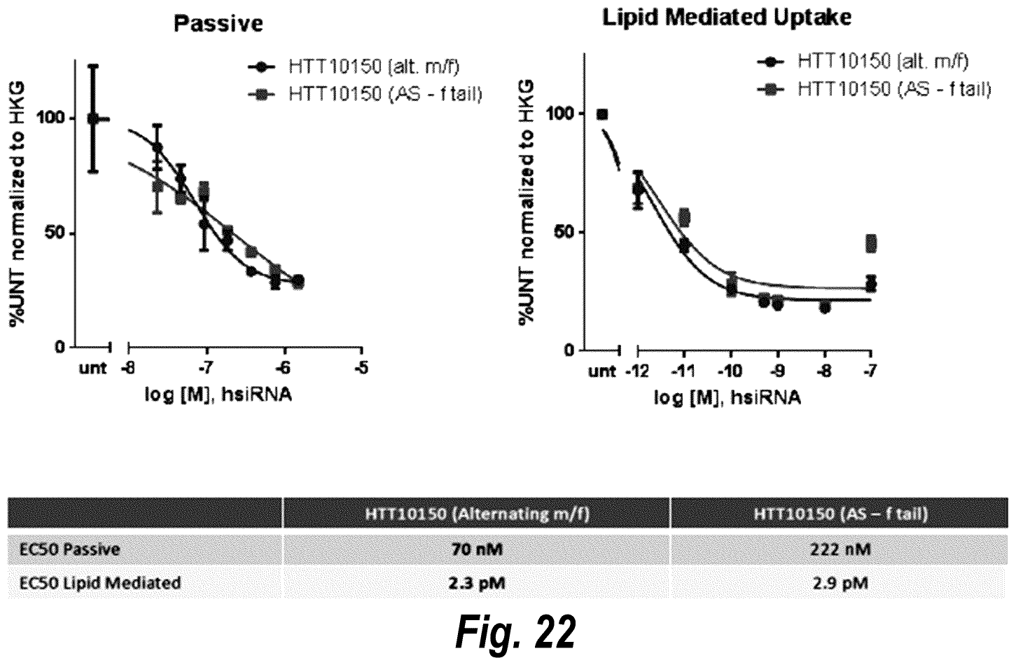

FIG. 22 depicts data showing that siRNA having a fluorinated antisense tail do not exhibit improved efficacy as compared to the corresponding siRNA having a fully alternating 2′-methoxy/2′-fluoro pattern.

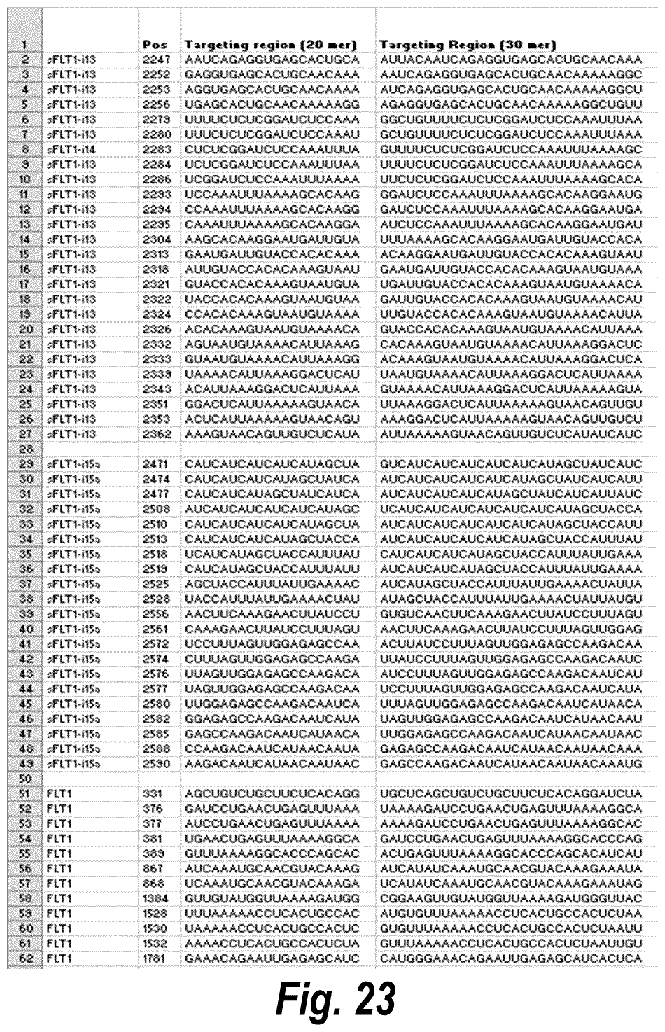

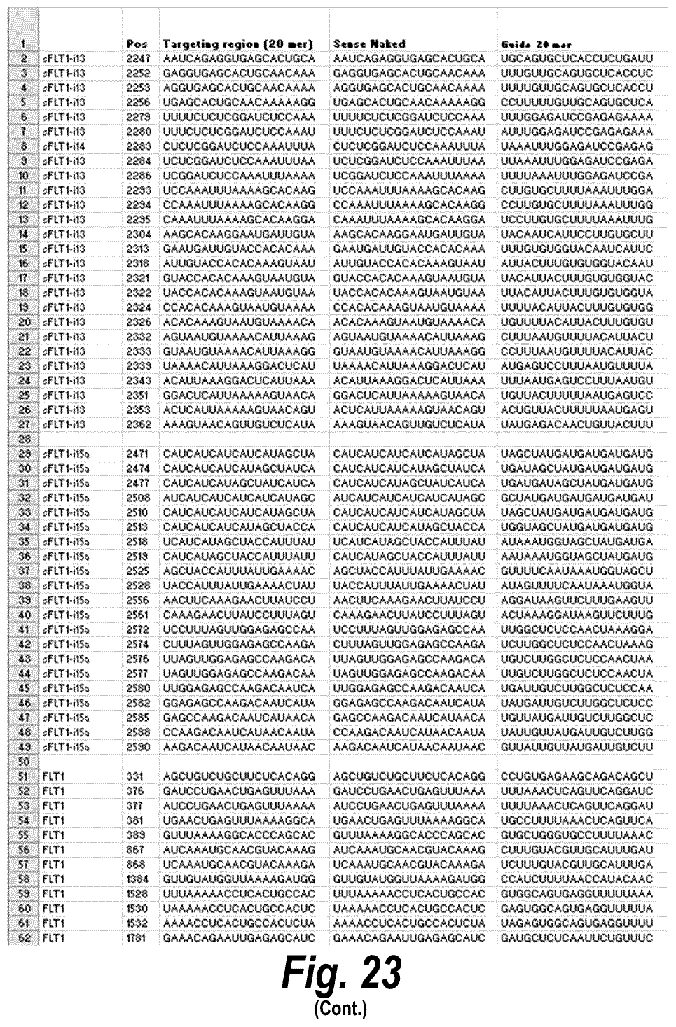

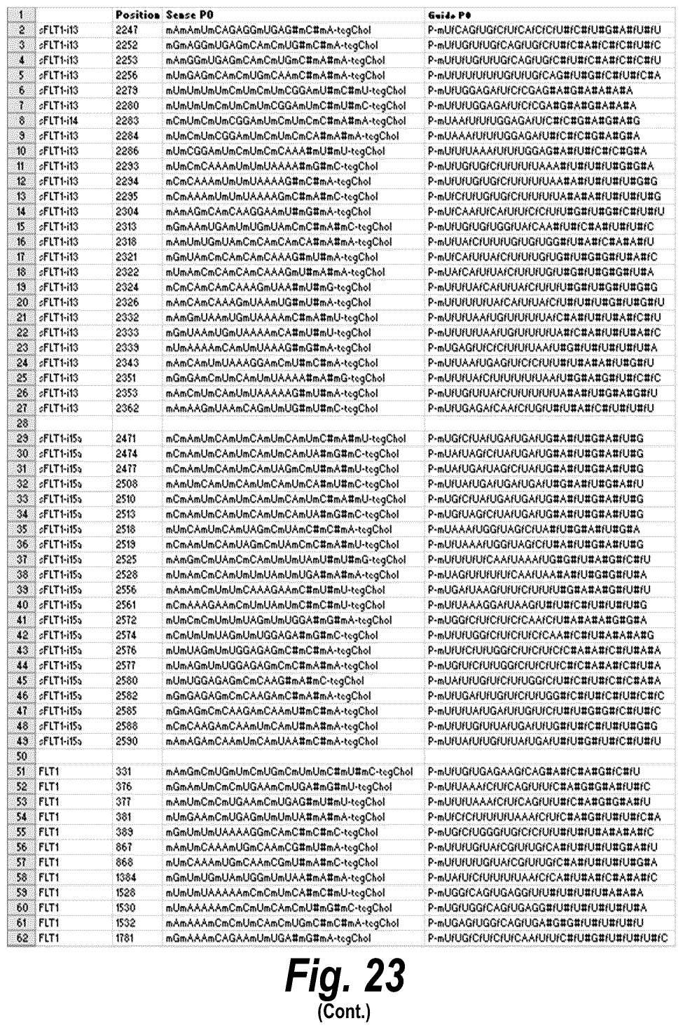

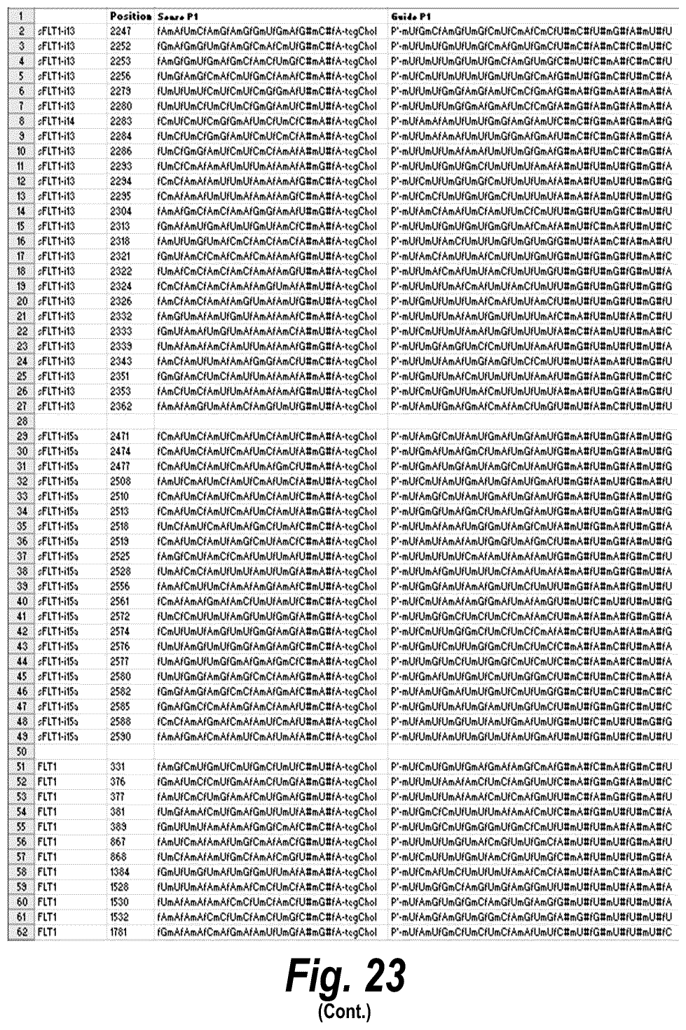

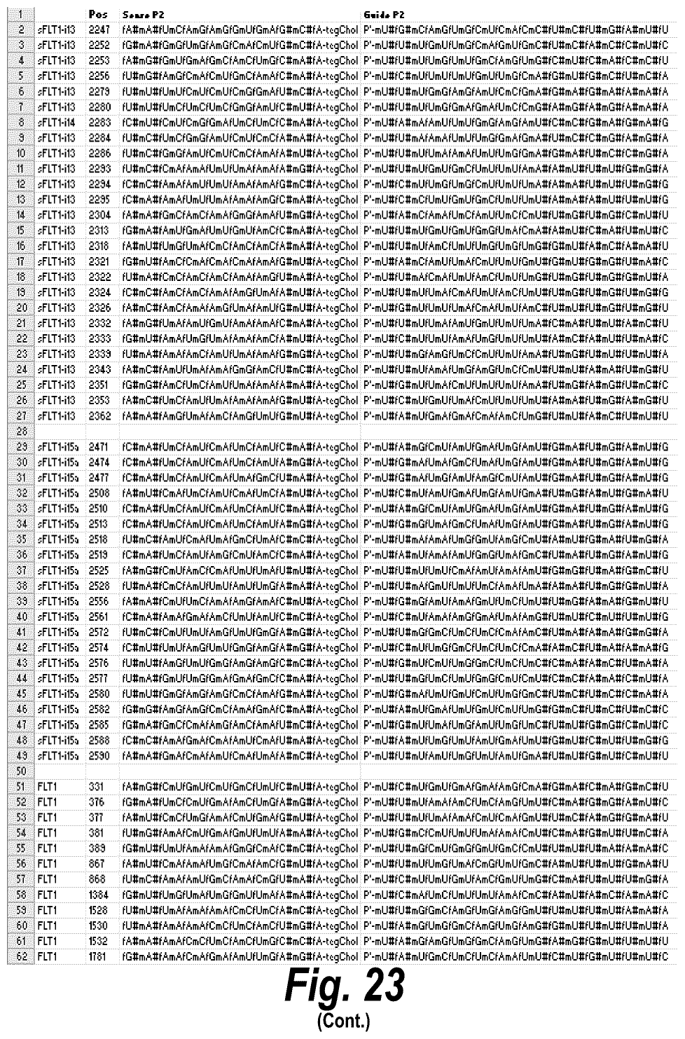

FIG. 23 depicts a list of siRNA having efficacy, representing different chemical scaffolds and unique sequences of I13 short, I13 long and I15a isoforms of sFlt1. In the table, “Guide” refers to the antisense strand; “C” represents cytidine; “U” represents uridine; “A” represents adenosine; “G” represents guanosine; “m” indicates a 2′-methoxy chemical modification; “f” indicates a 2′-fluoro chemical modification; “#” represents a phosphorothioate linker; “P” represents a 5′ phosphate; “teg” represents triethylene glycol; and “Chol” represents cholesterol. FIG. 23 discloses “Targeting region (20 mer)” sequences as SEQ ID NOS 22-80, “Targeting region (30 mer)” sequences as SEQ ID NOS 81-139, “Sense Naked” sequences as SEQ ID NOS 140-198, “Guide 20 mer” sequences as SEQ ID NOS 199-257, “Sense PO” sequences as SEQ ID NOS 258-316, “Guide P0” sequences as SEQ ID NOS 317-375, “Sense P1” sequences as SEQ ID NOS 376-434, “Guide P1” sequences as SEQ ID NOS 435-493, “Sense P2” sequences as SEQ ID NOS 494-552, “Guide P2” sequences as SEQ ID NOS 553-611, all respectively, in order of columns.

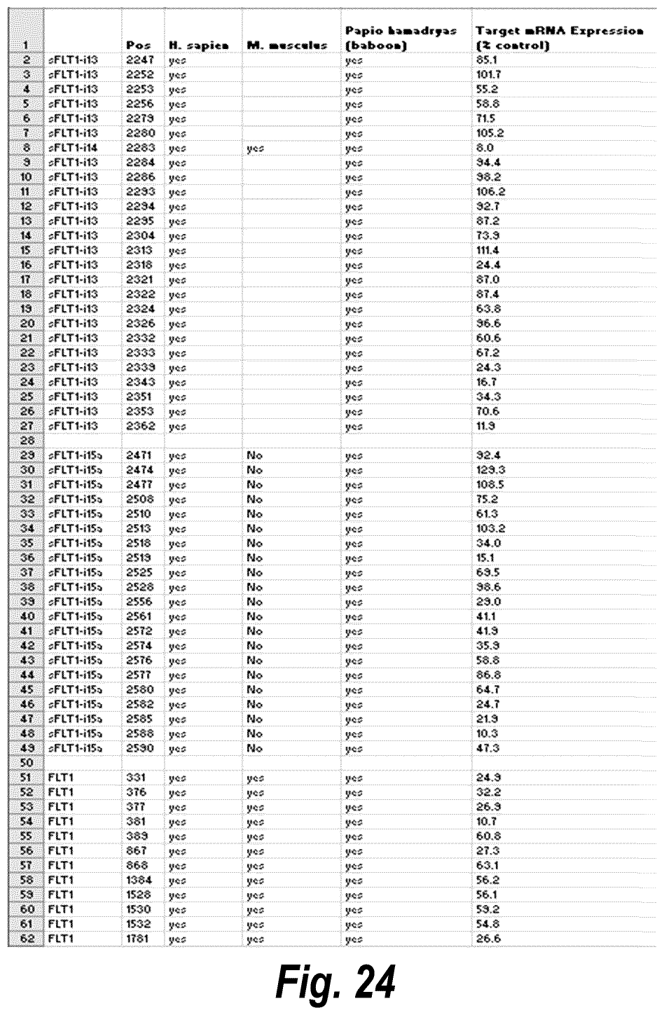

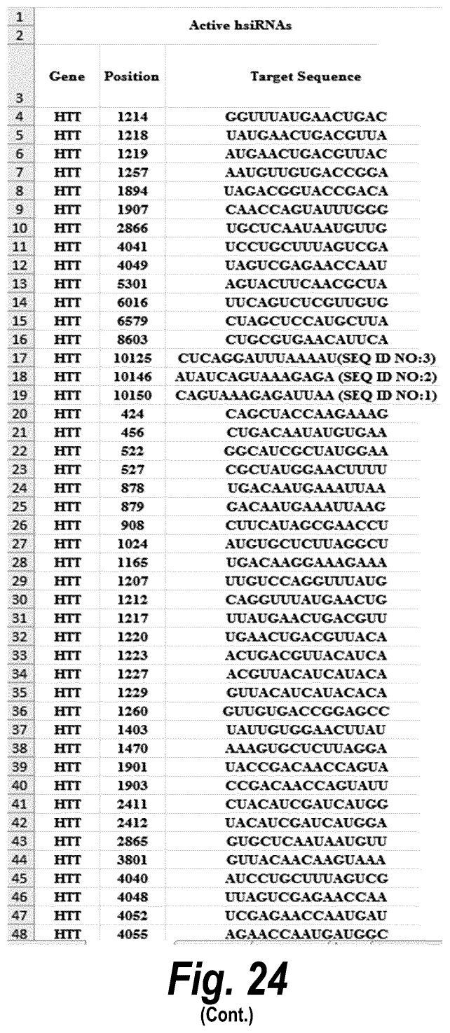

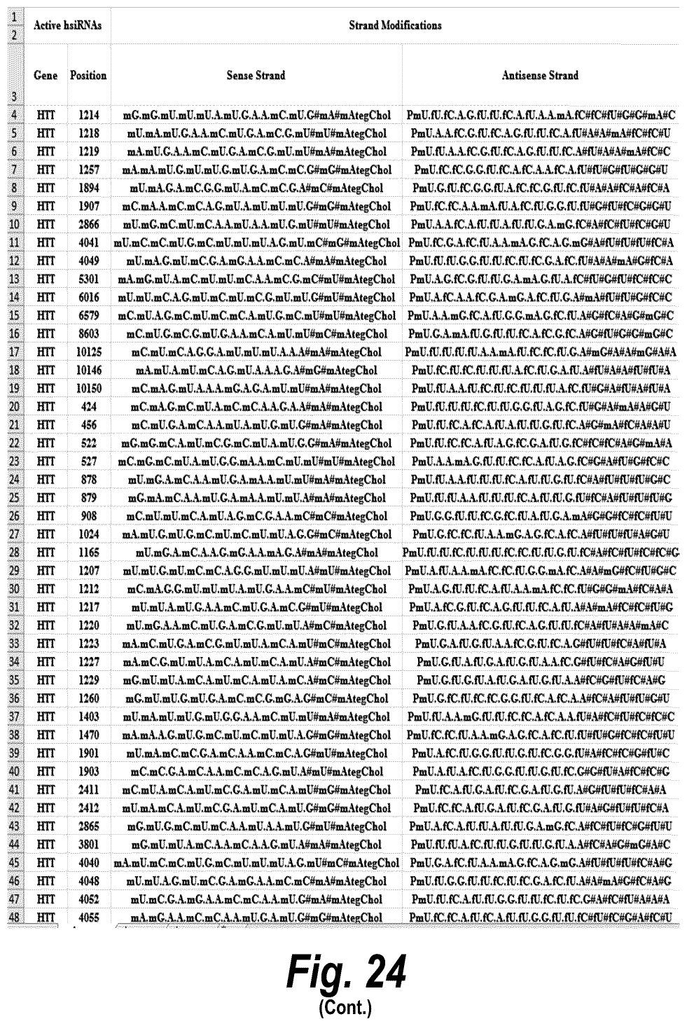

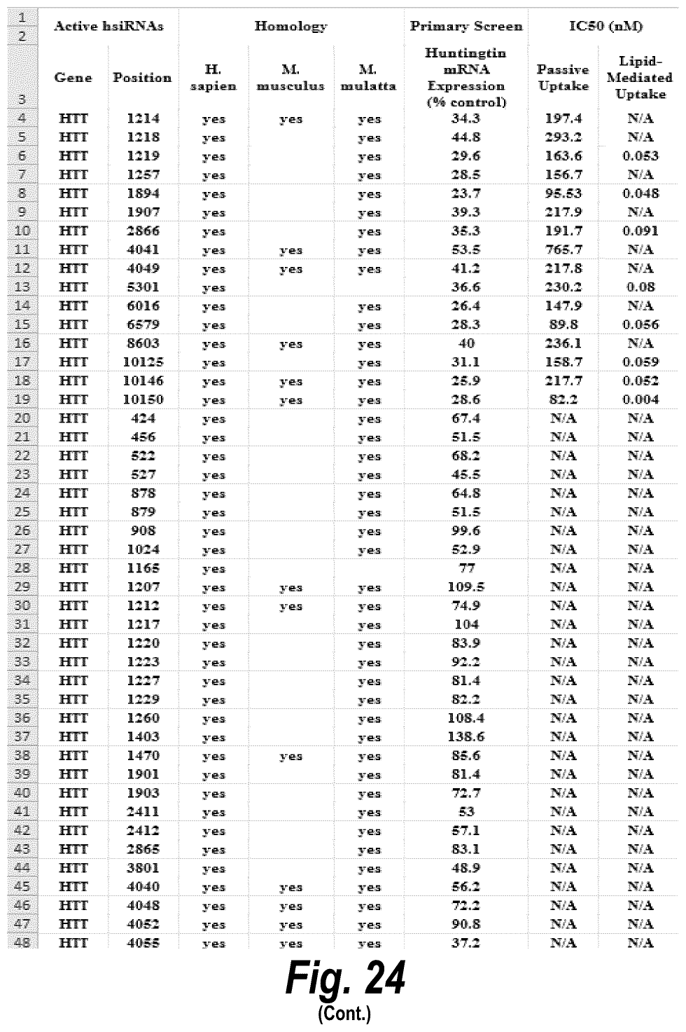

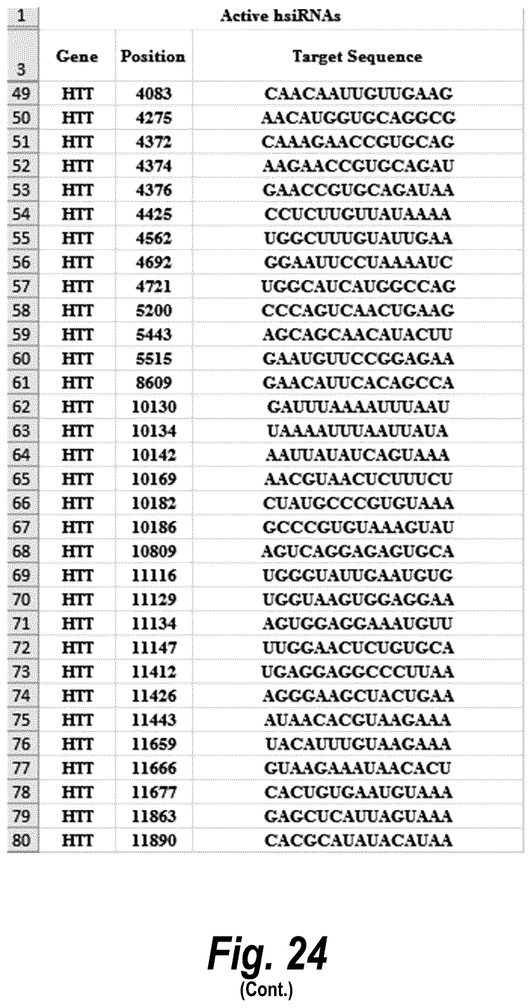

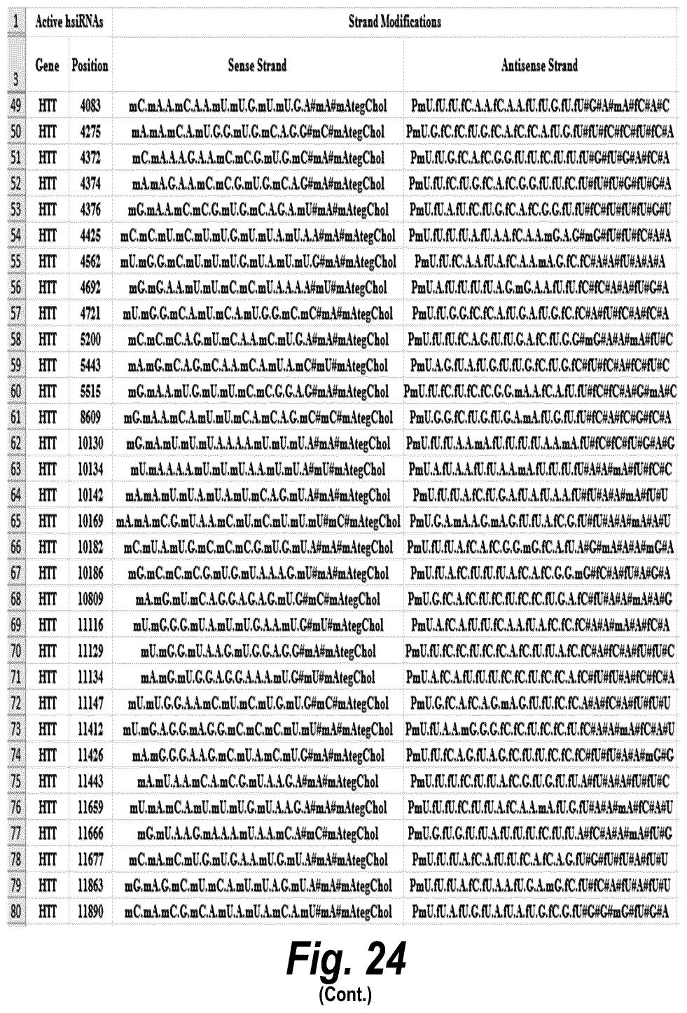

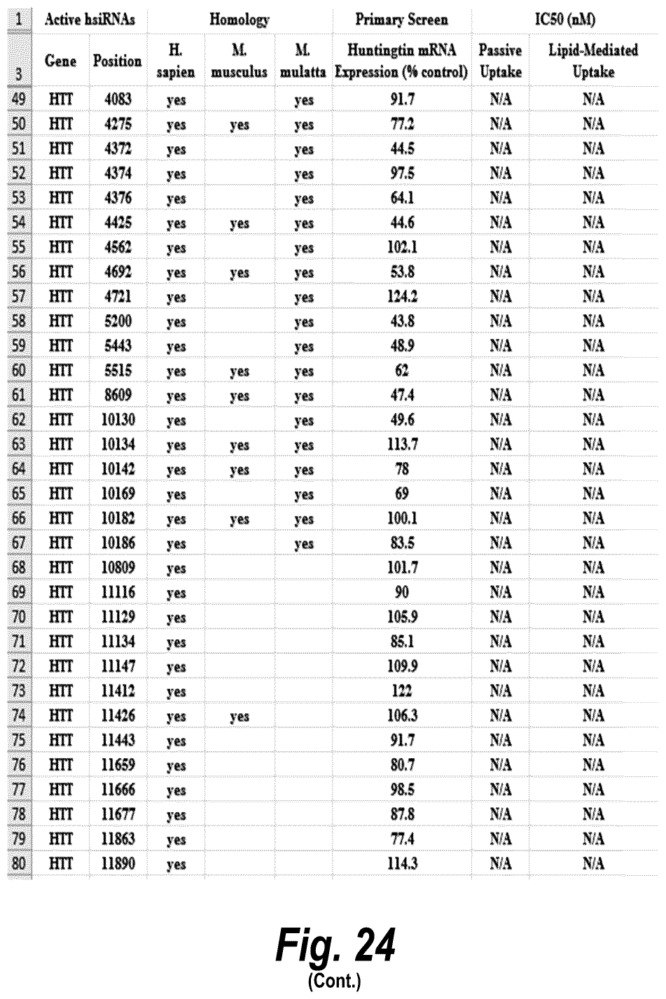

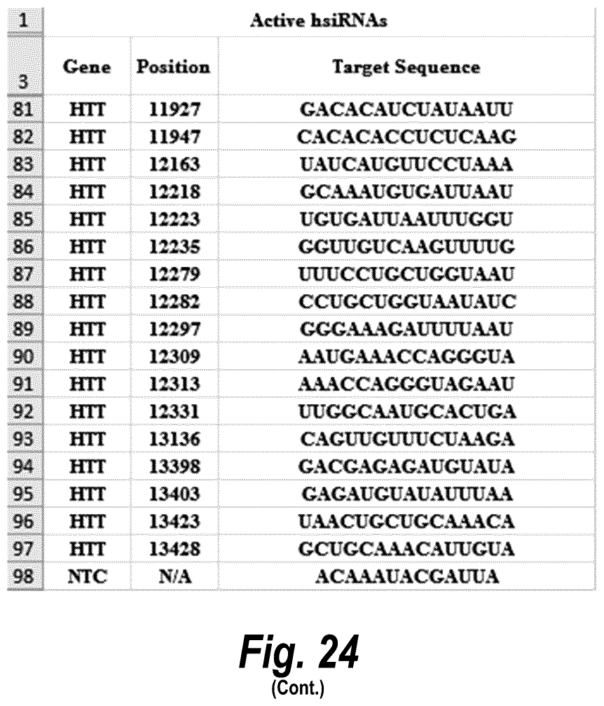

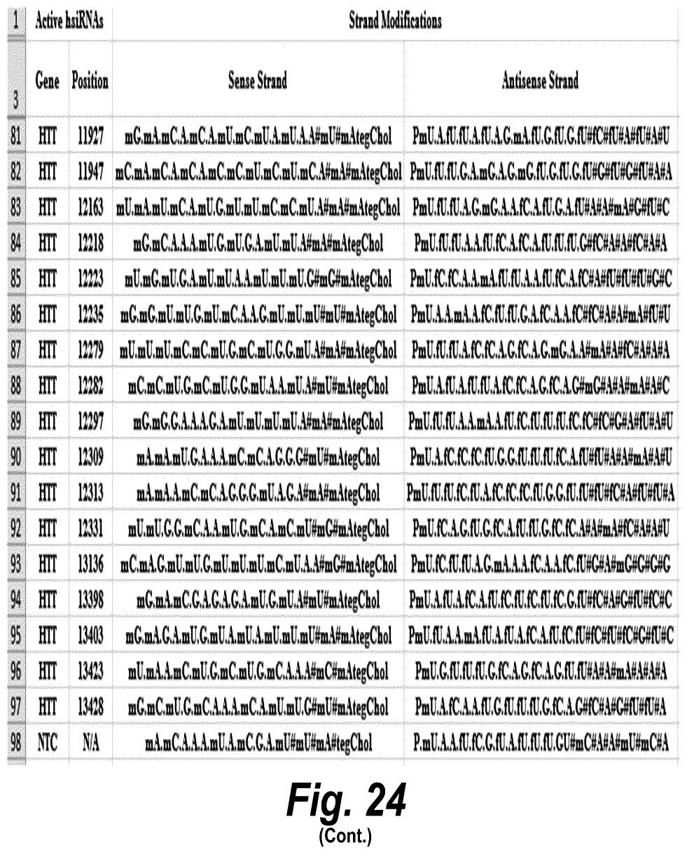

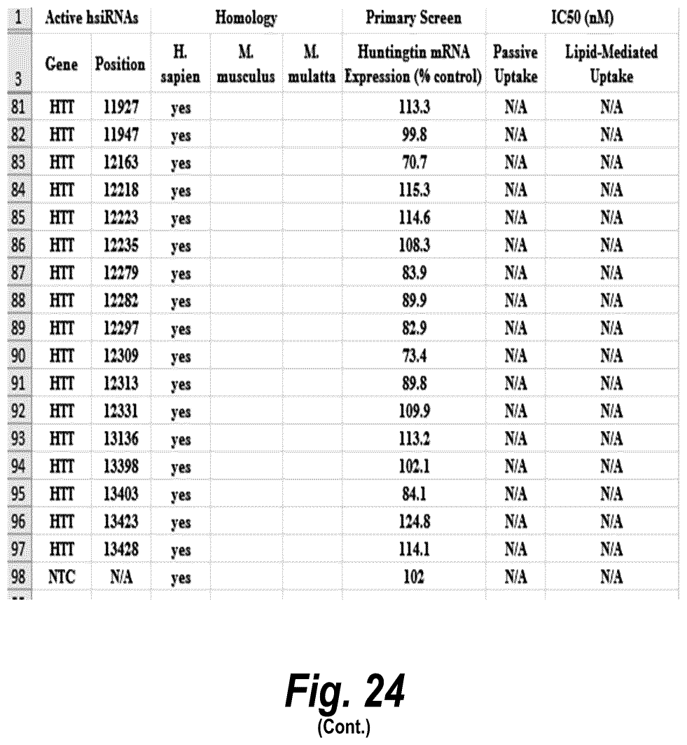

FIG. 24 depicts target sequences, modified oligonucleotides and their efficacy according to certain embodiments. In the table, “C” represents cytidine; “U” represents uridine; “A” represents adenosine; “G” represents guanosine; “m” indicates a 2′-methoxy chemical modification; “f” indicates a 2′-fluoro chemical modification; “#” represents a phosphorothioate linker; “P” represents a 5′ phosphate; “.” represents a phosphodiester linkage; “teg” represents triethylene glycol; and “Chol” represents cholesterol. FIG. 24 discloses “Target” sequences as SEQ ID NOS 612-624, 3, 2, 1, and 625-703, “Sense Strand” sequences as SEQ ID NOS 704-798, and “Antisense Strand” sequences as SEQ ID NOS 799-893, all respectively, in order of columns.

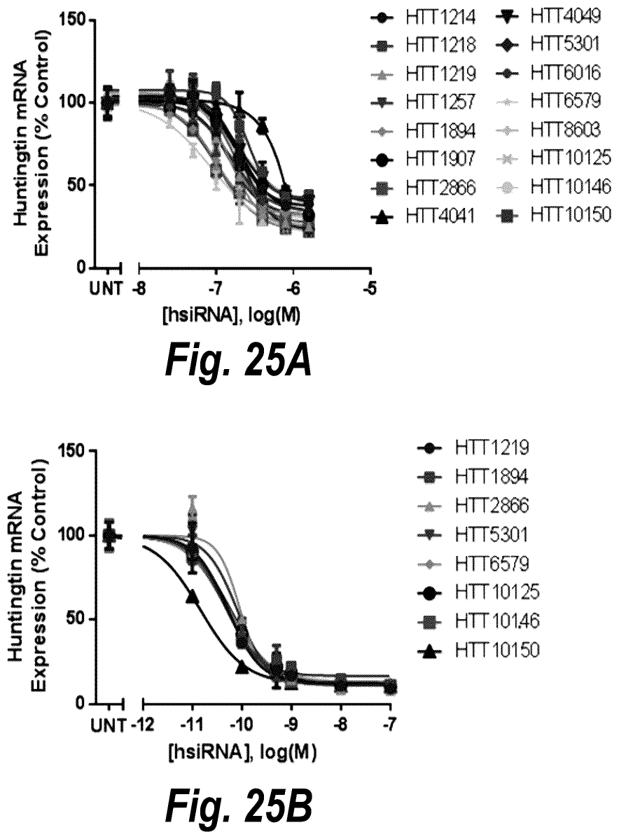

FIGS. 25A-25B depict graphically depict concentration-dependent silencing of huntingtin mRNA by HTT10150 in Hela cells. Level of huntingtin mRNA was measured using QUANTIGENE (Affymetrix) at 72 hours normalized to housekeeping gene, PPIB (cyclophillin B), and presented as percent of untreated control (n=3, mean+/−SD). UNT—untreated cells, NTC—non-targeting control. A) Dose response of 16 active sequences in passive uptake (no formulation). B) Dose response of eight selected sequences in lipid-mediated uptake (using Invitrogen LIPOFECTAMINE RNAIMAX Transfection Reagent). Dose response data was fitted using GraphPad Prism 6.03.

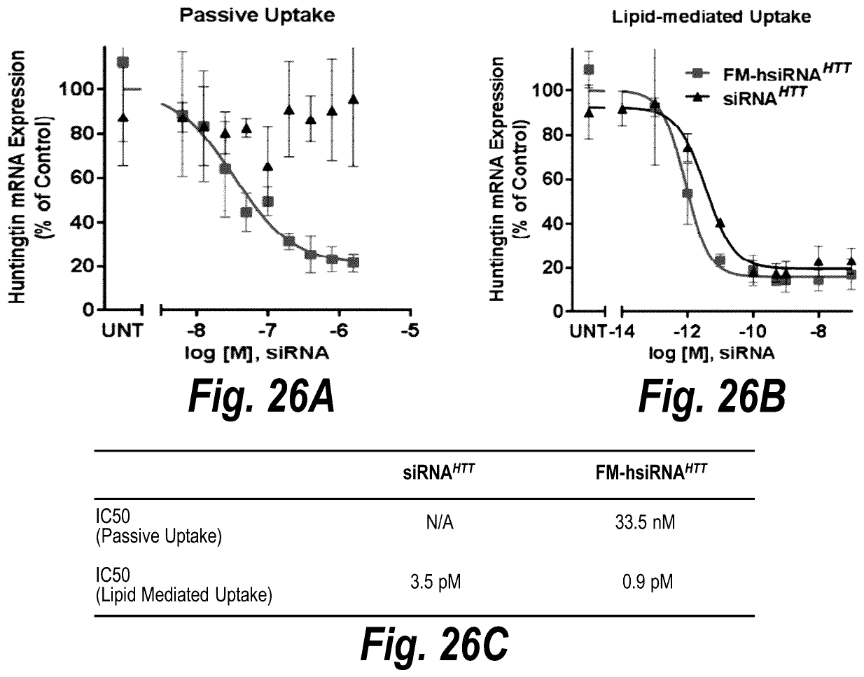

FIGS. 26A-26C depict concentration-dependent silencing of huntingtin mRNA by HTT10150, in both passive (A) and lipid-mediated delivery (B). Chemical modifications enable passive uptake without negative impact on siRNA RISC (RNA Induced Silencing Complex) entry. HeLa cells were incubated with modified (containing both hydrophobic and base chemical modifications) or unmodified HTT10150 at concentrations shown in the absence (A) and presence (B) of RNAIMAX. Level of huntingtin mRNA was measured using QUANTIGENE (Affymetrix) at 72 hours normalized to housekeeping gene, PPIB (cyclophillin B), and presented as percent of untreated control (n=3, mean+/−SD). UNT-untreated cells. IC50 values calculated (C) as described herein.,

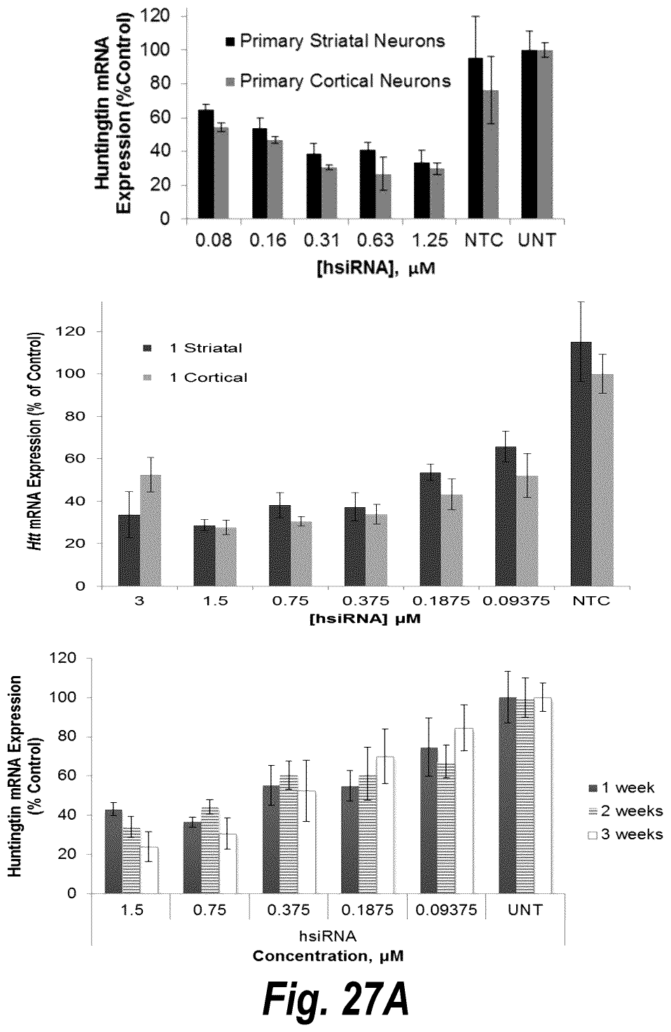

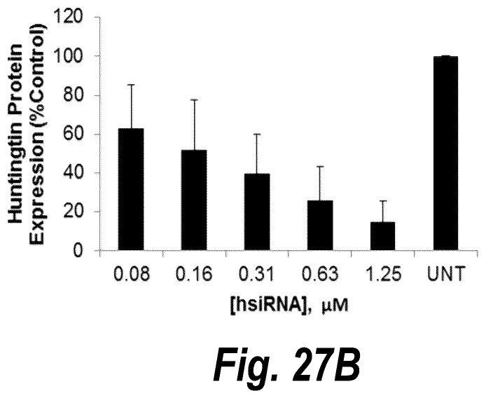

FIGS. 27A-27B graphically depict concentration-dependent silencing of huntingtin mRNA and protein by HTT10150 in primary neurons (passive uptake). Primary neurons were incubated with HTT10150 at concentrations shown. Level of huntingtin mRNA was measured using QUANTIGENE (Affymetrix) normalized to housekeeping gene, PPIB (cyclophillin B), and presented as percent of untreated control (n=3, mean+/−SD). UNT-untreated cells. (A) In primary cortical and striatal neurons, 1 week. (B) Huntingtin protein levels after one week incubation with HTT10150 were detected by western blot and normalized to β-Tubulin.

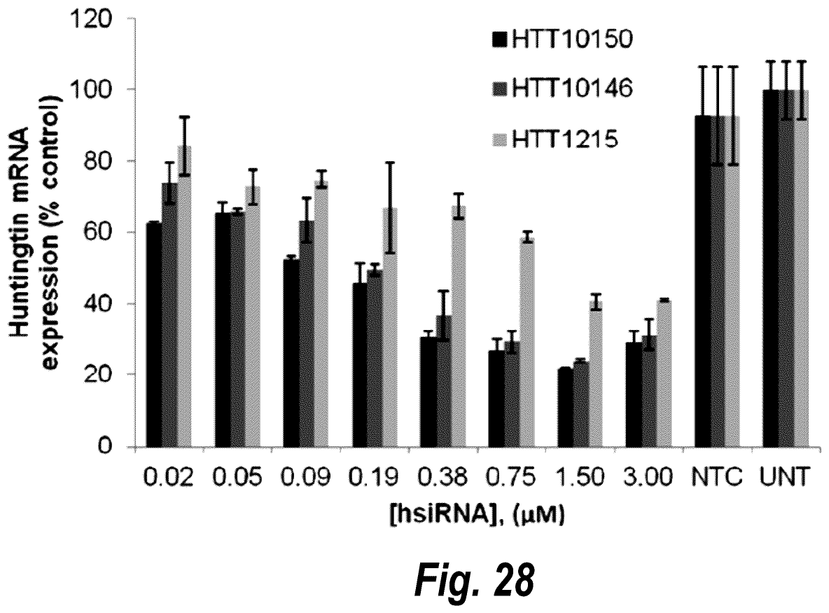

FIG. 28 depicts huntingtin mRNA levels. Primary cortical neurons were incubated with three HTT hsiRNA sequences HTT10150, HTT10146, and HTT1215 at concentrations shown. Level of huntingtin mRNA was measured using QUANTIGENE (Affymetrix) normalized to housekeeping gene, PPIB (cyclophillin B), and presented as percent of untreated control (n=3, mean+/−SD). UNT—untreated cells.

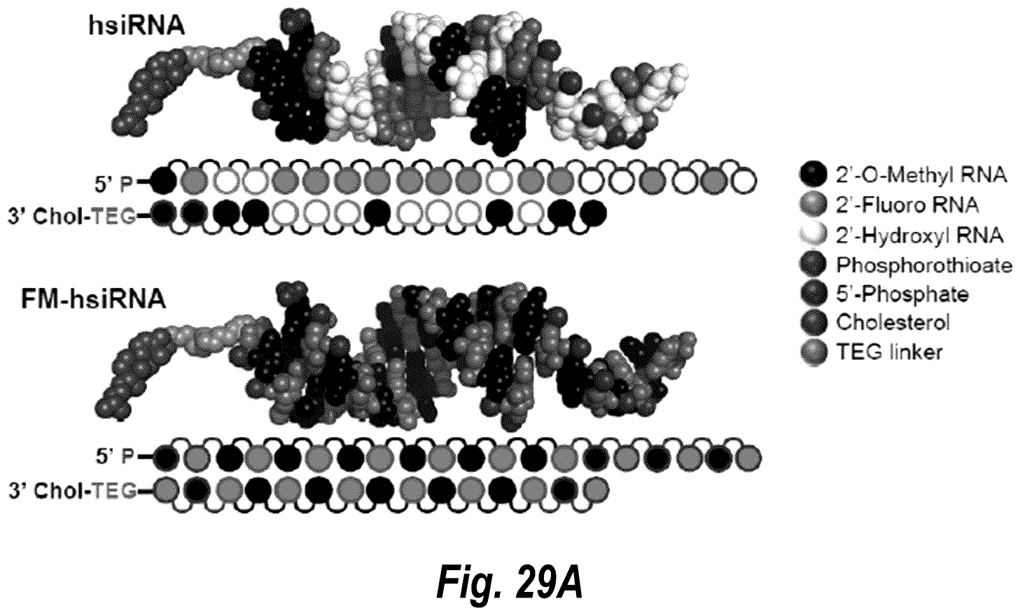

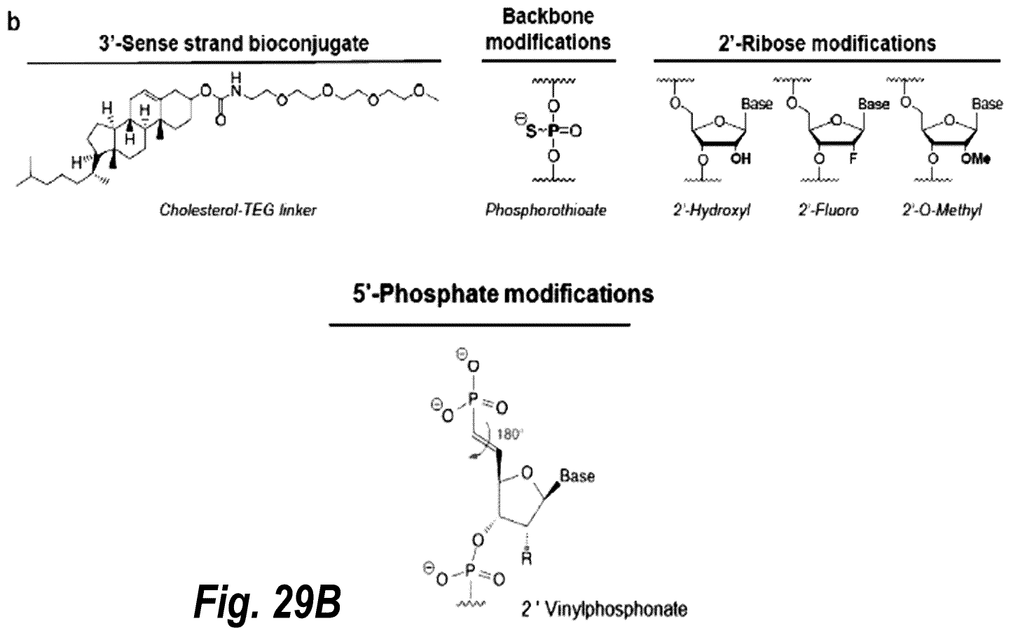

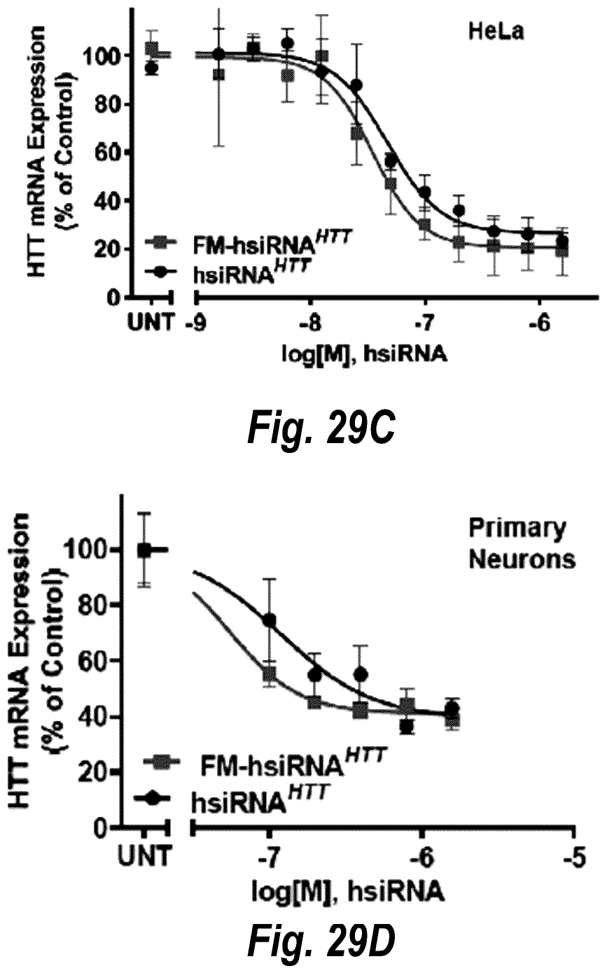

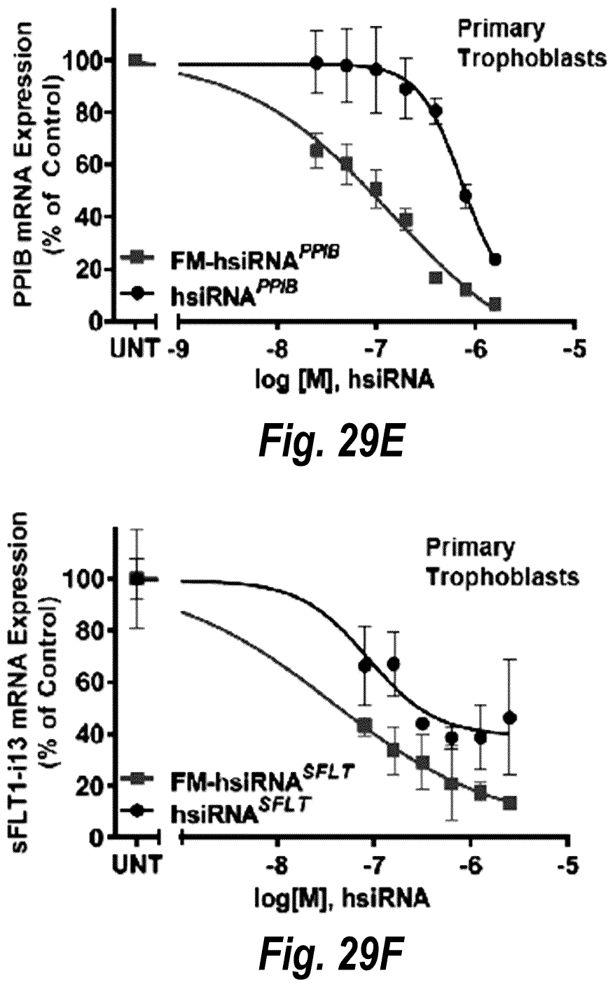

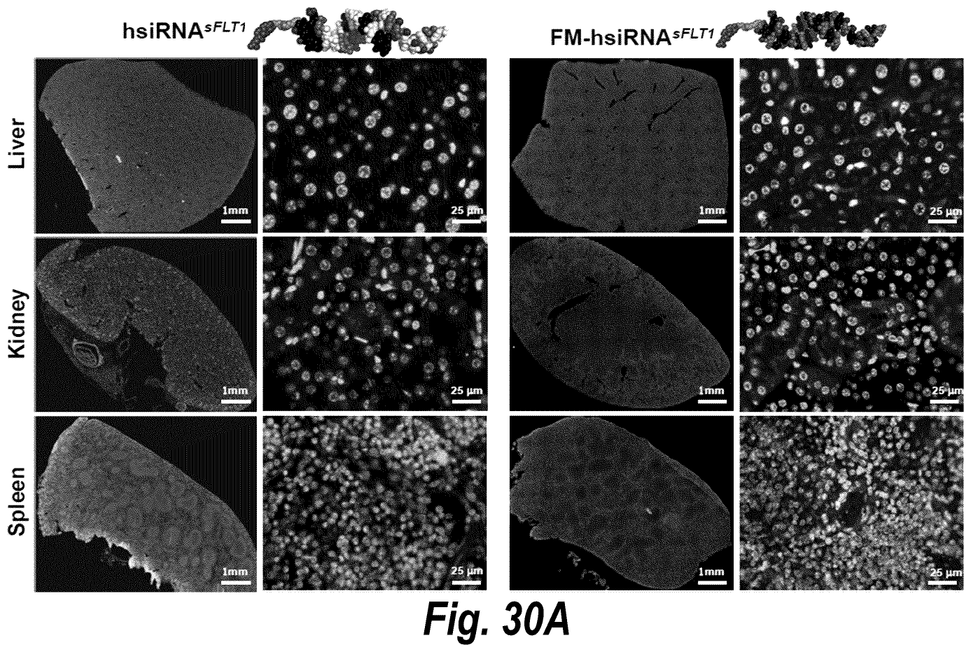

FIGS. 29A-29F depict hsiRNAs and their properties. (A) schematic structures of conventionally modified hsiRNA and fully modified hsiRNA (FM-hsiRNA). (B) modifications used in hsiRNAs. (C) hsiRNA^{HTT} efficacy in Hela cells. (D) hsiRNA^{HTT} efficacy in primary neurons. (E) hsiRNA^{PPIB} efficacy in primary trophoblasts. (F) hsiRNA^{SFLI} efficacy in primary trophoblasts.

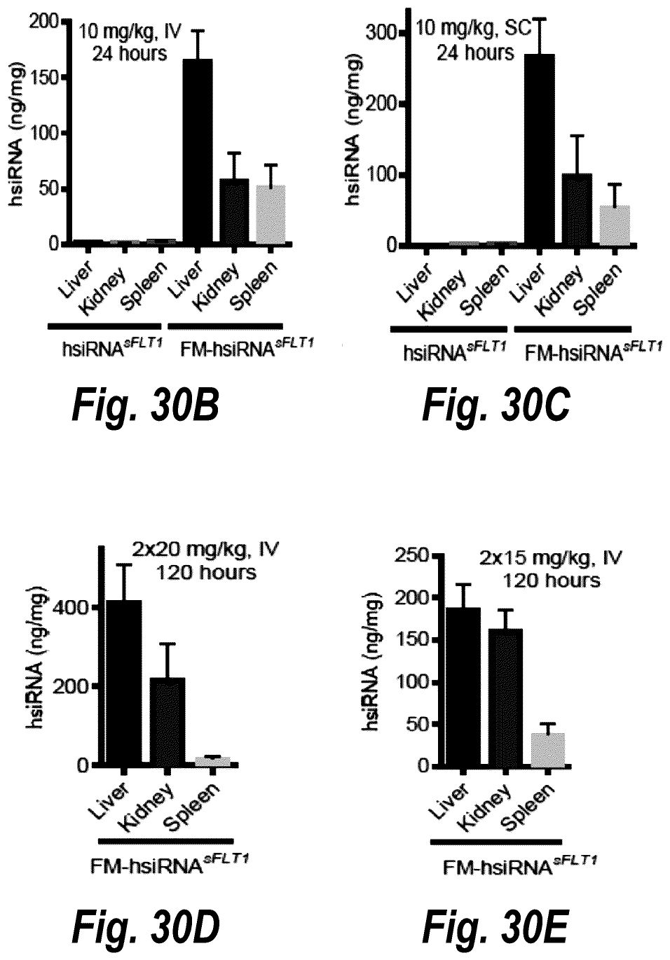

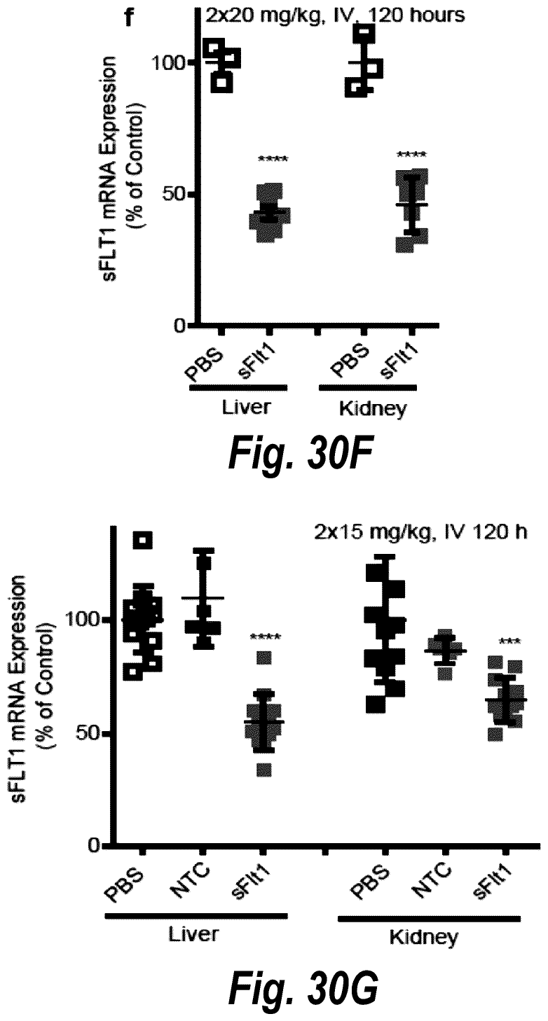

FIGS. 30A-30G depict full metabolic stabilization in conjugate-mediated siRNA delivery in vivo. (A) schematics of partially (hsiRNA) and fully metabolically stabilized hsiRNA-FMS compounds. (B) levels of hsiRNA^{sFLT} accumulation post-IV administration. (C) levels of hsiRNA^{sFLT} accumulation post-SC administration. (D) levels of FM-hsiRNA^{sFLT} accumulation post-IV administration. (E) levels of FM-hsiRNA^{sFLT} accumulation post-SC administration. (F) sFLT1 mRNA expression in liver and kidney 120 hours post-IV administration of hsiRNA^{sFLT}. (G) sFLT1 mRNA expression in liver and kidney 120 hours post-IV administration of FM-hsiRNA^{sFLT}.

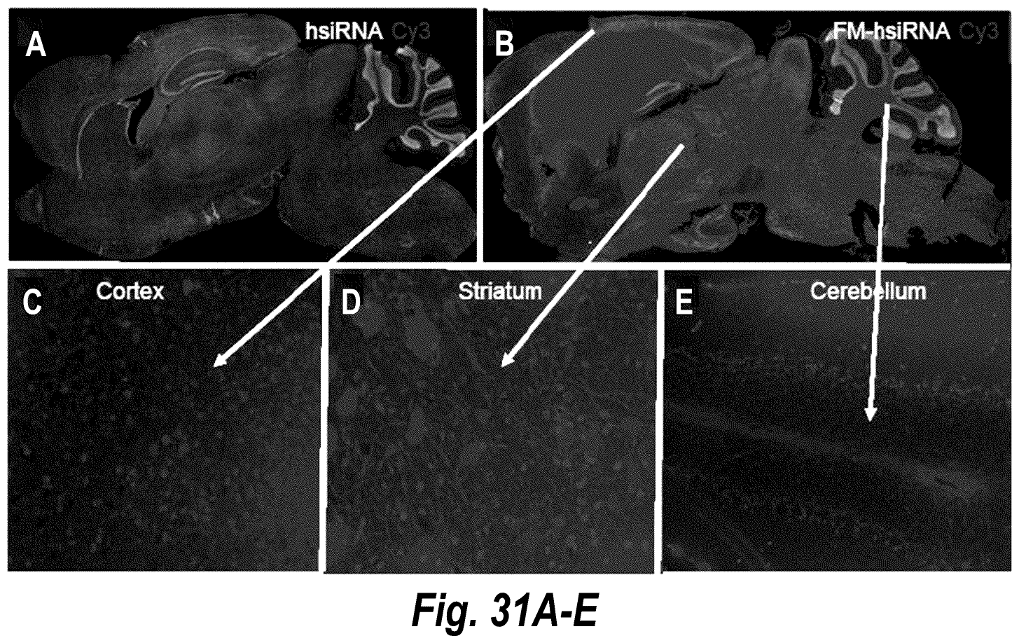

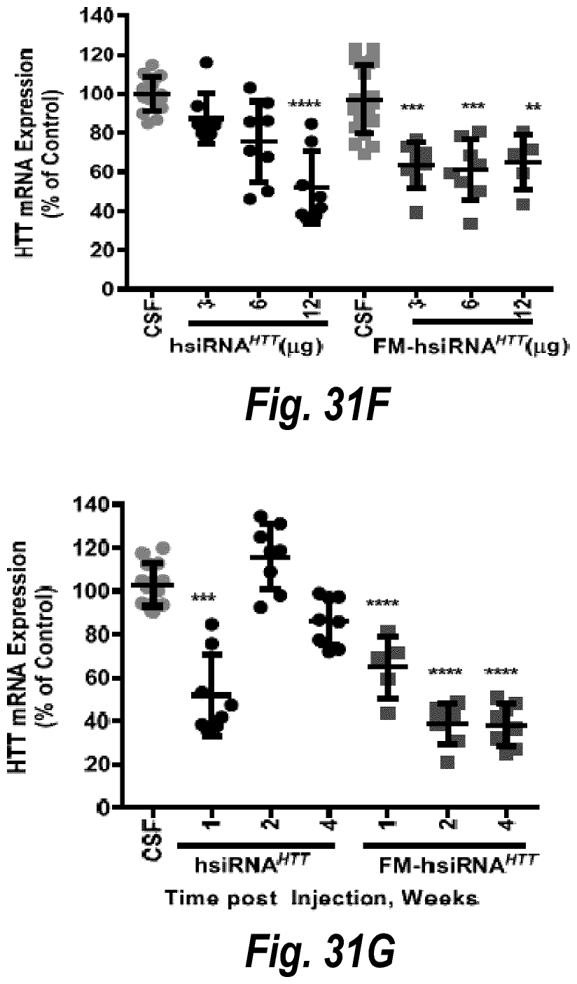

FIGS. 31A-31G depict results from intrastriatal administration of hsiRNA^{HTT} and FM-hsiRNA^{HTT}. (A) hsiRNA^{HTT} expression levels in a brain cross-section. (B) FM-hsiRNA^{HTT} expression levels in brain cross-section. (C) FM-hsiRNA^{HTT} expression levels in the cortex. (D) FM-hsiRNA^{HTT} expression levels in the striatum. (E) FM-hsiRNA^{HTT} expression levels in the cerebellum. (F) hsiRNA^{HTT} and FM-hsiRNA^{HTT} silencing using 3 μg, 6 μg and 12 μg doses. (G) hsiRNA^{HTT} and FM-hsiRNA^{HTT} silencing one-, two- and four-weeks post-injection.

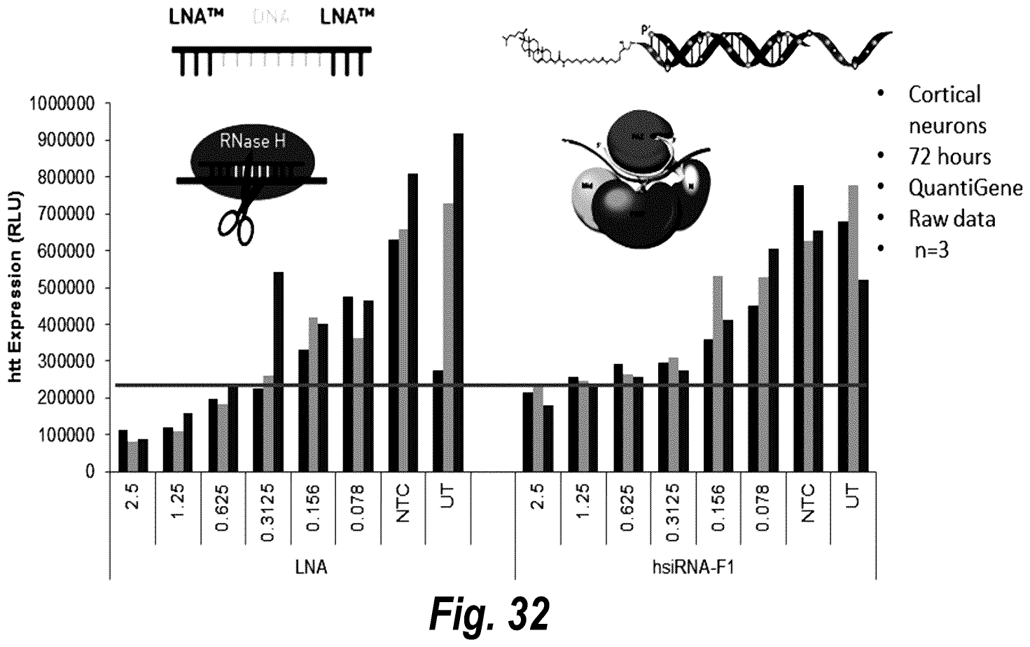

FIG. 32 depicts a comparison of hsiRNA^{HTT} (hsiRNA-F1) vs LNA-GAPMER silencing of Htt across a range of concentrations.

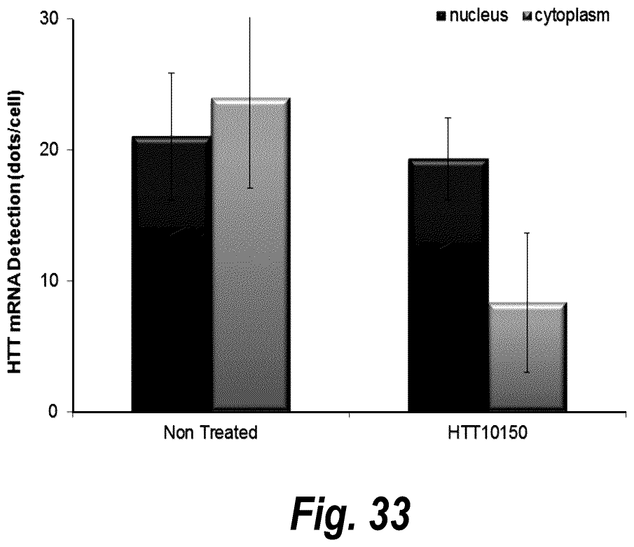

FIG. 33 depicts the preferential elimination of cytoplasmic Htt mRNA over nuclear Htt mRNA by HTT10150.

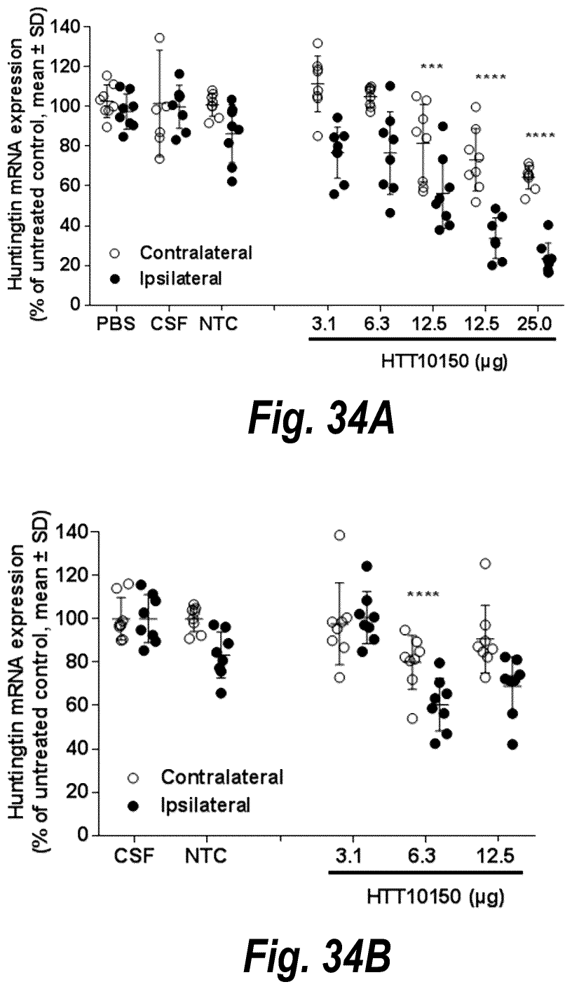

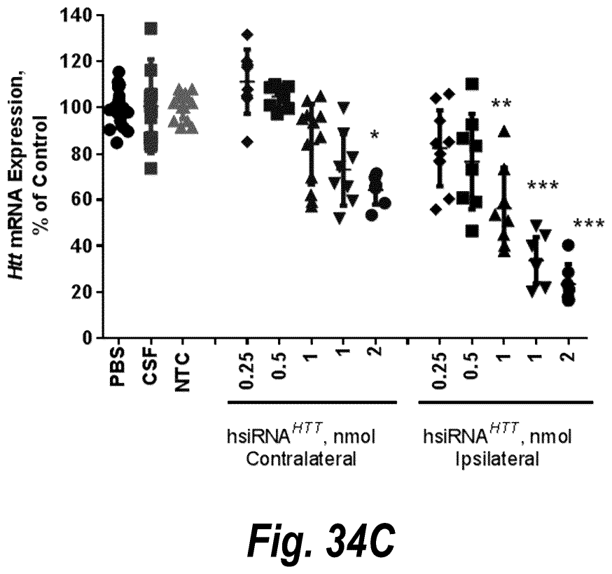

FIGS. 34A-34C depict results of single hsiRNA intrastriatal injections inducing dose-dependent silencing in vivo. (A) Huntingtin mRNA expression. (B) Huntingtin mRNA expression. (C) Htt mRNA expression.

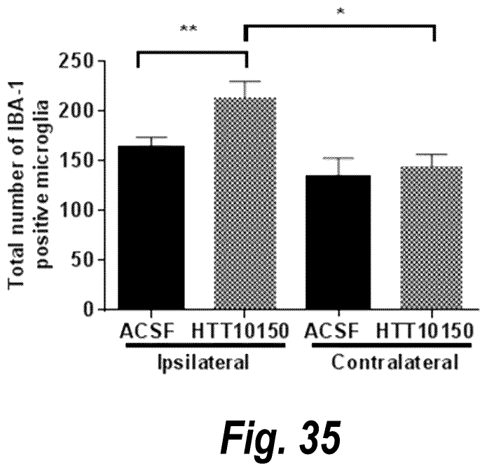

FIG. 35 depicts quantification of an inflammatory response at the site of injection of HTT10150.

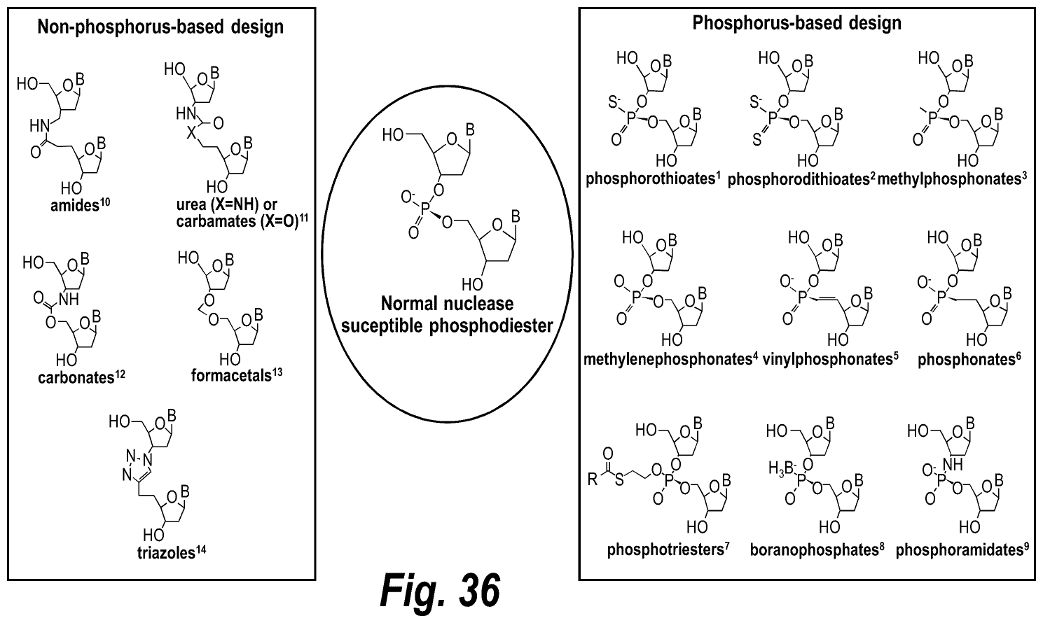

FIG. 36 depicts exemplary internucleotide linkages.

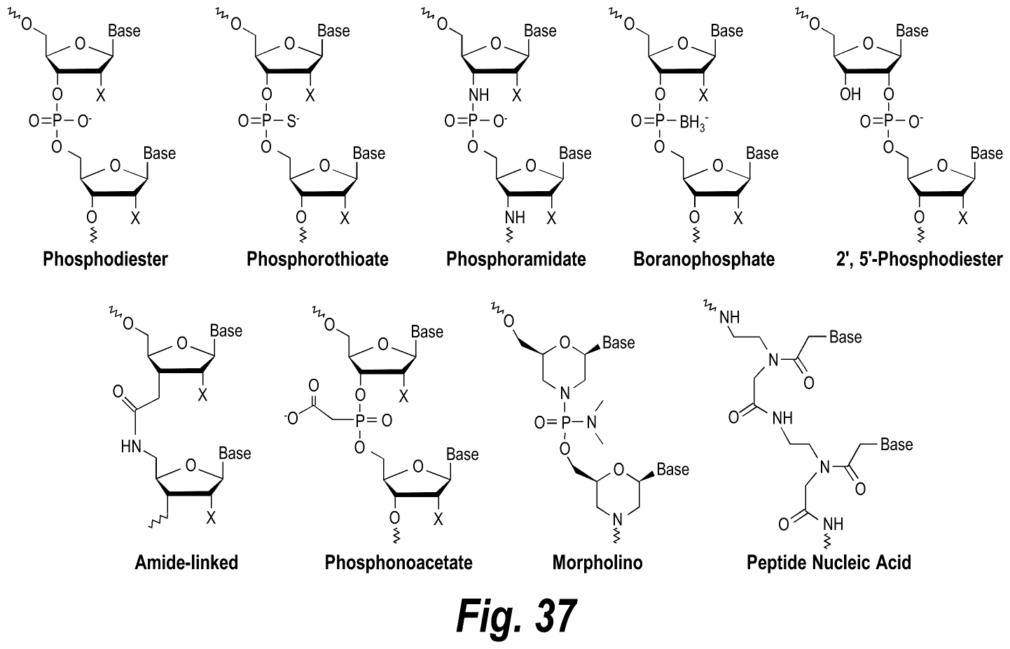

FIG. 37 depicts exemplary internucleotide backbone linkages.

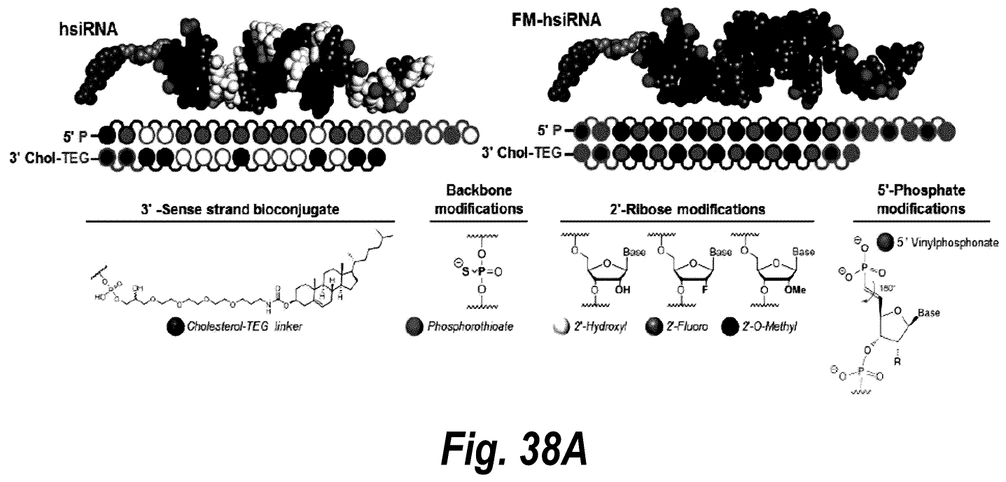

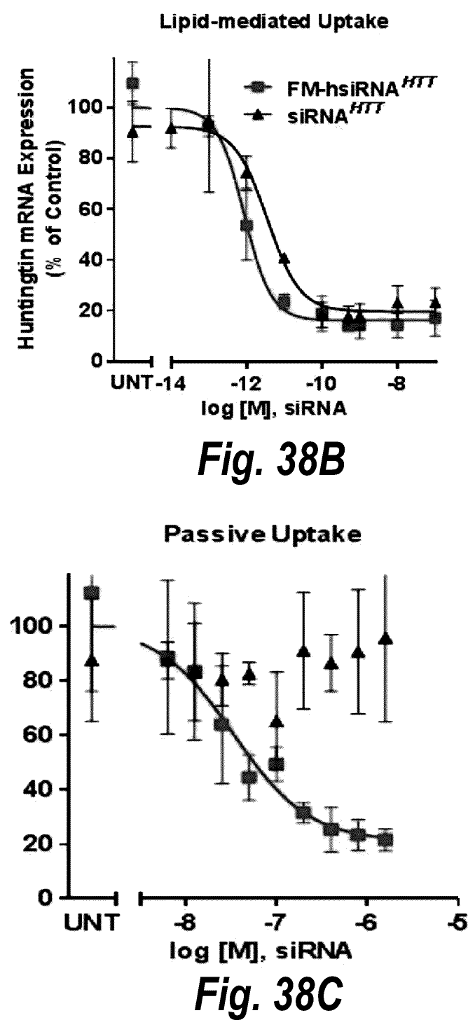

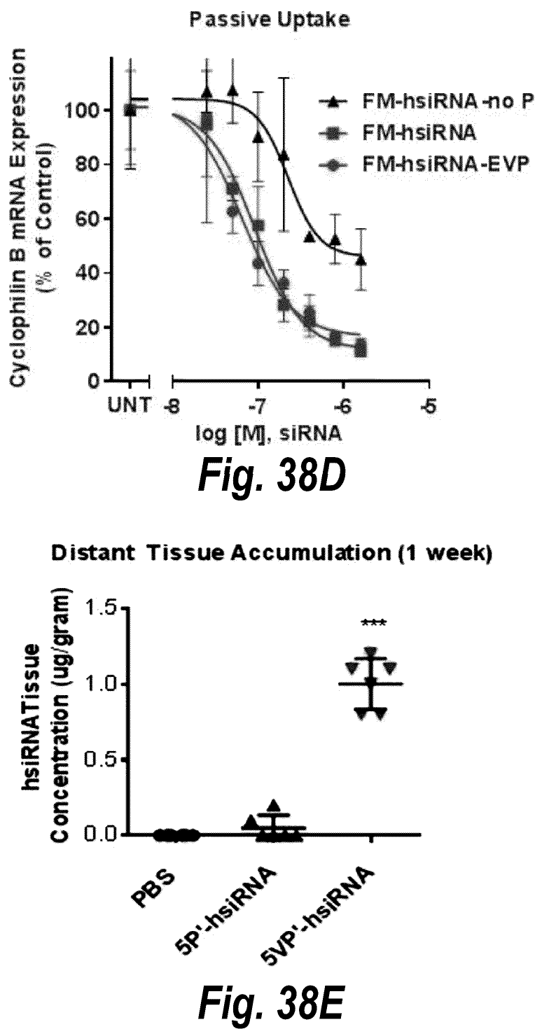

FIGS. 38A-38E depict fully metabolically stabilized hsiRNAs (FM-hsiRNAs). (A) Schematics of partially and fully modified hsiRNAs. (B) hsiRNA and FM-hsiRNA have equal ability to enter RISC (HeLa, 72 hours, QUANTIGENE). (C) FM-hsiRNA, but not naked siRNA, supports passive delivery. (D) Metabolically stable 5′-E-VP is as active as 5′-P. (E) 5′-E-VP enables sustained delivery to distant tissues (7 days post injection, PNA assay).

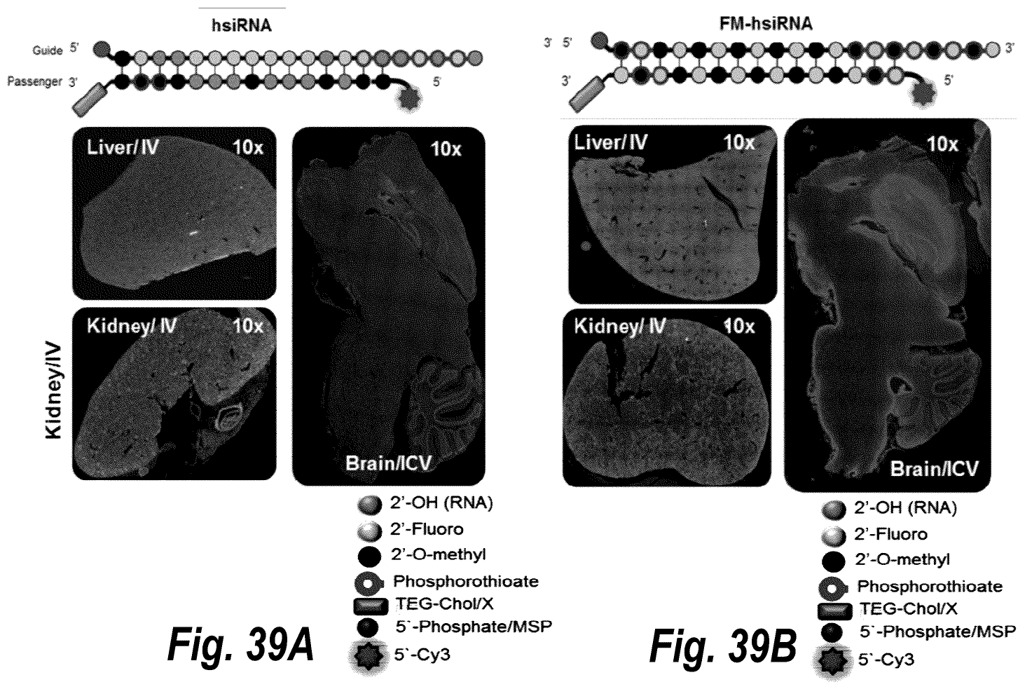

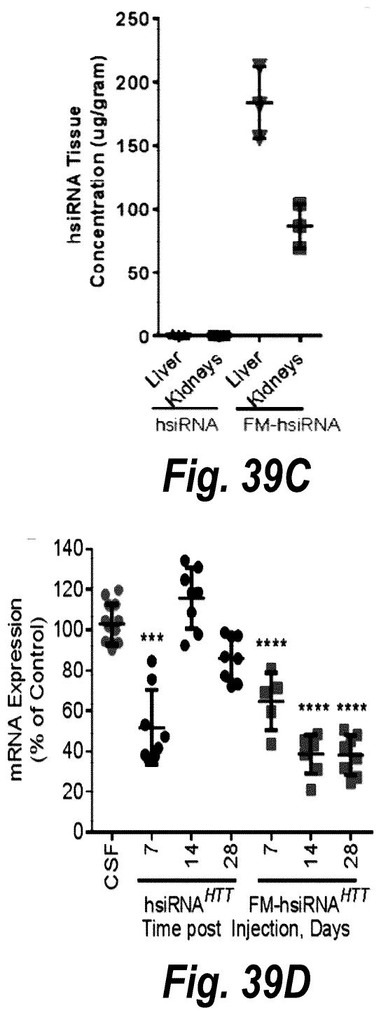

FIGS. 39A-39D depict conjugate mediated siRNA delivery and duration of effect in vivo. hsiRNA (A) and fully modified FM-hsiRNA (B) were injected IV (10 mg/kg) or ICV (60 ug) and distribution evaluated by microscopy (10×, Leica, Dapi, blue, Nuclei, Cy3, red, hsiRNA). Full stabilization dramatically enhances tissues retention. (C). Intact guide strand quantification analyzed by PNA assay (n=3, mean+SEM) in livers and kidneys 5 days after IV injection. (D) FM-hsiRNAs silences Htt mRNA in mouse striatum 1 month after injection (IS, 12 ug), QUANTIGENE. Partially modified hsiRNAs loses silencing after 1 week.

### DETAILED DESCRIPTION

In a first aspect, provided herein is compound (I): an oligonucleotide of at least 16 contiguous nucleotides, said oligonucleotide having a 5′ end, a 3′ end and complementarity to a target, wherein:
(1) the oligonucleotide comprises alternating 2′-methoxy-ribonucleotides and 2′-fluoro-ribonucleotides;
(2) the nucleotides at positions 2 and 14 from the 5′ end are not 2′-methoxy-ribonucleotides;
(3) the nucleotides are connected via phosphodiester or phosphorothioate linkages; and
(4) the nucleotides at positions 1-6 from the 3′ end, or positions 1-7 from the 3′ end, are connected to adjacent nucleotides via phosphorothioate linkages.

In one embodiment, the oligonucleotide has sufficient complementarity to the target to hybridize. In certain embodiments, the complementarity is >95%, >90%, >85%, >80%, >75%, >70%, >65%, >60%, >55% or >50%. In one embodiment, the oligonucleotide has perfect complementarity to the target.

In one embodiment of the oligonucleotide, the target is mammalian or viral mRNA. In another embodiment, the target is an intronic region of said mRNA. In another embodiment, the target is a 5′ UTR region of said mRNA. In another embodiment, the mRNA corresponds to a portion of soluble Flt1 (sFlt1). In a particular embodiment, the mRNA corresponds to a portion (e.g., an intronic region) of sFlt i13 (e.g., sFlt-i13 long or sFlt-i13 short). In another particular embodiment, the mRNA corresponds to a portion (e.g., an intronic region) of sFlt i15a. In another embodiment, the mRNA corresponds to a portion of the Huntingtin gene (e.g., a mutant Huntingtin gene).

In a second aspect, provided herein is a double-stranded, chemically-modified nucleic acid, comprising a first oligonucleotide compound (I) and a second oligonucleotide compound (II), wherein:
(1) a portion of the first oligonucleotide is complementary to a portion of the second oligonucleotide;
(2) the second oligonucleotide comprises alternating 2′-methoxy-ribonucleotides and 2′-fluoro-ribonucleotides;
(3) the nucleotides at positions 2 and 14 from the 3′ end of the second oligonucleotide are 2′-methoxy-ribonucleotides; and
(4) the nucleotides of the second oligonucleotide are connected via phosphodiester or phosphorothioate linkages.

In one embodiment, the first oligonucleotide is the antisense strand and the second oligonucleotide is the sense strand.

In one embodiment, the double-stranded nucleic acid comprises one or more mismatch within the complementary portions of the first and second oligonucleotide. In a particular embodiment, the double-stranded nucleic acid contains one mismatch within the complementary portions of the first and second oligonucleotide.

In one embodiment of the nucleic acid, the second oligonucleotide is linked to a hydrophobic molecule at the 3′ end of the second oligonucleotide. In one embodiment, the linkage between the second oligonucleotide and the hydrophobic molecule comprises polyethylene glycol. In a particular embodiment, the linkage between the second oligonucleotide and the hydrophobic molecule comprises triethylene glycol.

In another embodiment of the nucleic acid, the nucleotides at positions 1 and 2 from the 3′ end of second oligonucleotide are connected to adjacent nucleotides via phosphorothioate linkages. In yet another embodiment, the nucleotides at positions 1 and 2 from the 3′ end of second oligonucleotide, and the nucleotides at positions 1 and 2 from the 5′ end of second oligonucleotide, are connected to adjacent ribonucleotides via phosphorothioate linkages.

In a third aspect, provided herein is an oligonucleotide having the structure of compound (Ia):

X(-K-B-K-A)ⱼ(-S-B-S-A)ᵣ(-S-B)ₜ-OR (Ia)
wherein:
X is selected from the group consisting of:

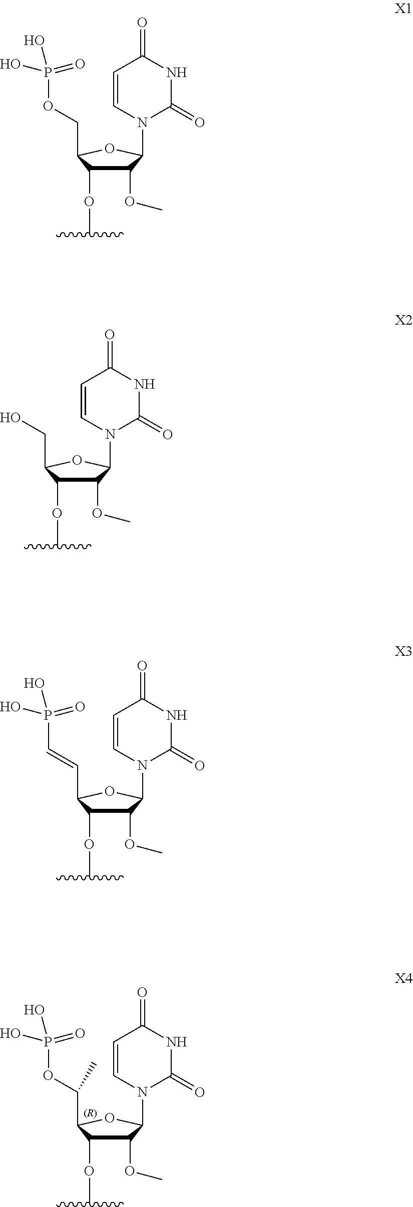

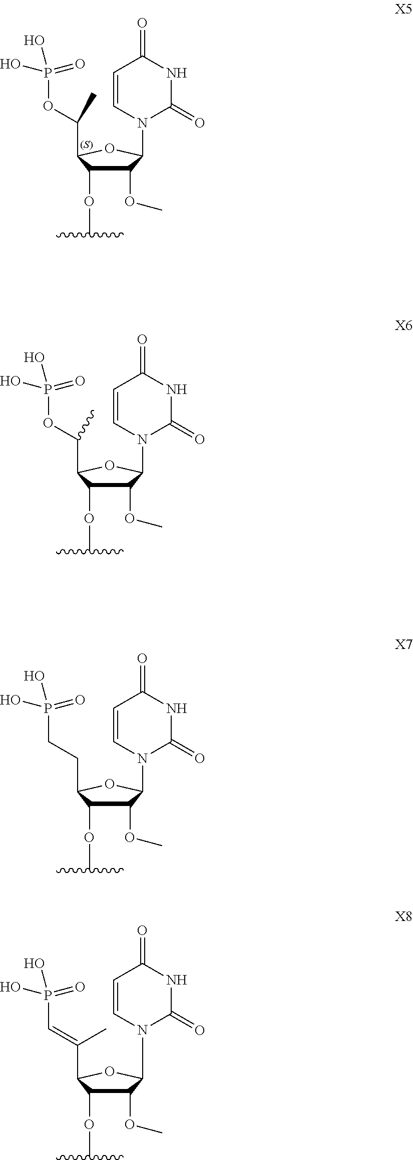

A, for each occurrence, independently is a 2′-methoxy-ribonucleotide; B, for each occurrence, independently is a 2′-fluoro-ribonucleotide; K, for each occurrence independently is a phosphodiester or phosphorothioate linker;
S is a phosphorothioate linker;
R, for each occurrence, independently is selected from hydrogen and a capping group (e.g., an acyl group such as acetyl);
j is 4, 5, 6 or 7;
r is 2 or 3; and
t is 0 or 1.

In a particular embodiment, R is hydrogen. In another particular embodiment, X is X1. In still another particular embodiment, X is X3.

In one embodiment, the oligonucleotide of compound (Ia) has the structure of compound (Ib):

X-A(-S-B-S-A)ₘ(-P-B-P-A)ₙ(-P-B-S-A)_{q}(-S-B-S-A)ᵣ(-S-B)ₜ-OR (Ib)
wherein:
X is as defined above;
A, for each occurrence, independently is a 2′-methoxy-ribonucleotide;
B, for each occurrence, independently is a 2′-fluoro-ribonucleotide;
S is a phosphorothioate linker;
P is a phosphodiester linker;
R is as defined above;
m is 0 or 1;
n is 4, 5 or 6;
q is 0 or 1;
r is 2 or 3; and
t is 0 or 1.

In a first particular embodiment of compound (Ib), m is 0; n is 6; q is 1; r is 2; and t is 1. See, e.g., species P1 of FIG. 7 and species HTT10150-ASP-P1 of FIG. 17.

In a second particular embodiment of compound (Ib), m is 1; n is 5; q is 1; r is 2; and t is 1. See, e.g., species P2 of FIG. 7.

In a third particular embodiment of compound (Ib), m is 1; n is 5; q is 0; r is 3; and t is 1. See, e.g., species HTT10150-ASP-P2 of FIG. 17.

In a particular embodiment, R is hydrogen. In another particular embodiment, X is X1. In still another particular embodiment, X is X3.

In a fourth aspect, provided herein is a double-stranded, chemically-modified nucleic acid comprising a first oligonucleotide and a second oligonucleotide, wherein:
(1) the first oligonucleotide is an oligonucleotide as described herein (e.g., compound (I), (Ia) or (Ib));
(2) a portion of the first oligonucleotide is complementary to a portion of the second oligonucleotide; and
(3) the second oligonucleotide has the structure of compound (IIa):

C-L-B(-S-A-S-B)_{m′}(-P-A-P-B)_{n′}(-P-A-S-B)_{q′}(-S-A)_{r′}(-S-B)_{t′}-OR (IIa)
wherein:
C is a hydrophobic molecule;
A, for each occurrence, independently is a 2′-methoxy-ribonucleotide;
B, for each occurrence, independently is a 2′-fluoro-ribonucleotide;
L is a linker comprising an ethylene glycol chain, an alkyl chain, a peptide, RNA, DNA, a phosphodiester, a phosphorothioate, a phosphoramidate, an amide, a carbamate, or a combination thereof;
S is a phosphorothioate linker;
P is a phosphodiester linker;
R, for each occurrence, independently is selected from hydrogen and a capping group (e.g., an acyl group such as acetyl);
m′ is 0 or 1;
n′ is 4, 5 or 6;
q′ is 0 or 1;
r′ is 0 or 1; and
t′ is 0 or 1.

In one embodiment, L is a linker comprising 0-20, 0-10 or 0-4 repeat units of ethyleneglycol. In a particular embodiment, L is a linker comprising 3 repeat units of ethyleneglycol (i.e., triethylene glycol). In another particular embodiment, L is selected from L1, L2 and L3:

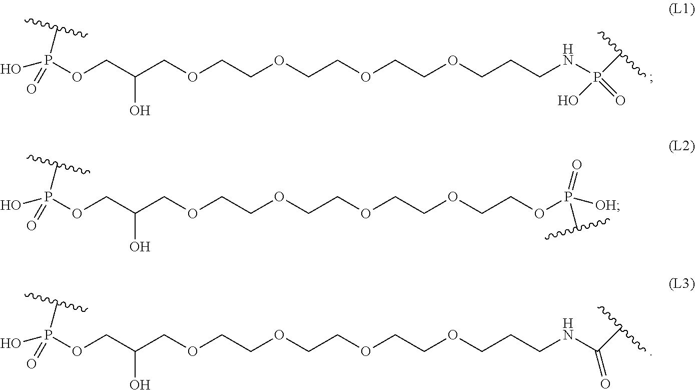

In one embodiment of the double-stranded nucleic acid, the hydrophobic molecule is cholesterol. In another embodiment, the first oligonucleotide has 3-7 more ribonucleotides than the second oligonucleotide. In another particular embodiment, each R is hydrogen.

In one embodiment, the double-stranded nucleic acid comprises one or more mismatch within the complementary portions of the first and second oligonucleotide. In a particular embodiment, the double-stranded nucleic acid contains one mismatch within the complementary portions of the first and second oligonucleotide.

In one embodiment, the double-stranded nucleic acid comprises 11-16 base pair duplexes, wherein the nucleotides of each base pair duplex have different chemical modifications (e.g., one nucleotide has a 2′-fluoro modification and the other nucleotide has a 2′-methoxy). In a particular embodiment, the double-stranded nucleic acid has 15 base pair duplexes.

In one embodiment of the double-stranded nucleic acid, the first oligonucleotide has structure: X(-S-B-S-A)(-P-B-P-A)₅(-P-B-S-A)(-S-B-S-A)₂(-S-B)-OR; and the second oligonucleotide has the structure: C-L-B(-S-A-S-B)(-P-A-P-B)₅(-S-A)(-S-B)-OR. See, e.g., species P2 of FIG. 7. In a particular embodiment, the double-stranded nucleic acid has the structure of compound (IIIa):

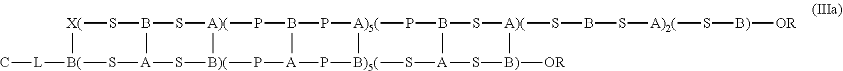

wherein each | represents a hydrogen bonding interaction (i.e., a base-pairing interaction).

In a particular embodiment of compound (IIIa), the first oligonucleotide comprises the sequence 5′ UAAAUUUGGAGAUCCGAGAG 3′ (SEQ ID NO: 4); the second oligonucleotide comprises the sequence 3′ AUUUAAACCUCUAGG 5′ (SEQ ID NO: 5); X is X3; and C is cholesterol. In a further embodiment, R is hydrogen. In a further embodiment, L comprises triethylene glycol. In a particular embodiment, L is L3.

In another particular embodiment of compound (IIIa), the first oligonucleotide comprises the sequence 5′ UAUAAAUGGUAGCUAUGAUG 3′ (SEQ ID NO: 6); the second oligonucleotide comprises the sequence 3′ AUAUUUACCAUCGAU 5′ (SEQ ID NO: 7); X is X3; and C is cholesterol. In a further embodiment, R is hydrogen. In a further embodiment, L comprises triethylene glycol. In a particular embodiment, L is L3.

In another particular embodiment of compound (IIIa), the first oligonucleotide comprises the sequence 5′ UUAAUCUCUUUACUGAUAUA 3′ (SEQ ID NO: 8); the second oligonucleotide comprises the sequence 3′ AAUUAGAGAAAUGAC 5′ (SEQ ID NO: 9); X is X3; and C is cholesterol. In a further embodiment, R is hydrogen. In a further embodiment, L comprises triethylene glycol. In a particular embodiment, L is L3.

In another embodiment of the double-stranded nucleic acid, the first oligonucleotide has structure: X(-P-B-P-A)₆(-P-B-S-A)(-S-B-S-A)₂(-S-B)-OR; and the second oligonucleotide has the structure: C-L-B(-S-A-S-B)(-P-A-P-B)₆-OR. See, e.g., species P1 of FIG. 7. In a particular embodiment, the double-stranded nucleic acid has the structure of compound (IIIb)):

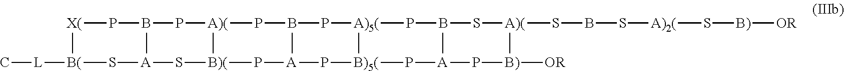

wherein each | represents a hydrogen bonding interaction (i.e., a base-pairing interaction).

In a particular embodiment of compound (IIIb), the first oligonucleotide comprises the sequence 5′ UAAAUUUGGAGAUCCGAGAG 3′ (SEQ ID NO: 4); the second oligonucleotide comprises the sequence 3′ AUUUAAACCUCUAGG 5′ (SEQ ID NO: 5); X is X3; and C is cholesterol. In a further embodiment, R is hydrogen. In a further embodiment, L comprises triethylene glycol. In a particular embodiment, L is L3.

In another particular embodiment of compound (IIIb), the first oligonucleotide comprises the sequence 5′ UAUAAAUGGUAGCUAUGAUG 3′ (SEQ ID NO: 6); the second oligonucleotide comprises the sequence 3′ AUAUUUACCAUCGAU 5′ (SEQ ID NO: 7); X is X3; and C is cholesterol. In a further embodiment, R is hydrogen. In a further embodiment, L comprises triethylene glycol. In a particular embodiment, L is L3.

In another particular embodiment of compound (IIIb), the first oligonucleotide comprises the sequence 5′ UUAAUCUCUUUACUGAUAUA 3′ (SEQ ID NO: 8); the second oligonucleotide comprises the sequence 3′ AAUUAGAGAAAUGAC 5′ (SEQ ID NO: 9); X is X3; and C is cholesterol. In a further embodiment, R is hydrogen. In a further embodiment, L comprises triethylene glycol. In a particular embodiment, L is L3.

In another embodiment of the double-stranded nucleic acid, the first oligonucleotide has structure: X(-S-B-S-A (-P-B-P-A)₅(-S-B-S-A)₃(-S-B)-OR; the second oligonucleotide has structure: C-L-B(-S-A-S-B)(-P-A-P-B)₅(-S-A-S-B)-OR; and the nucleic acid has the structure of Formula (IIIc):

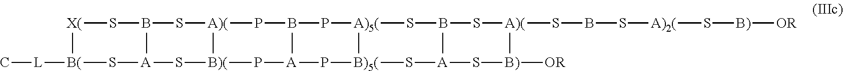

wherein each | represents a hydrogen bonding interaction (i.e., a base-pairing interaction).

In another particular embodiment of compound (IIIc), the first oligonucleotide comprises the sequence 5′ UUAAUCUCUUUACUGAUAUA 3′ (SEQ ID NO: 8); the second oligonucleotide comprises the sequence 3′ AAUUAGAGAAAUGAC 5′ (SEQ ID NO: 9); X is X3; and C is cholesterol. In a further embodiment, R is hydrogen. In a further embodiment, L comprises triethylene glycol. In a particular embodiment, L is L3.

In certain embodiments, compounds (I), (Ia) and (Ib) comprise a sequence corresponding to a “Guide PO” species of FIG. 23 or a an “Antisense Strand” of FIG. 24. In certain embodiments, compounds (II) and (IIa) comprise a sequence corresponding to a “Sense PO” species of FIG. 23 or a “Sense Strand” species of FIG. 24.

In another aspect, provided herein is a composition comprising a first nucleic acid of compound (IIIa), wherein the first oligonucleotide comprises the sequence 5′ UAAAUUUGGAGAUCCGAGAG 3′ (SEQ ID NO: 4); the second oligonucleotide comprises the sequence 3′ AUUUAAACCUCUAGG 5′ (SEQ ID NO: 5); X is X3; L is L3; and C is cholesterol; and a second nucleic acid of compound (IIIa), wherein the first oligonucleotide comprises the sequence 5′ UAUAAAUGGUAGCUAUGAUG 3′ (SEQ ID NO: 6); the second oligonucleotide comprises the sequence 3′ AUAUUUACCAUCGAU 5′ (SEQ ID NO: 7); X is X3; L is L3; and C is cholesterol.

### DEFINITIONS

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base cytosine (e.g., cytidine or a chemically-modified derivative thereof),

By “soluble FLT1 (sFLT1)” (also known as sVEGF-R1) is meant a soluble form of the FLT1 receptor that has sFLT1 biological activity (e.g., e.g., sFlt1-i13 short, sFlt1-i13 long and/or sFlt1-i15a). The biological activity of an sFLT1 polypeptide may be assayed using any standard method, for example, by assaying for one or more clinical symptoms of PE, eclampsia and/or HELLP, by assaying sFLT1 mRNA and/or protein levels, by assaying sFLT1 binding to VEGF and the like. sFLT1 proteins lack the transmembrane domain and the cytoplasmic tyrosine kinase domain of the FLT1 receptor. sFLT1 proteins can bind to VEGF and PIGF bind with high affinity, but cannot induce proliferation or angiogenesis and are therefore functionally different from the Flt-1 and KDR receptors. sFLT1 was initially purified from human umbilical endothelial cells and later shown to be produced by trophoblast cells in vivo. As used herein, sFlt-1 includes any sFlt-1 family member or isoform, e.g., sFLT1-i13 (e.g., FLT1-i13 short and/or sFLT1-i13 long (sFLT1_v1), sFlt1-i15a (sFLT1_v2), sFLT1-e15a, sFLT1_v3, sFLTI_v4 and the like.

By “trophoblast” is meant the mesectodermal cell layer covering the blastocyst that erodes the uterine mucosa and through which the embryo receives nourishment from the mother. Trophoblast cells contribute to the formation of the placenta.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide which may be derivatized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2′ OH-group may be replaced by a group selected from H, OR, R, F, Cl, Br, I, SH, SR, NH2, NHR, NR2, COOR, or OR, wherein R is substituted or unsubstituted C1-C6 alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

In some embodiments, the compounds, oligonucleotides and nucleic acids described herein may be modified to comprise the internucleotide linkages provided in FIG. 36. In particular embodiments, the compounds, oligonucleotides and nucleic acids described herein comprise internuclotide linkages selected from phosphodiester and phosphorothioate.

It is understood that certain internucleotide linkages provided herein, including, e.g., phosphodiester and phosphorothioate, comprise a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion.

In some embodiments, the compounds, oligonucleotides and nucleic acids described herein may be modified to comprise the internucleotide backbone linkages provided in FIG. 37.

In certain embodiments, provided herein are compounds comprising a phosphate moiety (e.g., X1, X4, X5 and X6), a phosphonate moiety (e.g., X3, X7 and X8). These moieties will be partially or completely ionized as a function of the moiety's pKa and the pH of the environment. It is understood that negatively charged ions will be balanced by a cationic moiety, e.g., an alkali metal such as sodium or potassium, an alkali earth metal such as calcium or magnesium, or an ammonium or guanidinium ion.

### Pharmaceutical Compositions and Methods of Administration

In one aspect, provided herein is a pharmaceutical composition comprising a therapeutically effective amount of one or more compound, oligonucleotide, or nucleic acid as described herein, and a pharmaceutically acceptable carrier. In one embodiment, the pharmaceutical composition comprises one or more double-stranded, chemically-modified nucleic acid as described herein, and a pharmaceutically acceptable carrier. In a particular embodiment, the pharmaceutical composition comprises one double-stranded, chemically-modified nucleic acid as described herein, and a pharmaceutically acceptable carrier. In another particular embodiment, the pharmaceutical composition comprises two double-stranded, chemically-modified nucleic acids as described herein, and a pharmaceutically acceptable carrier.

In a particular embodiment, the pharmaceutical composition comprises a first nucleic acid of compound (IIIa), wherein the first oligonucleotide comprises the sequence 5′ UAAAUUUGGAGAUCCGAGAG 3′ (SEQ ID NO: 4); the second oligonucleotide comprises the sequence 3′ AUUUAAACCUCUAGG 5′ (SEQ ID NO: 5); X is X3; and C is cholesterol; and a second nucleic acid of compound (IIIa), wherein the first oligonucleotide comprises the sequence 5′ UAUAAAUGGUAGCUAUGAUG 3′ (SEQ ID NO: 6); the second oligonucleotide comprises the sequence 3′ AUAUUUACCAUCGAU 5′ (SEQ ID NO: 7); X is X3; and C is cholesterol.

The invention pertains to uses of the above-described agents for prophylactic and/or therapeutic treatments as described Infra. Accordingly, the modulators (e.g., RNAi agents) of the present invention can be incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

### Methods of Treatment

In one aspect, the present invention provides for both prophylactic and therapeutic methods of treating a subject at risk of (or susceptible to) a disease or disorder caused, in whole or in part, by secreted Flt1 protein. In one embodiment, the disease or disorder is a liver disease or disorder. In another embodiment, the disease or disorder is a kidney disease or disorder. In one embodiment, the disease or disorder is a placental disease or disorder. In one embodiment, the disease or disorder is a pregnancy-related disease or disorder. In a preferred embodiment, the disease or disorder is a disorder associated with the expression of soluble Flt1 protein and in which amplified expression of the soluble Flt1 protein leads to clinical manifestations of PE (preeclampsia), postpartum PE, eclampsia and/or HELLP (i.e., HELLP syndrome).

In another aspect, the present invention provides for both prophylactic and therapeutic methods of treating a subject at risk of (or susceptible to) a disease or disorder caused, in whole or in part, by a gain of function mutant protein. In one embodiment, the disease or disorder is a trinucleotide repeat disease or disorder. In another embodiment, the disease or disorder is a polyglutamine disorder. In a preferred embodiment, the disease or disorder is a disorder associated with the expression of huntingtin and in which alteration of huntingtin, especially the amplification of CAG repeat copy number, leads to a defect in huntingtin gene (structure or function) or huntingtin protein (structure or function or expression), such that clinical manifestations include those seen in Huntington's disease patients.

“Treatment,” or “treating,” as used herein, is defined as the application or administration of a therapeutic agent (e.g., a RNA agent or vector or transgene encoding same) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by administering to the subject a therapeutic agent (e.g., an RNAi agent or vector or transgene encoding same). Subjects at risk for the disease can be identified by, for example, any or a combination of diagnostic or prognostic assays as described herein. Administration of a prophylactic agent can occur prior to the manifestation of symptoms characteristic of the disease or disorder, such that the disease or disorder is prevented or, alternatively, delayed in its progression.

Another aspect of the invention pertains to methods treating subjects therapeutically, i.e., alter onset of symptoms of the disease or disorder. In an exemplary embodiment, the modulatory method of the invention involves contacting a cell expressing a gain-of-function mutant with a therapeutic agent (e.g., a RNAi agent or vector or transgene encoding same) that is specific for one or more target sequences within the gene, such that sequence specific interference with the gene is achieved. These methods can be performed in vitro (e.g., by culturing the cell with the agent) or, alternatively, in vivo (e.g., by administering the agent to a subject).

An RNA silencing agent modified for enhance uptake into neural cells can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of RNA agent (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of RNA silencing agent per kg of bodyweight. The unit dose, for example, can be administered by injection (e.g., intravenous or intramuscular, intrathecally, or directly into the brain), an inhaled dose, or a topical application. Particularly preferred dosages are less than 2, 1, or 0.1 mg/kg of body weight.

Delivery of an RNA silencing agent directly to an organ (e.g., directly to the brain, spinal column, placenta, liver and/or kidneys) can be at a dosage on the order of about 0.00001 mg to about 3 mg per organ, or preferably about 0.0001-0.001 mg per organ, about 0.03-3.0 mg per organ, about 0.1-3.0 mg per eye or about 0.3-3.0 mg per organ. The dosage can be an amount effective to treat or prevent a neurological disease or disorder (e.g., Huntington's disease) or a liver-, kidney- or pregnancy-related disease or disorder (e.g., PE, postpartum PE, eclampsia and/or HELLP). In one embodiment, the unit dose is administered less frequently than once a day, e.g., less than every 2, 4, 8 or 30 days. In another embodiment, the unit dose is not administered with a frequency (e.g., not a regular frequency). For example, the unit dose may be administered a single time. In one embodiment, the effective dose is administered with other traditional therapeutic modalities.

In one embodiment, a subject is administered an initial dose, and one or more maintenance doses of an RNA silencing agent. The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen may last for a period of time which will vary depending upon the nature of the particular disease, its severity and the overall condition of the patient. In preferred embodiments the dosage may be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

In one aspect, provided herein is a method of treating or managing preeclampsia, post-partum preeclampsia, eclampsia or HELLP syndrome comprising administering to a subject in need of such treatment or management a therapeutically effective amount of a compound, oligonucleotide, or nucleic acid as described herein, or a pharmaceutical composition comprising said compound, oligonucleotide, or nucleic acid.

In another aspect, provided herein is a method of treating or managing Huntington's disease comprising administering to a patient in need of such treatment or management a therapeutically effective amount of a compound, oligonucleotide, or nucleic acid as described herein, or a pharmaceutical composition comprising said compound, oligonucleotide, or nucleic acid.

### Design of siRNA Molecules

In some embodiments, an siRNA molecule of the invention is a duplex consisting of a sense strand and complementary antisense strand, the antisense strand having sufficient complementary to an htt mRNA to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand.

Generally, siRNAs can be designed by using any method known in the art, for instance, by using the following protocol:

1. The siRNA should be specific for a target sequence, e.g., a target sequence set forth in FIG. 23. In one embodiment, the target sequence is found in sFlt1. In one embodiment, a target sequence is found in a mutant huntingtin (htt) allele, but not a wild-type huntingtin allele. In another embodiment, a target sequence is found in both a mutant huntingtin (htt) allele, and a wild-type huntingtin allele. In another embodiment, a target sequence is found in a wild-type huntingtin allele. The first strand should be complementary to the target sequence, and the other strand is substantially complementary to the first strand. (See FIG. 23 for exemplary sense and antisense strands.) In one embodiment, the target sequence is outside the expanded CAG repeat of the mutant huntingin (htt) allele. In another embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus in one embodiment, the invention includes nucleic acid molecules having 35-55% G/C content.

2. The sense strand of the siRNA is designed based on the sequence of the selected target site. Preferably the sense strand includes about 19 to 25 nucleotides, e.g., 19, 20, 21, 22, 23, 24 or 25 nucleotides. More preferably, the sense strand includes 21, 22 or 23 nucleotides. The skilled artisan will appreciate, however, that siRNAs having a length of less than 19 nucleotides or greater than 25 nucleotides can also function to mediate RNAi. Accordingly, siRNAs of such length are also within the scope of the instant invention provided that they retain the ability to mediate RNAi. Longer RNA silencing agents have been demonstrated to elicit an interferon or Protein Kinase R (PKR) response in certain mammalian cells which may be undesirable. Preferably the RNA silencing agents of the invention do not elicit a PKR response (i.e., are of a sufficiently short length). However, longer RNA silencing agents may be useful, for example, in cell types incapable of generating a PKR response or in situations where the PKR response has been down-regulated or dampened by alternative means.

The siRNA molecules of the invention have sufficient complementarity with the target sequence such that the siRNA can mediate RNAi. In general, siRNA containing nucleotide sequences sufficiently identical to a target sequence portion of the target gene to effect RISC-mediated cleavage of the target gene are preferred. Accordingly, in a preferred embodiment, the sense strand of the siRNA is designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The invention has the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition.

Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet your criteria for evaluation.

Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C)=2(#of A+T bases)+4(#of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to target any of the target sequences described supra. Said siRNAs comprise an antisense strand which is sufficiently complementary with the target sequence to mediate silencing of the target sequence. In certain embodiments, the RNA silencing agent is a siRNA.

In certain embodiments, the siRNA comprises a sense strand comprising a sequence set forth in FIG. 23 or 24, and an antisense strand comprising a sequence set forth in FIG. 23 or 24.

Sites of siRNA-mRNA complementation are selected which result in optimal mRNA specificity and maximal mRNA cleavage.

### siRNA-Like Molecules

siRNA-like molecules of the invention have a sequence (i.e., have a strand having a sequence) that is “sufficiently complementary” to a target sequence of a htt mRNA to direct gene silencing either by RNAi or translational repression. siRNA-like molecules are designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g. within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G: U) or a mismatched base pair (G:A, C:A, C:U, G:G, A:A, C:C, U:U). In a further preferred embodiment, the “bulge” is centered at nucleotide positions 12 and 13 from the 5′ end of the miRNA molecule.

### Gene Silencing Oligonucleotides

In certain exemplary embodiments, gene expression (i.e., htt gene expression) can be modulated using oligonucleotide-based compounds comprising two or more single stranded antisense oligonucleotides that are linked through their 5′-ends that allow the presence of two or more accessible 3′-ends to effectively inhibit or decrease htt gene expression. Such linked oligonucleotides are also known as Gene Silencing Oligonucleotides (GSOs). (See, e.g., U.S. Pat. No. 8,431,544 assigned to Idera Pharmaceuticals, Inc., incorporated herein by reference in its entirety for all purposes.)

The linkage at the 5′ ends of the GSOs is independent of the other oligonucleotide linkages and may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 5′ terminal nucleotide.

GSOs can comprise two identical or different sequences conjugated at their 5′-5′ ends via a phosphodiester, phosphorothioate or non-nucleoside linker. Such compounds may comprise 15 to 27 nucleotides that are complementary to specific portions of mRNA targets of interest for antisense down regulation of gene product. GSOs that comprise identical sequences can bind to a specific mRNA via Watson-Crick hydrogen bonding interactions and inhibit protein expression. GSOs that comprise different sequences are able to bind to two or more different regions of one or more mRNA target and inhibit protein expression. Such compounds are comprised of heteronucleotide sequences complementary to target mRNA and form stable duplex structures through Watson-Crick hydrogen bonding. Under certain conditions, GSOs containing two free 3′-ends (5′-5′-attached antisense) can be more potent inhibitors of gene expression than those containing a single free 3′-end or no free 3′-end.

In some embodiments, the non-nucleotide linker is glycerol or a glycerol homolog of the formula HO—(CH₂)ₒ—CH(OH)—(CH₂)ₚ—OH, wherein o and p independently are integers from 1 to about 6, from 1 to about 4 or from 1 to about 3. In some other embodiments, the non-nucleotide linker is a derivative of 1,3-diamino-2-hydroxypropane. Some such derivatives have the formula HO—(CH₂)ₘ—C(O)NH—CH₂—CH(OH)—CH₂—NHC(O)—(CH₂)ₘ—OH, wherein m is an integer from 0 to about 10, from 0 to about 6, from 2 to about 6 or from 2 to about 4.

Some non-nucleotide linkers permit attachment of more than two GSO components. For example, the non-nucleotide linker glycerol has three hydroxyl groups to which GSO components may be covalently attached. Some oligonucleotide-based compounds of the invention, therefore, comprise two or more oligonucleotides linked to a nucleotide or a non-nucleotide linker. Such oligonucleotides according to the invention are referred to as being “branched.”

In certain embodiments, GSOs are at least 14 nucleotides in length. In certain exemplary embodiments, GSOs are 15 to 40 nucleotides long or 20 to 30 nucleotides in length. Thus, the component oligonucleotides of GSOs can independently be 14, 15, 16, 17, 18, 19, 20, 21, 22, 23, 24, 25, 26, 27, 28, 29, 30, 31, 32, 33, 34, 35, 36, 37, 38, 39 or 40 nucleotides in length.

These oligonucleotides can be prepared by the art recognized methods such as phosphoramidite or H-phosphonate chemistry which can be carried out manually or by an automated synthesizer. These oligonucleotides may also be modified in a number of ways without compromising their ability to hybridize to mRNA. Such modifications may include at least one internucleotide linkage of the oligonucleotide being an alkylphosphonate, phosphorothioate, phosphorodithioate, methylphosphonate, phosphate ester, alkylphosphonothioate, phosphoramidate, carbamate, carbonate, phosphate hydroxyl, acetamidate or carboxymethyl ester or a combination of these and other internucleotide linkages between the 5′ end of one nucleotide and the 3′ end of another nucleotide in which the 5′ nucleotide phosphodiester linkage has been replaced with any number of chemical groups.

### Modified RNA Silencing Agents

In certain aspects of the invention, an RNA silencing agent (or any portion thereof) of the invention as described supra may be modified such that the activity of the agent is further improved. For example, the RNA silencing agents described in Section II supra may be modified with any of the modifications described infra. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

### 1) Modifications to Enhance Target Discrimination

In certain embodiments, the RNA silencing agents of the invention may be substituted with a destabilizing nucleotide to enhance single nucleotide target discrimination (see U.S. application Ser. No. 11/698,689, filed Jan. 25, 2007 and U.S. Provisional Application No. 60/762,225 filed Jan. 25, 2006, both of which are incorporated herein by reference). Such a modification may be sufficient to abolish the specificity of the RNA silencing agent for a non-target mRNA (e.g. wild-type mRNA), without appreciably affecting the specificity of the RNA silencing agent for a target mRNA (e.g. gain-of-function mutant mRNA).

In preferred embodiments, the RNA silencing agents of the invention are modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide is preferred because it has relatively minor effect on the stability of the RNA duplex or the duplex formed by the guide strand of the RNA silencing agent and the target mRNA. Exemplary universal nucleotide include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In particularly preferred embodiments, the universal nucleotide is an inosine residue or a naturally occurring analog thereof.

In certain embodiments, the RNA silencing agents of the invention are modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In RNA silencing agents having two strands or strand portions (e.g. siRNAs and shRNAs), the destabilizing nucleotide may be introduced in the strand or strand portion that does not contain the specificity-determining nucleotide. In preferred embodiments, the destabilizing nucleotide is introduced in the same strand or strand portion that contains the specificity-determining nucleotide.

### 2) Modifications to Enhance Efficacy and Specificity

In certain embodiments, the RNA silencing agents of the invention may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of the siRNA (e.g., a siRNA designed using the methods of the invention or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of an RNA silencing agent is enhanced by lessening the base pair strength between the antisense strand 5′ end (AS 5′) and the sense strand 3′ end (S 3′) of the RNA silencing agent relative to the bond strength or base pair strength between the antisense strand 3′ end (AS 3′) and the sense strand 5′ end (S ′5) of said RNA silencing agent.

In one embodiment, the asymmetry of an RNA silencing agent of the invention may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of an RNA silencing agent of the invention may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of an RNA silencing agent of the invention may be enhanced such that there is at least one wobble base pair, e.g., G:U, between the 5′ end of the first or antisense strand and the 3′ end of the sense strand portion. In another embodiment, the asymmetry of an RNA silencing agent of the invention may be enhanced such that there is at least one base pair comprising a rare nucleotide, e.g., inosine (I). Preferably, the base pair is selected from the group consisting of an I:A, I:U and I:C. In yet another embodiment, the asymmetry of an RNA silencing agent of the invention may be enhanced such that there is at least one base pair comprising a modified nucleotide.

3) RNA Silencing Agents with Enhanced Stability

The RNA silencing agents of the present invention can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In a preferred aspect, the invention features RNA silencing agents that include first and second strands wherein the second strand and/or first strand is modified by the substitution of internal nucleotides with modified nucleotides, such that in vivo stability is enhanced as compared to a corresponding unmodified RNA silencing agent. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In a preferred embodiment of the present invention, the RNA silencing agents may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. Particularly, the ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Particularly preferred modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moieties of the instant invention. Additional modified residues include, deoxy-abasic, inosine, N3-methyl-uridine, N6,N6-dimethyl-adenosine, pseudouridine, purine ribonucleoside and ribavirin. In a particularly preferred embodiment, the 2′ moiety is a methyl group such that the linking moiety is a 2′-O-methyl oligonucleotide.

In an exemplary embodiment, the RNA silencing agent of the invention comprises Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

In another exemplary embodiment, the RNA silencing agent of the invention comprises Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254:1497-1500).

Also preferred are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. It should be noted that the above modifications may be combined.

In other embodiments, cross-linking can be employed to alter the pharmacokinetics of the RNA silencing agent, for example, to increase half-life in the body. Thus, the invention includes RNA silencing agents having two complementary strands of nucleic acid, wherein the two strands are crosslinked. The invention also includes RNA silencing agents which are conjugated or unconjugated (e.g., at its 3′ terminus) to another moiety (e.g. a non-nucleic acid moiety such as a peptide), an organic compound (e.g., a dye), or the like). Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

### 4) Modifications to Enhance Cellular Uptake

In other embodiments, RNA silencing agents may be modified with chemical moieties, for example, to enhance cellular uptake by target cells (e.g., neuronal cells). Thus, the invention includes RNA silencing agents which are conjugated or unconjugated (e.g., at its 3′ terminus) to another moiety (e.g. a non-nucleic acid moiety such as a peptide), an organic compound (e.g., a dye), or the like. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In a particular embodiment, an RNA silencing agent of invention is conjugated to a lipophilic moiety. In one embodiment, the lipophilic moiety is a ligand that includes a cationic group. In another embodiment, the lipophilic moiety is attached to one or both strands of an siRNA. In an exemplary embodiment, the lipophilic moiety is attached to one end of the sense strand of the siRNA. In another exemplary embodiment, the lipophilic moiety is attached to the 3′ end of the sense strand. In certain embodiments, the lipophilic moiety is selected from the group consisting of cholesterol, vitamin E, vitamin K, vitamin A, folic acid, or a cationic dye (e.g., Cy3). In an exemplary embodiment, the lipophilic moiety is a cholesterol. Other lipophilic moieties include cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

### 5) Tethered Ligands

Other entities can be tethered to an RNA silencing agent of the invention. For example, a ligand tethered to an RNA silencing agent to improve stability, hybridization thermodynamics with a target nucleic acid, targeting to a particular tissue or cell-type, or cell permeability, e.g., by an endocytosis-dependent or- independent mechanism. Ligands and associated modifications can also increase sequence specificity and consequently decrease off-site targeting. A tethered ligand can include one or more modified bases or sugars that can function as intercalators. These are preferably located in an internal region, such as in a bulge of RNA silencing agent/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a RNA silencing agent to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an RNA silencing agent to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine has an increased affinity for the HIV Rev-response element (RRE). In some embodiments the guanidine analog (the guanidinoglycoside) of an aminoglycoside ligand is tethered to an RNA silencing agent. In a guanidinoglycoside, the amine group on the amino acid is exchanged for a guanidine group. Attachment of a guanidine analog can enhance cell permeability of an RNA silencing agent. A tethered ligand can be a poly-arginine peptide, peptoid or peptidomimetic, which can enhance the cellular uptake of an oligonucleotide agent.

Exemplary ligands are coupled, preferably covalently, either directly or indirectly via an intervening tether, to a ligand-conjugated carrier. In exemplary embodiments, the ligand is attached to the carrier via an intervening tether. In exemplary embodiments, a ligand alters the distribution, targeting or lifetime of an RNA silencing agent into which it is incorporated. In exemplary embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and may also improve nuclease resistance of the resultant natural or modified RNA silencing agent, or a polymeric molecule comprising any combination of monomers described herein and/or natural or modified ribonucleotides. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly (L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g, cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C₁₀, C₁₁, C₁₂, C₁₃, C₁₄, C₁₅, C₁₆, C₁₇, C₁₈, C₁₉, or C₂₀ fatty acids) and ethers thereof, e.g., C₁₀, C₁₁, C₁₂, C₁₃, C₁₄, C₁₅, C₁₆, C₁₇, C₁₈, C₁₉, or C₂₀ alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA silencing agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the RNA silencing agent into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney. In a preferred embodiment, the lipid based ligand binds HSA. A lipid-based ligand can bind HSA with a sufficient affinity such that the conjugate will be preferably distributed to a non-kidney tissue. However, it is preferred that the affinity not be so strong that the HSA-ligand binding cannot be reversed. In another preferred embodiment, the lipid based ligand binds HSA weakly or not at all, such that the conjugate will be preferably distributed to the kidney. Other moieties that target to kidney cells can also be used in place of or in addition to the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the RNA silencing agent, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to an RNA silencing agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

### EXAMPLES

### Example 1

### Background and Significance of Preeclampsia (PE)

Overwhelming evidence from epidemiological and experimental studies now indicates that PE is caused by elevated levels of “soluble decoy” proteins (soluble FLT1s (sFLT1s)) from the Flt1 gene (VEGFR1) in the mother's blood stream (Young, B. C., Levine, R. J. & Karumanchi, S. A. Pathogenesis of preeclampsia. Annual review of pathology 5, 173-192 (2010); Maynard, S. E. et al. Excess placental soluble fms-like tyrosine kinase 1 (sFlt1) may contribute to endothelial dysfunction, hypertension, and proteinuria in preeclampsia. The Journal of clinical investigation 111, 649-658 (2003); Levine, R. J. et al. Circulating angiogenic factors and the risk of preeclampsia. The New England journal of medicine 350, 672-683 (2004); Heydarian, M. et al. Novel splice variants of sFlt1 are upregulated in preeclampsia. Placenta 30, 250-255 (2009)). FLT1 is a receptor tyrosine kinase (RTK) predominantly expressed in the placenta. A general mechanism for RTK modulation is production of truncated, secreted forms of the receptor that act as dominant negative regulators of the overall signaling pathway. Ligand sequestration by such soluble decoys inhibits intracellular signaling by the full-length receptor, thereby desensitizing the system to ligand concentration (Vorlova, S. et al. Induction of antagonistic soluble decoy receptor tyrosine kinases by intronic polyA activation. Molecular cell 43, 927-939 (2011).). In the case of FLT1, the soluble decoys are expressed from truncated mRNAs generated by polyadenylation within two introns (i13 and i15) upstream of the exons encoding the full length FLT1 (fl-FLT1) transmembrane (TM) and kinase domains.

In mammals, FLT1 is predominantly expressed in the placenta, with human placental Flt1 mRNA levels being 10-100 times higher than those observed in other adult tissues (Cerdeira, A. S. & Karumanchi, S. A. Angiogenic factors in preeclampsia and related disorders. Cold Spring Harbor perspectives in medicine 2 (2012)). Whereas the full-length isoform predominates in all tissues in non-pregnant adult humans (Id.), placental expression is dominated by three truncated isoforms, sFlt1-i13 short, sFlt1-i13 long and sFlt1-i15a, all of which encode sFLT1 proteins. This same pattern of high Flt1 in placenta and low expression in other non-pregnant adult tissues is observed in rodents. However, because rodents lack the intron 14 polyadenylation site, they only express a single soluble decoy form: sFlt1-i13. In PE, both full-length (fl-Flt1) and truncated Flt1 mRNAs accumulate to higher levels in the placenta than in normal pregnancies, with the truncated isoforms being even more pronounced. These changes at the mRNA level likely explain the significant rise in sFLT1 proteins in the maternal bloodstream during PE.

1.1 Applicability of siRNAs for Treatment of PE

siRNA-based therapeutics were designed for the treatment of PE. Both preclinical and clinical data support decreasing sFLT1 as a valid therapeutic strategy for prolonging PE pregnancies (Thadhani, R. et al. Pilot study of extracorporeal removal of soluble fms-like tyrosine kinase 1 in preeclampsia. Circulation 124, 940-950 (2011)). Further, the unique region specific to each sFLT1 protein is very small, with only a handful of unique amino acids being appended to each C-terminus. This small target size hinders development of conventional drugs (e.g., small molecules and antibodies) targeting only sFLT1s and not fl-FLT1. On the other hand, the target window at the RNA level is much larger, with the i13 and i15 mRNA isoforms having 435 and 567 unique bases, respectively, neither of which are present in fl-Flt1 mRNA. Because RNAi requires a target size of only 19-22 nucleotides, this was determined to be more than sufficient nucleotide space in which to design multiple isoform-selective siRNAs. From a clinical perspective, the possibility that a single dose delivered subcutaneously will be sufficient to prevent runaway sFLT1 expression for several weeks could make treatment simple and affordable.

Novel chemically-modified oligonucleotides known as self-delivering hydrophobically modified siRNAs (hsiRNAs) (FIG. 1A) could provide the most significant advantage for a cost effective therapeutic. While their current cost of chemical synthesis ($200 per gram, with approximately $20 per dose at 1 mg/kg dose levels) is relatively high, the price is expected to decrease dramatically (10-50 fold) with a kg-level scale-up. Further, hsiRNAs can be fully synthesized using solid support chemistry in less than 10 hours. Like other oligonucleotides, dried hsiRNAs are highly stable, can be stored for extensive time (i.e., years) at ambient temperature, and can be brought into solution just prior to injection. Further, hsiRNA half-life in vivo is of sufficient duration that a single intravenous dose is well suited for a two to six week inhibition of sFLt1 production.

The ONTs that neutralize sFlt1 described herein are the first novel preeclampsia therapy based on a mechanistic understanding of the disease, and could be cost-effectively and easily administered throughout the world.

### 1.2 Pilot Product Target Profile for RNAi-based Treatment of PE

Special considerations for developing an RNAi-based treatment for PE are discussed below.

1.3 Multiple sFLT1 mRNA Isoforms

By performing polyadenylation site sequencing (PAS-Seq (Heyer, E. E., Ozadam, H., Ricci, E. P., Cenik, C. & Moore, M. J. An optimized kit-free method for making strand-specific deep sequencing libraries from RNA fragments. Nucleic Acids Res 43, e2 (2015))) on total RNA from multiple normal and PE placentas, it was determined that PE placentas overexpress i13 and i15 sFLT1 variants with, i15 being responsible for 55% of reads and i13 responsible for approximately 45% of reads. Without intending to be bound by scientific theory, the intrinsic variability in isoform ratios in different samples indicates that targeting both isoforms might be the best option to cover the majority of PE patients. Thus, the candidate drug product was defined as an equimolar mixture of two hsiRNAs: one targeting both short and long sFLT1-i13 and another targeting sFlt1-i15a. The FDA has already allowed an siRNA mixture to be defined as a single drug entity when the component siRNAs are identically formulated or chemically modified and their PK/PD profiles are very similar (e.g., multi-siRNA formulations targeting VEGF-A/KSP (Tabernero, J. et al. First-in-humans trial of an RNA interference therapeutic targeting VEGF and KSP in cancer patients with liver involvement. Cancer Discovery 3, 406-417 (2013)); HBV (Wooddell, C. I. et al. Hepatocyte-targeted RNAi Therapeutics for the Treatment of Chronic Hepatitis B Virus Infection. Molecular Therapy: The Journal of the American Society of Gene Therapy 21, 973-985 (2013)), Arrowhead, etc.). Although using a mixture adds complexity to CMC (Chemistry, Manufacturing and Controls), this is outweighed by the advantage that the mixture will allow treatment of wider PE populations independent of isoform variant overexpression ratios. In certain embodiments, a mixture of two candidates is administrated subcutaneously (SC) in saline as an excipient.

In certain embodiments, the desired level of sFLT1 silencing is only 30-40%, as a higher degree of silencing might be disadvantageous. Preliminary data indicated that a 10-20 mg/kg dose produced >50% silencing in mice, so lesser silencing may simply be achieved with lower dosing. Because the desired product profile is a one-time injection, however, higher doses might be required to extend effect duration. Thus, in certain embodiments, i13 or i15 may be used alone as a clinical candidate.

### 1.4 Overall Safety and Toxicity Considerations

ONT-related toxicity can be due to target-specific effects (e.g., too much silencing of sFlt1 isoforms), target-independent effects (i.e., unintentional silencing of non-target mRNAs) or class-related chemistry-specific events. The ability to target the i13 and i15 variants separately dramatically reduces the chances of any major target-related toxicity. Further, the i13 and i15 variants are placenta- and pregnancy-specific, with low or undetectable expression in other adult tissues. Therefore, clinically limiting toxicity will most likely be target-independent. These types of effects include siRNA off-targeting, RNA-based induction of the innate immune response, and general toxicity related to the chosen mode of delivery (e.g., hydrophobic modifications in combination with phosphorothioates). The most advanced bioinformatics was employed up-front upfront to optimize oligonucleotide design to minimize potential off-target events (Uchida, S. ct al. An integrated approach for the systematic identification and characterization of heart-enriched genes with unknown functions. BMC Genomics 10, 100 (2009)). Further, all riboses in the seed sequence (i.e., nucleotides 2-8 of the guide strand) were 2′-F and 2′-O-methyl modified, which modifications by themselves are well-established to minimize off-target events (Jackson, A. L. et al. Position-specific chemical modification of siRNAs reduces “off-target” transcript silencing. RNA 12, 1197-1205 (2006)). While evaluation of off-targeting signatures could be established in vitro and in mouse samples using microarray profiling (Jackson, A. L. et al. Position-specific chemical modification of siRNAs reduces “off-target” transcript silencing. RNA 12, 1197-1205 (2006); Anderson, E., Boese, Q., Khvorova, A. & Karpilow, J. Identifying siRNA-induced off-targets by microarray analysis. Methods in molecular biology 442, 45-63 (2008); Anderson, E. M. et al. Experimental validation of the importance of seed complement frequency to siRNA specificity. RNA 14, 853-861 (2008); Birmingham, A. et al. 3′ UTR seed matches, but not overall identity, are associated with RNAi off-targets. Nat Methods 3, 199-204 (2006); Fedorov, Y. et al. Off-target effects by siRNA can induce toxic phenotype. RNA 12, 1188-1196 (2006)). Because the overlap between siRNA off-targeting signatures in tissue culture/animal models and humans is generally minimal (Burchard, J. et al. MicroRNA-like off-target transcript regulation by siRNAs is species specific. RNA 15, 308-315 (2009)), the value of such studies is questionable. For each sFLT1 isoform, two different sequences were selected for in vivo evaluation (one lead and one back-up) (FIG. 3). If the lead fails due to off-targeting-induced toxicity, the second sequence is used as a backup (Jackson, A. L. & Linsley, P. S. Recognizing and avoiding siRNA off-target effects for target identification and therapeutic application. Nature Reviews. Drug Discovery 9, 57-67 (2010)). As there is currently no formal guidance specific to siRNA therapeutics, the standard recommendation for NCE (New Chemical Entity) development, including demonstrating safety in two animal models (Hughes M, I. J., Kurtz A, et al. (ed. C. N. Sittampalam G S, Nelson H, et al., editors) (Eli Lilly & Company and the National Center for Advancing Translational Sciences, Bethesda (MD); 2012)), is followed.

The lead compounds were fully chemically-modified (meaning no unmodified riboses remained) using an alternating 2′-O-methyl/2′-F pattern. The combination of 2′ OMe/2′-F is known to block innate immune response activation (Nair, J. K. et al. Multivalent N-Acetylgalactosamine-Conjugated siRNA Localizes in Hepatocytes and Elicits Robust RNAi-Mediated Gene Silencing. Journal of the American Chemical Society (2014)). Lack of interferon pathway activation was confirmed with an in vitro human whole blood cytokine activation assay looking at IL-1β, IL-1RA, IL-6, IL-8, IL-10, IL-12(p70), IP-10, G-CSF, IFN-γ, MCP-1, MIP-1α, MIP-1β, and TNF-α (Bio-Plex Pro Magnetic Cytokine Assay; BioRad Laboratories) and in vivo (after injection in mice) looking at G-CSF, TNF, IL-6, IP-10, KC, and MCP-1 (Cytokine/Chemokine Magnetic Bead Panel; Millipore) (Kumar, V. et al. Shielding of Lipid Nanoparticles for siRNA Delivery: Impact on Physicochemical Properties, Cytokine Induction, and Efficacy. Molecular Therapy. Nucleic acids 3, e210 (2014)).

Without intending to be bound by scientific theory, based on data from other oligonucleotide chemistries (Wooddell, C. I. et al. Hepatocyte-targeted RNAi Therapeutics for the Treatment of Chronic Hepatitis B Virus Infection. Molecular Therapy: The Journal of the American Society of Gene Therapy 21, 973-985 (2013); Coelho, T. et al. Safety and efficacy of RNAi therapy for transthyretin amyloidosis. The New England Journal of Medicine 369, 819-829 (2013)), dose limiting toxicity is most likely related to liver function. Preliminary studies determined that up to 50% of the injected dose of the hsiRNAs accumulated in liver, with delivery being specific to endothelial, kupffer and stellate cells, not hepatocytes (FIG. 4). With other phosphorothioate-containing oligonucleotides, slight reversible elevation of liver enzymes and mild reversible injection side reactions have been noted as side effects (Frazier, K. S. Antisense Oligonucleotide Therapies: The Promise and the Challenges from a Toxicologic Pathologist's Perspective. Toxicologic pathology 43, 78-89 (2015)), but usually this liver enzyme elevation is only observed after long-term continuous dosing with high dose levels. Because this treatment is necessarily short-term (just one or two injections over a period of one to two months) and does not target hepatocytes, liver toxicity may not be an issue. Nonetheless, these concerns will be studied in detail.

Development of any therapeutic targeting pregnant women has additional safety considerations. A major concern is potential transfer of hsiRNAs to the fetus and any possible toxicity this might cause. In preliminary studies, no detectable oligonucleotide transfer to the fetus was observed using fluorescent microscopy, or using a highly sensitive PNA (Peptide Nucleic Acid)-based quantitative assay (FIG. 4). Nor were any effects on fetal growth, number of miscarriages, placental histology or other teratogenic effects observed.

### 1.5 Assay and Model Systems to Evaluate Lead Compounds

### Fluorescence Microscopy Evaluation of In Situ Tissue Distribution

hsiRNA variants with a Cy3 or Cy5.5 (lower auto-fluorescence) dye attached through a non-degradable linker to the 5′ end of sense (passenger) strand were synthesized. This compound was biologically stable with no detectable Cy3 cleavage within 24 hours. The fluorescent sense strand hybridized to its complementary guide strand (thus forming a double-stranded hsiRNA) was administrated to animals and oligonucleotide distribution patterns were examined in 4 μm tissue sections also stained with DAPI or/and cell type selective antibodies. Parallel sections could be stained with standard histology markers enabling detailed histology mapping. Because hsiRNAs are already heavily hydrophobically modified, dye addition has little effect on overall hydrophobicity and therefore minimal impact on oligonucleotide distribution. This assay allowed rapid evaluation of tissue and cell-type distribution and was complemented by a PNA-based quantitative assay for direct guide strand detection.

### PNA Hybridization for Quantitative Guide Strand Detection in Tissue Lysates

To enable direct quantification of intact guide stand in tissues, a novel assay was developed and implemented wherein the guide strand was hybridized to a fully complementary Cy3-labeled PNA (peptide nucleic acid) oligonucleotide, and the corresponding duplex was separated from excess single stranded PNA by HPLC (FIG. 5). Since PNA is non-charged and has extremely tight binding to the guide strand, it out-competes both the hsiRNA sense strand and any endogenous target sequences. Fluorescence detection of the Cy3-PNA:guide hybrid provided a direct measure of guide strand abundance in tissue lysates. In conjunction with an HPLC auto injector, this assay enabled guide strand quantification in hundreds of samples overnight. The assay was also highly sensitive, with a limit of detection less than 10 fmole/gram, and hybrids containing full-length, partially degraded, 5′-phosphorylated and 5′-dephosphorylated guide strand can all be quantified as separate peaks or shoulders in the HPLC trace. Because this assay could detect both labeled and unlabeled compounds, it can be directly transitioned to future CRO's for clinical sample analysis.

QUANTIGENE (Affymetrix) Assay for Direct Detection of Flt1 mRNA Variants in Cells and Tissues

QUANTIGENE is a highly sensitive 96-well based assay in which mRNA is directly detected through signal amplification directly from tissue and/or cell lysates. By linking this direct detection assay to a 192 well automatic TissueLyser, a high-throughput version was developed which enabled processing of dozens of samples per animal. Thus, quantitative data on expression of targeted and housekeeping genes was generated in many animals at once. In pilot studies, n=8 was sufficient to detect 40% modulation of sFlt1 mRNA isoform expression with 80% confidence.

ELISA (#MVR100, R&D Systems) for Detection of sFLT1 Proteins in Conditioned Media and Blood

This 96-well based assay required only 10 μL of biological fluid per sample. This assay has been optimized over many years for both in vitro and in vivo studies. It is clinically compatible and allows for evaluation of circulating sFLT1 protein levels without animal sacrifice, and will be particularly useful for non-human primate studies.

### Normal Mouse Pregnancy Model

The sFlt1-i13 variants are expressed during mouse pregnancy with i13 levels exponentially increasing from days 14-19. Perfect homology between the sFLT1-i13-2283 compound and the i13 mouse variant allows the study both of efficacy and of safety in this simple rodent model.

### Preeclampsia Models

Reduced Uterine Perfusion Pressure (RUPP) model of placental ischemia and hypoxia model of preeclampsia is used as described further below.

### Baboon Wild-Type Pregnancy Model

The sFlt1-i15a variant is not expressed in rodents during pregnancy, thus overall combination efficacy and safety will be evaluated in wild-type pregnant baboons using ELISA, a non-invasive assay as readout of efficacy.

### Preliminary Data

A simple and cost-effective PE therapeutic using RNAi to limit excess placental expression of sFLT1 proteins was developed. For this to work, the following objectives were achieved: (1) appropriate siRNA targeting sites in sFlt1 mRNAs were identified; (2) whether RNA silencing was possible in the placenta using generalized (i.e., intravenous or subcutaneous) delivery was determined; and (3) novel siRNA chemistries were developed that would enable preferential delivery to placental trophoblasts, the cell type responsible for excess sFLT1 production.

Using tissue-specific RNA-Seq data available from the Human Protein Atlas (See proteinatlas.org) and PAS-Seq data from multiple normal and PE human placentas, it was determined that, while the full length (fl) isoform predominates in all tissues in non-pregnant adult humans, placental expression is dominated by three truncated isoforms, sFlt1-i13-short, sFlt1-i13-long and sFlt1-i15a, generated by polyadenylation within introns 13 and 15, respectively. Targeting the intronic regions with hsiRNAs enabled selective silencing of truncated isoforms without interfering with fl-Flt1 mRNA abundance.

A novel type of siRNA chemistry was developed that enabled efficient delivery to endothelial cells and demonstrated selective trafficking to the labyrinth region of the placenta (i.e., to trophoblasts, the cell type responsible for sFLT1 expression). Without any additional formulation, up to 12% of the injected dose accumulated in the placenta with no detectable fetal transfer. This technology is the first demonstration of selective labyrinth targeting by any ONT, enabling silencing of sFLT1 protein at it major site of expression.

Over 50 siRNA variants were designed and screened (See FIG. 23). Hyper-functional, fully chemically-modified hsiRNAs were identified that selectively targeted the i13 and i15 isoforms without interfering with fl-FLT1 expression (FIG. 3). Using these hsiRNAs, efficient silencing of i13 and i15 was demonstrated in primary human trophoblasts with no formulation (FIG. 2B). A combination of sFLT1-i13-2283 and sFLT-i15a-2519 hsiRNAs was selected as the lead candidate for treatment of PE (FIG. 3).

It was determined that in-tissue compound concentrations in pregnant mice could reach 100 μg/gram with a single subcutaneous (SC) or intravenous (IV) injection, producing more than 50-80% reduction in sFlt1-i13 mRNA (FIGS. 3 and 4, respectively). Without intending to be bound by scientific theory, with this level of delivery, silencing is expected to persist for weeks in humans, and thus a limited number of injections to be necessary. Indeed, just one SC injection could be sufficient to silence sFLT1 for several weeks, resulting in significant PE pregnancy extension, possibly even to full-term.

### Example 2

### Hydrophobically Modified siRNAs (hsiRNA): Fully Chemically-Modified siRNA/Antisense Hybrids

A panel of chemistries and formulations were considered as potential approaches for placental delivery. These included LNA antisense, LNPs, chol-conjugates/DPC GalNacs and hsiRNA. hsiRNAs by far exceeded other chemistries in placental delivery (discussed further infra) and were selected for further investigation. The efficiency of hsiRNA uptake in primary trophoblasts was evaluated. Efficient uptake by all cells upon addition of Cy3-labeled compound to the media was observed. The hsiRNAs are asymmetric compounds, with a short duplex region (e.g., 15 base-pairs) and a single-stranded fully phosphorothioated tail, where all bases are fully modified using alternating 2′-F/2′-O-methyl pattern (providing stabilization and avoidance of PKR response), and the 3′ end of the passenger strand (i.e., sense strand) is conjugated to a hydrophobic moiety via a linker (e.g., TEG-Cholesterol). The hydrophobic moiety promotes quick membrane association, while the single-stranded phosphorothioated tail is essential for cellular internalization by a mechanism similar to that used by conventional antisense oligonucleotides (D. M. Navaroli, J. C., L. Pandarinathan, K. Fogarty, C., Standley, L. L., K. Bellve, M. Prot, A. Khvorova and & Corvera, S. Self-delivering therapeutic siRNA internalization through a distinct class of early endosomes. PNAS, under review, (2015)). Addition of Cy3-labeled hsiRNA to any cultured cell type shows quick and efficient internalization through an EE1 related part of the endocytosis pathway. A previous version of this technology (Byrne, M. et al. Novel Hydrophobically Modified Asymmetric RNAi Compounds (sd-rxRNA) Demonstrate Robust Efficacy in the Eye. Journal of Ocular Pharmacology and Therapeutics: The Official Journal of the Association for Ocular Pharmacology and Therapeutics (2013)), where only 50% of bases are 2′F/2′-O-methyl modified, is in Phase II clinical trials for dermal fibrosis.

A chemical modification pattern that does not interfere with primary RISC entry was developed. A wide range of chemical variations were generated and an alternating 2′F/2′-O-methyl pattern was identified that optimally configures the guide strand to adopt a geometry that closely mimics that of an individual strand in an A-form RNA duplex. The A-form RNA duplex is recognized by the RISC complex and supports proper positioning of the target mRNA within the cleavage site (Ameres, S. L., Martinez, J. & Schroeder, R. Molecular basis for target RNA recognition and cleavage by human RISC. Cell 130, 101-112 (2007); Schirle, N. T., Sheu-Gruttadauria, J. & MacRae, I. J. Gene Regulation. Structural basis for microRNA targeting. Science 346, 608-613 (2014)). By starting the alternating pattern with a 5′-phosphorylated 2′-O-methyl ribose (a 5′ phosphate is necessary for PIWI domain interaction, Ago2 recognition), the 2′F modifications are placed in even numbered positions 2-14. Positions 2 and 14 were previously shown to be intolerant of bulkier 2′-ribose modifications (Jackson, A. L. et al. Position-specific chemical modification of siRNAs reduces “off-target” transcript silencing. RNA 12, 1197-1205 (2006); Kenski, D. M. et al. siRNA-optimized Modifications for Enhanced In Vivo Activity. Molecular therapy. Nucleic Acids 1, e5 (2012)).

These fully chemically stabilized compounds were at least as or more effective as naked siRNA in RISC entry and represent the first complete chemical modification pattern with no negative impact on RISC function. This discovery was transformative for the PE project, as complete chemical stabilization is absolutely essential for tissue accumulation upon systemic administration. FIG. 7 shows that no full-length compound could be detected in mouse placentas 24 hours post administration of a version wherein 40% of the riboses were still 2′-OH (P0 chemistry). In comparison, both fully 2′-F/2′-O-methyl modified versions (P1 and P2 chemistries) accumulated to above therapeutically efficacious levels (FIG. 7). Another benefit of non-RNA containing siRNAs is case of manufacturing—their DNA-like chemistry with no necessity for orthogonal ribose protection shortens de-protection procedures and increases coupling efficiencies. Finally, complete elimination of all 2′-OH groups helps with avoidance of the innate immune response, which relies mainly on 2′-OH interactions (Alexopoulou, L., Holt, A. C., Medzhitov, R. & Flavell, R. A. Recognition of double-stranded RNA and activation of NF-kappaB by Toll-like receptor 3. Nature 413, 732-738 (2001); Choc, J., Kelker, M. S. & Wilson, I. A. Crystal structure of human toll-like receptor 3 (TLR3) ectodomain. Science 309, 581-585 (2005)).

The discovery of this modification pattern has redefined an established paradigm for therapeutic siRNA design. Partial modification of siRNAs with 2′-O-methyl and 2′-fluoro dramatically increases their stability in vitro, leading to the errant assumption that partial modification would sufficiently stabilize oligonucleotides in vivo. However, this assumption is false (FIG. 39A, B; see Hassler et al., 2016 Nature Biotech). Visual (fluorescence; FIG. 39B) and quantitative (PNA-based; FIG. 39C) assays show that chemical modification of every 2′-OH (FM-hsiRNA) significantly enhances stability, accumulation, and retention of compounds in most tissues upon systemic (intravenous) or CSF (intracerebroventricular, ICV) administration. Partially stabilized hsiRNAs (40% of ribose's retain 2′-OH) performed poorly in comparison to FM-hsiRNAs. Moreover, CNS injection of FM-hsiRNA supports maximal silencing at least one month after injection (FIG. 39D, longer term studies ongoing)—a significant enhancement over the weeklong duration of silencing by partially modified compounds (FIG. 39D).

Replacing every 2′-OH group with 2′-O-methyl or 2′-fluoro has two additional benefits. The absence of a 2′-OH simplifies synthesis—the DNA-like chemistry eliminates the need for orthogonal ribose protection, thereby shortening the deprotection procedure and increasing the coupling efficiency. Moreover, the absence of 2′-OH groups minimizes innate immune activation 27 28, which is essential for enhancing therapeutic index.

Metabolic stability studies were performed on FM-hsiRNA and it was found that the primary degradation product (90% within 2 hours of systemic administration) of FM-hsiRNA results from removal of the 5′ phosphate from the guide strand. Without a 5′ phosphate, the compound cannot bind RISC and is, therefore, inactive (FIG. 38C). Consequently, the 5′ phosphate was replaced with its stable stereo-compatible analog 5′-(E)-vinylphosphonate (5′-E-VP) (also referred to herein as X3). The 5′-E-VP-modified hsiRNA is equally active as 5′-P-hsiRNA (FIG. 38C), but it significantly enhances retention of guide strand in tissues distal to the injection site after one week (FIG. 38D). The 5′-E-VP modification is expected to further increase duration of effect in vivo.

### Example 3

### hsiRNAs Enabled Selective Delivery to Placental Labyrinth Trophoblasts with No Detectable Fetal Transfer

To evaluate hsiRNA distribution in vivo, normal pregnant mice (day 15) were injected with Cy3-labeled sFlt-i13-2283 hsiRNA and distribution examined at by two independent assays. Gross tissue fluorescence microscopy revealed that most of the oligonucleotides accumulated to three tissues: liver endothelium, kidney endothelium and placental labyrinth (FIG. 4). Without intending to be bound by scientific theory, this distribution profile was most likely defined by a combination of blood flow/filtration rate and the cholesterol receptor concentration on cell surfaces. Using the novel FDA-compliant PNA-hybridization assay described above, it was demonstrated that overall drug concentration in placenta exceeded efficacious levels (approximately 100 ng/gram) by orders of magnitude upon a single 10 mg/kg injection (FIG. 4). This level of tissue delivery was roughly the same for IV and SC administration, with approximately 50%, 10% and 12% of the compound distributing to liver, kidney and placenta, respectively, 24 hours post-injection (FIG. 4). Interestingly, only half of this was cleared from the liver (slightly more in kidney) after five days, indicating that a single administration might be sufficient to induce long-term silencing.

In addition to comparing the impact of full 2′-F/2′-O-methyl modification on PK (pharmacokinetics), the phosphorothioate (PS) content was slightly altered. While the P1 chemistry had PS linkages at the 3′-ends of both strands (for a total of 8), the P2 chemistry incorporated another two PS's at the 5′ end of each strand (for a total of 12). Terminal PS linkages provided a defense against exonucleases, and so are essential for long-term stability in extremely aggressive nuclease environments. Overall, these two chemistries were comparable in levels of oligonucleotides delivery at 24 hours s(FIG. 7), but might have different degradation profiles after long term tissue exposure, affecting duration of the silencing effect. They also have slightly different liver:placenta distribution ratios, which might also be somewhat affected by the route of administration (FIG. 7).

3.1. Selection and Identification of Lead Candidate: i13/i15 Mix and Efficacy in Primary Trophoblasts

The i13 and i15 Flt1 mRNA isoforms contained 435 and 567 unique nucleotides, respectively, not present in fl-Flt1 mRNA. Unfortunately, the majority of this sequence space was dominated by homo-polymeric repeats and regions of high GC content, neither of which are targetable by RNAi. Undeterred, a panel of more than 50 hsiRNAs was designed against any feasible targetable sequence using standard siRNA design parameters (Birmingham, A. et al. A protocol for designing siRNAs with high functionality and specificity. Nature protocols 2, 2068-2078 (2007)) including assessment of GC content, specificity and low seed compliment frequency (Anderson, E. M. et al. Experimental validation of the importance of seed complement frequency to siRNA specificity. RNA 14, 853-861 (2008)), elimination of sequences containing miRNA seeds, and examination of thermodynamic bias (Khvorova, A., Reynolds, A. & Jayasena, S. D. Functional siRNAs and miRNAs exhibit strand bias. Cell 115, 209-216 (2003); Schwarz, D. S. et al. Asymmetry in the assembly of the RNAi enzyme complex. Cell 115, 199-208 (2003)). FIG. 3B shows the targeting positions of hsiRNAs identified to be highly functional.

In the design criteria, targeting sites with perfect homology in other primates were favored to simplify both formal toxicology and efficacy studies in non-human primates and the baboon PE model described below. The mouse expresses only an i13 variant. Luckily, the most efficacious hsiRNA, sFLT1-i13-2283, happened to have perfect complementarity to the mouse i13 isoform, enabling direct in vivo efficacy and toxicity evaluation of this compound in both normal and PE mouse pregnancy models. FIG. 3C shows a table with targeting sites and IC50 values of the best compounds identified to efficiently silence the i13 and i15 isoforms. IC50 values for efficacious compounds ranged between 40-100 nM in both HeLa cells and primary human trophoblasts.

FIG. 1C shows an example of the dose response of sFLT1-i13-2283 in primary human trophoblasts used for IC50 value calculation. It is important to emphasize that silencing with hsiRNAs was achieved upon addition of non-formulated compound to the trophoblast media. The level of mRNA knockdown was determined at 72 hours using the above-described QUANTIGENE assay. To control for any potential non-specific effects, i13 or i15 levels were always normalized to a housekeeping gene. A Non-Targeting-Control (NTC) of identical chemistry was used in all experiments to control for chemical class effects. The levels of full length Flt1 mRNA were not affected (FIG. 1D). To evaluate silencing at the protein level, sFLT1 concentration in conditioned medium was measured using ELISA (QUANTIKINE FLT1, MVR100, R&D Systems) (FIG. 1B).

To move forward, two hsiRNA pairs were selected: sFLT1-i13-2283 (5′ CTCTCGGATCTCCAAATTTA 3′ (SEQ ID NO: 10))/sFLT-i15a-2519 (5′ CATCATAGCTACCATTTATT 3′ (SEQ ID NO: 11)) and sFLT1-i13-2318 (5′ ATTGTACCACACAAAGTAAT 3′ (SEQ ID NO: 12))/sFLT-i15a-2585 (5′ GAGCCAAGACAATCATAACA 3′ (SEQ ID NO: 13)) (FIG. 1C). The first pair was the lead drug candidate and was used in all studies. The second pair was a backup. While sequence-specific toxicity is unlikely to be an issue, a backup compound combination that was readily available in case of any sequence-dependent toxicity appeared was desired. In summary, functional hydrophobically-modified siRNAs that selectively target sFlt1-i13 and sFlt1-i15a isoforms were identified. Efficient internalization and silencing of the corresponding targets in primary human trophoblasts was determined at both the mRNA and protein levels.

These data indicate that novel siRNA chemistry has been developed that enables efficient delivery to placental trophoblasts, the primary site of sFLT1 overexpression during PE, and allowed potent silencing of circulating sFLT1 upon systemic administration.

### Example 4

### Reduction of Huntingtin in Both Primary Neurons and Mouse Brain with Unformulated, Stabilized, Hydrophobic siRNAs

The use of hydrophobically modified ASO-siRNA hybrids, which have the potential to offer both better efficacy and distribution in vivo and knockdown in primary neurons in vitro, was explored. The huntingtin gene was used as a target for mRNA knockdown. Huntington's disease is monogenic (Mangiarini, L. et al. Exon 1 of the HTT gene with an expanded CAG repeat is sufficient to cause a progressive neurological phenotype in transgenic mice. Cell 87, 493-506 (1996)) with a number of cellular mechanisms leading to disease pathology (Zuccato, C., Valenza, M. & Cattaneo, E. Molecular Mechanisms and Potential Therapeutical Targets in Huntington's Disease. Physiological Reviews 90, 905-981 (2010)) making it an excellent candidate for possible future oligonucleotide therapeutics.

A panel of hydrophobically modified siRNAs targeting the Huntingtin gene was developed. See FIG. 24. Efficacy and potency was observed both in primary neurons in vitro, and in vivo in mouse brain upon a single low dose injection without any formulation for delivery. These compounds combine a number of different chemical and structural modifications found both in earlier model siRNAs and hsiRNAs, as well as in ASOs. These properties, which include stabilizing base modifications, cholesterol conjugation, and a fully phosphorothioated single stranded tail, make these hsiRNAs excellent tools for studying gene function in hard-to-target primary cells and organs that can be adapted for use in a number of different biologically relevant systems.

4.1 hsiRNA—Hydrophobically Modified siRNA/Antisense Hybrids Were Efficiently Internalized by Primary Neurons

The hsiRNAs were asymmetric compounds, with a short duplex region (e.g., 15 base-pairs) and single-stranded fully phosphorothioated tail. All pyrimidines in these compounds were 2′-Fluoro or 2′-O-Methyl modified (providing stabilization), and the 3′ end of the passenger strand was conjugated to TEG-Cholesterol (FIG. 1A, FIG. 8). The cholesterol conjugate enabled quick membrane association, while the single stranded phosphorothioated tail was necessary for cellular internalization by a mechanism similar to the one used by conventional antisense oligonucleotides. Addition of Cy3-labeled hsiRNA to primary cortical neurons resulted in immediate (within minutes) cellular association (FIG. 1B). Interestingly, the uptake was first observed preferentially in dendrites, followed by re-localization to the cellular body (FIG. 9). The uptake was uniform across all cells in the dish, affirming efficient internalization. Notably, approximately 60% of htt-mRNA was found to be localized in the nuclei (data not shown).

4.2 Identification of hsiRNAs Targeting Huntingtin

A panel of 94 hsiRNA compounds (FIG. 24) targeting huntingtin mRNA was designed and synthesized. These sequences spanned the gene and were selected to comply with standard siRNA design parameters (Birmingham, A. et al. A protocol for designing siRNAs with high functionality and specificity. Nat Protoc 2, 2068-2078 (2007)) including assessment of GC content, specificity and low seed compliment frequency (Anderson, E. M. et al. Experimental validation of the importance of seed complement frequency to siRNA specificity. RNA 14, 853-861 (2008)), elimination of sequences containing miRNA seeds, and examination of thermodynamic bias (Khvorova, A., Reynolds, A. & Jayasena, S. D. Functional siRNAs and miRNAs Exhibit Strand Bias. Cell 115, 209-216 (2003); Schwarz, D. S. et al. Asymmetry in the Assembly of the RNAi Enzyme Complex. Cell 115, 199-208 (2003)). More than 50% of bases were chemically modified, to provide in vivo stability and minimization of immune response (Judge, A., Bola, G., Lee, A. & MacLachlan, I. Design of Noninflammatory Synthetic siRNA Mediating Potent Gene Silencing in Vivo. Molecular Therapy 13, 494-505 (2006)). The modifications imposed additional restrictions on sequence space, reducing the hit rate. Impact on Huntingtin mRNA expression was measured after 72 hours exposure to 1.5 μM hsiRNA (passive uptake, no formulation) in Hela cells by QUANTIGENE assay with 7% of sequences showing more than 70% silencing. At 1.5 μM hsiRNA, 24 hsiRNAs reduced Htt mRNA levels to less than 50% of control levels, including 7 hsiRNAs that reduced Htt mRNA levels below 30% of control. Functional target sites were spread across the gene with the exception of the distal part of the 3′UTR, later explained by preferential expression of the shorter htt isoform in Hela cells (Li, S. H. et al. Huntington's disease gene (IT15) is widely expressed in human and rat tissues. NEURON 11, 985-993 (1993)). IC50 values were identified for sixteen active sequences, selected based on primary screen activity and cross-species conservation (FIG. 25). IC50 values ranged from 90 to 766 nM in passive uptake (no formulation) and from 4 to 91 pM in lipid-mediated uptake (FIG. 24). Fully chemically-optimized active compounds were readily identified, indicating that a much smaller library should be sufficient in future screens for other genes, although hit rate is likely to be variable from target to target. The hsiRNA targeting position 10150 (HTT10150 (i.e., 5′ CAGUAAAGAGAUUAA 3′ (SEQ ID NO: 9))) was used for further studies. To ensure that the hsiRNA chemical scaffold did not negatively impact efficacy and potency of HTT10150, the modified and unmodified versions of the compound were tested in both passive and lipid-mediated silencing assays (FIG. 26). As expected, only the modified sequence was successful at cellular delivery and Htt silencing by passive uptake (IC50=33.5 nM), while both the modified and unmodified compounds showed similar IC50 values in lipid mediated delivery (0.9 pM and 3.5 pM respectively) suggesting that the hsiRNA scaffold modifications did not interfere with RNA-Induced Silencing Complex (RISC) loading.

4.3 Potent and Specific Gene Silencing With Unformulated hsiRNAs in Primary Neurons

HTT10150 was further tested for mRNA silencing in primary neurons isolated from FVBN mice. Efficacy was seen at both 72 hours and one week following simple unformulated compound addition to cortical neurons (FIG. 27A) with maximum silencing (70%) observed at the 1.25 μM concentration. hsiRNA^{HTT} treatment of cortical neurons preferentially eliminated cytoplasmic, over nuclear, Htt mRNA (FIG. 33). HTT10150 also showed similar silencing in primary striatal neurons (FIG. 27B). Protein levels were measured after one week by Western blot, confirming mRNA data with 85% reduction of protein upon treatment with 1.25 μM of compound. HTT10150 hsiRNA did not affect the expression levels of housekeeping controls (Ppib and Tubb1) or the overall viability of primary neuronal cells, as measured by the ALAMARBLUE assay, up to a 2 μM concentration. Similar results were obtained with another hsiRNA targeting Htt mRNA, supporting that the observed phenomena is not unique to HTT10150. In other experiments, a slight impact on cell viability was observed at 3 μM.

To evaluate duration of effect upon a single HTT10150 treatment, the silencing was measured at one week, two week, and three week intervals. The half-life of the loaded RISC complex was weeks (Song, E. et al. Sustained Small Interfering RNA-Mediated Human Immunodeficiency Virus Type 1 Inhibition in Primary Macrophages. Journal of Virology 77, 7174-7181 (2003)), and silencing was expected to be long lasting in non-dividing cells. Indeed, single treatment with hsiRNAs was sufficient to induce htt silencing at all times tested. Three weeks was the longest the primary neurons could be maintained in culture. Other systems will be used for longer-term experiments.

To demonstrate the general applicability of hsiRNAs as a tool for neuronal gene silencing, and to confirm this chemistry scaffold as valid for neuronal delivery, similar experiments were performed with several other hsiRNAs targeting HTT and with one targeting the house-keeping gene PPIB (Cyclophilin B) (FIG. 28). Silencing as high as 70 and 90% was achieved with HTT and PPIB, respectively.

In summary, these data demonstrate that hydrophobically modified siRNA is a simple and straightforward approach for gene silencing in primary neurons, and can be adapted for multiple gene targets.

4.4 hsiRNA Distribution In Vivo in Mouse Brain Upon Single Injection

hsiRNAs are efficiently internalized by different types of neurons in vitro. The selected hsiRNA, HTT10150, was further evaluated for its potential to silence gene expression in the brain in vivo. To determine the distribution profile of HTT10150 upon in vivo administration, 12.5 μg of Cy3 labelled hsiRNA (See FIG. 7 for sequence) was injected intrastriatally and, after 24 hours, the brain was perfused, sectioned, and oligonucleotide distribution was visualized by fluorescence microscopy (Leica DM5500-DFC365FX). The artificial CSF injected samples processed concurrently were used to set up microscopic imaging settings to control for background tissue epifluorescence.

The majority of compound showed a steep gradient of diffusion away from the injection site, with most of the ipsilateral striatum being covered. Interestingly, hsiRNAs were detected on the non-injected side (contralateral) side of the brain (both cortex and striatum), although relative concentrations appeared much lower. Higher magnification images showed significant association of hsiRNA with fiber tracks, most likely due to the presence of a hydrophobic modification. This aspect of hsiRNA may make it useful as a labelling reagent to visualize brain signalling architecture. In addition to fiber tracks and neurite labelling, hsiRNA could be detected as punctate staining in the perinuclear space of different cell types, including neurons, as evident from co-localization with NeuN (neuronal marker) stained cells only 24 hours after injection.

4.5 hsiRNA Efficacy In Vivo in Mouse Brain Upon Single Injection

To determine HTT10150 efficacy in vivo, wild type FVBN mice were dosed intrastriatally with a single injection of between 3 and 25 μg (0.1-0.9 mg/kg) of compound and mRNA silencing was examined both ipsilateral and contralateral to the injection site. Eight animals were dosed per treatment group and three individual punches were taken from each side of the striatum for mRNA and protein quantification. Level of huntingtin expression were measured by QUANTIGENE Assay and normalized to a housekeeping gene.

Statistical analysis was performed by one-way ANOVA comparison against CSF or PBS control with Bonferroni corrections for repeat measures using GraphPad Prism (Online methods for details). All groups induced silencing that was significant against CSF, PBS, and non-targeting control treated animals. At the site of administration (ipsilateral side), dose-dependent silencing reaching statistical significance was observed at all concentrations. The 25 μg treatment induced 77% silencing (p<0.0001), and the 12.5 μg treatment was repeated with two groups of animals on different days and showed statistically significant silencing of 66% and 42%.

While initial distribution studies showed a steep gradient of diffusion away from the injection site with a minimal amount of compound migrating to the contralateral side, treatment with the higher doses of 25 μg and 12.5 μg resulted in statistically significant silencing (p<0.0001) on the non-injected side. However, the level of silencing was significantly less (only 36% for the 25 μg group) than on the treated side of the brain.

To further measure HTT10150 efficacy in vivo, dose-response studies were performed in wild type FVB/NJ mice injected intrastriatally with 3.1, 6.3, 12.5, or 25 μg of HTT10150. As controls, mice were injected with a non-targeting control hsiRNA (NTC), artificial CSF, or PBS. In punch biopsies taken from the ipsilateral and contralateral striatum, HTT10150 reduced Htt mRNA levels in a dose-dependent manner (FIG. 34).

The Htt mRNA is significantly reduced in the ipsilateral side of striatum. Robust dose-dependent silencing was observed with up to 77% (one way Anova p<0.0001) reduction in Htt mRNA expression level at high dose expression levels. Interestingly, statistically significant, but less pronounced, silencing was observed in the contralateral striatum and cortex. The silencing reaches statistical significance with both one-way and two-way Anova (values for two-way Anova are presented in FIG. 34). While some level of fluorescence was detectable in these brain regions with high laser intensity, it is technically challenging to detect as it is very close to the tissue autofluoresence and thus has not been described herein. It is clear that levels of silencing effect are at least correlative to the sharp gradient of distribution from the side of injection.

The Htt mRNA silencing is observed with HTT10150 but not with non-targeting control or a CSF (FIG. 34). In addition, the HTT10150 does not affect expression of several housekeeping genes (PPIB, HPRT). In combination, this is indicative of silencing being caused by HTT10150 mRNA silencing and not by off-target effects.

In summary, these data show that a single intrastriatal injection of hsiRNA is sufficient to induce potent gene silencing around the site of administration. This effect was reproducible across different treatment groups and independent experiments.

4.6 Neuronal Viability Following Single hsiRNA Injection in Mouse Brain

Cholesterol modification of non-modified, naked siRNA has previously been used for improvement of siRNA brain distribution, with toxicity at high doses being identified as a potential limitation. To evaluate the degree of non-specific chemistry related effects on the brain, DARPP32 expression, an established marker for dopamine receptor expression on medium spiny neurons in the striatum and representative of neuronal viability, was investigated. Additionally, potential induction of an immune response was performed by assessing the extent of microglia activation upon hsiRNA injection.

To assess innate immune response activation by hsiRNAs in vivo, IBA1-positive microglial cells were quantified in brain sections from mice injected with 12.5 μg HT10150 or artificial CSF. IBA-1 is specific to microglial cells and is up-regulated following injury to the brain, allowing resting and activated microglia to be distinguished. Total microglia counts showed only 25% increase in the ipsilateral striatum at five days post injection indicating a lack of any major inflammatory response (FIG. 35).

No significant impact on DARPP32 expression was observed for doses up to 12.5 μg suggesting persistent neuronal viability. Similarly, minimal microglial activation was visualized at the 12.5 μg dose indicative of a limited immune response in the presence of the modified hsiRNA. The 25 μg dose did induce some reduction in DARPP32 just around the site of injection indicative of toxicity and establishing the maximum dose levels for this chemical scaffold upon the indicated route of administration. A 10-12.5 μg single administration of hsiRNA efficiently silenced HTT mRNA in three, well powered, independent studies with robust silencing of 62, 42 and 52% without toxicity. These data indicate that this technology can be widely used for functional studies of other neurologically significant targets.

4.7 hsiRNA^{HTT}, but not an LNA-GAPMER Oligonucleotide, Exhibits a Silencing Plateau

A silencing plateau is observed only with RNAi (cytoplasmic; hsiRNA-F1) but not RNAseH (predominantly nuclear; LNA-GAPMER) compounds. The observed silencing plateau (FIG. 32) is specific to the HTT gene.

### 4.8 Discussion

This study demonstrates that the use of hydrophobically modified siRNA for delivery to primary cells is a valuable tool to enable functional and genomic studies of neuronal pathways and neurological disorders.

The ability to cause gene silencing in primary neurons without the use of toxic formulation has a significant impact on neuroscience research, facilitating a more in depth study of neurological disorders in the context of primary cell lines, and ultimately providing a more relevant understanding of in vivo function and pathology. Most neuronal studies are done in stable cell lines due to case of delivery and cell maintenance, but using artificial cell systems can lead to artifacts in the data that can be attributed to manipulation of these cell lines, a problem that can be avoided by using primary cells (Cheung, Y.-T. et al. Effects of all-trans-retinoic acid on human SH-SY5Y neuroblastoma as in vitro model in neurotoxicity research. NeuroToxicology 30, 127-135 (2009); Gilany, K. et al. The proteome of the human neuroblastoma cell line SH-SY5Y: An enlarged proteome. Biochimica et Biophysica Acta (BBA)—Proteins and Proteomics 1784, 983-985 (2008); Lopes, F. M. et al. Comparison between proliferative and neuron-like SH-SY5Y cells as an in vitro model for Parkinson disease studies. Brain Research 1337, 85-94 (2010); Zhang, W. et al. Cyclohexane 1,3-diones and their inhibition of mutant SOD1-dependent protein aggregation and toxicity in PC12 cells. BIOORGANIC & MEDICINAL CHEMISTRY 1-17 (2011). doi:10.1016/j.bmc.2011.11.039). Current methods for delivering siRNA to primary neurons include the use of lentiviral vectors, Adeno-Associated Viruses (AAV), or Lipofectamine™-mediated transfection (Karra, D. & Dahm, R. Transfection Techniques for Neuronal Cells. Journal of Neuroscience 30, 6171-6177 (2010)). By conjugating a hydrophobic moiety such as cholesterol directly to the siRNA itself and by utilizing an additional single stranded phosphorothioated tail for enhanced uptake, it has been demonstrated herein that, not only can siRNA be delivered efficiently into primary neurons in vitro with minimal toxicity, but also remains a potent silencer of mRNA.

Without intending to be bound by scientific theory, one of the major advantages of RNAi over antisense technology is that the loaded RISC is expected to remain active for a long period of time in non-dividing cells (Bartlett, D. W. Insights into the kinetics of siRNA-mediated gene silencing from live-cell and live-animal bioluminescent imaging. Nucleic Acids Research 34, 322-333 (2006)). Additionally, a limited number of loaded RISCs are sufficient for the induction of RNAi-mediated silencing (Stalder, L. et al. The rough endoplasmatic reticulum is a central nucleation site of siRNA-mediated RNA silencing. The EMBO Journal 32, 1115-1127 (2013)). The data presented herein demonstrates silencing for up to three weeks in vitro in primary cortical neurons upon a single treatment with hsiRNA, supporting the notion that RNAi-mediated silencing can be both efficient and long lasting. The data presented herein also shows that these compounds can be used to target multiple regions in two different genes, which demonstrates the adaptability of hsiRNA for the study of alternative neurological pathways and diseases.

While a single intra-striatal injection of hsiRNA resulted in potent gene silencing near the injection site in vivo, the effect was not evenly spread throughout the brain. Although limited, spread to other areas of the brain (demonstrated by in vivo efficacy studies) could be happening through a number of mechanisms. These include movement in the CSF, spread via fiber tracts which were shown to have a large visual density of Cy3-labeled hsiRNA in distribution studies, or possibly through retrograde transport (Stewart, G. R. & Sah, D. Retrograde Transport of siRNA and Therapeutic Uses to Treat Neurological Disorders. United States Patent Application Publication US 2008/0039415 A1, 1-18 (2008)), although further studies will be conducted to determine the actual mechanism.

The technology presented herein is useful for understanding functional genomics of particular brain regions, as well as for studying relationships between brain regions. Additionally, the study of some neurological disorders (for example memory disorders (Samuelson, K. W. Post-traumatic stress disorder and declarative memory functioning: a review. Dialogues in Clinical Neuroscience 13, 346-351 (2011))) can benefit from limited and regionally targeted distribution and silencing. However, due to its distribution profile, hsiRNA as it currently exists is not a viable therapeutic for general neurological disorders like Huntington's disease. Multiple injections may work to increase overall silencing in small rodents, but in order to adapt this technology for use in larger animal brains and humans, and to achieve even and widespread distribution, other chemical modifications and therapeutic methods of delivery will be utilized. There are a number of ways in which this might be approached. First, chemical adjustments to the hsiRNA composition itself can be made. These include conjugating it to a different lipid, supplementing the backbone with additional phosphorothioate groups, or by addition of hydrophobic moieties to the nucleotides themselves (Vaught, J. D., Dewey, T. & Eaton, B. E. T7 RNA Polymerase Transcription with 5-Position Modified UTP Derivatives. J. Am. Chem. Soc. 126, 11231-11237 (2004)). All of these modifications could support a range of hydrophobicities that would allow for more improved distribution across a larger distance. Increased bioavailability could also be achieved with different modes of injection such as into the CSF instead of intrastriatally, increasing the likelihood of exposure to the whole brain. However, delivery via the CSF could favor localization of hsiRNA to brain regions other than the striatum, making it a less than ideal delivery method for the treatment of Huntington's disease. Another possibility is formulated delivery by packaging these hydrophobically modified siRNAs into exosomes and liposomes (less toxic than current Lipofectamine™ formulations) and using these natural and synthetic nanocarriers to deliver cargo in a more evenly distributed fashion (Alvarez-Erviti, L. et al. Delivery of siRNA to the mouse brain by systemic injection of targeted exosomes. Nat Biotechnol 1-7 (2011). doi:10.1038/nbt.1807; Marcus, M. & Leonard, J. FedExosomes: Engineering Therapeutic Biological Nanoparticles that Truly Deliver. Pharmaceuticals 6, 659-680 (2013)). However, potency and efficacy of the delivered hsiRNA still needs to be validated for these methods.

In conclusion, HTT10150 was efficient for targeting huntingtin mRNA in primary neurons in vitro and locally in the mouse brain in vivo. This compound did not require any formulation for delivery to primary cells and enabled gene functional studies for huntingtin as well as other targets, making it a very useful tool for the study of neurological disorders. Potential advances to this technology should allow for hsiRNA to function as a therapeutic treatment for Huntington's disease as well as other neurological diseases in the future.

### 4.9 Methods

### Cell Culture

HeLa cells were maintained in DMEM (Corning Cellgro) supplemented with 10% fetal bovine serum (Gibco) and 100 U/mL penicillin/streptomycin (Invitrogen) and grown at 37° C. and 5% CO₂. Cells were split every 2-5 days up to passage 15 and then discarded.

### Cell Culture for Passive Uptake

Cells were plated in DMEM with 6% FBS at 10,000 cells/well in 96-well tissue culture treated plates. hsiRNA was diluted in OptiMEM (Gibco) to 2× final concentration and 50 μL diluted hsiRNA was added to 50 μL of cells for 3% FBS final. Cells were incubated for 72 hours at 37° C. and 5% CO₂.

### Cell Culture for Lipid-Mediated Uptake

Cells were plated in DMEM with 6% FBS at 10,000 cells/well in 96-well tissue culture treated plates. hsiRNA was diluted in OptiMEM to 4× final concentration. LIPOFECTAMINE RNAIMAX Transfection Reagent (Invitrogen #13778150) was diluted to 4× final concentration (final=0.3 μL/25 μL/well). RNAIMAX and hsiRNA were mixed 1:1 and 50 μL was added to 50 μL of cells for 3% FBS final. Cells were incubated for 72 hours at 37° C. and 5% CO₂.

### Preparation of Primary Neurons

Primary cortical neurons were obtained from E15.5 mouse embryos of WT (FVBN) mice. Pregnant females were anesthetized by IP injection of Avertin (250 mg/kg weight) followed by cervical dislocation. Embryos were removed and transferred into a Petri dish with ice-cold DMEM/F12 medium (Invitrogen). Brains were removed and meninges were carefully detached. Cortices were isolated and transferred into a 1.5-ml tube with pre-warmed papain solution for 25 minutes at 37° C. and 5% CO₂ to dissolve tissue. Papain solution was prepared as follows: papain (Worthington #54N15251) was dissolved in 2 mL HibernateE (Brainbits) and 1 mL EBSS (Worthington). Separately, DNase (Worthington #54M15168) was re-suspended in 0.5 mL HibernateE. Then, 0.25 mL of re-suspended DNase was transferred to re-suspended papain for the final solution. After the 25 minute incubation, papain solution was removed and 1 mL NbActiv4 (Brainbits) supplemented with 2.5% FBS was added to the tissue. The cortices were then dissociated by pipetting up and down with a fire polished, glass Pasteur pipet. Cortical neurons were counted and plated at 1×10⁶ cells/ml. For live-cell imaging studies, culture plates were pre-coated with poly-L-lysine (Sigma #P4707) and 2×10⁵ cells were added to the glass center of each dish. For silencing assays, neurons were plated on poly-L-lysine pre-coated 96-well plates (BD BIOCOAT #356515) at 1×10⁵ cells per well. After overnight incubation at 37° C. and 5% CO₂ an equal volume of NbActiv4 (Brainbits) supplemented with anti-mitotics, 0.484 μL/mL of 5′UtP (Sigma #U6625) and 0.2402 μL/mL of 5′FdU (Sigma #F3503), to prevent the growth of non-neuronal cells, was added to neuronal cultures. Half of the volume of media was replaced every 48 hours (with new NbActiv4 with anti-mitotics) until the neurons were treated with siRNA. Once the cells were treated, media was not removed, only added. All subsequent media additions contained anti-mitotics.

mRNA Quantification

mRNA was quantified using the QUANTIGENE 2.0 Assay (Affymetrix #QS0011). Cells were lysed in 250 μL diluted lysis mixture (Affymetrix #13228), 1 part lysis mixture, 2 parts H₂O, with 0.167 μg/μL proteinase K (Affymetrix #QS0103) for 30 minutes at 55° C. Cell lysates were mixed thoroughly and 40 μL (approximately 8000 cells) of lysate were added to the capture plate along with 40 μL additional diluted lysis mixture without proteinase K. Probe sets were diluted as specified in the Affymetrix protocol. For HeLa cells, 20 μL of human HTT or PPIB probe set (Affymetrix #SA-50339, #SA-10003) was added to appropriate wells for a final volume of 100 μL. For primary neurons, 20 μL of mouse HTT or PPIB probe set (Affymetrix #SB-14150, #SB-10002) was used.

Tissues were treated similarly, using 300 μL of Homogenizing Buffer (Affymetrix #10642) with 2 μg/μL proteinase K for a 5 mg tissue punch. Tissues were then homogenized in 96-well plate format on the QIAGEN TissueLyser II and 40 μL were added to the capture plate. Probe sets were diluted as specified in the Affymetrix protocol and 60 μL of either HTT or PPIB probe sets (Affymetrix #SB-14150, #SB-10002) were added to each well of the capture plate for a final volume of 100 μL. For DARPP32 quantification, only 10 μL of tissue sample and 30 μL of homogenizing buffer were added to each well with 60 μL of mouse Ppp1r1b probe set (Affymetrix #SB-21622). Signal was amplified according to the Affymetrix protocol. Luminescence was detected on either the Veritas Luminometer or the Tecan M 1000.

### Live Cell Staining

To monitor live cell hsiRNA uptake, cells were plated at a density of 2×10⁵ cells per 35 mm glass-bottom dish as described in the preparation of primary neurons above. Prior to imaging, cell nuclei were stained in phenol red free NbActiv4 using NUCBLUE (Molecular Probes by Life Technologies #R37605) as indicated by the manufacturer. Imaging was performed in phenol red free NbActiv4. Cells were treated with 0.5 μM of Cy3-labeled hsiRNA, and live cell imaging was performed over time. All live cell confocal images were acquired with a Zeiss confocal microscope and images were processed using ImageJ (1.47v) software.

### Immunohistochemistry/Immunofluorescence

For distribution studies, brains were injected with 1 nmol (12.5 μg) of Cy3-labeled hsiRNA. After 24 hours, mice were sacrificed and brains were removed and sent to the DERC Morphology Core at UMASS Medical School to be embedded in paraffin and sliced into 4 μm sections and mounted on glass slides. Sections were de-parafinized for 8 minutes in xylene two times. Sections were then rehydrated with serial ethanol dilutions (100%, 95%, 80%) for 4 minutes each, then washed twice for two minutes with PBS. For NueN staining, slides were boiled for 5 minutes in antigen retrieval buffer and then left to sit at room temperature for 20 minutes, followed by a 5-minute wash with PBS. Slides were then blocked with 5% normal goat serum in PBS+0.05% Tween20 for 1 hour and washed once with PBS+0.05% Tween20 for 5 minutes. Primary antibody (1:1000 dilution in PBS+0.05% Tween20) was added to slides for a 1 hour incubation followed by three 5-minute washes with PBS+0.05% Tween20. Secondary antibody (1:1000 dilution in PBS+0.05% Tween20) was added to slides for a 30-minute incubation in the dark followed by three 5-minute washes with PBS+0.05% Tween20. Slides were then stained with DAPI (Molecular Probes by Life Technologies #D3571), diluted to 250 ng/ml in PBS, for one minute followed by three 1-minute washes with PBS. Mounting media and coverslips were applied to slides and left to dry over night before imaging on Leica DM5500-DFC365FX microscope at indicated magnification.

For toxicity and microglia activation studies extracted, perfused brains were sliced into 40 μm sections on the Leica 2000T Vibratome in ice cold PBS. Immunohistochemistry was performed on every 6th section against DARPP32 (Millipore, 1:10,000 dilution) and IBA-1 (Millipore, 1:500 dilution). Sections were mounted and visualized by light microscopy. Four images were taken at 20× in the striatum of both injected and non-injected sides of each section. The number of DARPP32 positive neurons was quantified using ImageJ. Activated microglia was quantified by morphology of stained cells for IBA-1.

### Animals, Stereotaxic Injections

Wild-type (FVBN) mice received microinjections by stereotactic placement into the right striata (coordinates (relative to bregma) were 1.0 mm anterior, 2.0 mm lateral, and 3.0 mm ventral). Animals were deeply anesthetized prior to injection with 1.2% Avertin. For both toxicity (DARPP32) and efficacy studies, mice received injections of either PBS or artificial cerebrospinal fluid (2 μL per striata, N=8 mice), 12.5 μg of NTC hsiRNA (2 μL of 500 μM stock solution per striata, N=8 mice), 25 μg of HTT10150 hsiRNA (2 μL of 1 mM stock solution per striata, N=8 mice), 12.5 μg of HTT10150 hsiRNA (2 μL of 500 μM stock solution per striata, N=16 mice total, two sets of 8 mice on two different days), 6.3 μg of HTT10150 hsiRNA (2 μL of 250 μM stock solution per striata, N=8 mice), or 3.1 μg of HTT10150 hsiRNA (2 μL of 125 μM stock solution per striata, N=8 mice) and euthanized 5 days later. Brains were harvested and three 300 μm coronal sections were made. One 2 mm punch was taken per side (injected and non-injected) for each section and placed in RNAlater (Ambion #AM7020) for 24 hours at 4° C. Each punch was processed as an individual sample for the QUANTIGENE assay analysis. All animal procedures were approved by the University of Massachusetts Medical School Institutional Animal Care and Use Committee (IACUC, protocol number A-2411).

### Statistical Analysis

Data analyses were done using GraphPad Prism 6 version 6.04 software (GraphPad Software, Inc., San Diego, CA). For concentration dependent curve IC50s, a curve was fitted using log (inhibitor) vs. response—variable slope (four parameters). The bottom of the curve was set to be no less than zero and the top of the curve was set to be no greater than 100. For each independent mouse experiment, the level of knockdown at each dose was normalized to the mean of the control group, which was the non-injected side of the PBS or artificial CSF groups, so that all data were expressed as a proportion of the control. In vivo data were analyzed using the Kruskal-Wallis test (one-way ANOVA) with Bonferroni corrections for multiple comparisons. Differences in all comparisons were considered significant at P-values less than 0.05.

### Cell Culture for Passive Uptake (Primary Screen and Dose Response)

Cells were plated in DMEM (Gibco) with 6% FBS (Gibco) at 10,000 cells/well in 96-well tissue culture treated plates. HsiRNA was diluted in OptiMEM (Gibco) to 2× final concentration and 50 uL diluted hsiRNA was added to 50 μL of cells for 3% FBS final. Cells were incubated for 72 hours at 37C and 5% CO₂.

### Cell Culture for Lipid-Mediated Uptake

Cells were plated in DMEM (Gibco) with 6% FBS (Gibco) at 10,000 cells/well in 96-well tissue culture treated plates. HsiRNA was diluted in OptiMEM (Gibco) to 4× final concentration. LIPOFECTAMINE RNAIMAX Transfection Reagent (Invitrogen CAT #13778150) was diluted to 4× final concentration (final=0.3 μL/25 μL/well). RNAIMAX and hsiRNA were mixed 1:1 and 50 μL was added to 50 uL of cells for 3% FBS final. Cells were incubated for 72 hours at 37C and 5% CO₂.

mRNA Quantification

mRNA was quantified using the QUANTIGENE 2.0 Assay (Affymetrix QS0011). Cells were lysed in 250 μL diluted lysis mixture, 1 part lysis mixture, 2 parts H2O, with 0.167 μg/μL proteinase K (Affymetrix QS0103) for 30 minutes at 55 C. Cell lysates were mixed thoroughly and 40 μL (˜8000 cells) of lysate were added to capture plate along with 40 μL additional diluted lysis mixture without proteinase K. Tissues were treated similarly, using 300 μL of Homoginizing Buffer (Affymetrix) with 2 μg/μL proteinase K for a 5 mg tissue punch. Tissues were then homogenized in 96-well plate format on Qaigen TissueLyzer and 40 μL were added to capture plate. Probe sets were diluted as specified in Affymetrix protocol and 20 μL of either HTT or PPIB probes (Affymetrix: SA-50339, SA-10003) were added to each well of capture plate for final volume of 100 μL. Signal was amplified according to manufacture protocol. Luminescence was detected on either the Veritas Luminometer or the Tecan M 1000.

### Live Cell Staining and Brain Sections Immunostaining

For live cell uptake monitoring, cells were plated at a density of 2×10⁵ cells per 35 mm glass-bottom dish and grown overnight. Prior to imaging, cell organelles were stained in HBSS (Gibco) using staining reagents purchased from Life Technologies unless specified: cell nuclei, endoplasmic reticulum and lysosomes were respectively stained using the NUCBLUE Live READYPROBE, ER-TRACKER Green (Bodipy FL Glibenclamide) and LYSOTRACKER Deep Red reagents as indicated by the manufacturer. Imaging was performed in non-supplemented DMEM without phenol red (Invitrogen). Cells were treated with 0.5 μM of Cy3-labeled hsiRNA, and live cell imaging was performed over time.

### Confocal Imaging

All confocal images were acquired with a CSU10B Spinning Disk Confocal System scan head (Solamere Technology Group) mounted on a TE-200E2 inverted microscope (Nikon) with a 60× Plan/APO oil lens and a Coolsnap HQ2 camera (Roper). Images were processed using ImageJ (1.47v) software. Number of neurons without or with hsiRNA was counted using ImageJ software. Brain sections images were acquired with a z-axis spacing of 1 μm.

### Probe Validation

HTT detection probe sets were validated in neurons. Two types of probe sets (exon-exon—identical hybridization sequence (exon 27-35); and exon-exon—different hybridization sequence (exon 27-35 and exon 60-67)) were used to validate specificity. It was observed that the majority of detected signal from the probes is specific to htt-mRNA (data not shown). Two additional types of probe-sets (exon-intron—different hybridization sequence (exon 27-35 and intron 60-61); and exon-exon—difference hybridization sequence (exon 27-35 and exon 60-67)) were used to validate that the nuclear signal is not intron specific. It was observed that the intron-specific probe shows little overlap in the nuclei specific to transcription sites, and that the exon-specific probes show a higher degree of overlap.

### Example 5

### Full Metabolic Stabilization is Essential for Conjugate-Mediated siRNA Delivery In Vivo

Small interfering RNA (siRNA)-based drugs require chemical modifications/formulation to promote stability, minimize innate immunity, and enable delivery to target tissues. Partially modified siRNAs (up to 70% of bases modified) are typically used to explore the effectiveness of bioconjugates for RNAi delivery. The data disclosed herein shows that full modification (100% of bases modified) is absolutely essential for conjugate-mediated siRNA delivery systemically. Full modification dramatically improved distribution, potency and duration of affect upon local administration. Tissues, including liver and kidney, retained two orders of magnitude higher levels of fully modified hydrophobic siRNAs (FM-hsiRNA), which supports robust silencing of targets.

Screening a panel of small, asymmetric, fully modified variants based on an alternating 2′-methoxy, 2′-fluoro pattern, a scaffold was identified which was successfully applied to 100% of tested compounds without compromising silencing efficacy. Thus, fully modified, asymmetric siRNAs provided a scaffold upon which to discover new chemistries that promote siRNA delivery and expand the clinical utility of RNAi.

This example compares side-by-side the impact of fully modified versus conventionally modified siRNA scaffolds on conjugate-mediated in vivo distribution and efficacy. Hydrophobically (e.g., cholesterol) modified asymmetric siRNAs were used as an example (FIG. 29). Cholesterol conjugation to partially modified siRNAs results in robust cellular uptake in vitro (Khvorova A., S. W., Kamens J., Samarsky D., Woolf T., Cardia J. Reduced size self-delivering RNAi compounds. USA patent (2014); Lorenz, C., Hadwiger, P., John, M., Vornlocher, H. P. & Unverzagt, C. Steroid and lipid conjugates of siRNAs to enhance cellular uptake and gene silencing in liver cells. Bioorg Med Chem Lett 14, 4975-4977, doi:10.1016/j.bmcl.2004.07.018S0960-894X(04)00908-4 [pii] (2004)) and potent silencing locally in vivo (Byrne, M. et al. Novel hydrophobically modified asymmetric RNAi compounds (sd-rxRNA) demonstrate robust efficacy in the eye. Journal of ocular pharmacology and therapeutics: the official journal of the Association for Ocular Pharmacology and Therapeutics 29, 855-864, doi:10.1089/jop.2013.0148 (2013)), but only marginal systemic efficacy (Soutschek, J. et al. Therapeutic silencing of an endogenous gene by systemic administration of modified siRNAs. Nature 432, 173-178, doi:10.1038/nature03121 (2004)), thus representing a good model for evaluation of the potential impact role of complete siRNA modification on delivery.

Non-modified RNA degrades quickly by combination of endonucleases and exonucleases; thus both internal and terminal modifications are necessary for stability. Complete chemical modification of siRNAs can interfere with RNA Induced Silencing Complex (RISC) interactions, but several configurations have been reported to have activity compatible with naked compounds, although in a context of only limited number of sequences (Deleavey, G. F. et al. The 5′ binding MID domain of human Argonaute2 tolerates chemically modified nucleotide analogues. Nucleic acid therapeutics 23, 81-87, doi:10.1089/nat.2012.0393 (2013); Stokman, G., Qin, Y., Racz, Z., Hamar, P. & Price, L. S. Application of siRNA in targeting protein expression in kidney disease. Advanced drug delivery reviews 62, 1378-1389, doi:10.1016/j.addr.2010.07.005 (2010)).

Inability to cleave the sense strand is one of the limiting factors for fully chemically modified siRNA RISC entry. Use of an asymmetric scaffold (15 bases in the sense strand, 20 bases in the guide strand) lowered the duplex Tm, and thus cased the sense dissociation required for efficient RISC loading. In addition, the presence of a single-stranded, fully phosphorothioated tail enhanced conjugated mediated cellular internalization of these type of compounds by a mechanism similar to conventional antisense compounds.

The efficacy of a panel of fully modified hsiRNA variants was compared based on patterns reported by the Dagma and Bhat laboratories. The initial screen was performed in the context of a huntingtin-targeting hsiRNA identified recently (Alterman et al., 2015, Molecular Therapy, under review). It was demonstrated that an alternating 2′-fluoro, 2′-methoxy pattern, starting with chemically phosphorylated 2′-methoxy-modified U in the 5′ position of the guide strand performed the best, although several other configurations were nearly as functional (FIG. 29). It was determined to be important to start the modification pattern with the chemically phosphorylated 2′-methoxy in position one of the antisense strand. Starting the same pattern with 2′-fluoro was shown to have a detrimental impact on efficacy, at least in some of the sequences. Without intending to be bound by scientific theory, this was likely related to placement of 2′-methoxy in positions 2 and 14 of the guide strand, which are not well tolerated in the context of heavily modified duplexes. Chemical phosphorylation of the guide strand was also determined to be essential as terminal 2′-methoxy U was not a good substrate for intracellular kinases. In addition, terminal phosphorothioates were added on both the 3′ and 5′ ends of the oligonucleotide to provide additional exonuclease resistance. FIG. 29 shows the structure and PyMOL model of the most optimal configuration compared to a conventionally modified hsiRNA.

This chemical modification pattern was applied to several previously identified functional hsiRNA sequences, and demonstrated similar or improved efficacy (FIGS. 29C-29D). Interestingly, the most profound improvement effect was observed in primary trophoblasts in suspension, where cholesterol-mediated uptake took longer and, thus, relative impact of additional stabilization was more significant. In addition, this chemical modification pattern was applied to several published sequences targeting Tie-2 and Sod1, with similar success.

To generalize that these phenomena were relevant to other conjugates, the efficacy of partially and fully modified GalNac conjugated siRNAs was investigated in primary hepatocytes. It was demonstrated that similarly, fully metabolically stabilized compounds were significantly more active in hepatocytes than partially modified compounds.

Generally, the introduction of chemical modifications often has negative impacts on siRNA efficacy, with naked, hyper-functional siRNA losing efficacy in the context of extensive chemical modification patterns. The A-form RNA helix necessary for efficient recognition by the RISC complex is favored by C3′-endo ribose confirmation, preferentially adopted by 2′-fluoro and 2′-methoxy modifications. Alternating modifications modulates thermodynamic stability essential for efficient RISC entry, which has been studied in detail for 2′-FANA and 2′-fluro hybrids previously.

Partial siRNA modification (all pyrimidines) resulted in a dramatic enhancement of stability in vitro (increased from minutes to days in up to 50% FBS or human serum, which is similar to the enhancement in stability reported for fully modified siRNAs. In vivo, oligonucleotides distribute through the tissues and are constantly exposed to an aggressive nuclease environment, which conditions are impossible to mimic in vitro. Thus, stability of these modification patterns might quite different.

To evaluate the impact of complete modification on hydrophobic siRNAs efficacy and distribution in vivo, 10 mg/kg of partially modified and fully modified Cy 3-labelled hsiRNAs were administered to mice intravenously (IV) and subcutaneously (SC) (FIG. 30). Twenty four hours later, tissue distribution was evaluated by fluorescent microscopy, and levels of guide strand tissue accumulation were measured quantitatively using HPLC-based separation of the PNA-based assay. The PNA assay was a simple high throughput assay enabling quantitative evaluation of oligonucleotide retention in the tissues, and was adopted from the one described and used in Axon labs for clinical samples evaluation.

First, a dramatic enhancement of fluorescence retention and distribution to main organs was observed with fully metabolically stabilized compounds (FIG. 30A). While levels of fluorescence after a single injection of partially modified hsiRNAs was marginal and mainly limited to liver and kidney, full metabolic stabilization resulted in dramatic oligonucleotide accumulation in liver, kidney and spleen, as well as efficient distribution to other tissues, including fat and skin. The levels of accumulation by IV and SC administration were compared, and they were found to be similar (FIGS. 30B and 30C). Interestingly, the FM-hsiRNAs preferentially accumulated in endothelial cells and macrophages, and only secondarily accumulated in hepatocytes. Thus, hydrophobically modified compound differed in distribution from GalNac, which preferentially delivered to liver hepatocytes.

When measured quantitatively, the results were even more striking. While with partially modified compound had minimal levels of intact guide strand detected in tissues at 24 hours, fully modified compounds accumulated to levels of approximately 200 ng/mg in liver, kidney and spleen (FIG. 30B, 30C) and at ng/mg levels in several other tissues. Strikingly, with FM-hsiRNA, a similar level of intact guide strand was detected five days after administration, accounting for close to 80% of the injected dose (FIG. 30). This indicated that fully modified compounds were not only delivered to tissues, but retained in the tissues for extended periods of time.

These data are consistent with previously published attempts to use hydrophobic modifications (cholesterol, fatty acids, etc.) for systemic delivery of partially modified siRNAs, where repetitive dosing of high concentrations of compounds, i.e., 50-80 mg/kg, was necessary to detect any silencing activity.

As the levels of liver and kidney accumulation exceeded levels that are generally necessary to induce silencing (usually above 10-20 ng/mg), it was confirmed whether observed robust delivery resulted in functional silencing. Hydrophobically modified compounds preferentially delivered to endothelia, thus soluble isoforms of FLT1 (VEGFR1), which is preferentially expressed in endothelia, was targeted using recently identified hsiRNA compounds targeting sFLT1 (Turanov et al, 2015, prepared for publication).

Compounds were administered to two different mouse strains. In both cases, systemic administration of FM-hsiRNA induced robust silencing of sFTL1 in liver and kidney measured five days after administration. In a first study, the silencing was compared to PBS injected animals. In a second study to control for potential chemistry-related effects, both PBS and non-targeting control (NTC) of the same chemical composition was used. Only sFLT1 targeting FM-hsRNA, not the NTC, lowered the sFLT1 expression.

All oligonucleotides distributed preferentially to the liver, thus demonstrating liver efficacy as was expected. In addition, similar levels of compounds accumulated in the kidney, resulting in productive silencing. There is minimal data in the art on kidney siRNA delivery (Stokman, G., Qin, Y., Racz, Z., Hamar, P. & Price, L. S. Application of siRNA in targeting protein expression in kidney disease. Advanced drug delivery reviews 62, 1378-1389, doi:10.1016/j.addr.2010.07.005 (2010)). Partially modified (alternating 2′-methoxys) were studied for kidney proximal tubule delivery. A majority was cleared after four hours, and not detectable after 24 hours (Molitoris, B. A. et al. siRNA Targeted to p53 Attenuates Ischemic and Cisplatin-Induced Acute Kidney Injury. Journal of the American Society of Nephrology: JASN 20, 1754-1764, doi:10.1681/ASN.2008111204 (2009)).

Thus, full modification of siRNA was determined to be absolutely essential for systemic delivery and efficacy in vivo. In spite of the decade of effort in medicinal chemistry, the liver is currently the only target of non-formulated miRNAs having in vivo efficacy. Without intending to be bound by scientific theory, data presented herein might provide a partial explanation for this fact. As a vast majority of attempts to explore different conjugates (e.g., peptides, antibodies, small molecules, hydrophobic modifications, etc.) were performed in the context of partially modified siRNAs, the lack of in vivo stabilization might have been one of the major factors contributing to lack of robust efficacy; limiting the time available for the conjugates to promote uptake. It is also possible that the siRNAs described in the art that exhibited no or limited efficacy could have dramatically enhanced efficacy using the scaffolds as described herein.

While it is clear that partially modified hydrophobic siRNAs are not active systemically, they can induce robust gene silencing in vivo upon local administration to tissues such as eye (Byrne (Supra)), skin (Khvorova (Supra)) and brain (Alterman et al, Molecular Therapy, under review). To evaluate the impact of full modification on siRNA efficacy upon local administration, conventional and fully modified siRNAs were injected intraventricularly into cerebrospinal fluid (CSF). Similarly to systemic administration, the use of fully modified hsiRNAs upon CSF infusion dramatically enhanced both the levels of oligonucleotide retention in a tissue, as well as the degree of distribution throughout the brain (FIG. 31A). With CY3 labelled FM-hsiRNA, compounds distributed to the cortex, striatum, cerebellum and other brain tissues (FIGS. 31 B-D), and a dramatic amount was retained around the injected ventricle. With conventional, partially modified hsiRNA, small amounts were detected around the ventricle and immediately adjusted tissues, but overall distribution was limited.

To test the potential impact of full stabilization on in vivo efficacy upon local administration, dose response and duration of effect of two types of compounds administrated directly (intrastriatally) in the brain were investigated.

When injected into the striatum, partially modified hsiRNA induced potent silencing (FIG. 31) at dose levels of 10 μg and above. Silencing disappeared at 6 μg and 3 μg. With fully modified hsiRNAs, similar levels of silencing were observed at all doses tested (FIG. 31F), indicating that fully modified compounds were more potent in vivo upon local administration.

When compared the duration of effect, the difference was even more striking. While partially modified induced silencing disappeared at two weeks, fully modified compounds continued to silence HTT gene for one, two and four weeks (FIG. 31G), with levels of modulation that appeared unaffected. Thus, locally, full chemical modification resulted in a dramatic induction of both potency and, more importantly, of duration of effect.

In summary, described herein is an siRNA scaffold having an alternating 2′-fluro, 2′-methoxy modification pattern applied to an asymmetric structural frame. As described herein, this scaffold can be successfully applied to a wide range of previously identified functional siRNAs compounds. The chemical modification pattern did not interfere with RISC entry, and it resulted in fully chemically modified compounds which were recognized by RISC complex as effectively as native RNAs. The exact impact of this chemistry on RISC cleavage kinetics will be investigated using single molecule approaches. Without intending to be bound by scientific theory, it is likely that use of a shorter (e.g., 15 bases) sense strand may case dissociation of the uncleavable sense strand required for RISC loading, thus alleviating one of the limiting steps in RISC loading of fully stabilized compounds.

When administrated systemically, fully modified hsiRNAs accumulated in a vast range of tissues, including the liver, kidneys, spleen, fat, skin, etc., with confirmed silencing in the liver and kidneys. Robust efficacy in kidneys as demonstrated herein is a first example of conjugate-mediated delivery to this highly clinically relevant organ, opening options for development of novel therapies for kidney related diseases and disorders. In addition, full modification dramatically enhanced potency and, most importantly, enhanced the duration of effect of hydrophobically modified siRNAs in vivo upon local administration in the brain. Single injections resulted in silencing lasting for at least a month and likely longer, indicating that this chemistry provides a promise of long-term silencing upon single administration. It was surprising that with partially modified compounds, silencing lasted only for one week. In general, a loaded RISC complex is long lived, especially in non-dividing cells like neurons. Indeed, in primary neuronal cultures, a single treatment induced silencing for at least three weeks. The data described herein indicates that half-life of loaded RISC complex (at least for the sequences tested) in vivo in the brain is shorter than originally expected.

These data demonstrate that full chemical modification (i.e., additional stabilization) is a major contributor to in vivo efficacy of conjugate-delivered siRNAs. The data provided herein shows that simple, fully modified asymmetric siRNA scaffolds can be used for screening vast number of conjugation modalities, hopefully resulting in major advances to expand clinical utilization of RNAi beyond the liver.

### INCORPORATION BY REFERENCE

The contents of all cited references (including literature references, patents, patent applications, and websites) that maybe cited throughout this application are hereby expressly incorporated by reference in their entirety for any purpose, as are the references cited therein. The disclosure will employ, unless otherwise indicated, conventional techniques of immunology, molecular biology and cell biology, which are well known in the art.

The present disclosure also incorporates by reference in their entirety techniques well known in the field of molecular biology and drug delivery. These techniques include, but are not limited to, techniques described in the following publications:
Atwell et al. J. Mol. Biol. 1997, 270:26-35;
Ausubel et al. (eds.), CURRENT PROTOCOLS IN MOLECULAR BIOLOGY, John Wiley &Sons, NY (1993);
Ausubel, F. M. et al. eds., SHORT PROTOCOLS IN MOLECULAR BIOLOGY (4th Ed. 1999) John Wiley & Sons, NY. (ISBN 0-471-32938-X);
CONTROLLED DRUG BIOAVAILABILITY, DRUG PRODUCT DESIGN AND PERFORMANCE, Smolen and Ball (eds.), Wiley, New York (1984);
Giege, R. and Ducruix, A. Barrett, CRYSTALLIZATION OF NUCLEIC ACIDS AND PROTEINS, a Practical Approach, 2nd ea., pp. 20 1-16, Oxford University Press, New York, New York, (1999);
Goodson, in MEDICAL APPLICATIONS OF CONTROLLED RELEASE, vol. 2, pp. 115-138 (1984);
Hammerling, et al., in: MONOCLONAL ANTIBODIES AND T-CELL HYBRIDOMAS 563-681 (Elsevier, N.Y., 1981;
Harlow et al., ANTIBODIES: A LABORATORY MANUAL, (Cold Spring Harbor Laboratory Press, 2nd ed. 1988);
Kabat et al., SEQUENCES OF PROTEINS OF IMMUNOLOGICAL INTEREST (National Institutes of Health, Bethesda, Md. (1987) and (1991);
Kabat, E. A., et al. (1991) SEQUENCES OF PROTEINS OF IMMUNOLOGICAL INTEREST, Fifth Edition, U.S. Department of Health and Human Services, NIH Publication No. 91-3242;
Kontermann and Dubel eds., ANTIBODY ENGINEERING (2001) Springer-Verlag. New York. 790 pp. (ISBN 3-540-41354-5).
Kriegler, Gene Transfer and Expression, A Laboratory Manual, Stockton Press, NY (1990);
Lu and Weiner eds., CLONING AND EXPRESSION VECTORS FOR GENE FUNCTION ANALYSIS (2001) BioTechniques Press. Westborough, MA. 298 pp. (ISBN 1-881299-21-X).
MEDICAL APPLICATIONS OF CONTROLLED RELEASE, Langer and Wise (eds.), CRC Pres., Boca Raton, Fla. (1974);
Old, R. W. & S. B. Primrose, PRINCIPLES OF GENE MANIPULATION: AN INTRODUCTION TO GENETIC ENGINEERING (3d Ed. 1985) Blackwell Scientific Publications, Boston. Studies in Microbiology; V.2:409 pp. (ISBN 0-632-01318-4).
Sambrook, J. et al. eds., MOLECULAR CLONING: A LABORATORY MANUAL (2d Ed. 1989) Cold Spring Harbor Laboratory Press, NY. Vols. 1-3. (ISBN 0-87969-309-6).
SUSTAINED AND CONTROLLED RELEASE DRUG DELIVERY SYSTEMS, J. R. Robinson, ed., Marcel Dekker, Inc., New York, 1978
Winnacker, E .L. FROM GENES TO CLONES: INTRODUCTION TO GENE TECHNOLOGY (1987) VCH Publishers, NY (translated by Horst Ibelgaufts). 634 pp. (ISBN 0-89573-614-4).

1-30. (cancel)

31. A method of treating or managing a disease or a disorder comprising administering to a subject in need of such treatment or management a therapeutically effective amount of a pharmaceutical composition, wherein the pharmaceutical composition comprises one or more double-stranded nucleic acids (dsNAs) and a pharmaceutically acceptable carrier, wherein the one or more dsNAs comprises a first oligonucleotide and a second oligonucleotide, each with a 5′ end and a 3′ end, wherein:
the first oligonucleotide comprises at least 16 contiguous nucleotides and complementarity to a target;
the nucleotides at positions 2 and 14 from the 5′ end of the first oligonucleotide are not 2′-methoxy-ribonucleotides;
nucleotides at positions 1-6 from the 3′ end, or positions 1-7 from the 3′ end, of the first oligonucleotide are connected to adjacent nucleotides via phosphorothioate linkages;
the nucleotides of the first oligonucleotide are connected via phosphodiester or phosphorothioate linkages:
the nucleotides of the second oligonucleotide are connected via phosphodiester or phosphorothioate linkages;
the nucleotides at positions 2 and 14 from the 3′ end of the second oligonucleotide are 2′-methoxy-ribonucleotides;
the first oligonucleotide and the second oligonucleotide comprise alternating 2′-methoxy-ribonucleotides and 2′-fluoro-ribonucleotides; and
a portion of the first oligonucleotide is complementary to a portion of the second oligonucleotide.

32. (canceled)

33. The method of claim 31, wherein the first oligonucleotide has perfect complementarity to the target.

34. The method of claim 31, wherein the target is mammalian or viral mRNA.

35. The method of claim 34, wherein the target is an intronic region of the mRNA.

36. The method of claim 31, wherein the second oligonucleotide is linked to a hydrophobic molecule at the 3′ end of the second oligonucleotide.

37. The method of claim 36, wherein the linkage between the second oligonucleotide and the hydrophobic molecule comprises polyethylene glycol.

38. The method of claim 37 wherein the linkage between the second oligonucleotide and the hydrophobic molecule comprises triethylene glycol.

39. The method of claim 31, wherein the nucleotides at positions 1 and 2 from the 3′ end of the second oligonucleotide are connected to adjacent nucleotides via phosphorothioate linkages.

40. The method of claim 31, wherein the nucleotides at positions 1 and 2 from the 3′ end of second oligonucleotide, and the nucleotides at positions 1 and 2 from the 5′ end of second oligonucleotide, are connected to adjacent ribonucleotides via phosphorothioate linkages.

41. The method of claim 31, wherein the one or more dsNAs comprises a first dsNA and a second dsNA, wherein:
the first dsNA has a structure of compound (IIIa): 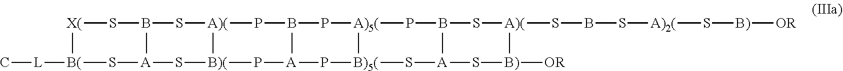
comprising: (1) a first oligonucleotide strand comprising the sequence 5′ UAAAUUUGGAGAUCCGAGAG 3′ (SEQ ID NO: 4) and (2) a second oligonucleotide strand comprising the sequence 3′ AUUUAAACCUCUAGG 5′ (SEQ ID NO: 5), wherein X is 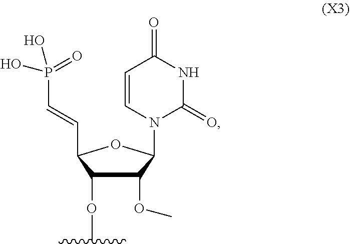
C is cholesterol, and each | represents a hydrogen bonding interaction; and
the second dsNA has a structure of compound (IIIa): 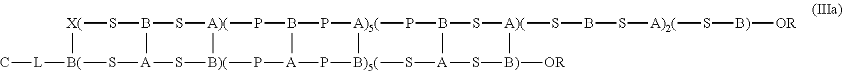
comprising: (1) a third oligonucleotide strand comprising the sequence 5′ UAUAAAUGGUAGCUAUGAUG 3′ (SEQ ID NO: 6) and (2) a fourth oligonucleotide strand comprising the sequence 3′ AUAUUUACCAUCGAU 5′ (SEQ ID NO: 7), wherein X is 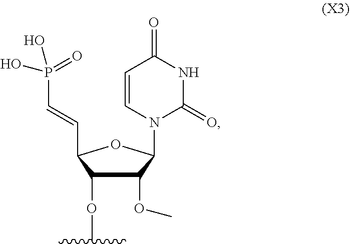
C is cholesterol, and each | represents a hydrogen bonding interaction; wherein:
A, for each occurrence, independently is a 2′-methoxy-ribonucleotide;
B, for each occurrence, independently is a 2′-fluoro-ribonucleotide;
L is a linker comprising an ethylene glycol chain, an alkyl chain, a peptide, RNA, DNA, a phosphodiester, a phosphorothioate, a phosphoramidate, an amide, a carbamate, or a combination thereof;
S is a phosphorothioate linker;
P is a phosphodiester linker; and
R, for each occurrence, independently is hydrogen or a capping group.

42. The method of claim 31, wherein the disease or the disorder comprises a liver disease or disorder, a kidney disease or disorder, a placental disease or disorder, a pregnancy-related disease or disorder, and a disorder associated with the expression of soluble Flt1 protein, or a combination thereof.

43. The method of claim 31, wherein the disease or the disorder comprises preeclampsia, post-partum preeclampsia, eclampsia, or HELLP syndrome.

44. A method of treating or managing a disease or disorder comprising administering to a subject in need of such treatment or management a therapeutically effective amount of a pharmaceutical composition, wherein the pharmaceutical composition comprises one or more double-stranded nucleic acids (dsNAs) and a pharmaceutically acceptable carrier, wherein the one or more dsNAs comprises a first oligonucleotide and a second oligonucleotide, wherein:
(1) the first oligonucleotide has a structure of compound (Ib):
X-A(-S-B-S-A)ₘ(-P-B-P-A)ₙ(-P-B-S-A)_{q}(-S-B-S-A)ᵣ(-S-B)ₜ-OR (Ib)
wherein:
X is selected from the group consisting of: 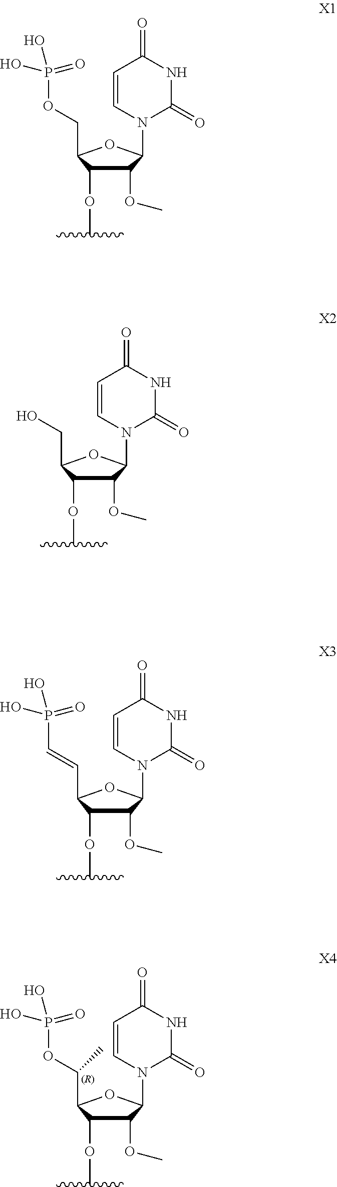 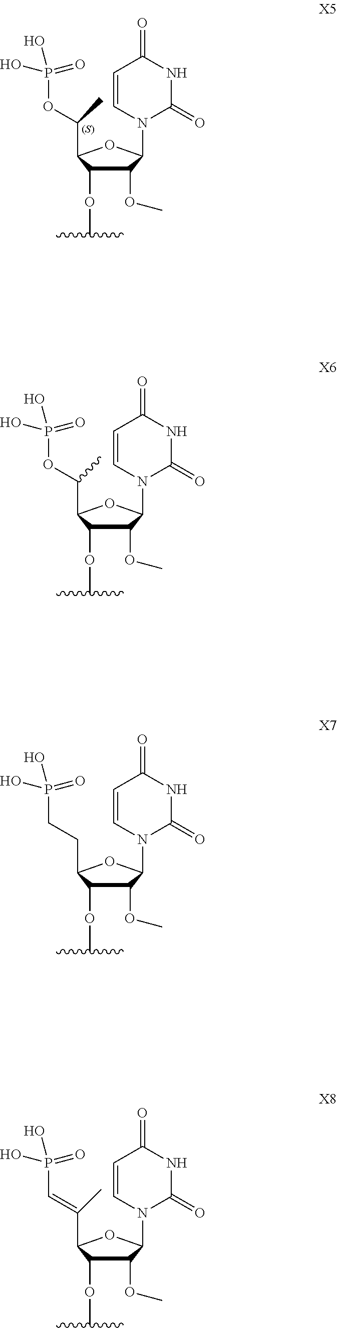
A, for each occurrence, independently is a 2′-methoxy-ribonucleotide;
B, for each occurrence, independently is a 2′-fluoro-ribonucleotide;
S is a phosphorothioate linker;
R, for each occurrence, independently is selected from hydrogen and a capping group;
P is a phosphodiester linker;
m is 0 or 1;
n is 4, 5 or 6;
q is 0 or 1;
r is 2 or 3; and
t is 0 or 1;
(2) a portion of the first oligonucleotide is complementary to a portion of the second oligonucleotide; and
(3) the second oligonucleotide has a structure of compound (IIa):
C-L-B(-S-A-S-B)_{m′}(-P-A-P-B)_{n′}(-P-A-S-B)_{q′}(-S-A)_{r′}(-S-B)_{t′}-OR (IIa)
wherein:
C is a hydrophobic molecule;
A, for each occurrence, independently is a 2′-methoxy-ribonucleotide;
B, for each occurrence, independently is a 2′-fluoro-ribonucleotide;
L is a linker comprising an ethylene glycol chain, an alkyl chain, a peptide, RNA, DNA, a phosphodiester, a phosphorothioate, a phosphoramidate, an amide, a carbamate, or a combination thereof;
S is a phosphorothioate linker;
P is a phosphodiester linker;
R, for each occurrence, independently is hydrogen or a capping group;
m′ is 0 or 1;
n′ is 4, 5 or 6;
q′ is 0 or 1;
r′ is 0 or 1; and
t′ is 0 or 1.

45. The method of claim 44, wherein the second oligonucleotide is linked to a hydrophobic molecule at the 3′ end of the second oligonucleotide, optionally wherein the hydrophobic molecule is cholesterol.

46. The method of claim 44, wherein the first oligonucleotide has 3-7 more ribonucleotides than the second oligonucleotide.

47. The method of claim 46, wherein:
the first oligonucleotide has a structure:
X(-S-B-S-A)(-P-B-P-A)₅(-P-B-S-A)(-S-B-S-A)₂(-S-B)-OR;
the second oligonucleotide has a structure:
C-L-B(-S-A-S-B)(-P-A-P-B)₅(-S-A)(-S-B)-OR; and
the one or more dsNAs each separately has a structure of compound (IIIa): 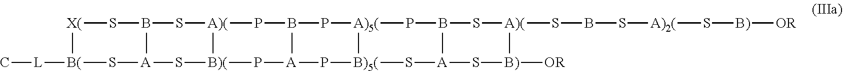
wherein each | represents a hydrogen bonding interaction.

48. The method of claim 47, wherein:
the first oligonucleotide comprises a sequence 5′ UAAAUUUGGAGAUCCGAGAG 3′ (SEQ ID NO: 4);
the second oligonucleotide comprises a sequence 3′ AUUUAAACCUCUAGG 5′ (SEQ ID NO: 5);
X is X3; and
C is cholesterol.

49. The method of claim 47, wherein:
the first oligonucleotide comprises a sequence 5′ UAUAAAUGGUAGCUAUGAUG 3′ (SEQ ID NO: 6);
the second oligonucleotide comprises a sequence 3′ AUAUUUACCAUCGAU 5′ (SEQ ID NO: 7);
X is X3; and
C is cholesterol.

50. The method of claim 47, wherein:
the first oligonucleotide comprises a sequence
(SEQ ID NO: 8)
5′ UUAAUCUCUUUACUGAUAUA 3′;
the second oligonucleotide comprises a sequence 3′ AAUUAGAGAAAUGAC 5′ (SEQ ID NO: 9);
X is X3; and
C is cholesterol.

51. The method of claim 44, wherein:
the first oligonucleotide has a structure:
X(-P-B-P-A)₆(-P-B-S-A)(-S-B-S-A)₂(-S-B)-OR;
the second oligonucleotide has the structure:
C-L-B(-S-A-S-B)(-P-A-P-B)₆-OR; and
the one or more dsNAs each separately has a structure of compound (IIIb): 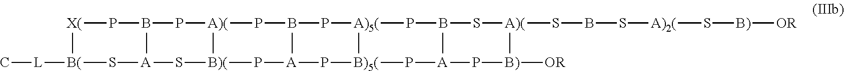
wherein each | represents a hydrogen bonding interaction.

52. The method of claim 51, wherein:
the first oligonucleotide comprises a sequence
(SEQ ID NO: 4)
5′ UAAAUUUGGAGAUCCGAGAG 3′;
the second oligonucleotide comprises a sequence 3′ AUUUAAACCUCUAGG 5′ (SEQ ID NO: 5);
X is X3; and
C is cholesterol.

53. The method of claim 51, wherein:
the first oligonucleotide comprises a sequence
(SEQ ID NO: 6)
5′ UAUAAAUGGUAGCUAUGAUG 3′;
the second oligonucleotide comprises a sequence 3′ AUAUUUACCAUCGAU 5′ (SEQ ID NO: 7);
X is X3; and
C is cholesterol.

54. The method of claim 51, wherein:
the first oligonucleotide comprises a sequence
(SEQ ID NO: 8)
5′ UUAAUCUCUUUACUGAUAUA 3′;
the second oligonucleotide comprises a sequence 3′ AAUUAGAGAAAUGAC 5′ (SEQ ID NO: 9);
X is X3; and
C is cholesterol.

55. The method of claim 44, wherein:
the first oligonucleotide has a structure:
X(-S-B-S-A)(-P-B-P-A)₅(-S-B-S-A)₃(-S-B)-OR;
the second oligonucleotide has a structure:
C-L-B(-S-A-S-B)(-P-A-P-B)₅(-S-A-S-B)-OR; and
the one or more dsNAs each separately has a structure of compound (IIIc): 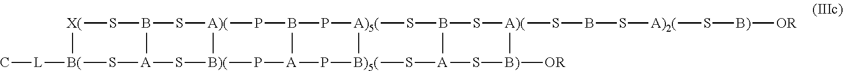
wherein each | represents a hydrogen bonding interaction.

56. The method of claim 55, wherein:
the first oligonucleotide comprises a sequence
5′ UUAAUCUCUUUACUGAUAUA 3′ (SEQ ID NO: 8);
the second oligonucleotide comprises a sequence 3′ AAUUAGAGAAAUGAC 5′ (SEQ ID NO: 9);
X is X3; and
C is cholesterol.

57. The method of claim 44, wherein the one or more dsNAs comprises a first dsNA and a second dsNA, wherein:
the first dsNA has a structure of compound (IIIa): 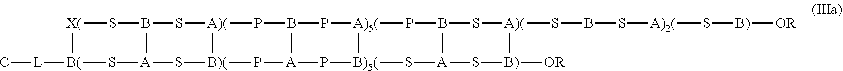
comprising: (1) a first oligonucleotide strand comprising the sequence 5′ UAAAUUUGGAGAUCCGAGAG 3′ (SEQ ID NO: 4) and (2) a second oligonucleotide strand comprising the sequence 3′ AUUUAAACCUCUAGG 5′ (SEQ ID NO: 5), wherein X is X3, C is cholesterol, and each | represents a hydrogen bonding interaction; and
the second dsNA has a structure of compound (IIIa): 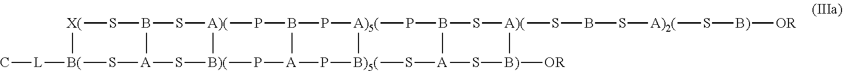
comprising: (1) a third oligonucleotide strand comprising the sequence 5′ UAUAAAUGGUAGCUAUGAUG 3′ (SEQ ID NO: 6) and (2) a fourth oligonucleotide strand comprising the sequence 3′ AUAUUUACCAUCGAU 5′ (SEQ ID NO: 7), wherein X is X3; C is cholesterol, and each | represents a hydrogen bonding interaction.

58. The method of claim 44, wherein the disease or the disorder comprises a liver disease or disorder, a kidney disease or disorder, a placental disease or disorder, a pregnancy-related disease or disorder, and a disorder associated with expression of a soluble Flt1 protein, or a combination thereof.

59. The method of claim 44, wherein the disease or the disorder comprises preeclampsia, post-partum preeclampsia, eclampsia, or HELLP syndrome.
